SEQUENCE LISTING (187 sequences)

SEQ ID NO: 1 - DNA, 1869 bp, Arabidopsis thaliana
atggctactttcaatgttctttgtagcaatcgatttagattcaatggaggttattctccggagaaatttaatagaaaggtgtcttcgaggagtagtgagcttaatgtttgtgtctctagaatcaggactcaatcgtttagttgcaggagacttggtgggtttatggaaattggtgaaaccagattaggggttattagagttcatggcgattctcgtaatagattttcttgtaatagcgagattaagcgacttgtgactggtgattacggagataaggagactcgtattggtgaaaatggtagaaacaaagggaagagaagaaggttttctcttaggcttcgacctagattacgattagtgagaatgagacttggcagatttgatttcagagcatcaatggaagattttcgatattttttgaaaaagaatctcaaaagagtgatcttgtccaccggtgtggctttaatctttggactgtgttatttgttcttgaggttaactgctgttccgtccccatccattgttccttactcagatttcgttacgaatcttcgaggtggttctgtttcaaaggttttgctcgaagaaggctcacgtcgaatctattacaacactgatgagaatgttgaggttgttgatgatgttcacaagtctgaaactttagaagatccggcaatccaaattgatggcggcacagtgacagaagctgttacgaaagacgatactccgcgaaaggttcgagctttgcctcctgtgtggaagtatgtgacaagaaaagtcgatcatgatgagaagtttcttcttagtttaatgagagagaaaggcattacttatagttcagctcctcaatccgcattgatgtcaatgagaactactttgataacaatcatctctctgtggattcctttgacacctcttatgtggcttctttatcggcaactctcagcatctaacagccctgcgaagaaacgacggtctaaaaatcccactgttggcttcgatgatgttgagggtgttgattctgctaaagacgagctcgtggagatagtctcatgtcttcaaggaagtataaactacaaaaaactaggagcaagattaccgagaggtgtgcttttggtcggtccaccagggaccggtaaaaccttgttggctcgggctgtagctggagaagcgggagttccatttttctctgtctccgctagtgagtttgttgaattgtttgttggaagaggcgcagcgcgaatcagagatcttttcaatgcagcaaggaaaaactcaccttccattatattcattgatgagcttgatgcggttggaggaaaacgcggtaggagtttcaacgatgaacgtgaccagacgttaaaccagttgcttactgagatggatggattcgagtcagacacgaaagtcattgtaattgcagcaactaaccgaccagaagcgttagactcggctctttgtcggcctggaaggttctctagaaaagttttggttgcagaaccagaccaagaaggccgtcggaaaatcttggccatacatctgagagacgttcctttagaagaagatgcatttctcatatgtgatctggttgcttctttaactcccggatttgtaggtgctgatctcgccaatattgtcaatgaagctgcattgcttgctgctcgtagaggcggagaagcggtggcacgggaagatataatggaagctatagagagagcgaagttcgggatcaatgataaggaagcgaggccaaggacgttaggaaatgaactcagcaagatgttcccatggatgccatctttggctagaagaaacggaccagatcaagacggtttacaaggaccgttaggttatcaaactctcagctaa

SEQ ID NO: 2 - protein, 622 aa, Arabidopsis thaliana
MATFNVLCSNRFRFNGGYSPEKFNRKVSSRSSELNVCVSRIRTQSFSCRRLGGFMEIGETRLGVIRVHGDSRNRFSCNSEIKRLVTGDYGDKETRIGENGRNKGKRRRFSLRLRPRLRLVRMRLGRFDFRASMEDFRYFLKKNLKRVILSTGVALIFGLCYLFLRLTAVPSPSIVPYSDFVTNLRGGSVSKVLLEEGSRRIYYNTDENVEVVDDVHKSETLEDPAIQIDGGTVTEAVTKDDTPRKVRALPPVWKYVTRKVDHDEKFLLSLMREKGITYSSAPQSALMSMRTTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESDTKVIVIAATNRPEALDSALCRPGRFSRKVLVAEPDQEGRRKILAIHLRDVPLEEDAFLICDLVASLTPGFVGADLANIVNEAALLAARRGGEAVAREDIMEAIERAKFGINDKEARPRTLGNELSKMFPWMPSLARRNGPDQDGLQGPLGYQTLS

SEQ ID NO: 3 - DNA, 1944 bp, Populus tremula
atggcttctttttctgctgtttgcaataacgggttcttgaattttaaactgaactctggagtcaataatggaaatactaaatctttgaagagatattcaactatttgttacagtccagttttgatcccttctttgggactttgcagtgctggcaaactccaacatttgttgctttggaggagcaagtttaggtcatcattttatgatggaaaaaccggggttcatttatcggggttttgtggtggttgtaagtctcaaaatggtttgtcttgtaagagctcatttaggccattgaggagtgaaaaaagtggagatgaggagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataaaaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatcccgtcgtatatactataacacagactctgtgggtactgaaaatactgaaaactcagaagacaaatcttcagtattaaatcttccaaatgagaatgcagctgaaacagttgcagtagaaggtgttgtgagcaaaactggtctggcatcaagggtagatgtatttaagaaattttcaaggccacgaacttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcaagaaagaggcgatcaaataatcaggcagtcacttttgatgatgtggagggtgttgatgttgctaagatagaactcatggagatagttttgtgtctgcaaggagctatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcggcaagaaaatcatctccatcaattatattcatcgatgaacttgatgcagttggggggaaacgtggtagaagtttcaatgatgaacgggatcaaacactaaaccagttgctgactgaaatggatggttttgagtcagaaatgaaagtggtggttattgcagcaactaataggccggaagctttggatccagccctttgccggcctggtcgcttttcaagaaaggtagttgttggagaaccagatgaagaaggaaggagaaagatcttggctgtacatttgagaggggttcctttagatgaagatgcagatcttatctgcaatctagttgcttcactgacaccaggctttgtaggtgctgatcttgcaaacatcattaatgaagctgctttacttgctgctcgaagaggtggagacattgtgacaagggaagatgtaatggaagcaattgaaagagcaaagtttggaatcagggacaggcaactaaggcccagtacaataagcaaagagctgggcaaattgttcccatggattccttcgttgatgggaacgaatgacacaagacaggatggtttgcaaggctctttaggttaccagaccctgagctga

SEQ ID NO: 4 - protein, 647 aa, Populus tremula
MASFSAVCNNGFLNFKLNSGVNNGNTKSLKRYSTICYSPVLIPSLGLCSAGKLQHLLLWRSKFRSSFYDGKTGVHLSGFCGGCKSQNGLSCKSSFRPLRSEKSGDEESHLRKRGNGNLRKRFSLRLKTRLRLLKIRLKGVSIRSMLNDFGMFLRRNIKRMTLYTSISVALGMCYLFLRLTALPSPKIVPYSELIASLQNGYVTNVLFEEGSRRIYYNTDSVGTENTENSEDKSSVLNLPNENAAETVAVEGVVSKTGLASRVDVFKKFSRPRTSTPEWQFSTRKVDRDEKFLLTLMRAKGTAYSSAPQSILMSIRSLLITIISLWIPLTPMMWLLYRQLSAANSPARKRRSNNQAVTFDDVEGVDVAKIELMEIVLCLQGAMNYQKLGAKLPRGVLLVGPPGTGKTLLARAVAGEAGVPFFSVSASEFVELFVGRGAARIRDLFNAARKSSPSIIFIDELDAVGGKRGRSFNDERDQTLNQLLTEMDGFESEMKVVVIAATNRPEALDPALCRPGRFSRKVVVGEPDEEGRRKILAVHLRGVPLDEDADLICNLVASLTPGFVGADLANIINEAALLAARRGGDIVTREDVMEAIERAKFGIRDRQLRPSTISKELGKLFPWIPSLMGTNDTRQDGLQGSLGYQTLS

SEQ ID NO: 5 - DNA, 1076 bp, Arabidopsis thaliana
actaacctgaagagactcctcctagcattggtttcagtctccttgtgatcaagctccctaatgacacaatcatagaccaatatatcccaatagtgaaatttataccatcaaagtataaacaggttctcaaagtaaaatcctaaatctagtaacgtcgatcgtccaaaaactgtaaccagtaatcataatcagcagaaacgtgatcgtaaaaaatccaggagaaattaaaactcacctaggtttagtcagatcaaaagattcgtaaccaaattcctcgatgggaattgttggaattcgatggaaccgaagaagataaccaaaacgaaagaaatacaacgttggatggagatcacatctgggaactcatacgcgtatattactatggtggtggtgactcaatctttgaatttttcatccgcttgaagaaagtgatgtttttcttttttccttgccggagaaaatatgagtccagccagaaaaataataattcaagtgaagccagcacgaaacgacgccgtcgagagagtattattatgattaaatggtggttatgccaacttgctaagtttggtgacaactaaaagattctattttctaaattgagcacgattttgggtaattagaggctttatcacgacgattatgaatttatgattcataaagttttaggaacaaaacaatttaattgggtatatatatatatataggcgtcaaattaattataccaattcaaaaaaatcagtaacctattttttaaaataagcccaaaatgaaagatagcccaatagctaaaaggcccatgaggccatgactcgatttataatgcccaatgcttattcatacagtttttcctgttctgtgttttgttttttatctgtcaaccaaaaaagagcttttgcgaactcttcttatcctttcaatttaacagagtcatcagcttgaagacgaaagcctttctaattccaatcttcttcacttgtatgacacgttcgtctactcttgcgcttggtcggttttcttaggatttttgcacaagaatgatcaacgcatgattcggattccaggatagagaatcc

SEQ ID NO: 6 - DNA, 2001 bp, Populus tremula
gactaaggcttttcaaaatctttgcgtatgatacccaaagattttggagggttattaatggattgaaatttattgttttttgtttgtatgtgttggactatcttttagaagtatggtcaaagagagggttataacccaccatctctaggcttagtcaagcgctaagccaaaatgcatatgggtccgacgaacttccagacccgccatatttaggctcagccaattgctgagcctagatgtttatcggtccagcgagttgtcagacccaccatctctaagctcagccaagtgttaagcccagacatgtatgggtcttatgagctgccaaacccaccatctctagcctgagccaagcgatgaccttagacatgtatgggtctggcgagctgtcacacccaacatctctgagcgaagccaagtactgagcctaaatgtgtataggtccgacgagttgtcaaacccaaaatctctaaacctagccaaacattgagcttagacgtgtatgagtctagtgagctgctagactcactatttacaggctcagctaagtgctgaacctagacacacacaggttcgacaatctatcagactcatcgtcttccaacgtctatgagtccggcaatctaccatactcattagtgccaagcatatgctttctttaactctaaaagaatctcaagctctctgatgggatcgtgggttagagatattttatcatgacgagtcagatatatttttatcctaataaaatagggatatcttatcttaatacgtaagagattttttctcttgacaagacgtagatattttgctaatacgccaaccctttttctttcttggaatgaagagagggatctcatgtgctattaaagaaagaaaaagacaaccttcaggcaggttcaattttcttctctctatttttacaatagtatatctattctaaaaagctatttttcttaaaagtataattaatctcccttttaactgatattaatttaattgttagagaatcattaaatatataaaaggaagacttttgcaggtaacaagcatgatctaacaactactctaccaataaaaaattatttagattatcaaaattaaaagattaacaatattattccaggccatcattcatcccatgggattgaaataacccatcagtaggcgctttgttttgcgttcctttccaacctagttttctgcccttcatattttttcgattagttcttgctcatcgatctttaagagtgaagaccccggtccctagaatatccggcaaaatgcagtataaaaggctcatcttatctctttcctttggcaatctgatggcttaggtttttccttgtgcatgtttgcacgtagttttatggattgaatgaatgagaacatagatcttatcactttagatattatttttccaattcatataacgttgttcataagaattggctcaataaatttaatttaaatattttttgaataaaataatatttcaatagcataaataatacattaaaatagacaagtttttttattttctcaattttaaagagttttaaaatctgagataaaactcgaaacgtttttttaatatgacagatccatagatcaatgctaatggagttgctaagccttaaatactgaaaggactcaactgatcaaattagtaatatggggactcaactatagtagaggcaccacacgtgtgattattgctatagagtgaaaaaaacccaatccacgggttcttataaacccaaaaaaagtttcttatcttcaccgctgaacaagcacaaatacagtagttgcccattcctcagtttgtttccagtttctacaaaaaccaggtaagaaccacgtcaattctttaaggtctcgacggtttatagcaggttcttatgtatgatttcttgcggaatcccaaatgacaggttcatgcttgtaatggatatttcttgattttcttggtaaagctagttgttgttgtcttgtagaggc

SEQ ID NO: 7 - DNA, 3387 bp, Artificial Sequence
  pAtFtsHi3::AtFtsHi3::HA construct
actaacctgaagagactcctcctagcattggtttcagtctccttgtgatcaagctccctaatgacacaatcatagaccaatatatcccaatagtgaaatttataccatcaaagtataaacaggttctcaaagtaaaatcctaaatctagtaacgtcgatcgtccaaaaactgtaaccagtaatcataatcagcagaaacgtgatcgtaaaaaatccaggagaaattaaaactcacctaggtttagtcagatcaaaagattcgtaaccaaattcctcgatgggaattgttggaattcgatggaaccgaagaagataaccaaaacgaaagaaatacaacgttggatggagatcacatctgggaactcatacgcgtatattactatggtggtggtgactcaatctttgaatttttcatccgcttgaagaaagtgatgtttttcttttttccttgccggagaaaatatgagtccagccagaaaaataataattcaagtgaagccagcacgaaacgacgccgtcgagagagtattattatgattaaatggtggttatgccaacttgctaagtttggtgacaactaaaagattctattttctaaattgagcacgattttgggtaattagaggctttatcacgacgattatgaatttatgattcataaagttttaggaacaaaacaatttaattgggtatatatatatatataggcgtcaaattaattataccaattcaaaaaaatcagtaacctattttttaaaataagcccaaaatgaaagatagcccaatagctaaaaggcccatgaggccatgactcgatttataatgcccaatgcttattcatacagtttttcctgttctgtgttttgttttttatctgtcaaccaaaaaagagcttttgcgaactcttcttatcctttcaatttaacagagtcatcagcttgaagacgaaagcctttctaattccaatcttcttcacttgtatgacacgttcgtctactcttgcgcttggtcggttttcttaggatttttgcacaagaatgatcaacgcatgattcggattccaggatagagaatccatggctactttcaatgttctttgtagcaatcgatttagattcaatggaggttattctccggagaaatttaatagaaaggtgtcttcgaggagtagtgagcttaatgtttgtgtctctagaatcaggactcaatcgtttagttgcaggagacttggtgggtttatggaaattggtgaaaccagattaggggttattagagttcatggcgattctcgtaatagattttcttgtaatagcgagattaagcgacttgtgactggtgattacggagataaggagactcgtattggtgaaaatggtagaaacaaagggaagagaagaaggttttctcttaggcttcgacctagattacgattagtgagaatgagacttggcagatttgatttcagagcatcaatggaagattttcgatattttttgaaaaagaatctcaaaagagtgatcttgtccaccggtgtggctttaatctttggactgtgttatttgttcttgaggttaactgctgttccgtccccatccattgttccttactcagatttcgttacgaatcttcgaggtggttctgtttcaaaggttttgctcgaagaaggctcacgtcgaatctattacaacactgatgagaatgttgaggttgttgatgatgttcacaagtctgaaactttagaagatccggcaatccaaattgatggcggcacagtgacagaagctgttacgaaagacgatactccgcgaaaggttcgagctttgcctcctgtgtggaagtatgtgacaagaaaagtcgatcatgatgagaagtttcttcttagtttaatgagagagaaaggcattacttatagttcagctcctcaatccgcattgatgtcaatgagaactactttgataacaatcatctctctgtggattcctttgacacctcttatgtggcttctttatcggcaactctcagcatctaacagccctgcgaagaaacgacggtctaaaaatcccactgttggcttcgatgatgttgagggtgttgattctgctaaagacgagctcgtggaggtaaaaagatacgtaaatgcaacgtttatagaattaagtttgaagtccaacctccatgtgttctgtttttctttgtagatagtctcatgtcttcaaggaagtataaactacaaaaaactaggagcaagattaccgagaggtgtgcttttggtcggtccaccagggaccggtaaaaccttgttggctcgggctgtagctggagaagcgggagttccatttttctctgtctccgctagtgagtttgttgaattgtttgttggaagaggcgcagcgcgaatcagagatcttttcaatgcagcaaggaaaaactcaccttccattatattcattgatgagcttgatgcggttggaggaaaacgcggtaggagtttcaacgatgaacgtgaccagacgttaaaccaggtaaagaatcaacatttagtacccttttaaccatgtagactccattgctgatatacttcctttgtagttgcttactgagatggatggattcgagtcagacacgaaagtcattgtaattgcagcaactaaccgaccagaagcgttagactcggctctttgtcggcctggaaggttctctagaaaagttttggttgcagaaccagaccaagaaggccgtcggaaaatcttggccatacatctgagagacgttcctttagaagaagatgcatttctcatatgtgatctggttgcttctttaactcccggatttgtaggtgctgatctcgccaatattgtcaatgaagctgcattgcttgctgctcgtagaggtcagtctttaagtgatcttttaacagccctttcttgatttaagtagttattcgatctggttgcttcccaggtttgatcttttgggatgaagctgcggtttttggcttaacagacactagcgatttcttgattgttttcaggcggagaagcggtggcacgggaagatataatggaagctatagagagagcgaagttcgggatcaatgataaggaagcgaggccaaggacgttaggaaatgaactcagcaagatgttcccatggatgccatctttggctagaagaaacggaccagatcaagacggtttacaaggaccgttaggttatcaaactctcagcaagggtgggcgcgccgacccagctttcttgtacaaagtggttgataacagcgggttaattaacatcttttacccatacgatgttcctgactatgcgggctatccgtatgacgtcccggactatgcaggatcctatccatatgacgttccagattacgct

SEQ ID NO: 8 - DNA, 3827 bp, Artificial Sequence
  35S::AtFtsHi3::GFP construct
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaatcaaacaagtttgtacaaaaaagcaggctccgcggccgcccccttcaccatggctactttcaatgttctttgtagcaatcgatttagattcaatggaggttattctccggagaaatttaatagaaaggtgtcttcgaggagtagtgagcttaatgtttgtgtctctagaatcaggactcaatcgtttagttgcaggagacttggtgggtttatggaaattggtgaaaccagattaggggttattagagttcatggcgattctcgtaatagattttcttgtaatagcgagattaagcgacttgtgactggtgattacggagataaggagactcgtattggtgaaaatggtagaaacaaagggaagagaagaaggttttctcttaggcttcgacctagattacgattagtgagaatgagacttggcagatttgatttcagagcatcaatggaagattttcgatattttttgaaaaagaatctcaaaagagtgatcttgtccaccggtgtggctttaatctttggactgtgttatttgttcttgaggttaactgctgttccgtccccatccattgttccttactcagatttcgttacgaatcttcgaggtggttctgtttcaaaggttttgctcgaagaaggctcacgtcgaatctattacaacactgatgagaatgttgaggttgttgatgatgttcacaagtctgaaactttagaagatccggcaatccaaattgatggcggcacagtgacagaagctgttacgaaagacgatactccgcgaaaggttcgagctttgcctcctgtgtggaagtatgtgacaagaaaagtcgatcatgatgagaagtttcttcttagtttaatgagagagaaaggcattacttatagttcagctcctcaatccgcattgatgtcaatgagaactactttgataacaatcatctctctgtggattcctttgacacctcttatgtggcttctttatcggcaactctcagcatctaacagccctgcgaagaaacgacggtctaaaaatcccactgttggcttcgatgatgttgagggtgttgattctgctaaagacgagctcgtggaggtaaaaagatacgtaaatgcaacgtttatagaattaagtttgaagtccaacctccatgtgttctgtttttctttgtagatagtctcatgtcttcaaggaagtataaactacaaaaaactaggagcaagattaccgagaggtgtgcttttggtcggtccaccagggaccggtaaaaccttgttggctcgggctgtagctggagaagcgggagttccatttttctctgtctccgctagtgagtttgttgaattgtttgttggaagaggcgcagcgcgaatcagagatcttttcaatgcagcaaggaaaaactcaccttccattatattcattgatgagcttgatgcggttggaggaaaacgcggtaggagtttcaacgatgaacgtgaccagacgttaaaccaggtaaagaatcaacatttagtacccttttaaccatgtagactccattgctgatatacttcctttgtagttgcttactgagatggatggattcgagtcagacacgaaagtcattgtaattgcagcaactaaccgaccagaagcgttagactcggctctttgtcggcctggaaggttctctagaaaagttttggttgcagaaccagaccaagaaggccgtcggaaaatcttggccatacatctgagagacgttcctttagaagaagatgcatttctcatatgtgatctggttgcttctttaactcccggatttgtaggtgctgatctcgccaatattgtcaatgaagctgcattgcttgctgctcgtagaggtcagtctttaagtgatcttttaacagccctttcttgatttaagtagttattcgatctggttgcttcccaggtttgatcttttgggatgaagctgcggtttttggcttaacagacactagcgatttcttgattgttttcaggcggagaagcggtggcacgggaagatataatggaagctatagagagagcgaagttcgggatcaatgataaggaagcgaggccaaggacgttaggaaatgaactcagcaagatgttcccatggatgccatctttggctagaagaaacggaccagatcaagacggtttacaaggaccgttaggttatcaaactctcagctacaagggtgggcgcgccgacccagctttcttgtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 9 - DNA, 3966 bp, Artificial Sequence
  2kbNatPromoter::1056CDS Populus tremula FtSHi3 native promoter construct
gactaaggcttttcaaaatctttgcgtatgatacccaaagattttggagggttattaatggattgaaatttattgttttttgtttgtatgtgttggactatcttttagaagtatggtcaaagagagggttataacccaccatctctaggcttagtcaagcgctaagccaaaatgcatatgggtccgacgaacttccagacccgccatatttaggctcagccaattgctgagcctagatgtttatcggtccagcgagttgtcagacccaccatctctaagctcagccaagtgttaagcccagacatgtatgggtcttatgagctgccaaacccaccatctctagcctgagccaagcgatgaccttagacatgtatgggtctggcgagctgtcacacccaacatctctgagcgaagccaagtactgagcctaaatgtgtataggtccgacgagttgtcaaacccaaaatctctaaacctagccaaacattgagcttagacgtgtatgagtctagtgagctgctagactcactatttacaggctcagctaagtgctgaacctagacacacacaggttcgacaatctatcagactcatcgtcttccaacgtctatgagtccggcaatctaccatactcattagtgccaagcatatgctttctttaactctaaaagaatctcaagctctctgatgggatcgtgggttagagatattttatcatgacgagtcagatatatttttatcctaataaaatagggatatcttatcttaatacgtaagagattttttctcttgacaagacgtagatattttgctaatacgccaaccctttttctttcttggaatgaagagagggatctcatgtgctattaaagaaagaaaaagacaaccttcaggcaggttcaattttcttctctctatttttacaatagtatatctattctaaaaagctatttttcttaaaagtataattaatctcccttttaactgatattaatttaattgttagagaatcattaaatatataaaaggaagacttttgcaggtaacaagcatgatctaacaactactctaccaataaaaaattatttagattatcaaaattaaaagattaacaatattattccaggccatcattcatcccatgggattgaaataacccatcagtaggcgctttgttttgcgttcctttccaacctagttttctgcccttcatattttttcgattagttcttgctcatcgatctttaagagtgaagaccccggtccctagaatatccggcaaaatgcagtataaaaggctcatcttatctctttcctttggcaatctgatggcttaggtttttccttgtgcatgtttgcacgtagttttatggattgaatgaatgagaacatagatcttatcactttagatattatttttccaattcatataacgttgttcataagaattggctcaataaatttaatttaaatattttttgaataaaataatatttcaatagcataaataatacattaaaatagacaagtttttttattttctcaattttaaagagttttaaaatctgagataaaactcgaaacgtttttttaatatgacagatccatagatcaatgctaatggagttgctaagccttaaatactgaaaggactcaactgatcaaattagtaatatggggactcaactatagtagaggcaccacacgtgtgattattgctatagagtgaaaaaaacccaatccacgggttcttataaacccaaaaaaagtttcttatcttcaccgctgaacaagcacaaatacagtagttgcccattcctcagtttgtttccagtttctacaaaaaccaggtaagaaccacgtcaattctttaaggtctcgacggtttatagcaggttcttatgtatgatttcttgcggaatcccaaatgacaggttcatgcttgtaatggatatttcttgattttcttggtaaagctagttgttgttgtcttgtagaggccaagtttgtacaaaaaagcagatggcttctttttctgctgtttgcaataacgggttcttgaattttaaactgaactctggagtcaataatggaaatactaaatctttgaagagatattcaactatttgttacagtccagttttgatcccttctttgggactttgcagtgctggcaaactccaacatttgttgctttggaggagcaagtttaggtcatcattttatgatggaaaaaccggggttcatttatcggggttttgtggtggttgtaagtctcaaaatggtttgtcttgtaagagctcatttaggccattgaggagtgaaaaaagtggagatgaggagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataaaaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatcccgtcgtatatactataacacagactctgtgggtactgaaaatactgaaaactcagaagacaaatcttcagtattaaatcttccaaatgagaatgcagctgaaacagttgcagtagaaggtgttgtgagcaaaactggtctggcatcaagggtagatgtatttaagaaattttcaaggccacgaacttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcaagaaagaggcgatcaaataatcaggcagtcacttttgatgatgtggagggtgttgatgttgctaagatagaactcatggagatagttttgtgtctgcaaggagctatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcggcaagaaaatcatctccatcaattatattcatcgatgaacttgatgcagttggggggaaacgtggtagaagtttcaatgatgaacgggatcaaacactaaaccagttgctgactgaaatggatggttttgagtcagaaatgaaagtggtggttattgcagcaactaataggccggaagctttggatccagccctttgccggcctggtcgcttttcaagaaaggtagttgttggagaaccagatgaagaaggaaggagaaagatcttggctgtacatttgagaggggttcctttagatgaagatgcagatcttatctgcaatctagttgcttcactgacaccaggctttgtaggtgctgatcttgcaaacatcattaatgaagctgctttacttgctgctcgaagaggtggagacattgtgacaagggaagatgtaatggaagcaattgaaagagcaaagtttggaatcagggacaggcaactaaggcccagtacaataagcaaagagctgggcaaattgttcccatggattccttcgttgatgggaacgaatgacacaagacaggatggtttgcaaggctctttaggttaccagaccctgagctga

SEQ ID NO: 10 - DNA, 3008 bp, Artificial Sequence
  p35S::1056CDS Populus tremula FtSHi3 35S promoter construct
gatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcagcaagtttgtacaaaaaagcagatggcttctttttctgctgtttgcaataacgggttcttgaattttaaactgaactctggagtcaataatggaaatactaaatctttgaagagatattcaactatttgttacagtccagttttgatcccttctttgggactttgcagtgctggcaaactccaacatttgttgctttggaggagcaagtttaggtcatcattttatgatggaaaaaccggggttcatttatcggggttttgtggtggttgtaagtctcaaaatggtttgtcttgtaagagctcatttaggccattgaggagtgaaaaaagtggagatgaggagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataaaaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatcccgtcgtatatactataacacagactctgtgggtactgaaaatactgaaaactcagaagacaaatcttcagtattaaatcttccaaatgagaatgcagctgaaacagttgcagtagaaggtgttgtgagcaaaactggtctggcatcaagggtagatgtatttaagaaattttcaaggccacgaacttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcaagaaagaggcgatcaaataatcaggcagtcacttttgatgatgtggagggtgttgatgttgctaagatagaactcatggagatagttttgtgtctgcaaggagctatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcggcaagaaaatcatctccatcaattatattcatcgatgaacttgatgcagttggggggaaacgtggtagaagtttcaatgatgaacgggatcaaacactaaaccagttgctgactgaaatggatggttttgagtcagaaatgaaagtggtggttattgcagcaactaataggccggaagctttggatccagccctttgccggcctggtcgcttttcaagaaaggtagttgttggagaaccagatgaagaaggaaggagaaagatcttggctgtacatttgagaggggttcctttagatgaagatgcagatcttatctgcaatctagttgcttcactgacaccaggctttgtaggtgctgatcttgcaaacatcattaatgaagctgctttacttgctgctcgaagaggtggagacattgtgacaagggaagatgtaatggaagcaattgaaagagcaaagtttggaatcagggacaggcaactaaggcccagtacaataagcaaagagctgggcaaattgttcccatggattccttcgttgatgggaacgaatgacacaagacaggatggtttgcaaggctctttaggttaccagaccctgagctga

SEQ ID NO: 11 - DNA, 1881 bp, Aquilegia coerulea
  > Aqcoe3G076400
atgtccaactatggagtcatgggtgttcaggtttcttgttttaatcaatcaatgatgttttataatggagataaggatagatttcatgtaggaaaattacaatctttggagaaatatagaagttttggtagtgcagtttctatttcatgttcgagtttattgtgtttttcttcttggaggagtagtagatgtgaagttagaaatgaatcattgggtaagaagaagattaggtcacagattgatacaagcagagaaaatcgaaagacccattcagggaaaagtggaagtagtgaatcaaaaaagcgattttctttgagattgagacctagattgcgtcttttgtcaatgaggttgaaaagggtatcaattcgagcagtgggagaggagtttaagatattattgggtaagaatttgaagaaggttactctttcgacatttatttctttgctcgtgggtctatgctacttatttctgaagttaacaagtgtgcctttgccaagggctgtaccatattcagagttaatactgaatcttcaaactgggtctgtgtcgaatgttctcttcgaagaggggtctcgccgtatattctacaacacggtttctaagagttcagataattcacagttgttggagaaccaatcaccatctggagatatatctgagaaagtggatgatactactgtggggggagacgagacagaaaatcccgttgtacccggtgttaatattttggaaaaaatttctgggaaaaaagattctactccagagtggcagtactctacaaggaagattgaccatgatgaaaattttcttcttactctgatgagagaaaagggaactgcgtatagctcagctccacaatccgcactaatgtcagtgagaaacattttgataactatattgtctctgtggattcctttatttcctttgatgtggttgctttatcggcaactttctgctgccagtagtccagcaaaaaagcggcgacccactaatcaaattgtcagctttaacgacgttgagggtgttgatactgctaaggcagagcttttggagattgtattgtgtctacaaggatctatcaattacagcaaattgggagcaaagttacctagaggcgtattgcttgtgggacccccaggaacagggaaaacattattagcccgtgctgttgctggagaagcaggggtaccatttttcacagtttctgcaagtgaatttgtagagatgtttgttggacgaggagcagcacgcatccgggacctttttagtgttgcacgtaagtatgcaccatctatcatattcattgacgagctcgatgctgtgggaggaaagcgtggtagaagtttcaatgatgagcgagatcaaacacttaaccagctgcttacagaaatggatggatttgagacagacatgaaagttgttgtaattgcagcaactaataggccagaagcattagattcagctctctgtcgccctggtcgcttctcgaggaaagtatatgttggtgaaccagatgaggatggaaggagaaaaatattggctgtacatttgaggggagtccctttagaggaagacccccatcttatttgcaatctagttgcatctctcaccaaaggatttgtgggagctgatttagcaaacctcgttaatgaggctgctcttcttgctgctcgaagaggtggtgagactgtggatagagaagatattatggatgctattgaaagggcaaagtttggaataaataataagcaggtgactcctagcaccataaaacgtgaacttggaaaattatttccatggatgccatctcttactaaaaagaatgatgcaaaagaagatgggttggaagggcctataggttaccagactcttagttga

SEQ ID NO: 12 - protein, 626 aa, Aquilegia coerulea
  > Aqcoe3G076400
MSNYGVMGVQVSCFNQSMMFYNGDKDRFHVGKLQSLEKYRSFGSAVSISCSSLLCFSSWRSSRCEVRNESLGKKKIRSQIDTSRENRKTHSGKSGSSESKKRFSLRLRPRLRLLSMRLKRVSIRAVGEEFKILLGKNLKKVTLSTFISLLVGLCYLFLKLTSVPLPRAVPYSELILNLQTGSVSNVLFEEGSRRIFYNTVSKSSDNSQLLENQSPSGDISEKVDDTTVGGDETENPVVPGVNILEKISGKKDSTPEWQYSTRKIDHDENFLLTLMREKGTAYSSAPQSALMSVRNILITILSLWIPLFPLMWLLYRQLSAASSPAKKRRPTNQIVSFNDVEGVDTAKAELLEIVLCLQGSINYSKLGAKLPRGVLLVGPPGTGKTLLARAVAGEAGVPFFTVSASEFVEMFVGRGAARIRDLFSVARKYAPSIIFIDELDAVGGKRGRSFNDERDQTLNQLLTEMDGFETDMKVVVIAATNRPEALDSALCRPGRFSRKVYVGEPDEDGRRKILAVHLRGVPLEEDPHLICNLVASLTKGFVGADLANLVNEAALLAARRGGETVDREDIMDAIERAKFGINNKQVTPSTIKRELGKLFPWMPSLTKKNDAKEDGLEGPIGYQTLS

SEQ ID NO: 13 - DNA, 3171 bp, Ananas comosus
  > Aco015290
atggctcttttctctttgatctctgctaatggcgtagtgattcctcgtagctctcgcttaaatggtgtagtaaacgcttttcctagctttgggaggagtagaatcctctcttcttcgtctcatctctcttgccctagttcaaagcttccacctttggtgggtttttgttgtagaaaccgatacagtgttaggaagcaatgttgtagggtagttgtagcagagattgataggggtgggaaggagaaacatacccatttggggattcaggggaaacccagttcgcggaaacgacggttttcgcttaggttgagaccgcggcttcggattctttattatagatggagaagagtgtccattagtgggcttattgaggatgctcagactacgctgaggggaattgggagaaagattagtcctgccacgttgattccttttgctttgggattgtttttcttggtattgaagtccacttcgaagaatccttcaaaagaggttccctattcggatttggtattgaatcttcaaagaggacaggtatcttcagttctttttgaggaggactctcgccgtatatacttcaacggatattctaatgaggaggatggttcaaagtcgacggttgagagtgaaaatggtaaagtttccactttgaggacaagttcaaaaccgaagtggcggtactacgcgagaaagatcgaccatgacgagaacttcctacttggtttgatgagagagaaaggaatcacttatgggtccgctcctcaatctggatcgaaattattgatgagcatcgtgaccacactgcttactttgtggatttctttagttcctttagtatggtttcttcagcgccagctttatgcagggaataatgcagcgagaaagcggcgtcctagtaagcaaatggtcagttttgatgatgtggagggtgttgatgctgccaaggaggagcttgtggagattgtaagttgcttgcaaggatcgataaactacaagaagctgggagcaacattgccaaggggcgtgcttcttgtgggccctccaggaactggaaaaactttactagctcgtgcagtggcaggagaagctgggattcctttcttctccatttctgctagtgaatttgttgaagtctttgttggaagaggagcggcacgtgtgagagagctttttagagaggcgaaagaattcgcaccatcgattatcttcattgatgagcttgatgcggtgggagggaagcgtggtagaagctttaatgatgagagagatcaaacccttaaccaggcactcatgagttggtatactttcctccagttactcactgaaatggatgggtttgagtctgaggcgaaagtgattgttatcggggcgaccaatagaccagaggcattagattctgctctctgtcgaccaggtcggttttcaagaaaggttttagttggagagcctgatcaagaagggcgtaaaaagattctaagtgttcatctaagaagagttcctttggaggaggagcctgagataatttgcaatttaattgcttctctcaccccaggatttgtaggggccgatcttgccaacattgtcaatgaggctgctcttcttgctgctcgaaggggtggtaagacggtgacccgcgaagacattatggatgcaatcgaacgggagaagtttggggttaatggccgaccattaggtcctaaagctgaaagcaaaggccttgcggaactatttccatggtttcctacttttgcaggaagaaatggcggaaaaggagatggtttgcaaggtaggatgcggatttactacgaaagaccaaccttgagaaatgcagtatcaaatttcacaggaatatttactgaaacactaaaattgaaggttctccgggtatcagtaaatacgcatccttcagaaaagagctgcaagaatcagaaaatgtataccaccaggcccctctccaccttcaggggatctcatggcgagcggcgatccgaagagccattctcgggctccctttttctcgacgaccagagcgagttcaacatcttatcatcctcttgctgctgggcgaaaaacgtgaaggaacccctcaagaatcttccattccctcaagataggatcatcaccatcaaatactccccacgaggtgaaaagaatattaaaagggctaataaggcgacagtcttcttcgtgccagtcttaaatcaaccatcgtcggcgtcatttaaccgataccatgttatcattgcaaaaggaagaaataaggggaaagtatatgtgtgctctaaagagggcatcggtgcatgttgctcatgccgcggggaaggagacgtgaaaccaagaccttttgatcatcgcgacgtacaccaacaaatggaaattgtgtgtgagaaagaaggattattcaccgccaagtcggtagctcacgatggccaacctccatggttactaggaagcaagtattggaaagcttatgcttcgaaaccgaagaattgtcacttaagtgaggccttggggcttaggcaaccccttcgttcatgtttacccgatttaaacttcccgatacacgcacgagattgtccgaaagttactatcggtaaatggtattgcccatttgtgtttgttaaggaagcaggcggtttggatgttcaaatgaagcgcgccttatattacgagatgactctcgagcaattttggaaggagatttacacgtgcgagaacaacagcggcgaagagagaacggtggaggtgaacgcgctcgtcgagagcaaagccgtttttgtagatggtaaagaagtgggacaagataacaagcgccgccgcgatggtttcatatggtttgcgccttttgaatcgaaaccgaagggtgttgggttgagcttggctatacgggagagaatgatatgggagcaaaagaagggaggatggattggtgatgatgaagatgaagaggtggagaggattgtgagggaatttggaggagagaataatatgtggaggaggtttgcttgctatatcttggtggagaggtatgttctcaagagggtggatgggagcttggctttgacttttgattttaggcatcttattaagttcagaaccaaatgggagatccttcttgtggcattaagatcccgtatccagcacctcgataagggccaacggcttatttga

SEQ ID NO: 14 - protein, 1056 aa, Ananas comosus
  > Aco015290
MALFSLISANGVVIPRSSRLNGVVNAFPSFGRSRILSSSSHLSCPSSKLPPLVGFCCRNRYSVRKQCCRVVVAEIDRGGKEKHTHLGIQGKPSSRKRRFSLRLRPRLRILYYRWRRVSISGLIEDAQTTLRGIGRKISPATLIPFALGLFFLVLKSTSKNPSKEVPYSDLVLNLQRGQVSSVLFEEDSRRIYFNGYSNEEDGSKSTVESENGKVSTLRTSSKPKWRYYARKIDHDENFLLGLMREKGITYGSAPQSGSKLLMSIVTTLLTLWISLVPLVWFLQRQLYAGNNAARKRRPSKQMVSFDDVEGVDAAKEELVEIVSCLQGSINYKKLGATLPRGVLLVGPPGTGKTLLARAVAGEAGIPFFSISASEFVEVFVGRGAARVRELFREAKEFAPSIIFIDELDAVGGKRGRSFNDERDQTLNQALMSWYTFLQLLTEMDGFESEAKVIVIGATNRPEALDSALCRPGRFSRKVLVGEPDQEGRKKILSVHLRRVPLEEEPEIICNLIASLTPGFVGADLANIVNEAALLAARRGGKTVTREDIMDAIEREKFGVNGRPLGPKAESKGLAELFPWFPTFAGRNGGKGDGLQGRMRIYYERPTLRNAVSNFTGIFTETLKLKVLRVSVNTHPSEKSCKNQKMYTTRPLSTFRGSHGERRSEEPFSGSLFLDDQSEFNILSSSCCWAKNVKEPLKNLPFPQDRIITIKYSPRGEKNIKRANKATVFFVPVLNQPSSASFNRYHVIIAKGRNKGKVYVCSKEGIGACCSCRGEGDVKPRPFDHRDVHQQMEIVCEKEGLFTAKSVAHDGQPPWLLGSKYWKAYASKPKNCHLSEALGLRQPLRSCLPDLNFPIHARDCPKVTIGKWYCPFVFVKEAGGLDVQMKRALYYEMTLEQFWKEIYTCENNSGEERTVEVNALVESKAVFVDGKEVGQDNKRRRDGFIWFAPFESKPKGVGLSLAIRERMIWEQKKGGWIGDDEDEEVERIVREFGGENNMWRRFACYILVERYVLKRVDGSLALTFDFRHLIKFRTKWEILLVALRSRIQHLDKGQRLI

SEQ ID NO: 15 - DNA, 1842 bp, Arabidopsis halleri
  > Araha.2210s0001
atggctactttcaatcttctttgtagcaatcgatttctattcaaaggaggttattctccggagaaatctattagaaagttgtcttcgaggagttgtgagcttaatgtttgtgtagctagaaccaggaggaatcaatcgtttagttgcaggagacttggtgggtttctggaaattggtgataattgtcgaaatgggtttgtttgtaatagcgagattaagcgacttgtgagtggtgattacggagataaagagactcgtattggtgaaaatggtagaaacaaagggaagagaagaaggttttctcttaggcttcgacctagattacgattagtgagaatgagacttgggagatttgatttcagagcatcaatggaagattttcgatattttttgaaaaagaatatcaaaagagtgatcttgtccaccggtgtggctttaatctttggactgtgttatttgttcttgaggttaactgctgttccgtccccatccattgttccttattcagatttcgtaacgaatcttcgaggtggttctgtttcaaaggttttgctcgaagaaggctctcgtcgaatctattacaacactgatgagaatgttgaggttgttgatgatcttcacaagtctgaaactttagaagatccggcaatccaaattgatggcggcacagtgacggaagctgttacgaaagacgatactccgcgaaaggttcgagctttgactcctgtgtggaagtatgtgacaagaaaagtcgatcatgatgagaagtttcttcttagcttaatgagagagaaaggcattacttatagttcagctcctcaatctgcattgatgtcaatgagaactactttgataacaatcatctctctgtggattcctttgacacctcttatgtggcttctttatcggcaactctcggcatctaacagccctgcgaagaaacgacggtctaaaaatcccacggttggattcgatgatgttgagggtgttgattctgccaaagatgagctcgtggagatagtgtcatgtcttcaaggaagtataaactacaaaaaattaggagcaagattaccgagaggtgtgcttttggtcggtccaccagggactggtaaaaccttgttggctcgggctgtagctggagaagcgggagttccgtttttctctgtttccgctagtgagtttgttgaattgtttgttggaagaggcgcagcgcgaatcagagatcttttcaatgcagccaggaaaaactcaccttccattatattcattgatgagcttgatgcggttggaggaaaacgtggtaggagtttcaatgatgaacgtgaccagacgctaaaccagttgcttactgagatggatggattcgagtcagacacaaaagttattgtaattgcagcaaccaaccgaccagaagcgttagactcagctctttgtcgacctggaaggttctctagaaaagttgtggttgcagaaccagaccaagaaggccgtcggaaaatcttggccgtacatttgagagacgtccctctagaagaagatgcatttctcatatgtgatctggttgcttctttaactcccggattcgtaggtgctgatctcgccaacattgtcaatgaagctgcattgctagctgctcgaagaggaggagaagcggtggcacgggaagatataatggaagcgatagagagggcaaaattcgggatcaatgataaggaagtgaggccaaggacattaggaaatgagctaagcaagctgttcccatggatgccatctttagctagaaggaacggaccagatcaagacggtttacaaggaccgttaggctatcaaactctcagctaa

SEQ ID NO: 16 - protein, 613 aa, Arabidopsis halleri
  > Araha.2210s0001
MATFNLLCSNRFLFKGGYSPEKSIRKLSSRSCELNVCVARTRRNQSFSCRRLGGFLEIGDNCRNGFVCNSEIKRLVSGDYGDKETRIGENGRNKGKRRRFSLRLRPRLRLVRMRLGRFDFRASMEDFRYFLKKNIKRVILSTGVALIFGLCYLFLRLTAVPSPSIVPYSDFVTNLRGGSVSKVLLEEGSRRIYYNTDENVEVVDDLHKSETLEDPAIQIDGGTVTEAVTKDDTPRKVRALTPVWKYVTRKVDHDEKFLLSLMREKGITYSSAPQSALMSMRTTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESDTKVIVIAATNRPEALDSALCRPGRFSRKVVVAEPDQEGRRKILAVHLRDVPLEEDAFLICDLVASLTPGFVGADLANIVNEAALLAARRGGEAVAREDIMEAIERAKFGINDKEVRPRTLGNELSKLFPWMPSLARRNGPDQDGLQGPLGYQTLS

SEQ ID NO: 17 - DNA, 1857 bp, Amaranthus hypochondriacus
  > AHYPO_002963
atggcttctttttgccttgtttctaattgtgtatcagtgggcaatcccaaaagctataaactttatcctttgagagctaaatctgttgatagatgtatatataggtctcaatctgtattgcctaaatatgattgtgcttcttttcatccattaaaatctcaattttactgtaatttggtttgttgtagtaatatagggttaattagtgatggaaataggaaaaataaagttgcccatttggaaagtagggaattaattagggtaagtcggaaattttcattgagattaaggcctaggttgaggctattgtctactagattgaaaagggtttcgattcgatcagcttttaatgattttggaatgtttttgagaaagcatatgaagaaatttacccttgctacttcaatttcatttgcacttgggatatgttatttgtttgtaaagataatggcaattccaaacccgaaaactgttccttattcggatttagttttaagcatccaaaacgggtctgctacgaaagttttgtttgaagaaggttcacgacgaatttacttcaatacaaagcctccttgtgttgaatccccttgtttagaggaagaaaatgtgcttttagagggtgatcagaatgaaaatgtggtgggtgaatctgttgaggtagctgatgggtccacacaagttaagggtacccttgtgcctgtaagaagtttgaggaaaaaacccgtagttcccgagtggcaatatgctacaaggaaggttgataatgatgaaagctttcttttaggtcttatgagagaaaagggtattgtatatagctcggctcctcaatcggtgttaatgtcgttcaagagtattttactaactgtcttgacattatggatccccttggctcctatgatgtggcttctttatcgtcaactttacgcttcgaatgggcctgcaaagaaacgaaagcctagtgaccggactgttagctttgacgatgttgaaggcgttgatagtgcaaaagttgagctcatggagatagtatcatgcatgcaaggatctatcaattataacaaattaggagcaaggttacctagaggtgttctgttggtaggtcctccaggaacaggaaaaaccttactagctcgtgctgtggctggtgaagctggtgtaccattcttctctgtatccgccagtgaatttgtagagttgtttgtcgggaggggagctgcacgtattagagatctttttagcgttgcaaggaaatcttcaccgtcaattatattcattgatgaacttgatgctgttggaggaaagcgtggtaggagtttcaatgatgaacgtgatcagaccctcaaccagttgctgacagaaatggatggtttcgagtcagatacaaaggtggttgttattgctgcaacaaaccgacctgaagcattggatcaggctctttgccgtcctggtcgcttctcgaggaaagtagttgtaggagaacccgacgaagaagggaggaaaaagatactggcggtgcattttagggatgttccgctagacgaaaacaagcaactaatctgtgaccttgttgcttccctcacccaaggtctcgttggtgctgatcttgccaatattgtcaatgaatctgcattactcgctgctcgcagaggtggagaagcagtatcgagagaagatataatggaagccatagagagggcaaagttcggtatcaacaataagcaaattctagcgacttcgagtatgattggcaaggagctagggaagctattcccatggataccttctattataggaaagactgaaacaagaacagatggatttcaagagcctatgggttatcaaacattaagctcatga

SEQ ID NO: 18 - protein, 618 aa, Amaranthus hypochondriacus
  > AHYPO_002963
MASFCLVSNCVSVGNPKSYKLYPLRAKSVDRCIYRSQSVLPKYDCASFHPLKSQFYCNLVCCSNIGLISDGNRKNKVAHLESRELIRVSRKFSLRLRPRLRLLSTRLKRVSIRSAFNDFGMFLRKHMKKFTLATSISFALGICYLFVKIMAIPNPKTVPYSDLVLSIQNGSATKVLFEEGSRRIYFNTKPPCVESPCLEEENVLLEGDQNENVVGESVEVADGSTQVKGTLVPVRSLRKKPVVPEWQYATRKVDNDESFLLGLMREKGIVYSSAPQSVLMSFKSILLTVLTLWIPLAPMMWLLYRQLYASNGPAKKRKPSDRTVSFDDVEGVDSAKVELMEIVSCMQGSINYNKLGARLPRGVLLVGPPGTGKTLLARAVAGEAGVPFFSVSASEFVELFVGRGAARIRDLFSVARKSSPSIIFIDELDAVGGKRGRSFNDERDQTLNQLLTEMDGFESDTKVVVIAATNRPEALDQALCRPGRFSRKVVVGEPDEEGRKKILAVHFRDVPLDENKQLICDLVASLTQGLVGADLANIVNESALLAARRGGEAVSREDIMEAIERAKFGINNKQILATSSMIGKELGKLFPWIPSIIGKTETRTDGFQEPMGYQTLSS

SEQ ID NO: 19 - DNA, 1851 bp, Arabidopsis lyrata
  > AL3G11750
atggctactttcaatgttctttgtagcaatcggtttctattcaaaggtggttattctccggataaatctattagaaagttgtcttcgaggagttgtgagcttaatgtttgtgtagctagaaccaggaggaatcaatcgtttagttgcaggagacttggtgggtttctggaatttggtgataccagattaggggttagaaatgggtttgtttgtaatagcgagattaagcgacttgtgagtggtgattacggagataaagagactcgtattggtgaaaatggtagaaacaaagggaagagaagaaggttttctcttaggcttcgacctagattacgattagtgagaatgagacttgggagatttgatttcagagcatcaatggaagattttcgatattttttgaaaaggaatatcaaaagagtgatcttgtccaccggtgtggctttaatctttggactgtgttatttgttcttgaggttaactgctgttccgtccccatccattgttccttattcagatttcgtaacgaatcttcgaggtggttctgtttcaaaggttttgctcgaagaaggctctcgtcgaatctattacaacactgatgagaatgttgaggttgttgatgatgttcacaagtctgaaactttagaagatccggcaatccaaattgatggcggcacagtgagggaagctgttacgaaagacggtactccgcgaaaggttcgagctttgactcctgtgtggaagtatgtgacaagaaaagtcgatcatgatgagaagtttcttcttagcttaatgagagagaaaggcattacttatagttcggctcctcaatctgcattgatgtcaatgagaactactttgataacaatcatctctctgtggattcctttgacacctcttatgtggcttctttatcggcaactctcagcatctaacagccctgcgaagaaacgacggtctaaaaatcccactgttggcttcgatgatgttgagggtgttgattctgctaaagacgagctcgtggagatagtctcatgtcttcaaggaagtataaactacaaaaaactaggagcaagattaccgagaggtgtgcttttggtcggtccaccagggaccggtaaaaccttgttggctcgggctgtagctggagaagcgggagttccatttttctctgtctccgctagtgagttcgttgaattgtttgttggaagaggcgcagcgcgaatcagagatcttttcaatgcagccaggaaaaactcaccttccattatattcattgatgagcttgatgcggttggaggaaaacgtggtaggagtttcaatgatgaacgtgaccagacgctaaaccagttgcttactgagatggatggattcgagtcagacacgaaagttattgtaattgcagcaactaaccgaccagaagcgttagactcagctctttgtcggcctggaaggttctctagaaaagttgtggttgcagaaccagaccaagaaggccgtcggaaaatcttggccgtacatttgagagacgtccctctagaagaagatgcatttctcatatgtgatctggttgcttctttaactcccggattcgtaggtgctgatctcgccaacattgtcaatgaagctgcattgctagctgctcgaagaggaggagaagcggtggcacgggaagatataatggaagcgatagagagggcgaaattcgggatcaatgataaggaagtgaggccaaggacattaggaaatgagctaagcaagctgttcccatggatgccatctttagctagaaggaacggaccagatcaagacggtttacaaggaccgttaggctatcaaactctcagctaa

SEQ ID NO: 20 - protein, 616 aa, Arabidopsis lyrata
  > AL3G11750
MATFNVLCSNRFLFKGGYSPDKSIRKLSSRSCELNVCVARTRRNQSFSCRRLGGFLEFGDTRLGVRNGFVCNSEIKRLVSGDYGDKETRIGENGRNKGKRRRFSLRLRPRLRLVRMRLGRFDFRASMEDFRYFLKRNIKRVILSTGVALIFGLCYLFLRLTAVPSPSIVPYSDFVTNLRGGSVSKVLLEEGSRRIYYNTDENVEVVDDVHKSETLEDPAIQIDGGTVREAVTKDGTPRKVRALTPVWKYVTRKVDHDEKFLLSLMREKGITYSSAPQSALMSMRTTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESDTKVIVIAATNRPEALDSALCRPGRFSRKVVVAEPDQEGRRKILAVHLRDVPLEEDAFLICDLVASLTPGFVGADLANIVNEAALLAARRGGEAVAREDIMEAIERAKFGINDKEVRPRTLGNELSKLFPWMPSLARRNGPDQDGLQGPLGYQTLS

SEQ ID NO: 21 - DNA, 1929 bp, Amborella trichopoda
  > evm_27.TU.AmTr_v1.0_scaffold00006.221
atggctgctcttcgtgtatcattgagctctggtatcgagcttctccatatttgtagctgcaccagtaagcttattccagggcccaataaagatttccatggaatctctttgagcttcagaggaaaacctagggttcagttaggaaggcttcgaacgccggcaccttctgtaactccttctttgctcacaagtggtaatgattcaatgaattttgtgcatttgatgaattcatacgaatctctctctgggaagaaaacccaattcaggatttgggcacatggtgagtctaacgagagaatgagcggtggtttgaaaaagcgtttcttgcttggtttgaaaaagcgtttctcgcttcggttgaggcctcgcttgcgattgttatcggctaagctgaagagagaattctccatggagagactagaagaaatgttggtactgttgcgcaggaattcaaagagagttactctctccactttaatggcattagtgttgggtttgtgttatgggtttctcaagctaacagcaatgcctcaacctaaaacagtgccatattcaaatttgatcacaaaccttcaatcaggctgtgtttccaaggttcttttcgaagagggttcccgtcggatatatttcaatataaacactgattctcatattgattccaagtcatttgatgatgtttcatcaatatcggagagtaaaaaagtgggttttcatgaatcagaagctgcttcatctgagaaagctgcttcatttgagaacaatagtaagaaatttgggtttggtgagaaaggcaaagctcgggggcaaacagagtggcagtacctgaccagaaaaatcgaccatgatgagaattttctacttggtttgatgagagagacaggaactatgtatagctcagctccccagtcagttctcatgtctatgaggagtgtgttgatcactattttatctctttggattccattgactcctttgatgtggctgctctatcggcaactatctactgctaatagtccagcaaagaggcgtcgaaccaccagtaaaactgtgagttttgatgacgtggagggggttgatgccgcaaaatcagagctcatggagatagtatcatgcttacaaggatcaattaattacaacaaactcggggcaaagttgccaagaggtgtgcttttggtggggcctccaggaacagggaaaacattactagcacgtgcagtggctggagaagcaggagtgccctttttctctgtctctgctagtgaatttgtggagttgtttgttggaagaggtgcagctcgaatcagagacctcttcaatgtagcaaggaaatgttcaccatcaatcatttttattgatgagcttgatgcagttgggggaaaacgaggaagaagtttcaatgatgaacgggaccaaacactaaatcagttattgacagaaatggatgggtttgagtcagatatgaaggtggttgttattgctgcaaccaatagaccagaagcattggatccagctctctgcaggcctggccggttttctagaaaggtattggtcagggaaccagatgaaagtggaaggagaaagatcttggctgttcatttacgaggagttccgttggaggaggatccaaatgtgatctgtaatctagttgcctctctcacccatggttttgtcggagctgatcttgcaaacatagttaatgaggctgccttgcttgctgctcggagaggtggtgaattggtgaccagagaagatataatggaggcagtagaaagagcaaagtttggcattagtgaccggccaacaaatgaaaactcaataaccaaagagcttggaaagttgtttccatggatgccctctcttgttggaaggactaatgttacaagagatgatggatggcagcaacctccaggctaccaaactctaagctaa

SEQ ID NO: 22 - protein, 642 aa, Amborella trichopoda
  > evm_27.TU.AmTr_v1.0_scaffold00006.221
MAALRVSLSSGIELLHICSCTSKLIPGPNKDFHGISLSFRGKPRVQLGRLRTPAPSVTPSLLTSGNDSMNFVHLMNSYESLSGKKTQFRIWAHGESNERMSGGLKKRFLLGLKKRFSLRLRPRLRLLSAKLKREFSMERLEEMLVLLRRNSKRVTLSTLMALVLGLCYGFLKLTAMPQPKTVPYSNLITNLQSGCVSKVLFEEGSRRIYFNINTDSHIDSKSFDDVSSISESKKVGFHESEAASSEKAASFENNSKKFGFGEKGKARGQTEWQYLTRKIDHDENFLLGLMRETGTMYSSAPQSVLMSMRSVLITILSLWIPLTPLMWLLYRQLSTANSPAKRRRTTSKTVSFDDVEGVDAAKSELMEIVSCLQGSINYNKLGAKLPRGVLLVGPPGTGKTLLARAVAGEAGVPFFSVSASEFVELFVGRGAARIRDLFNVARKCSPSIIFIDELDAVGGKRGRSFNDERDQTLNQLLTEMDGFESDMKVVVIAATNRPEALDPALCRPGRFSRKVLVREPDESGRRKILAVHLRGVPLEEDPNVICNLVASLTHGFVGADLANIVNEAALLAARRGGELVTREDIMEAVERAKFGISDRPTNENSITKELGKLFPWMPSLVGRTNVTRDDGWQQPPGYQTLS

SEQ ID NO: 23 - DNA, 1722 bp, Brachypodium distachyon
  > Bradi5g12800
atggcgtctctccccatcttatccgctaccgctgcttccctcccgcggccccacgtctgctgcttcaggaaccccgcctcttcttcttcgtgggcctgcttcgccgagagcaatggttgctgcggagtgcttgacgccgggcgttacgcgaggctgcggctgcggtggaggagccccgtccgtgccaaggtcggcgaggtggacagggacaagggcgccgatttggggctccgcccggaccgcaagagcctgcggcggcgcctgaggctcaggccgaggctgcgcctgctgtcgtggcggctccagcgcctgtcgccacgagagctcgcgggcgacgccgcggcggcgttgcggcgggccctccgtcgcgtgcctcccccggcggtcgcgtcgatcgtcctgggggctctcctcctcgccgcgcggctcacgctgcccaagaactcggcccgggaagtggcctactcggacctcctggcggggctccgcgcgggcgccgtgaccgccgtcgcgttcgaggaggactcgcgccgcatctacttcaacaaggccgaggacatcggcagcggtgacgacgagggcgataccggtaaaggcgccgccacagcgaagtggccgtactacgcgaggagggtgccgcatgatgaggggttcctgctggggttaatgcgggagggcggggtggactaccgatcggcaccacggccggcggggaggctgctggtggacatgttgagtacgctgctcactctgtgggtctcattgctgccgatgatgtggttcctacagaggcaaatgtcggctggcggcagtgcagacaagcggcgccggcccaggaagcagcaagtaggatttgatgatgtccagggcgtggatgaagccaaggaggagcttgtcgagatagtgagctgcttgcgcggttcactgaattacaagaagctgggagctaggttaccaaggggtgttctgcttgttgggcctccggggactgggaaaacactgctggctaaggcagttgcaggagaggctggaattccattcttccctgtttctgctagtgaatttgttgaagtttttgttggaagaggagcagcccgtgtcagggatttgttcaaggaagccaaagaagctgctccgtcaatcatttttattgatgaacttgatgctgtgggtggaagcagaggtagaagttttaatgatgagagggaccaaactctaaatcagctacttactgaaatggatggttttgactcggatgtgaaagttattgtcatggctgctactaatagacctaaagctctggattctgcactttgtcgacccggtcgcttctcaagaaaggtttttgttgccgtgcctaatttggagggccgcagaaatattctggctgttcatctcagagaagttcctttagaagaggatcttgaaataatttgtgatctggttgctaatgtgaccccaggattggttggtgcagatctagcaaatattgttaatgaggccgcgttactagctgctcgaagaggtggaaatactgtggctcgggaggatattatggatgccattgagagggagaaatatggggtcaacggtagacaaaaaggtcctgattctgaaagactagggcttaccaaattatttccatggttaactaaacctgggaataaaccatcaaacccagacgactttccaggacttttaggttaccatacattaagctaa

SEQ ID NO: 24 - protein, 573 aa, Brachypodium distachyon
  > Bradi5g12800
MASLPILSATAASLPRPHVCCFRNPASSSSWACFAESNGCCGVLDAGRYARLRLRWRSPVRAKVGEVDRDKGADLGLRPDRKSLRRRLRLRPRLRLLSWRLQRLSPRELAGDAAAALRRALRRVPPPAVASIVLGALLLAARLTLPKNSAREVAYSDLLAGLRAGAVTAVAFEEDSRRIYFNKAEDIGSGDDEGDTGKGAATAKWPYYARRVPHDEGFLLGLMREGGVDYRSAPRPAGRLLVDMLSTLLTLWVSLLPMMWFLQRQMSAGGSADKRRRPRKQQVGFDDVQGVDEAKEELVEIVSCLRGSLNYKKLGARLPRGVLLVGPPGTGKTLLAKAVAGEAGIPFFPVSASEFVEVFVGRGAARVRDLFKEAKEAAPSIIFIDELDAVGGSRGRSFNDERDQTLNQLLTEMDGFDSDVKVIVMAATNRPKALDSALCRPGRFSRKVFVAVPNLEGRRNILAVHLREVPLEEDLEIICDLVANVTPGLVGADLANIVNEAALLAARRGGNTVAREDIMDAIEREKYGVNGRQKGPDSERLGLTKLFPWLTKPGNKPSNPDDFPGLLGYHTLS

SEQ ID NO: 25 - DNA, 1722 bp, Brachypodium distachyon; identical to SEQ ID NO: 23
  > Bradi5g12800

SEQ ID NO: 26 - protein, 573 aa, Brachypodium distachyon; identical to SEQ ID NO: 24
  > Bradi5g12800

SEQ ID NO: 27 - DNA, 1740 bp, Brassica rapa
  > Brara.E03580
atggctactactttcaatgttctctgtacacatacatgggatgatcggtttcatcttaaagtagctaatcctccggatagattcagcagaacagtgtactcaaggattagcgagcttaatgttagtgtcgctcggacaaagaatctgtcgtttagttgcaattgcaaaagacttggtcggttttcttgtaataacgagattaagcgactagtgaatagtgattatggtgaaaacggtacaagcaaaggaacgagaagaagaaggttttctcttaggcttcgacctaggctacgtttactgagcatgagacttggcagattcgatctcagagcgtcactggaagacttcagattgtttttgagaaagaacatcaaaagagtgatcttatccaccggcgtggcggttatcttcggactttgttatctgttcttgaggctaaccgctgttccctctccatccattgtcccttactcagacttcgtcacgaatcttcgaggcggttctgtctctaaggttttgctcgaagaaggttctcgtcgaatctattacaacactgctgaggagaatgttgaggttgttgaagatgttacgaaagacgttggaaaggttcgagctttggctcctgtgtggaagtacgtgacgaggaaagtagatcacgatgagaagtttcttcttggtttgatgagggagaaagggattacttatagctcagctcctcaatctgcgttgaagtcaatgagaactgttttgttaactatcatcactctatggattcctttaactcctctcatgtggcttctctatcggcagctctcagcatctaacagccctgcgaagaaacgacggtctaaaaatcccactgttggattcgatgacgtggagggcgttgattctgctaaagacgagctcgttgagatagtgtcatgtcttcaaggaagtataaactacaagaagctaggagcaagattacctagaggcgtgcttttggtcggtccaccagggactggtaagaccttgttggctcgagctgtagccggagaggctggagttcccttcttctctgtttccgctagcgagtttgttgaaatgtttgtcggaagaggcgctgcgcgaatcagagatctcttcagtgcagccagaaagaactcaccttccattatattcatcgatgagctcgatgcggttggtggaaaacgcggtagaagtttcaacgatgagcgtgaccagacgctaaaccaattgcttactgagatggatggattcgagtcagacataaaagtgattgtaatcgcagcaactaaccgaccagaagcattagaccctgctctatgtcggccaggaagattctcgaggaaagttgtggttgcagaaccggaccaagaaggacgtaggaaaatcttggccgtacatttgagagacgttcctctagaagaagatgcgtttctcatatgtgatctggttgcttctctgactcccggattcgtaggtgctgatctggcaaatattgtcaatgaagctgcattgctcgctgctcgtagaggcggggaagctgtggcaagggaggatataatggaagctatagagagggcgaagtatgggatcaatgataaggaagtgaaggcaaggacgttgggaaatgagctcagcaagttgttcccgtggatgccatctttggctggaaggaacggaccagatcaagacggtttacaaggacccttaggttatcaatctcttagctaa

SEQ ID NO: 28 - protein, 579 aa, Brassica rapa
  > Brara.E03580
MATTFNVLCTHTWDDRFHLKVANPPDRFSRTVYSRISELNVSVARTKNLSFSCNCKRLGRFSCNNEIKRLVNSDYGENGTSKGTRRRRFSLRLRPRLRLLSMRLGRFDLRASLEDFRLFLRKNIKRVILSTGVAVIFGLCYLFLRLTAVPSPSIVPYSDFVTNLRGGSVSKVLLEEGSRRIYYNTAEENVEVVEDVTKDVGKVRALAPVWKYVTRKVDHDEKFLLGLMREKGITYSSAPQSALKSMRTVLLTIITLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVEMFVGRGAARIRDLFSAARKNSPSIIFIDELDAVGGKRGRSFNDERDQTLNQLLTEMDGFESDIKVIVIAATNRPEALDPALCRPGRFSRKVVVAEPDQEGRRKILAVHLRDVPLEEDAFLICDLVASLTPGFVGADLANIVNEAALLAARRGGEAVAREDIMEAIERAKYGINDKEVKARTLGNELSKLFPWMPSLAGRNGPDQDGLQGPLGYQSLS

SEQ ID NO: 29 - DNA, 1719 bp, brachypodium
  > Brast09G111400
atggcctctatccccaccttatccgctaccgctgcttccctcccgcggccccacgtctgctgcttcaggaaccccgcctcttctacttcgtgggcctgcttcgctgggagcaatggctgctgcggagtgcttgacgccgggaggctgcggctacggctgcgatggaggagccccgtccgtgccaaggtcagcgaggtggacaaggacaagggcgccgatttggggctcagcccggaccgcaagagcctgcggcggcgcctgaggctcaggccgcggctgcgcttgctgtcgtggcggctccggcgcctgtcgccacgagagctcgcgggcgacgccgcggcggcgttgcggcgggccgtccgtcgcgtgccgcccccggcggtcgcgtcgatcgtcctaggggctctcctcctcgccgcgcggctcacgctgcccaagaacccggcccgggaagtggcctactcggacctcctggctggactccgcgcgggcgccgtgaccgccgtcgcgttcgaggaggactcgcgccgcatctacttcaacaaggccgaggacatcggcagcggcgacgacgagggcgataccggtaaaggcgccgccacagcgaagtggccttattatgcgaggagggtgccgcatgacgaggggttcctgctggggttaatgcgggagggcggggtggactaccgatcggcaccacggccggcggggaggctgctggtggacatgttgagcacgctgttcacgctgtgggtctcattgctgccgatgatgtggttcctacagaggcagatgtcagctggcggcagtgcggacaagcggcgccggcccaggaagcagcgagtaggatttgacgatgtccagggcgtggatgaagccaaggaggagcttgtcgagatagtgagctgcttacgcggttcactgaattacaagaagctgggagctaggttacccaggggtgttctgcttgttgggcctccagggactgggaaaacactgctggctaaggcagttgcaggagaggctggaattccattcttccctgtttctgctagtgaatttgttgaagtttttgttggaagaggagcagcccgtgtcagggatttgttcaaggaagccaaagatgctgctccgtcgatcatttttattgatgaacttgatgctgtgggtggaagcagaggtagaagttttaatgatgagagggaccaaaccctaaatcagctgcttactgaaatggatggttttgactcggatgtgaaagttattgtcatggctgccactaatagacctaaagctctggattctgcactttgtcgacccggtcgcttctcaagaaaggtttttgttgccgtgcctgatttggagggccgcagaaagattctggctgttcatctcagagaagttcctttagaagaggatcttgaaataattagtgatctggttgctaatgtgaccccaggattggttggtgcagatctagcaaatattgttaatgaggctgcgttactagctgctcgaagaggtggaaatactgtggctcgggaggatattatggatgccatcgagagggagaaatatggggtcaatggtagacaaaagggtcctgatgctaaaagactaggccttaccaaattatttccatggttaactaaacctgggaataaaccatcaaacccagatgactttccaggacttttaggttaccatacattaagctaa

SEQ ID NO: 30 - protein, 572 aa, Brachypodium
  > Brast09G111400
MASIPTLSATAASLPRPHVCCFRNPASSTSWACFAGSNGCCGVLDAGRLRLRLRWRSPVRAKVSEVDKDKGADLGLSPDRKSLRRRLRLRPRLRLLSWRLRRLSPRELAGDAAAALRRAVRRVPPPAVASIVLGALLLAARLTLPKNPAREVAYSDLLAGLRAGAVTAVAFEEDSRRIYFNKAEDIGSGDDEGDTGKGAATAKWPYYARRVPHDEGFLLGLMREGGVDYRSAPRPAGRLLVDMLSTLFTLWVSLLPMMWFLQRQMSAGGSADKRRRPRKQRVGFDDVQGVDEAKEELVEIVSCLRGSLNYKKLGARLPRGVLLVGPPGTGKTLLAKAVAGEAGIPFFPVSASEFVEVFVGRGAARVRDLFKEAKDAAPSIIFIDELDAVGGSRGRSFNDERDQTLNQLLTEMDGFDSDVKVIVMAATNRPKALDSALCRPGRFSRKVFVAVPDLEGRRKILAVHLREVPLEEDLEIISDLVANVTPGLVGADLANIVNEAALLAARRGGNTVAREDIMDAIEREKYGVNGRQKGPDAKRLGLTKLFPWLTKPGNKPSNPDDFPGLLGYHTLS

SEQ ID NO: 31 - DNA, 1875 bp, Boechera stricta
  > Bostr.1460s0159
atggctactttcaatcttctttgtagcaatcgatttctatttaaaggaggtagttctcccgagaaatctagtagaaaggtgtcttcgaggagtagtgagcttaatgtttgtgtcgctagaacgaggaatcagtcgtttagttgcaggagacttggtgggtttctggaaattggtgaaaccagattaggggttagagttcatggcgattctcgaaatgggttttcttgtagtagtgaaattaagcgacttatgagtggtgattacggagataaggagactcgtattggtgaaaatggtagaaacaatgggaagagaagaagattttctcttaggcttcgaccaagattacgattagtgagcatgagacttgggagattccatttaagagcatcaatggaggattttcgactctttttgaaaaagaatttcaaaagagtgatcttatctactggtgtggctgtaatctttggactgtgttatttgttcttgaggttaaccgctgttccgtctccatctattgttccttactcagatttcgtaacgaatcttcgaggtggttccgtttcaaaggttttgctcgaagaaggttctcgtcgaatctattacaacactgatgagaatgttgaggttgttgaagacgtccacaagtctgaaactttagaagaaccggcaatccaaaatgatggccgcacaatggaaactgtgaccgaagctgttacgaaagatgttactccgcgaaaggttcgagctttgcctcctgtgtggaagtatgtgacaagaaaagtcgatcatgatgagaagtttcttcttagtttaatgagggagaaaggcattacttatagttcagctcctcaatctgcattgatgtcaatgagaagtactttgataacaatcatctctttgtggattcctttaacacctcttatgtggcttctttatcggcaactctcagcatctaacagccctgcgaaaaaacgacggtctaaaaatcccacggttggattcgatgatgttgagggtgttgattccgctaaagacgagctcgtggagatagtgtcatgtcttcaaggaagtataaactacaaaaaactaggagcaagattacctagaggtgtgcttttggtcggtccaccagggacaggtaaaaccttgttggctcgggctgtagctggagaggcgggagttccatttttctctgtttccgctagcgagttcgttgaaatgtttgttggaagaggcgcagcacgaatcagagatcttttcaattcagccaggaaaaactcaccttccattatatttattgatgagcttgatgcggttggaggaaaacgcggtaggagtttcaacgatgaacgtgaccagacactaaatcagttgcttaccgagatggatggatttgagtcagacacgaaagtcattgttattgcagcaactaaccggccagaatcattagacccggctctttgtcggcctggaaggttctctagaaaagttgtggttgcagaaccagaccaagaaggccgtcggaaaatcttggccgtacatctgagagacgtgcctctagaagaagatgcgtttctcatatgtgatctggttgcttctctaactcccggatttgtaggtgctgatctcgccaatattgttaatgaagctgcattgctcgctgctcgtagaggcggggaagtggtggcacgggaagatataatggaagcgatagagagggcgaaattcgggatcaatgataaggaagtgaggtcaaggacactaggaaatgagctgagcaagctgttcccatggatgccatctttggctagaaggaacggaccagatcaagacggtttacaaggaccgttaggctatcaaactctcagctaa

SEQ ID NO: 32 - protein, 624 aa, Boechera stricta
  > Bostr.1460s0159
MATFNLLCSNRFLFKGGSSPEKSSRKVSSRSSELNVCVARTRNQSFSCRRLGGFLEIGETRLGVRVHGDSRNGFSCSSEIKRLMSGDYGDKETRIGENGRNNGKRRRFSLRLRPRLRLVSMRLGRFHLRASMEDFRLFLKKNFKRVILSTGVAVIFGLCYLFLRLTAVPSPSIVPYSDFVTNLRGGSVSKVLLEEGSRRIYYNTDENVEVVEDVHKSETLEEPAIQNDGRTMETVTEAVTKDVTPRKVRALPPVWKYVTRKVDHDEKFLLSLMREKGITYSSAPQSALMSMRSTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVEMFVGRGAARIRDLFNSARKNSPSIIFIDELDAVGGKRGRSFNDERDQTLNQLLTEMDGFESDTKVIVIAATNRPESLDPALCRPGRFSRKVVVAEPDQEGRRKILAVHLRDVPLEEDAFLICDLVASLTPGFVGADLANIVNEAALLAARRGGEVVAREDIMEAIERAKFGINDKEVRSRTLGNELSKLFPWMPSLARRNGPDQDGLQGPLGYQTLS

SEQ ID NO: 33 - DNA, 1962 bp, Citrus clementina
  > Ciclev10011254m.g
atgagttctttttctgttgtttgcaatagtgggttcttggcaactcagcataacttaggtgtttatagtggaaaaatcaagtctttgcggagatattcttacagtagtcatacttcatttcgctttccttcagtgggattttataattgttgcaaaagttcacaatgcttgttaggttgtaacaataggtttaggaggtttctcaatggtggaatagttggagctcccttgttgagctttagtgtttgttgtaagtctcgaaatggggcgtcttgtaatagtgaaattaggccggtaatgagtggaaacagtggggataaagaaacccatttggggaaacgaggtaatgggaagttgagaaggcagtcttcgttaagattgcgtccacggttgcggttattagcactgagattgaagagggtttcgttgaggtcagtgttgaatgatgttgtgatgttcttgaggaagaatataaggagggtgactttgtccactttgatatcttttgtattggggatgtgctatttattcctgaaattgacagcatcaccatcaacaaaggttgttccatactcggacttgataatgagccttcagagtggctctgttacaaaagttttacttgaggaagggtctcgtcgtatatattataacacgaatttgcagggccctgatactcagattgcagaagaaaaatcacctgtagtgagtgagccagttgagaatgtagaagtaaatcaaccagttgaggatgtagccaatacagttgccaaaaatagtggtgcaagaacaggtcaggcagtgaatgtatggaagaaattctcaaggagtcagtcttctattccagagtggcaattttccaccaggaaaatagatcatgatgagaaatttcttcttagtttgatgagggaaaagggaataacatatagctcagctcctcaatctccattggcatcgatgagaaatattttaataactgtgatatctttgtggattcccttgtctcctttaatgtggcttctttatcgtcagctttctgctgctaatagcccagccaaaaagcgccggcctaacaaacagatggttggctttgatgatgtggagggtgttgatgctgctaaggttgagctcatggagatagtttcatgtcttcaaggagctataaactacaaaaaattaggagcaaaattacctcgaggtgtattgcttgtgggtccccctggaactgggaaaacattgcttgcccgtgcagtggccggtgaagcaggagtgccattttttactgtttctgccagtgaatttgtggagttatttgttggaagaggggcagctcgcattagagacctctttagtgcagcacgaaaatgtgcaccagcaataatattcattgatgaacttgacgcagttggaggaaagcgtggtagaagtttcaatgatgaacgtgatcaaacactaaatcagttgctgacagaaatggatggatttgagtcagatgcaaaagtggttgttattgcagcaactaacaggtcagaagcattggatccagccctatgtcgccctggtcgcttctctagaaaagtgcttgtaggagaacctgatgaggaaggaagaagaaagatcttggctgtacatttgagaggaattcctctagaagaagatacacagcttatctgcgatctagttgcttctctgacccctggctttgtgggtgctgatctagcaaatattgttaatgaagctgctttgcttgctgctcgtggaggcagtgaaactgtgacaagggaaaatataatggaagctatagaaagagcaaagtttggaatcaatgatagacagcagcggccgagtacaataagcaaagagcttggaaagctcttcccatggatgccatctctcatgggaagaaatgacacaaaacaagatggtttgcaagggcccatgggctatcaaactctaagctga

SEQ ID NO: 34 - protein, 653 aa, Citrus clementina
  > Ciclev10011254m.g
MSSFSVVCNSGFLATQHNLGVYSGKIKSLRRYSYSSHTSFRFPSVGFYNCCKSSQCLLGCNNRFRRFLNGGIVGAPLLSFSVCCKSRNGASCNSEIRPVMSGNSGDKETHLGKRGNGKLRRQSSLRLRPRLRLLALRLKRVSLRSVLNDVVMFLRKNIRRVTLSTLISFVLGMCYLFLKLTASPSTKVVPYSDLIMSLQSGSVTKVLLEEGSRRIYYNTNLQGPDTQIAEEKSPVVSEPVENVEVNQPVEDVANTVAKNSGARTGQAVNVWKKFSRSQSSIPEWQFSTRKIDHDEKFLLSLMREKGITYSSAPQSPLASMRNILITVISLWIPLSPLMWLLYRQLSAANSPAKKRRPNKQMVGFDDVEGVDAAKVELMEIVSCLQGAINYKKLGAKLPRGVLLVGPPGTGKTLLARAVAGEAGVPFFTVSASEFVELFVGRGAARIRDLFSAARKCAPAIIFIDELDAVGGKRGRSFNDERDQTLNQLLTEMDGFESDAKVVVIAATNRSEALDPALCRPGRFSRKVLVGEPDEEGRRKILAVHLRGIPLEEDTQLICDLVASLTPGFVGADLANIVNEAALLAARGGSETVTRENIMEAIERAKFGINDRQQRPSTISKELGKLFPWMPSLMGRNDTKQDGLQGPMGYQTLS

SEQ ID NO: 35 - DNA, 1860 bp, Capsella grandiflora
  > Cagra.1194s0060
atggctactttcaatgttctttgtagcaatcggtttctgttcaaaggggctaattctccggagaattctagcagaagggtgtcctccaggagtagtgagtttaatgtttgtgtcgctagagcgaggaaccaaacgtttagttgcaggaggctcggtgggtttcttgaaattggtgattccagattaggggttagatttcatggcgatactcgaaataaattttcttgtaatagtgagattaagcgacttttgagtggtgattacggagataataaggagtctcgtattggtgaagatggtagaagcaaaaggaagagaagaagattttcccttaggcttcgacctagattacgattggtgaccatgagacttgggagattcgatttcagagcatcaattgaggaatttcgattctttttaaaaaagaatttcaaaagagtggttttatcaactggtgtagctgtaatctttggactgtgttatttgttcttgaggttgaccgctgttccgtctccatccattgttccttactcagatttcgttaccaatcttcgaggtggttccgtttcaaaggttttgctcgaagaaggttctcgtcgaatctattacaatactaatgaggttgttgaagatgatcaaactttagaagaaccggcactccaaactgatggcagcacagcggaaactgtgaccgaagctgttacgaaagatgttactccgcggaaggttcgggctttgcatcctgtgtggaagtatatgacaagaaaagtcgatcatgatgagaagtttcttctttgtttaatgagggagaaaggcattacttatagctcagctcctcaatctgcattgatgtcaatgagaaccactttgataacaatcatctctctgtggattcctttaacacctcttatgtggcttctttaccggcaactctcagcatctaacagccctgcgaaaaaacgacggtctaaaaatcccacggttggatttgatgatgttgagggtgttgattctgctaaagacgagcttgtggagatagtgtcatgtcttcaaggaagtataaactacaaaaaactaggagcacgattacctagaggtgtgcttttggtcggtccaccagggaccggtaagaccttgttagcccgggctgttgctggagaggctggagttccatttttctcggtttccgctagtgaattcgttgaattgtttgttggaagaggtgcagcgcggatcagagatcttttcaatgctgccaggaaaaactcaccttccataatatttattgatgagcttgatgcggtgggaggaaaacggggtaggagtttcaacgatgaacgtgaccagacactaaaccagttgcttactgagatggatggattcgagtcagagacgaaaatcattgtaattgcagcaactaaccgaccagaagcattagacccggctctttgtcggcctggaaggttctctagaaaagttgtggttgcagaaccagaccaagaaggccgtaggaaaatcttggccgtacatctgagagacatccctctagaagaagatgcgttcctcatatgtgatttggttgcttctctaactcctggatttgtaggtgctgatctggccaatattgtcaatgaagctgcattgctcgctgctcgtagaggcggggaaacggtggcacgggaagatataatggaagcgatagagagagcaaagtttgggataaatgataaggaagtgaggtcaaagacattaggaaatgagctgagcaagctgttcccatggatgccatctttggctagaaggaacggaccagatcaagacggtttacaaggaccgttaggttatcaaagtctcagctag

SEQ ID NO: 36 - protein, 619 aa, Capsella grandiflora
  > Cagra.1194s0060
MATFNVLCSNRFLFKGANSPENSSRRVSSRSSEFNVCVARARNQTFSCRRLGGFLEIGDSRLGVRFHGDTRNKFSCNSEIKRLLSGDYGDNKESRIGEDGRSKRKRRRFSLRLRPRLRLVTMRLGRFDFRASIEEFRFFLKKNFKRVVLSTGVAVIFGLCYLFLRLTAVPSPSIVPYSDFVTNLRGGSVSKVLLEEGSRRIYYNTNEVVEDDQTLEEPALQTDGSTAETVTEAVTKDVTPRKVRALHPVWKYMTRKVDHDEKFLLCLMREKGITYSSAPQSALMSMRTTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESETKIIVIAATNRPEALDPALCRPGRFSRKVVVAEPDQEGRRKILAVHLRDIPLEEDAFLICDLVASLTPGFVGADLANIVNEAALLAARRGGETVAREDIMEAIERAKFGINDKEVRSKTLGNELSKLFPWMPSLARRNGPDQDGLQGPLGYQSLS

SEQ ID NO: 37 - DNA, 1905 bp, Carica papaya
  > evm.TU.supercontig_652.5
atggctgcattcgctgtcgtgtgcaatgatggacttttggttaatcaacaaatttctattgctggcaatagaagaacggaagccttgaagagacgtcaaagtgtctgttttagtaaaatccagaatcgcttatttggtggcactaagtttaaatcgcttaataatgggaaaactggagtttctttgttgggattttatgtttgttgtaaatctcaaagtgggttgtcttgtaacaatgaagttaggcaattgatgagtagaaatggtggagacaagcaggcccatttgggggagagagaaagtaatgatttgaggaagagattctcagtgaggctacgtccaaggttgcggttattaattcgaagaatcaagagagcttctgcgaggtcaattttggatggtattgggatgcttttgaggaggaacataaaaagggtgattttgtcagttgcgatatctgtggcactggggctgtgttatttgtttttgaaattgacggccttgccatcttcgaggatagttccatattccgacttcgtaacaagccttcaaacgggctctgttacaaatgttttacttgaagagggttcccgtcgtatatattataatttaaatgataagaacataaaaactgcacatgctttggaagaaaagtcgccagaaatgaacgcttctgtagaggagatagttgacacagttccagttgcaagagatgatggttcagaaactgaccaagctccaaaagctaatgtattgaagaaattgtcaagaagtaaagattccgtccctgagtggcagtattccactagaaaaatagatcatgatgagaaatttcttctcagtttgatgagagagaagggaatcacatacagctcagctcctcaatctgttttgcattccatgagaaatactttaataactataatctctctgtggattccattgactcctttgatgtggcttctttaccgtcaactttccgcagctaacaacccagcaagaaagaggcgacccaataatcagatggttagctttgacgatgtggagggtgtggatgctgccaagcaagagcttgttgagatagtttcatgcctacaaggagctataaactaccaaagactgggagcaaagttgcctagaggtttgttgttggtgggtccccctggaaccgggaaaacattactggctcgagcagtggctggggaagcaggagtgccatttttcactgtttctgccagcgaatttgtagaactgtttgttggaagaggagcagcacgcattagagatctttttaatgtagcaaagaaatatgcaccatctattatattcattgatgagcttgatgcagttggagggaagcggggtagaagtttcaatgatgaacgtgatcaaacgctgaaccagttgctcactgagatggatggttttgagtcagacatgaaggtgatagttattgcagcaactaatcgtccagaagcactggaccctgccctgtgccgccctggtcgtttctcaaggaaggttattgtaggtgaaccagatcaggagggaagaagaaagattttggcggtacatttgcggaaagttcctttggaggaagacatacatcttatttgtgatctagttgcttctctcaccccaggtttcgttggtgctgatcttgcaaatatcatcaacgaagctgctttacttgctgctcgtagaggtggtgaaatcgtgaccagggaagatataatgggatctattgaaagagcaaaatttggaataaatgataggccggtgagggctagtacaattagtaaggagctcgggaaactgtttccctggatgccatctttgatgggaaggagcaacccaggagaagatggtttactaggaccgtttggctatcaaactctcagctaa

SEQ ID NO: 38 - protein, 634 aa, Carica papaya
  > evm.TU.supercontig_652.5
MAAFAVVCNDGLLVNQQISIAGNRRTEALKRRQSVCFSKIQNRLFGGTKFKSLNNGKTGVSLLGFYVCCKSQSGLSCNNEVRQLMSRNGGDKQAHLGERESNDLRKRFSVRLRPRLRLLIRRIKRASARSILDGIGMLLRRNIKRVILSVAISVALGLCYLFLKLTALPSSRIVPYSDFVTSLQTGSVTNVLLEEGSRRIYYNLNDKNIKTAHALEEKSPEMNASVEEIVDTVPVARDDGSETDQAPKANVLKKLSRSKDSVPEWQYSTRKIDHDEKFLLSLMREKGITYSSAPQSVLHSMRNTLITIISLWIPLTPLMWLLYRQLSAANNPARKRRPNNQMVSFDDVEGVDAAKQELVEIVSCLQGAINYQRLGAKLPRGLLLVGPPGTGKTLLARAVAGEAGVPFFTVSASEFVELFVGRGAARIRDLFNVAKKYAPSIIFIDELDAVGGKRGRSFNDERDQTLNQLLTEMDGFESDMKVIVIAATNRPEALDPALCRPGRFSRKVIVGEPDQEGRRKILAVHLRKVPLEEDIHLICDLVASLTPGFVGADLANIINEAALLAARRGGEIVTREDIMGSIERAKFGINDRPVRASTISKELGKLFPWMPSLMGRSNPGEDGLLGPFGYQTLS

SEQ ID NO: 39 - DNA, 1860 bp, Capsella rubella
  > Carubv10016037m.g
atggctactttcaatgttctttgtagcaataggtttctgttcaaaggggctaattctccggagaattctagcagaaaggtgttttccaggagtagtgagtttaatgtttgtgtcgctagagcgaggaaccaaacgtttagttgcaggaggctcggtgggtttcttgaaattggtgattccagattaggggttagatttcatggcgatactcgaaataagttttcttgtaatagtgagattaagcgacttttgagtggtgattacggagataataaggagtctcgtattggtgaagatggtagaagcaaaaggaagagaagaagattttcccttaggcttcgacctagattacgattggtgaccatgagacttgggagattcgatttcagagcatcaattgaggaatttcgattctttttaaaaaagaatttcaaaagagtggttttatcaactggtgtagctgtaatctttggactgtgttatttgttcttgaggttgaccgctgttccgtctccatccattgttccttactcagatttcgttaccaatcttcgaggcggttccgtttcaaaggttttgctcgaagaaggttctcgtcgaatctattacaatactaatgaggttgttgaagatgatcaaactttagaagaaccggcactacaaactgatggcagcacagcggaaactgtgaccgaagctgttacgaaagatgttactccgcggaaggttcgagctttgcctcctgtgtggaagtatatgacaagaaaagtcgatcatgatgagaagtttcttcttggtttaatgagggagaaaggcattacttatagctcagctcctcaatctgcattgatgtcaatgagaaccactttgataacaatcatctctctgtggattcctttaacacctcttatgtggcttctttaccggcaactctcagcatctaacagccctgcgaaaaaacgacggtctaaaaatcccacggttggatttgatgatgttgagggtgttgattctgctaaagacgagcttgtggagatagtgtcatgtcttcaaggaagtataaactacaaaaaactaggagcacgattacctagaggtgtgcttttggtcggtccaccagggaccggtaagaccttgttagcccgggctgtcgctggagaggctggagttccatttttctcggtttccgctagtgaattcgttgaattgtttgttggaagaggtgcagcgcggatcagagatcttttcaatgctgccaggaaaaactcaccttccataatatttattgatgagcttgatgcggtcggaggaaaacggggtaggagtttcaacgatgaacgtgaccagacactaaaccagttgcttactgagatggatggattcgagtcagagacgaaaatcattgtaattgcagcaactaaccgaccagaagcattagacccggctctttgtcggcctggaaggttctctagaaaagttgtggttgcagaaccagaccaagaaggccgtaggaaaatcttggccgtacatctgagagacatccctctagaagaagatgcgttcctcatatgtgatttggttgcttctctaactcctggatttgtaggtgctgatctggccaatattgtcaatgaagctgcattgctcgctgctcgtagaggcggggaaacggtggcacgggaagatataatggaagcgatagagagagcaaagtttgggataaatgataaggaagtgaggtcaaagacattaggaaatgagctgagcaagctgttcccatggatgccatctttggctagaaggaacggaccagatcaagacggtttacaaggaccgttaggttatcaaagtctcagctag

SEQ ID NO: 40 - protein, 619 aa, Capsella rubella
  > Carubv10016037m.g
MATFNVLCSNRFLFKGANSPENSSRKVFSRSSEFNVCVARARNQTFSCRRLGGFLEIGDSRLGVRFHGDTRNKFSCNSEIKRLLSGDYGDNKESRIGEDGRSKRKRRRFSLRLRPRLRLVTMRLGRFDFRASIEEFRFFLKKNFKRVVLSTGVAVIFGLCYLFLRLTAVPSPSIVPYSDFVTNLRGGSVSKVLLEEGSRRIYYNTNEVVEDDQTLEEPALQTDGSTAETVTEAVTKDVTPRKVRALPPVWKYMTRKVDHDEKFLLGLMREKGITYSSAPQSALMSMRTTLITIISLWIPLTPLMWLLYRQLSASNSPAKKRRSKNPTVGFDDVEGVDSAKDELVEIVSCLQGSINYKKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESETKIIVIAATNRPEALDPALCRPGRFSRKVVVAEPDQEGRRKILAVHLRDIPLEEDAFLICDLVASLTPGFVGADLANIVNEAALLAARRGGETVAREDIMEAIERAKFGINDKEVRSKTLGNELSKLFPWMPSLARRNGPDQDGLQGPLGYQSLS

SEQ ID NO: 41 - DNA, 1600 bp, Cucumis sativus
  > Cucsa.238330
agggaacaacaatttgagaagaaagtttggtttaagattacgcccaagattgagattgttatctagaagattgaaaactgtttctgttaggtcaactttgaacaatgtagggacgtttattaggaagaatgttagaaaagttaccctttctgcttcagtttctattgttttggcgatttgctatttgtttctgaaaataacagcagtcccaccttccaagaatgttccatattcagatttcattaccagtcttcagagtggaactgtcactaaagttttactcgaggaggggtctcgtcgtatatactttaatcgaagttttacagaatcgattcaaggtttagatggtcaaccattagcagtagctgtagatagttctaatggaagtgataagggaatgagtgaagattcaagaactgttcaagccccaagaacgaatttattgacaaaaatttcaagaaggaaaagccgtgtcgcaattcctgagtggcaattctcgactaggaaagtagatcgcgatgaaaagtttctgcttggtttgatgagagaaaagggaactacttacagttctgcacctcagtcagttttgatgtcgatgagaactacattgataacaatcatatctctatggattcctttaattcctctcatgtggcttctctatcgtcaactttctgcatcgaatacaactgcaaaaaagcgacaaccaaacagccccatggttggatttgaagacgtggaaggcgttgatggtgctaaagtggagctcatggaggtagtatcatgccttcaaggagctatgaactaccaaaaactaggagcaaagcttcctaggggtgtcctgttagtaggacctcctggaactgggaaaacactccttgcacgagctgtggcaggagaggcaggggttccgtttttcagtgtttctgccagtgaatttgttgaaatgtttgttggaagaggggcagcacgtattagagacctttttaatgtggcaagaaaatgtgcaccatcaatcgtattcatagatgaacttgatgcggttggagggaaacggggtagaagtttcaatgatgaacgtgaccaaacattaaaccagctgttgactgaaatggatggattcgagtcggatatgaaagtgattgttatagcagcaactaacagacccgaagcattggatcccgccctatgtagaccaggtagattctctagaaaagttctcgtaggcgaacctgatgaagaaggaaggagaaaaatcttggcagtacatttaagagaagttccgctcgaggaagacctcaacttaatctgcagtctagtagcagcactcacacctggttatgtaggcgctgatctagcaaatattgttaacgaatcagcactactcgcagctcgtagaggtggcgaaatgatatcgagggaagatttaatgggagccattgaaagatcaaaatttgggatcaacagcaagcaactaaggtctaatacaataagcaatgagcttgaaagactttttccatggatgacatctctaatgggaagaaatgacaaaagagtggatccatcacaagggccattaggctatcaaactttgagctga

SEQ ID NO: 42 - protein, 532 aa, Cucumis sativus
  > Cucsa.238330
GNNNLRRKFGLRLRPRLRLLSRRLKTVSVRSTLNNVGTFIRKNVRKVTLSASVSIVLAICYLFLKITAVPPSKNVPYSDFITSLQSGTVTKVLLEEGSRRIYFNRSFTESIQGLDGQPLAVAVDSSNGSDKGMSEDSRTVQAPRTNLLTKISRRKSRVAIPEWQFSTRKVDRDEKFLLGLMREKGTTYSSAPQSVLMSMRTTLITIISLWIPLIPLMWLLYRQLSASNTTAKKRQPNSPMVGFEDVEGVDGAKVELMEVVSCLQGAMNYQKLGAKLPRGVLLVGPPGTGKTLLARAVAGEAGVPFFSVSASEFVEMFVGRGAARIRDLFNVARKCAPSIVFIDELDAVGGKRGRSFNDERDQTLNQLLTEMDGFESDMKVIVIAATNRPEALDPALCRPGRFSRKVLVGEPDEEGRRKILAVHLREVPLEEDLNLICSLVAALTPGYVGADLANIVNESALLAARRGGEMISREDLMGAIERSKFGINSKQLRSNTISNELERLFPWMTSLMGRNDKRVDPSQGPLGYQTLS

SEQ ID NO: 43 - DNA, 1962 bp, Citrus sinensis
  > orange1.1g043863m.g
atgagttctttttctgttgtttgcaatagtgggttcttggcaactcagcataacttaggtgtttatagtggaaaaatcaagtctttgcggagatattcttacagtagtcatacttcattttgctttccttcagtgggattttataattgttgcaaaagttcacaatgcttgttaggttgtaacaataggtttaggaggtttctcaatggtggaatagttggagctcccttgttgagctttagtgtttgttgtaagtctcgaaatggggcgtcttgtagtagtgaaattaggccggtaatgagtggaaacagtggggataaagaaacccatttggggaaaagaggtaatgggaagttgagaaggcggtcttcattaagattgcgtccacggttgcggttattagcactgagattgaagagggtttcgttgaggtcagtgttgaatgatgttgtgatgttcttgaggaagaatataaggagggtgactttgtccactttgatatcctttgtattggggatgtgctatttattcctgaaattgacagcatcaccatcgacaaaggttgttccatactcggacttgataacgagccttcagagtggctctgttacaaaagttttacttgaggaagggtctcgacgtatatattataacacgaatttgcagggccctgatactcagattgcagaagaaaaatcacctgtagtaagtgagccagttgagaatgtagaagtaaatcaaccagttcaggatgtagccaatacagttgccaaaaacagtggtgcaagaacaggtcaggcagtgaatgtatggaagaaattctcaaggagtcagtcttctattccagagtggcaattttccaccaggaaaatagatcatgatgagaaatttcttcttagtttgatgagggaaaaggggataacatatagctcagctcctcaatctccattggcatcgatgagaaatattttaataaccgtgatatctttgtggattcccttgtctcctttaatgtggcttctttatcgtcagctttctgctgctaatagcccagccaaaaagcgccggcctaacaaacagatggttggctttgatgatgtggagggtgttgatgctgctaaggttgagctcatggagatagtttcatgtcttcaaggagctataaactacaaaaaattaggagcaaaattacctcgaggtgtattgcttgtgggtccccctggaactgggaaaacattgcttgcccgtgcagtggccggtgaagcaggagtgccgttttttactgtttctgccagtgaatttgtggagttatttgttggaagaggggcagctcgcattagagacctctttagtgcagcacgaaaatgtgcaccagcaataatattcattgatgaacttgacgcagttggaggaaagcgtggtagaagtttcaatgatgaacgtgatcaaacactaaatcagttgctgacagaaatggatggatttgagtcagatgcaaaagtggttgttattgcagcaactaacaggccagaagcattggatccagccctatgtcgccctggtcgcttctctagaaaagtgcttgtaggagaacctgatgaggaaggaagaagaaagatcttggctgtacatttgagaggaattcctctagaagaagatacacagcttatctgcgatctagttgcttctctgacccctggctttgtgggtgctgatctagcaaatattgtcaatgaagctgctttgctagctgctcgtggaggcagtgaaactgtgacaagggaaaatataatggaagctatagaaagagcaaagtttggaatcaatgatagacagcagcggccgagtacaataagcaaagagcttggaaagctcttcccatggatgccatctctcatgggaagaaatgacacaaaacaagatagtttgcaagggcccatgggctatcaaactctaagctga

SEQ ID NO: 44 - protein, 653 aa, Citrus sinensis
  > orange1.1g043863m.g
MSSFSVVCNSGFLATQHNLGVYSGKIKSLRRYSYSSHTSFCFPSVGFYNCCKSSQCLLGCNNRFRRFLNGGIVGAPLLSFSVCCKSRNGASCSSEIRPVMSGNSGDKETHLGKRGNGKLRRRSSLRLRPRLRLLALRLKRVSLRSVLNDVVMFLRKNIRRVTLSTLISFVLGMCYLFLKLTASPSTKVVPYSDLITSLQSGSVTKVLLEEGSRRIYYNTNLQGPDTQIAEEKSPVVSEPVENVEVNQPVQDVANTVAKNSGARTGQAVNVWKKFSRSQSSIPEWQFSTRKIDHDEKFLLSLMREKGITYSSAPQSPLASMRNILITVISLWIPLSPLMWLLYRQLSAANSPAKKRRPNKQMVGFDDVEGVDAAKVELMEIVSCLQGAINYKKLGAKLPRGVLLVGPPGTGKTLLARAVAGEAGVPFFTVSASEFVELFVGRGAARIRDLFSAARKCAPAIIFIDELDAVGGKRGRSFNDERDQTLNQLLTEMDGFESDAKVVVIAATNRPEALDPALCRPGRFSRKVLVGEPDEEGRRKILAVHLRGIPLEEDTQLICDLVASLTPGFVGADLANIVNEAALLAARGGSETVTRENIMEAIERAKFGINDRQQRPSTISKELGKLFPWMPSLMGRNDTKQDSLQGPMGYQTLS

SEQ ID NO: 45 - DNA, 1863 bp, Daucus carota
  > DCAR_026283
atggcatgtctacccattgtttcaaatgatgggttcttgatttctcagcgaaatattagggctagcagtgtaaaaattgaatcttggggtggatacagaggtgtttcttgctcatttgctaattcttgttgttattctggctgtttccctatattgggattgtacaattgctcgaaaactaggcagagattggtgttggatagtaagattaggtcggtgtcgaatgagaaggagaatacgaaaccccatttgggaaatagagttaatggtggcttaaagaagaaatttttgataagattgaggccaagattgaggttgttgtcgaggagattgagaatgggctcggtcaagtgtttgttggatagagtgagtacgtatgtgcgtaagaatttgaaaagagtgacgatttctgccgcggttactgttgtgttagggttgtgctttttgtttcttaagctgactgcggttccttccccgaaatttgttccgtattcggacttggttatgagcttgcagagtgggtcggtgacgagagttctgtttgaggagggatcgcggaggatatactacaatactgacgtgcaaagtggtgaaaatgctgaacaagttgaaggtgagtcggtgttgaggactgccgaggatggtaaatctgaaaacggggttgagggaattgaggcgtcaaggaatagtatgagtaaacagtcaagtatgttgaaaaagttggcgaagggaaaaccgtcggcaccactgtggcagtactccactagaaagatagatcatgatgagagctatcttcttagtttgatgagagaaagtggaacttcgtatgcatcagctcctcaatcagctcttgcatcagtacggagtatcatactcaccattttaactctttggattccactgacacctttaatgtggatcctttatcgtcagctttctgctagtaatagcccagcaaagaaaaaacgacctagcaatcaagtagtcagctttgaggacgtcgagggagttgatgctgcaaaagtagagctcatggagatagttttgtgtttgcaaggttctattgactacaccagactaggagcaaaattacctagaggagtgcttctagtgggtcctccgggaactgggaaaactttactagcccgtgctgtggcaggagaagctggagtgccattttttactgtgtctgccagtgaattcgtcgaaatgtttgttggaagaggagcagctcgtatcagagacctttttagtgttgcaaggaagagttcaccgtcgatcattttcatcgatgagcttgatgctgttgggggcaggcgtggaagaagtttcaatgatgaaagggaccaaacattgaaccagctgctgaccgaaatggatggctttgattcagaagtaaatgtgattgtcatagcagcaactaataggccagaagcattagatcccgctctttgccgtcctggacgtttctcaaggaaagttttggtaggagaaccagatgaagatggaaggcgcaagattttggctgtgcatctaagaggaattcctcttgaggaagatactggccttatctgtgatctagtagcatcagttacccagggatttgtaggtgctgatctagcaaacatcgtcaatgaagctgctcttctagctgctcgccgaggtagtgaatgtgtggcaagggaagatataatggcagcaatcgaaagagccaagtttgggataaatgataagcaactgactcgtaatacaataagcaaggaaataggaaagctctttccttggatgccatcttttataggcaagaatgacacgcagcggaatggaacacaaggacccttgggttatcaaacactgagctga

SEQ ID NO: 46 - protein, 620 aa, Daucus carota
  > DCAR_026283
MACLPIVSNDGFLISQRNIRASSVKIESWGGYRGVSCSFANSCCYSGCFPILGLYNCSKTRQRLVLDSKIRSVSNEKENTKPHLGNRVNGGLKKKFLIRLRPRLRLLSRRLRMGSVKCLLDRVSTYVRKNLKRVTISAAVTVVLGLCFLFLKLTAVPSPKFVPYSDLVMSLQSGSVTRVLFEEGSRRIYYNTDVQSGENAEQVEGESVLRTAEDGKSENGVEGIEASRNSMSKQSSMLKKLAKGKPSAPLWQYSTRKIDHDESYLLSLMRESGTSYASAPQSALASVRSIILTILTLWIPLTPLMWILYRQLSASNSPAKKKRPSNQVVSFEDVEGVDAAKVELMEIVLCLQGSIDYTRLGAKLPRGVLLVGPPGTGKTLLARAVAGEAGVPFFTVSASEFVEMFVGRGAARIRDLFSVARKSSPSIIFIDELDAVGGRRGRSFNDERDQTLNQLLTEMDGFDSEVNVIVIAATNRPEALDPALCRPGRFSRKVLVGEPDEDGRRKILAVHLRGIPLEEDTGLICDLVASVTQGFVGADLANIVNEAALLAARRGSECVAREDIMAAIERAKFGINDKQLTRNTISKEIGKLFPWMPSFIGKNDTQRNGTQGPLGYQTLS

SEQ ID NO: 47 - DNA, 1935 bp, Eucalyptus grandis
  > Eucgr.I02102
atggcttgtctttctgtcatttgcaataatgggttcttcaagtctcgagagctcctgggcgttcgggtcggaaaggccagttcttgtgggcatcggagtcttggcggcggccttgctctgtttcctcttgtgaagttccaaactttgcgcaaattcgaacgtgggtcgtcgtgtaatgctaactcgatgcaaaagctctctagacatgattggattcggcctttaaggtttcatggcggttctaagtggccccctggattgtcctgtagaagtagaattgagcccgggagcgatggatttagtgatattgagcggggttatgtgaggaaaggaagaagaggtattaggaggaaattctcgctgagattgcgcccgagattttggttgttggcctcgagatttaagagggtctcggctatgtccatgttgaatgatttgggggtgttcttgagaaagaatttgaagagagtgactctttcagcttcagtttctgtagcattgggtctttgttatatttttctgaaagtgacagctttgccatctccaaaacttgttccttactcagacttgattatgagccttcaagatggcaatgttactaaggttttgctcgaagaagggtctcgtcggatatattacaacgcgagctcaggaagtcaggtaaatgataaaattccagaggaaaagttgatggagagtaattctccaattggaactgcggttgatataactgaaaagaatgatgttgttccagctggaaagatgatcagtgcaagcatgcctaagaaatttttgagagcacgtgctgctatgccacagtggcaatatgccactcggaagattgatcatgatgaaaagtttcttctaaatttgatgagagaaaaaggaacaatttatagctcagcccctcagtcagtgttgatgtccgtcaggagtaccttgataactataatatcattgtggattcctttgactccattgatgtggcttctctatcgacaactttctgctgctaatagccctgcaaaaaagaggcagcctaatagtcagacagtcaactttgatgatgtcgagggtgttgatgctgctaaggttgagcttatggagatagtctcctgccttcaaggagcaattaactataaaaagctaggggctaagttgcctggaggggtactgctggtgggtcctcctggaactgggaagacattgctggcccgtgcagttgcgggagaagccggagtaccctttttcactgtttctgcgagtgagtttgtggagttgtttgttggaagaggagcagctcgcatcagagacttgtttactgctgcgagaaaatgttccccatcgataatatttatcgatgaactggatgctgttggcggaaagcgtggtagaagcttcaatgatgaacgcgaccaaacattaaaccagctgctgacagaaatggatggatttgagtcggacacaaaagtgattgttcttgcagcaaccaacagacctgaagcattagaccccgctctctgccgtcctggtcgcttctcgagaaaagttcttgtgggagagccggatgaggaaggaaggagaagaattttgggtgtacatctgagaggagttcctcttgatgaagacattgagattatatgtgatctagtagcttctcttaccccgggatttgtgggcgctgatcttgcaaatattgtgaatgaagcagctctgctagctgctcgtagaggtggtgaaacggtgttaagggaggatattatggaagcagttgaaagagctaaatatggtatcaatgatcagcaactaaggccgaatacaatcagcaaggagctaggaaaattgtttccttggattccatccataatgggaaggaatgacacaagagaagatggacttcaaggccccatgggttatcaaactctaagcggataa

SEQ ID NO: 48 - protein, 644 aa, Eucalyptus grandis
  > Eucgr.I02102
MACLSVICNNGFFKSRELLGVRVGKASSCGHRSLGGGLALFPLVKFQTLRKFERGSSCNANSMQKLSRHDWIRPLRFHGGSKWPPGLSCRSRIEPGSDGFSDIERGYVRKGRRGIRRKFSLRLRPRFWLLASRFKRVSAMSMLNDLGVFLRKNLKRVTLSASVSVALGLCYIFLKVTALPSPKLVPYSDLIMSLQDGNVTKVLLEEGSRRIYYNASSGSQVNDKIPEEKLMESNSPIGTAVDITEKNDVVPAGKMISASMPKKFLRARAAMPQWQYATRKIDHDEKFLLNLMREKGTIYSSAPQSVLMSVRSTLITIISLWIPLTPLMWLLYRQLSAANSPAKKRQPNSQTVNFDDVEGVDAAKVELMEIVSCLQGAINYKKLGAKLPGGVLLVGPPGTGKTLLARAVAGEAGVPFFTVSASEFVELFVGRGAARIRDLFTAARKCSPSIIFIDELDAVGGKRGRSFNDERDQTLNQLLTEMDGFESDTKVIVLAATNRPEALDPALCRPGRFSRKVLVGEPDEEGRRRILGVHLRGVPLDEDIEIICDLVASLTPGFVGADLANIVNEAALLAARRGGETVLREDIMEAVERAKYGINDQQLRPNTISKELGKLFPWIPSIMGRNDTREDGLQGPMGYQTLSG

SEQ ID NO: 49 - DNA, 1935 bp, Eucalyptus grandis; identical to SEQ ID NO: 47
  > Eucgr.I02102

SEQ ID NO: 50 - protein, 644 aa, Eucalyptus grandis; identical to SEQ ID NO: 48
  > Eucgr.I02102

SEQ ID NO: 51 - DNA, 1854 bp, Eutrema salsugineum
  > Thhalv10020311m.g
atggctacttttaatgttctctgtagtaatcgttttcatctcaaaggaggcaattttccggagaaatctagcagaatagtatcttcgcggtgtagtgagcttaatatttgtgtcgctaggacgaagaatcagtcgtttggttgcaggagacttggtgggtttttggagattggggaaactagatcagggtttagacttcacggcgattctcggaatgggtttgcttgtaatagcgagattaagcgactagtcaatggtgattacggagataaggagactcgtattggtgaaaatggtagaaacaaagagaagagaagaagattttctcttaggcttcgacctagattacgattgttgagcatgcgacttggaagattcgatttgagggcatcaattgaggatttccggttatttttgagaaagaatatcaaaagagtgatcctatctactggtgtggctgttatcttcggactgtgttatgtgttcttgaggttaaccgctgttccgtctccttccattgttccttactcagactttgtaacgaatcttcgaggtggttccgtttcaaaggttttgctcgaagaaggttctcgtcgaatctactacaacactaatgataatgttgaagatgatcacaagtctgaaacttcagaagaacctgcgatccaagtggaaaccgcgactgaagctactgccaaagacgttattatgccgcgaaaggttcgagctttgactcctgtgtggaagtatgtgacgagaaaagtagatcacgatgagaagtttcttcttagtttgatgagggagaaagggataacttatagttcagctcctcagtctgcattgatgtccatgagaaatactttgataacaatcatttctttatggattcctttaacacctcttatgtggcttctctatcggcaactctctgcatctaacagccctgcgaaaaaacgacgcactaaaaatcccactgttggattcgaggatgttgagggcgtggattctgctaaagacgagctcgtggagatagtctcatgtcttcaaggaagtataaactacagaaaactaggagcaagattaccaagaggtgtgcttttggttggtccaccggggaccggtaaaaccttgttggctcgggctgtagctggagaggcgggagttccgttcttctctgtttcggctagtgagttcgttgaattgtttgttggaagaggtgcagcgcgaatcagagatcttttcaatgcggccaggaaaaactcaccttccattatattcatcgatgagctcgatgcggttggaggaaaacgagggaggagtttcaacgatgaacgtgaccagacgttaaaccagctgcttactgaaatggatggttttgagtcagacacaaaagtcattgtaattgcagcaactaaccgaccagaagcgttagacgcggctctttgtcggccaggaaggttctcaagaaaagtggtggttgcagaaccagaccaagaaggccgtcgaaaaatcttggccgtacatctgagagacgtgccgctagaagaagacgcatttctcatatgtgatctcgttgcttctctaactcccggattcgtaggtgctgatctcgccaatattgtcaatgaagctgcattgctcgctgctcgtagaggcggagaagcggtggcacgggaagatataatggaagcgatagagagggcgaaatttgggatcaatgataaggaaatgagaccaaggactatcggaaatgagctcagcaggctgttcccatggatgccatctttggttgggaggaatggaccagatcaaggcggtttacaaggaccgttaggttatcaaactctcagctaa

SEQ ID NO: 52 - protein, 617 aa, Eutrema salsugineum
  > Thhalv10020311m.g
MATFNVLCSNRFHLKGGNFPEKSSRIVSSRCSELNICVARTKNQSFGCRRLGGFLEIGETRSGFRLHGDSRNGFACNSEIKRLVNGDYGDKETRIGENGRNKEKRRRFSLRLRPRLRLLSMRLGRFDLRASIEDFRLFLRKNIKRVILSTGVAVIFGLCYVFLRLTAVPSPSIVPYSDFVTNLRGGSVSKVLLEEGSRRIYYNTNDNVEDDHKSETSEEPAIQVETATEATAKDVIMPRKVRALTPVWKYVTRKVDHDEKFLLSLMREKGITYSSAPQSALMSMRNTLITIISLWIPLTPLMWLLYRQLSASNSPAKKRRTKNPTVGFEDVEGVDSAKDELVEIVSCLQGSINYRKLGARLPRGVLLVGPPGTGKTLLARAVAGEAGVPFFSVSASEFVELFVGRGAARIRDLFNAARKNSPSIIFIDELDAVGGKRGRSFNDERDQTLNQLLTEMDGFESDTKVIVIAATNRPEALDAALCRPGRFSRKVVVAEPDQEGRRKILAVHLRDVPLEEDAFLICDLVASLTPGFVGADLANIVNEAALLAARRGGEAVAREDIMEAIERAKFGINDKEMRPRTIGNELSRLFPWMPSLVGRNGPDQGGLQGPLGYQTLS

SEQ ID NO: 53 - DNA, 1938 bp, Fragaria vesca
  > gene03807-v1.0-hybrid
atggcttgtttctctgttgtttgcaatactgggttttaccctgtccataataatttaggagttcatggtggaaagcccaagtcttttggcaaattcagagacttttgttgtgattcatttgcttttagttctcctagatttgatataggttgcaaatttcaacatgggttggtctgggacagtgagctctggccagtcactaatggaaaaagtggagctcatttacagagatataataataatgatggtagtaaatcaaaacatgggttgcgttgttatagcaaaatagggccactgataaatgagaacagtgcaaataagcagatcaatttagggaaaaagggtggcagtaattcgaggaagaggttttcgttaagattgcgtccgagagtgcggttattagctttgaaattgagaagagtgtcgatccggtcaatgttgattgatgttgggatcttattgagaaataatagaagaaaagtgactctttttacttcgatttcgatggcactgggaatgtgctatctttttttaaggttaacagcagtgccgtctccaaaaatggtaccgtattcagaattggttacaagcatacggaatgagtctgtttcgaaagttctacttgaggagggatctcgtaggatatattataatacacactcttcccttgttgggaattctcagttgtcgaatgaagaattaccaagtgatcagactgagaatgtggccgatgaggttgcaagtgatgatggtcaaaaatcaggtcaaacattgaaccggaatgtattgaagaaattatcagtgtctcgatcttcagctcctgaatggcagttttccaccagaaaagtagatcacgatgagaaatttcttcttagtttgatgagagaaaaggggattacatatggctcagctcctcagtcagtgttgatgtcaatgaggactactttgataaccataatatctctgtggattccgttgatgccattgatgtggcttctttatcgtcaactttctgctgctagtagcccagctaaaaagcggcgacctgataatcaattggttgggttcgatgatgtggagggagttgatgctgccaaattagagcttatggagatagttttatgcttgcaaggagccattaactacaacaaactaggagcaaagttacctagaggtgtattgctggtgggtcctccaggaacagggaaaacactacttgctcgtgcagtggctggagaagcaggtgtaccgttttttactgtttctgccagtgaatttgttgagatgtttgttggaagaggggcagctcgcattagagacctatttaatgtggcaaggaaaaattcaccctcaatcatattcattgatgagattgatgcagttggaacaaagcgtggtagaagttttaacgatgagcgtgaccaaacactgaatcagttgctgacagaaatggatggatttgagtcagattcaaaagtagttgttgttgcagcgactaatcggcctgaagtattggatccagccctttgtaggccaggccgcttctcaagaaaagtagttgttggagaaccagacgaagatggaaggagaaagatcttggctgtacatttgagaggagttcctttggaggaagatgctaatcttatcagtaatcttatcgcttctctgacaccaggttttgtgggggctgatctcgcaaacattgtcaatgaagctgctttacttgctgctcgtaggggtggtgagagcgtgactagagaagatgtgatggaagcgattgaaagagcaaagtttggaatcaatgataagcgacttaggcccacaacaataagcaacgagctggggaaactcttcccatggatgccttctctgactggaaagaatagtacaagacaagatggatcgcaagggccattaggataccaagctctgagctga

SEQ ID NO: 54 - protein, 645 aa, Fragaria vesca
  > gene03807-v1.0-hybrid
MACFSVVCNTGFYPVHNNLGVHGGKPKSFGKFRDFCCDSFAFSSPRFDIGCKFQHGLVWDSELWPVTNGKSGAHLQRYNNNDGSKSKHGLRCYSKIGPLINENSANKQINLGKKGGSNSRKRFSLRLRPRVRLLALKLRRVSIRSMLIDVGILLRNNRRKVTLFTSISMALGMCYLFLRLTAVPSPKMVPYSELVTSIRNESVSKVLLEEGSRRIYYNTHSSLVGNSQLSNEELPSDQTENVADEVASDDGQKSGQTLNRNVLKKLSVSRSSAPEWQFSTRKVDHDEKFLLSLMREKGITYGSAPQSVLMSMRTTLITIISLWIPLMPLMWLLYRQLSAASSPAKKRRPDNQLVGFDDVEGVDAAKLELMEIVLCLQGAINYNKLGAKLPRGVLLVGPPGTGKTLLARAVAGEAGVPFFTVSASEFVEMFVGRGAARIRDLFNVARKNSPSIIFIDEIDAVGTKRGRSFNDERDQTLNQLLTEMDGFESDSKVVVVAATNRPEVLDPALCRPGRFSRKVVVGEPDEDGRRKILAVHLRGVPLEEDANLISNLIASLTPGFVGADLANIVNEAALLAARRGGESVTREDVMEAIERAKFGINDKRLRPTTISNELGKLFPWMPSLTGKNSTRQDGSQGPLGYQALS

SEQ ID NO: 55 - DNA, 1917 bp, Glycine max
  > Glyma.13G049800
atggcttgtttttctctaccttgcaatactgggtcatttgctaccccaactttgactcaaagaagatatttgggtgtgtgtggaggtttgtgcactaggtcatttgtttttacttctctagggtttaaccaatgcttcaaatttcaacatgagttggtgtggaacaaggagctagggtattgtggtagtagaagtagaattccctcattgagggttccttattgttgcaagactccacatggtgtttcaagtaaaattgagccacttgtgagtagaagcaagggtgagaggaagactcactatggtaagggtggaagtgatgggctgaggaagagattttcgttgagactgcggccaaggttacggttattggctatgagaatgaaaagggcttccattaggtccattttgaatgaggtggggatctttattcgtaagaacattagagcagtgacattttctgcttcaatttctaccgtgttctgcctttgtttcttgtttctaaaattgactacactcccccctcctaaaagtgttccatattccaacttgatcatcagccttcagaatggatatgttgaaaaggttttagttgaagaggggtcgcgacgcatatattacaatatgaagtcccagcatattgaaaatgatggggtttctggggaagaatctgaagtggcagatgtttcaattgataaggatgttgataagattgggagtgagggtacttctggagctggccaaactccagtggggaatgtactaaagaaattctccaagactcgagcttcaatccctgagtggcagtattccacaagaaaaatagatcatgatgaaaagttccttgttagtttgatgagagaaaaaggggttacatatagctctgcccctcaatctgtactaaggtcaatgaggagtactttgatcactgtgataacactgtggatacctttaattccattgatgtggcttctgtatcgccaactttctgctgctaacagtcccgcaaggaagcagagaccgaatggtcagacagttggatttgatgatgtggaaggcattgattctgcaaaagtagagctcatagagatagtttcatgtctgcaaggggatataaattatcaaaagctaggggcaaagttacctagaggtgtgttgctggttggtcctcctggaactgggaagacactacttgcacgtgcagtggcaggggaagctggtgtaccctttttcactgtatccgccagtgagtttgtggagttgtttgttggaagaggggcagctagaatcagagaccttttcaatgcagcaaggaaatttgcaccatcaatcatattcattgacgaacttgatgcagttggaggcaagcgtggaagaagtttcaatgatgaacgtgaccagacgttaaatcagttgctgacagaaatggatggatttgaatccgagatgagagtggttgtcattgcagcaactaaccggccagaagccttggatccagctctatgtcgccctggtcgtttctcaagaaaagtatatgtaggtgaacctgatgaagaaggaaggagaaagatattggctgtgcatctaagaggagttcctttggaggaggacactagcatcatttgccatttaattgcttctcttactactggtttagtaggtgctgatctggcaaatgttgtcaatgaggccgctttgcttgctgctcgtagaggtagtgaaactgtggcaagggaagatataatggaagctattgaaagagcgaaattcggaatcaatgacgaacaattgaggtctagtaaaataagcaaggagctaagcaagctattcccttggatgccatcattgatgggaaaaagtgagaggagacaggatgaccagcaaggaccattaggttatcaatccctgagttcatga

SEQ ID NO: 56 - protein, 638 aa, Glycine max
  > Glyma.13G049800
MACFSLPCNTGSFATPTLTQRRYLGVCGGLCTRSFVFTSLGFNQCFKFQHELVWNKELGYCGSRSRIPSLRVPYCCKTPHGVSSKIEPLVSRSKGERKTHYGKGGSDGLRKRFSLRLRPRLRLLAMRMKRASIRSILNEVGIFIRKNIRAVTFSASISTVFCLCFLFLKLTTLPPPKSVPYSNLIISLQNGYVEKVLVEEGSRRIYYNMKSQHIENDGVSGEESEVADVSIDKDVDKIGSEGTSGAGQTPVGNVLKKFSKTRASIPEWQYSTRKIDHDEKFLVSLMREKGVTYSSAPQSVLRSMRSTLITVITLWIPLIPLMWLLYRQLSAANSPARKQRPNGQTVGFDDVEGIDSAKVELIEIVSCLQGDINYQKLGAKLPRGVLLVGPPGTGKTLLARAVAGEAGVPFFTVSASEFVELFVGRGAARIRDLFNAARKFAPSIIFIDELDAVGGKRGRSFNDERDQTLNQLLTEMDGFESEMRVVVIAATNRPEALDPALCRPGRFSRKVYVGEPDEEGRRKILAVHLRGVPLEEDTSIICHLIASLTTGLVGADLANVVNEAALLAARRGSETVAREDIMEAIERAKFGINDEQLRSSKISKELSKLFPWMPSLMGKSERRQDDQQGPLGYQSLSS

SEQ ID NO: 57 - DNA, 1917 bp, Glycine max; identical to SEQ ID NO: 55
  > Glyma.13G049800

SEQ ID NO: 58 - protein, 638 aa, Glycine max; identical to SEQ ID NO: 56
  > Glyma.13G049800

SEQ ID NO: 59 - DNA, 1896 bp, Glycine max
  > Glyma.19G040200
atggcttgtttttcattaccttgcaatactgggtcatttgttaccccaactttggctcaaagaagatattttggggtatgtggaggtttgtgcactagttcatttgcttttacttctctagggtttaaccaatgcttcaaatttcaacatgagttggtttggagaagtagaattccctcattgagggttcctcattgttgcaagactccacatggtgtttcaagtaacaacaaaattgagccacttgtaagtagaagcaagggtgagaaaaagactcactatggtaaggatggaactgataggttgaagaagagattttcgttgagattgaggccgaggttacggttattggctatgagaatgaaaagggcttccattaagtccattttgaatgaattggggatccttattcgtaagaacattagagcagtggcgttttctgcttcaatttctactgtgttctgcctttgtttcttgtttctgaaattgactgcactcccccctccgaaatctgttccgtattctgacttgatcatcagccttcagaatggacatgttgaaaaggttttagttgaagaggggtctcgacgcatatattacaatatgaagtcccagaatattgaaaatgatccggtttctggggaagaatctgaagtggcagatgcttcaattgataaggatgttgataagattgggagtgagggtacttctaaagctggccaaactccagtggggaatgtactaaaaaaattctctaagactcgagcttcaatccctgagtggcagtattccacaagaaaaatagatcatgatggaaagttccttgttggtttgatgagagaaaaaggggttacgtatagctctgctcctcagtctgtattaatgtcaatgaggagtactttgatcactgtgataacactgtggatacctttaattccattgatgtggcttctgtatcgccaactttcggctgctaatagtcccgcaaggaagcagagaccaaatggtcagacagttggatttgatgatgtggaaggtgttgattctgcaaaagtagagctcgtggagatagtatcatgtctgcaaggggatataaattaccgaaagctaggggcaaagttacctagaggtgtgttgctggtgggtcctcctggaactgggaagacactacttgcgcgtgcagtggcaggggaagcaggtgtaccctttttcactgtatctgccagtgagtttgtggagttgtttgttggaagaggggcagctagaatcagagaccttttcaatgcagcaagaaaatttgcaccatcaatcatattcattgatgaacttgatgcagttggaggcaagcgtggaagaagtttcaatgatgaacgtgaccagacgttaaatcagttgctgacagaaatggatggatttgaatccgagatgagagtggttgtcattgcagcaactaatcggcccgaagccttggatccagccctatgtcgccctggtcgtttctcgagaaaagtatatgtaggtgaacctgatgaagaaggaaggagaaagatattggctgtgcatctaagaggagttcctttggaggaggacagtagcatcatttgccatttaattgcttctcttactactggtttagtaggtgctgatctggcaaatgttgtcaatgaagccgcgttgcttgctgctcgtagaggtagtgaaactgtggcaagggaagatataatggaagctatggaaagagcaaaattcggaatcagtgataaacaattgaggtcaagtaaaataagcaaggagctaagcaagctattcccatggatgccatcattgatgggaaaaagtgagaggagacaggatgacctgcaaggaccattaggttatcaatcactgagttcatga

SEQ ID NO: 60 - protein, 631 aa, Glycine max
  > Glyma.19G040200
MACFSLPCNTGSFVTPTLAQRRYFGVCGGLCTSSFAFTSLGFNQCFKFQHELVWRSRIPSLRVPHCCKTPHGVSSNNKIEPLVSRSKGEKKTHYGKDGTDRLKKRFSLRLRPRLRLLAMRMKRASIKSILNELGILIRKNIRAVAFSASISTVFCLCFLFLKLTALPPPKSVPYSDLIISLQNGHVEKVLVEEGSRRIYYNMKSQNIENDPVSGEESEVADASIDKDVDKIGSEGTSKAGQTPVGNVLKKFSKTRASIPEWQYSTRKIDHDGKFLVGLMREKGVTYSSAPQSVLMSMRSTLITVITLWIPLIPLMWLLYRQLSAANSPARKQRPNGQTVGFDDVEGVDSAKVELVEIVSCLQGDINYRKLGAKLPRGVLLVGPPGTGKTLLARAVAGEAGVPFFTVSASEFVELFVGRGAARIRDLFNAARKFAPSIIFIDELDAVGGKRGRSFNDERDQTLNQLLTEMDGFESEMRVVVIAATNRPEALDPALCRPGRFSRKVYVGEPDEEGRRKILAVHLRGVPLEEDSSIICHLIASLTTGLVGADLANVVNEAALLAARRGSETVAREDIMEAMERAKFGISDKQLRSSKISKELSKLFPWMPSLMGKSERRQDDLQGPLGYQSLSS

SEQ ID NO: 61 - DNA, 1920 bp, Gossypium raimondii
  > Gorai.001G203700
atggcttgtttcagtgttgtttgcaataatggggtttttgttagtaaagaaaacttggtgattgatagtggaaaacccaagtctatctggagaaatattagcttttcttatacttcattttgctttccttcgcttggaatatatagatataaccattgcaaaacccagaatttgtttttttctggtcataatagctttaggttaattagaaatggaagaagaaatgaggttccttcgttggcttttcaagtttgttgcaaggcacccgatgggttattgatgaggggaaatagtggagataaggaaacccaatttggaagaagaggaagtagtaatgtaagaaagagaatttcactaaggctgcgtccaaggttgcggttgttagcaataagaatgaagagagtttcggttaggtcgatgttaaatggtattgctgtctttctaagaaagaatataagaagagttagtctatacagtacaatatctctagcgttgggaatgtgctatttgttcttgaaattaactgcagtgccatctcctaagattgttccgtactcggagtttataacaagcctgcagagtagttctgtgaccaaagttttgcttgaagagggttcccgttgtatatatttcaacatgaattctaaaagtgctgaaaatacacaatctgaagaagaatcatcagctgggaatgattcaatagaaaacctaactggtatggctgcaagagaggatagtgtggtggatggtagacaattgcaaaaacaaggtctatttaagaaaatttcaagacctaaaacttctacttctgcatggatgtatttgacaagaaaaattgatcatgacgaaaagtatcttcttagtttgatgcgagaaaaaggaacaacttatagctcagctcctcagtcagttctaatgtcaatgaggagtactttaataactataatatccttgtggattcctttgactcctttgatgtggctgctgtaccgtcaactttctgctgcaaatagccctgcaaggaagcggcgaccaagtaatcaggttgttgggtttgatgatgtggagggagttgatactgctaaggtggagcttatggaggtagtttcatgccttcagggttctgtaaactaccaaaaactgggagcaaaattacctagaggtgtgttgctagtgggtccaccaggaacaggaaagacattactggctcgtgcagtagctggagaagctggagttcctttcttcagtgtatcagcaagtgaatttgtggaattatttgttggcagaggagcagctcgaatcagagatcttttcaatgtggcaaggaaatctgcaccatcgattatctttattgatgagcttgatgcagttggagggaagcgtggtagaagtttcaatgatgaacgagaccaaacattgaaccagttgctgactgaaatggatggatttgagtcagacatgaaagtagttgttattggagcaactaataggccagaagcactggatccagccctctgtcgccctggtcgcttctcaaggaaagtgttggtgggggaaccaaatgaggaaggacggaaaaagattttggctgtacatttgagaggagttcctctagaagaagacccacaacttatttctgagttagtggcttcactgactccaggttttgtaggtgctgatctcgcaaacattgtcaatgaagctgctttacttgctgctcgtcgaggtggtgaaactgtgacaagggaagatataatggaagctgttgagagagctaaatttgggattaatgatcgacagcctagtaccataggcaaggagcttgggaaactttttccatgggtgccctccttaatgggaaagagtgatccaagacaggatggcttgcaagggcctttgggctatcaaactctaagctga

SEQ ID NO: 62 - protein, 639 aa, Gossypium raimondii
  > Gorai.001G203700
MACFSVVCNNGVFVSKENLVIDSGKPKSIWRNISFSYTSFCFPSLGIYRYNHCKTQNLFFSGHNSFRLIRNGRRNEVPSLAFQVCCKAPDGLLMRGNSGDKETQFGRRGSSNVRKRISLRLRPRLRLLAIRMKRVSVRSMLNGIAVFLRKNIRRVSLYSTISLALGMCYLFLKLTAVPSPKIVPYSEFITSLQSSSVTKVLLEEGSRCIYFNMNSKSAENTQSEEESSAGNDSIENLTGMAAREDSVVDGRQLQKQGLFKKISRPKTSTSAWMYLTRKIDHDEKYLLSLMREKGTTYSSAPQSVLMSMRSTLITIISLWIPLTPLMWLLYRQLSAANSPARKRRPSNQVVGFDDVEGVDTAKVELMEVVSCLQGSVNYQKLGAKLPRGVLLVGPPGTGKTLLARAVAGEAGVPFFSVSASEFVELFVGRGAARIRDLFNVARKSAPSIIFIDELDAVGGKRGRSFNDERDQTLNQLLTEMDGFESDMKVVVIGATNRPEALDPALCRPGRFSRKVLVGEPNEEGRKKILAVHLRGVPLEEDPQLISELVASLTPGFVGADLANIVNEAALLAARRGGETVTREDIMEAVERAKFGINDRQPSTIGKELGKLFPWVPSLMGKSDPRQDGLQGPLGYQTLS

SEQ ID NO: 63 - DNA, 1809 bp, Kalanchoe fedtschenkoi
  > Kaladp0043s0308
atggctgcttcttcttatgttggtttcaattacaccagtggatttgttccttttacagaaaatttgaatgctcgcggaggcaatcgcagctgtttcattgtttctgcaaacgggattagaagtaggcataatccgtggtgtgcttcggtttcctttccattgttggggctgaaaaagtgttgcaattctccaagggggttgctgtgcagtagtaggtggccggtggagggaccgagcaatgtgaattcaagttcagggccgcggcctaggcggaggagactccctgtaaggtcagtgcttgatggggttgttacttttttgcgaaaaaatgcgaaaaaagcgactctttcagccctggtttctctggcgctgggagtttgctacattttcctgaaactatcagccgttcccaccccacaaattgtgccgtattcggatttggtttctagaatcagaaatggttccgttgccaaagtgttacttgaggaggggtctaagcgcatatattttaatgttaactcgcccagctctgaagagaacggcgttttagaggaaaaactctcctctaccgctgatatggaagaagaattgatgaagtcagtttcaagtgatggcattcaaagtagcagtcagatggcaggtgcaaatctgctcaaaaagttctcaaagacagggatccgatcttcttctcctgaatggcactactgtacaagaaaaattgatcatgatgagaagttccttctcagtttgatgagagaaaaaggaactagttacagctcggcccctccatctgtgctcacatcattcaggagcgttgtgataactatcttaaccctgtggattccgctcactcctttaatgtggcttctttaccgtcaactctctgctgccaatagccctgcaaagaaacgacgagctacaaatcaaatagtcacctttgatgatgtggaaggggtagatgctccaaaagttgagcttatggagatagttttgtgtatgcaaggagccatcaactaccaaaaacttggggctaagttacctagaggtgtgctactagtgggtccgccgggtactggtaaaacactacttgcacgcgctgtggctggggaagctggggtgccattctttacagtttcggccagtgaatttgtagaaatgtttgttggaagaggcgcagctcgcataagagacctcttcagtgctgccaggaaatgttctccgtccattatatttattgatgaacttgatgccgttggaggtaagcgtggaagaagcttcaacgatgaacgtgatcagaccctgaatcagttgttgaccgaaatggacggatttgagactgacacaaaggtagttgttctcgctgcaacaaatagaccagaagcattggatcctgctctctgccggcccggccgtttttcaaggaaagtagttgttggggaacctgatgaagacggccggagaaaaatattggcggtgcatttcagaggtgtcccgctggaggaagacagagatcttatgtgtagtcttgttgcttctgtcactcaagggtttgttggcgcagatcttgcaaacatcgtcaatgaagctgcactacttgctgctcgcagaggtggggaaaatgtggccagggaagatataatggaagccatagaaagagccaagtttggaatcagtgacaagcggcaatctcctggtgcactgagtaaagagattgagaaactgtttccatggatgccttccttgatgggaaaaaacaactcgaggcaggatgggtctgaaggccccctcagttatggaacaatgagttga

SEQ ID NO: 64 - protein, 602 aa, Kalanchoe fedtschenkoi
  > Kaladp0043s0308
MAASSYVGFNYTSGFVPFTENLNARGGNRSCFIVSANGIRSRHNPWCASVSFPLLGLKKCCNSPRGLLCSSRWPVEGPSNVNSSSGPRPRRRRLPVRSVLDGVVTFLRKNAKKATLSALVSLALGVCYIFLKLSAVPTPQIVPYSDLVSRIRNGSVAKVLLEEGSKRIYFNVNSPSSEENGVLEEKLSSTADMEEELMKSVSSDGIQSSSQMAGANLLKKFSKTGIRSSSPEWHYCTRKIDHDEKFLLSLMREKGTSYSSAPPSVLTSFRSVVITILTLWIPLTPLMWLLYRQLSAANSPAKKRRATNQIVTFDDVEGVDAPKVELMEIVLCMQGAINYQKLGAKLPRGVLLVGPPGTGKTLLARAVAGEAGVPFFTVSASEFVEMFVGRGAARIRDLFSAARKCSPSIIFIDELDAVGGKRGRSFNDERDQTLNQLLTEMDGFETDTKVVVLAATNRPEALDPALCRPGRFSRKVVVGEPDEDGRRKILAVHFRGVPLEEDRDLMCSLVASVTQGFVGADLANIVNEAALLAARRGGENVAREDIMEAIERAKFGISDKRQSPGALSKEIEKLFPWMPSLMGKNNSRQDGSEGPLSYGTMS

SEQ ID NO: 65 - DNA, 1551 bp, Kalanchoe laxiflora
  > Kalax.0058s0121
atgcttgatggggttgttacttttttgcgaaaaaatgcgaaaaaagtgactctttccgctctggtttctctggcgctgggagtttgctacattttcctgaaactatcagctgttcccaccccacaaattgtgccatattcggatttagtttctagaatcagaaatggttccgttgccaaagtgttacttgaggaggggtctaagcgcatatactttaatgtaaactcgcccagctctgaagagaacggccttttagaggaaaaactctcctctgccgctgatatggaagaagaattgatgaagtcagtttcaagtgatggcattcaaagtagcagtcagatggccgctgatatggaagaagaattgatgaagtcagtttcaagtgatggcattcaaagtagcagtcagatggcaggggtccgatcttcttctcctgagtggcagtactgcacaagaaaaattgatcatgatgagaagttccttctcagtttgatgagagaaaaaggaactagttacagctcggcccctccatctgtgctcacatcattcaggagcgttgtgataactatcttaaccctgtggattccgctcactcctttaatgtggcttctttaccgtcagctctctgctgccaatagccctgcaaagaaacgacgagctacaaatcaaatagtcacctttgatgatgtggaaggggtagatgctccaaaagttgagcttatggagatagttttgtgtatgcaaggagccatcaactaccaaaaacttggggctaagttacctagaggtgtgctactagtgggtccgccgggtactggtaaaacactacttgcacgcgctgtggctggggaagctggggtgccattctttacagtttcggccagtgaatttgtagaaatgtttgttggaagaggcgcagctcgcataagagacctcttcagtgctgccaggaaatgttctccgtcgattatatttattgatgaacttgatgccgtaggaggtaagcgtggaagaagcttcaacgatgaacgtgatcagaccctgaatcagttgttgaccgaaatggatggatttgagactgacacaaaggtagttgttctcgctgcaacaaatagaccagaagcattggatcctgctctctgccggcccggccgtttttcaaggaaagtagttgttggggaacctgatgaagacggccggagaaaaatattggcggtgcatttcagaggtgtcccgctggaggaagacagagatcttatgtgtagtcttgttgcttctgtcactcaagggtttgttggcgcagatcttgcaaacatcgtcaatgaagctgcactacttgctgctcgcagaggtggggaaaatgtggccagagaagatataatggaagccatagaaagagccaagtttggaatcagtgacaagcggcaatctcctggtgcactgagtaaagagattgagaaactgtttccatggatgccttccttgatgggaaaaaacaactcgaggcaggatggatctcaaggccccctcagttatggaacagtgagttga

SEQ ID NO: 66 - protein, 516 aa, Kalanchoe laxiflora
  > Kalax.0058s0121
MLDGVVTFLRKNAKKVTLSALVSLALGVCYIFLKLSAVPTPQIVPYSDLVSRIRNGSVAKVLLEEGSKRIYFNVNSPSSEENGLLEEKLSSAADMEEELMKSVSSDGIQSSSQMAADMEEELMKSVSSDGIQSSSQMAGVRSSSPEWQYCTRKIDHDEKFLLSLMREKGTSYSSAPPSVLTSFRSVVITILTLWIPLTPLMWLLYRQLSAANSPAKKRRATNQIVTFDDVEGVDAPKVELMEIVLCMQGAINYQKLGAKLPRGVLLVGPPGTGKTLLARAVAGEAGVPFFTVSASEFVEMFVGRGAARIRDLFSAARKCSPSIIFIDELDAVGGKRGRSFNDERDQTLNQLLTEMDGFETDTKVVVLAATNRPEALDPALCRPGRFSRKVVVGEPDEDGRRKILAVHFRGVPLEEDRDLMCSLVASVTQGFVGADLANIVNEAALLAARRGGENVAREDIMEAIERAKFGISDKRQSPGALSKEIEKLFPWMPSLMGKNNSRQDGSQGPLSYGTVS

SEQ ID NO: 67 - DNA, 1515 bp, Kalanchoe laxiflora
  > Kalax.0269s0004
atgcttgatggggttgttacttttttgcgaaaaaatgcgaaaaaagcgactctttccgccctggtttctctggcgctgggagtttgctacattttcctgaaactatcagccgttcccaccccacaaattgtgccgtattcggatttggtttctagaatcagaaatggttccgttgccaaactgttacttgaggaggggtctaagcgcatatattttaatgttaactcgcccagctctgaagagaacggcgttttagaggaaaaactctcctctaccgctgatatggaagaagaattgatgaagtcagtttcaagtgatggcattcaaagtagcagtcagatggcaggtgcaaatctgctgacaaagttctcaaagacagggatccgatcttcttctcctgaatggcagtactgtacaagaaaaattgatcatgatgagaagttccttctcagtttgatgagagaaaaaggaactagttacagctcggcccctccatctgtgctcacatcattcaggagcgttgtgataactatcttaaccctgtggattccgctcactcctttaatgtggcttctttaccgtcaactctctgctgccaatagccctgcaaagaaacgacgagctacaaatcaaatagtcacctttgatgatgtggaaggggtagatgctccaaaagttgagcttatggagatagttttgtgtatgcaaggagccatcaactaccaaaaacttggggctaagttacctagaggtgtgctactagtgggtccgccgggtactggtaaaacactacttgcacgcgctgtggctggggaagctggggtgccattctttacagtttcggccagtgaatttgtagaaatgtttgttggaagaggtgcagctcgcataagagacctcttcagtgctgccaggaaatgttctccgtcgattatatttattgatgaacttgatgccgttggaggtaagcgtggaagaagcttcaacgatgaacgtgatcagaccctgaatcagttgttgaccgaaatggacggatttgagactgacacaaaggtagttgttctcgctgcaacaaatagaccagaagcattggatcctgctctctgccggcccggccgtttttcaaggaaagtagttgttggggaacctgatgaagacggccggagaaaaatattggcggtgcatttcagaggtgtcccgctggaggaagacagagatcttatgtgtagtcttgttgcttctgtcactcaagggtttgttggcgcagatcttgcaaacatcgtcaatgaagctgcactacttgctgctcgcagaggtggggaaaatgtggccagggaagatataatggaagccatagaaagagccaagtttggaatcagtgacaagcggcaatctcctggtgcactgagtaaagagattgagaaactgtttccatggatgccttccttgatgggaaaaaacaactcgaggcaggatgggtctgaaggccccctcagttatggaacaatgagttga

SEQ ID NO: 68 - protein, 504 aa, Kalanchoe laxiflora
  > Kalax.0269s0004
MLDGVVTFLRKNAKKATLSALVSLALGVCYIFLKLSAVPTPQIVPYSDLVSRIRNGSVAKLLLEEGSKRIYFNVNSPSSEENGVLEEKLSSTADMEEELMKSVSSDGIQSSSQMAGANLLTKFSKTGIRSSSPEWQYCTRKIDHDEKFLLSLMREKGTSYSSAPPSVLTSFRSVVITILTLWIPLTPLMWLLYRQLSAANSPAKKRRATNQIVTFDDVEGVDAPKVELMEIVLCMQGAINYQKLGAKLPRGVLLVGPPGTGKTLLARAVAGEAGVPFFTVSASEFVEMFVGRGAARIRDLFSAARKCSPSIIFIDELDAVGGKRGRSFNDERDQTLNQLLTEMDGFETDTKVVVLAATNRPEALDPALCRPGRFSRKVVVGEPDEDGRRKILAVHFRGVPLEEDRDLMCSLVASVTQGFVGADLANIVNEAALLAARRGGENVAREDIMEAIERAKFGISDKRQSPGALSKEIEKLFPWMPSLMGKNNSRQDGSEGPLSYGTMS

SEQ ID NO: 69 - DNA, 1893 bp, Linum usitatissimum
  > Lus10003049.g
atgtccgctttctctctggtttgctctaccgggtcgttccgcactaaagagaacttgagaattagtagtgtcaagccgaggactgcgacgggatactcagctgccttttcgcattattggattccttctgttcaactttgcaagaaactagggtcattctgtagtgagaacaatggagctcgcttgtttggatttggtcttggttaccggcggtcttggcgtggggtttcttgtagcagcagaattgggccgttggtcagcagtagtaacaatggaggcgaaccaaccgagttgagaaaaaaaattagtaaagcgaggaggagattttcattgagactgcgccctaggttacggctgttgaggatgatgatgaagagaatacgagttaggtcagccataagtgatttcattttgtttcttaagaagaatttgagaagaagggaggcactttatgcttcaatctctattggattggcaatgtgctacttgtttctgagactggcaactgtgccatcttacaagattgttccttattctgacttgataggatccctgcagaatggttccgttagtaaagttttactcgaagaggggtctcgtcggatatattataacatagactttcctgagacctcaaataaccaaaacacagaagatcagtctccttctgtagctgtagaaggtgttgccttagatgatcagtctcaagctatagacgtttccaatgttgtagaaggtgtggcagctccacagccatcgagggctactcagttgggcaaggtatcattgaaacctcgagcatcgaaacctaagtggcagtttgccactaggaaaatggatcatgatgagaagtttctgcttagtttgttgaggcaaaaggggacgacgtatagctctgctccccaatcagtttggatgtcaatgaggagtttgctgttaactataatagctttatggattcctttaactcctgtgatgtggcttctataccgccagctttcggctgccaatagtcctgcaaagaagcggcgtccaactaatcgaactgtcacttttaatgacgtggatggtgttgatgctgctaaggcagaacttattgagatagtctcgtgccttcaaggagcgataaaataccaaacgctgggggcaaaactgcccagaggtgtgttgttggttggtcctccggggacgggaaagacattactggctcgtgcagtagcaggagaagctggagtcccctttttcagtgtctccgccagtgaatttgtggaaatgtttgttggaagaggagcagcacgcatcagagatctgtttaatgctgccaggaaatctgctccgtctatcatattcatcgacgaacttgatgcagttggaggaagacgtggtcgtagcttcaacgatgaacgagatcaaacactaaatcaggtgatagtggtggtgattgcagccacaaacaggccagaggcactggaccctgctctatgtcgtccggggagattctcgagaaaggtgttggtaggagaaccagatgaagaagggaggagaaagatattagcgatccatttgagggaagttcctctggaagaagacacggatcttatagccgatctagttgcttctttaactccaggtctagtcggtgcggatcttgcaaacatcgttaacgaggctgctttactcgctgccaggagaggtggtgagagtgtagcaagggaagatgtaatggaagccattgaaagagcaaagtttggaatcaatgataagaaactgaggccgagttcgataagcaaggagatagggaagctgttcccatgggttcttccatctttggcggggaagacacaaggtgattcgcaaggggaagggcctttaggttaccggacattgagctaa

SEQ ID NO: 70 - protein, 630 aa, Linum usitatissimum
  > Lus10003049.g
MSAFSLVCSTGSFRTKENLRISSVKPRTATGYSAAFSHYWIPSVQLCKKLGSFCSENNGARLFGFGLGYRRSWRGVSCSSRIGPLVSSSNNGGEPTELRKKISKARRRFSLRLRPRLRLLRMMMKRIRVRSAISDFILFLKKNLRRREALYASISIGLAMCYLFLRLATVPSYKIVPYSDLIGSLQNGSVSKVLLEEGSRRIYYNIDFPETSNNQNTEDQSPSVAVEGVALDDQSQAIDVSNVVEGVAAPQPSRATQLGKVSLKPRASKPKWQFATRKMDHDEKFLLSLLRQKGTTYSSAPQSVWMSMRSLLLTIIALWIPLTPVMWLLYRQLSAANSPAKKRRPTNRTVTFNDVDGVDAAKAELIEIVSCLQGAIKYQTLGAKLPRGVLLVGPPGTGKTLLARAVAGEAGVPFFSVSASEFVEMFVGRGAARIRDLFNAARKSAPSIIFIDELDAVGGRRGRSFNDERDQTLNQVIVVVIAATNRPEALDPALCRPGRFSRKVLVGEPDEEGRRKILAIHLREVPLEEDTDLIADLVASLTPGLVGADLANIVNEAALLAARRGGESVAREDVMEAIERAKFGINDKKLRPSSISKEIGKLFPWVLPSLAGKTQGDSQGEGPLGYRTLS

SEQ ID NO: 71 - DNA, 1590 bp, Linum usitatissimum
  > Lus10034097.g
atgaggatgaagagaatacgagttaggtcagccataagtgatttcattttgtttcttaagaagaatttgagaagaagggaggcactttatgcttcaatctctattggattggcaatgtgttacttgtttctgaggctggcagctgtgccatcttacaagattgttccttattctgacttgataggatccctgcagaatggttctgttagtaaagttttactcgaagaggggtctcgtcggatatattataacattgactctccagggacctcaaataaccaaaactcagaagatcagtctccttctgtagttgttcctaatgagtgtttagctggctcaggtcctgctgttgaaggtattgtcttagatgatcagtctcaagctgtagcaattgcagaaggcgttgcagcttcacaaccatcgaggggtgctcagttgggcaaggtattattaaaacctcgagcatcgaaacctaagtggcagtttgcgactaggaaaatggatcatgatgagaagtttctacttagtttgttgagggaaaaggggacaacttatagctctgctccccaatcagtttggatgtcaatgaggagtttgctgctaactataattgctttatggattcctttaactcctgtgatgtggcttctatatcgccagctttcggctgccaatagtcctgcaaagaagcggcggccaactaatcggactgtcacctttaatgacgtggatggtgttgatgctgctaaggcagaacttattgagatagtctcgtgccttcaaggagctataaaataccaaactctgggtgcaaaactacccagaggtgtattgttggttggtcctccggggacgggaaagacattgctggctcgtgcagtagcaggagaagccggagtcccatttttcagcgtctccgccagtgaatttgtggaaatgtttgttggaagaggagcagcacgcatcagagacctgtttaatgctgccaggaaatctgctccgtctattatattcatcgatgaacttgatgcagttggaggaagacgtggtcgtagcttcaacgatgaacgagatcaaacactaaatcagttgcttacagaaatggatggattcgagtcagagatgagagtggtggtgattgcagctacaaacagaccagaggcactggaccctgccctatgtcgtccggggagattctcgagaaaggtgttggtagaagaaccagatgaagaagggaggagaaagatattagccatacatttgagggaagttcctctggaagaagacgctgatcttatagctgatctaattgcttcgttaacaccaggtctcgtaggtgctgatcttgcaaacatcgttaatgaggctgctttactcgctgcgaggagaggtggtgagagtgtagcaagggaagatgtaatggaagccattgaaagagcgaagtttgggatcaatgataagaaactaaggccgagttcgataagcaaggagatagggaagctgttcccgtgggttctgccatctttagcgaggaagacacaaggtgatacgcaaggggaagggcctttaggttaccgaacattgagctaa

SEQ ID NO: 72 - protein, 529 aa, Linum usitatissimum
  > Lus10034097.g
MRMKRIRVRSAISDFILFLKKNLRRREALYASISIGLAMCYLFLRLAAVPSYKIVPYSDLIGSLQNGSVSKVLLEEGSRRIYYNIDSPGTSNNQNSEDQSPSVVVPNECLAGSGPAVEGIVLDDQSQAVAIAEGVAASQPSRGAQLGKVLLKPRASKPKWQFATRKMDHDEKFLLSLLREKGTTYSSAPQSVWMSMRSLLLTIIALWIPLTPVMWLLYRQLSAANSPAKKRRPTNRTVTFNDVDGVDAAKAELIEIVSCLQGAIKYQTLGAKLPRGVLLVGPPGTGKTLLARAVAGEAGVPFFSVSASEFVEMFVGRGAARIRDLFNAARKSAPSIIFIDELDAVGGRRGRSFNDERDQTLNQLLTEMDGFESEMRVVVIAATNRPEALDPALCRPGRFSRKVLVEEPDEEGRRKILAIHLREVPLEEDADLIADLIASLTPGLVGADLANIVNEAALLAARRGGESVAREDVMEAIERAKFGINDKKLRPSSISKEIGKLFPWVLPSLARKTQGDTQGEGPLGYRTLS

SEQ ID NO: 73 - DNA, 1848 bp, Musa acuminata
  > GSMUA_Achr9G18420_001
atgagcccgttctccttggtttcaactcatggggtttttagccctcaccattcttcttcaaattggcctagattgttctctcatagaaggagattgagcctgcaagaaggaaaaatcttgtctttggaagaaagacattgggatcttacttcgtcttcctctctttgttgtcgcaactcccgatgtctttctttatggggttcttcgcggagatttcggttattgtccaaatgtaataggattgtatttgcgaagattgataggagcagcaacgagaagcatacccattttgggattcgagggaagcctaggtcgagaaggcgattctccctgagattgcgccccaggctgcggttgctctcttatagattgaggagattgtcggttcaacgattggtggaaaatgctagaacagtgttgctcagaaactcgaagaagcttatgctatctgcttggttttctgttgccctaggggtgtgtttcttgttcttgaagtgcacagctagacctgcatcggtggatgttccatattcagattttgtatcggccctgaagagtggaagcgtgtcagctgttcaattcgaagaggattcccgctatatttactttaatcagcaatttgaagataatgagagtttgaagtcagtagaagaatcatcactgactgtcgatgtctcagatgtcagtgcaccaaaatcatctgaaagcattacaaatgaaagggagagaggtggtaaatctatctcaaagtggcaatactctacaagaaaagttgagcatgatgagagttttcttcttggtttgatgagagagagagggaccacatacagttctgctcctcaatctgcagtaaagttattgagaagtcttgttatcacattgattactttgtggatcccaatagccccaatgttgtggttgctttatcaccaattctatgctagcaatggccctgcaaagaagcggcggcctagtaagcagtctgtcagttttgatgatgtggagggagttgatgctgccaagctagaattgatggagatagtatcttgcttacaaggatcaatgaactacaaaaagttaggagcaaaattgcccacaggtgtgctgcttgtgggacctccaggcactgggaagacattactagctcgctcagttgctggagaggcaggagtcccttttttctcagtgtctgctagtgaatttgtagaactgtttgttggaagaggagctgcacgagtaagagacctttttaatgtggcaaaagaatgtgcaccgtcaatagtattcattgatgaacttgatgcagtgggaggaaagcgtggaagaagtttcaatgatgagcgggaccaaacacttaatcagttgctgacagaaatggatggttttgagtcggagacaaaagtgattgttattgcagcaacaaatagaccagaagcactagatcctgctctctgcaggccaggacgtttttcaaggaaagtgcttgtcggtgaaccagattttgaagggcgtaaaaaaattttggctgtccatctgagacaaataccactggaggaagaaattgagttaatctgtgaccttgttgcatctctcacagcaggttttgttggtgctgatctcgcaaacattgtcaatgaagctgctctgcttgctgctcgaagagatgctgagacggttaccagagatgatatgatggaggcaatagagcgagcaaagtttgggattaaagaaaggcgactgaatcttagtacagtaggcaagagtctgggtaaattgtttccatggattcctccatcaacaggaaaagatggtttccaaggccttatgggttaccaaactttgagctaa

SEQ ID NO: 74 - protein, 615 aa, Musa acuminata
  > GSMUA_Achr9G18420_001
MSPFSLVSTHGVFSPHHSSSNWPRLFSHRRRLSLQEGKILSLEERHWDLTSSSSLCCRNSRCLSLWGSSRRFRLLSKCNRIVFAKIDRSSNEKHTHFGIRGKPRSRRRFSLRLRPRLRLLSYRLRRLSVQRLVENARTVLLRNSKKLMLSAWFSVALGVCFLFLKCTARPASVDVPYSDFVSALKSGSVSAVQFEEDSRYIYFNQQFEDNESLKSVEESSLTVDVSDVSAPKSSESITNERERGGKSISKWQYSTRKVEHDESFLLGLMRERGTTYSSAPQSAVKLLRSLVITLITLWIPIAPMLWLLYHQFYASNGPAKKRRPSKQSVSFDDVEGVDAAKLELMEIVSCLQGSMNYKKLGAKLPTGVLLVGPPGTGKTLLARSVAGEAGVPFFSVSASEFVELFVGRGAARVRDLFNVAKECAPSIVFIDELDAVGGKRGRSFNDERDQTLNQLLTEMDGFESETKVIVIAATNRPEALDPALCRPGRFSRKVLVGEPDFEGRKKILAVHLRQIPLEEEIELICDLVASLTAGFVGADLANIVNEAALLAARRDAETVTRDDMMEAIERAKFGIKERRLNLSTVGKSLGKLFPWIPPSTGKDGFQGLMGYQTLS

SEQ ID NO: 75 - DNA, 2004 bp, Malus x domestica
  > MDP0000171044
atggcttgttgtttttctgtagtttccaatactgggttcttatctgtcccaaacaaattggaatttaatggcggaaagtccaagtctttggggaggtatagaggtttttactgtagttcatttggttttcggtcggcggggtttcataaatttcgaaactttcaacatgggttgttttggaacaatgagcttaggccactgatgaatggaaacaatggagtttggttaaagggattgaataattgttgtgattctagacatgggttgtgttgttacaataaaattgagccattgacaaatgcgaatagcgcgaataagcagatccatttcgggaagaaaggagacactaagttgaggtctttgaggaggcgattttctttgagattacgcccgaggttgcggtggttggctatgagagtgaaaagagttacaattcagtcagtgttgaatggtgttcgggccttcttgcaaaagaacataagacgagtgactgttgtttctttggtttctgtaatattggggctgagctatctgtttctgaagttaacagctttgccctctccgaaaatggttccatattccgaattggtaacaagcctgagaaatgagtctgttacgaaagttctacttgaagaggggtctcgtcgaatatattataacacaaactctcgcattgatagggatactcaattgtctgaaggagaattaccaagcgttcaaagtgagaatgtggccgataaagtaacaagtgatgatggttctcgatctagtcaagcactgaatacaaatgtattgagaaatttatcagcgactcgagcttcaacccctgactggcagtatttgactagaaaagtagatcatgatgagaaatttttacttagtttgatgagagagaaaggaattacatatagctcggctcctcaatcagttttgatgtcaatgaggactactttgataactataatatctctgtggattcctttgatgccattgatgtggcttctttatcgtcaactttcagctgctaatagcccagctagaaagcaacgacctgataagcagttggttggatttgaggatgttgagggagttgatgctgcgaaattggagcttatggagtgctgggaaggtatcccatggcttgcaacaagccctattacacatggcgatgagcctttcaggatagttttatgcttgcaaggagctattaactacaacaaactaggagcaaagttacccagaggtgtattgctggtgggtcctccaggaacagggaaaacattacttgcccgtgcagtggctggagaagcaggtgtaccatttttcagtgtttctgccagtgaatttgtggagatgtttgttggaagaggggcagctcgcattagagacctttttaatatggcaaggaaacattccccttcaattatattcattgatgagcttgatgcagttggaacgaagcgtggcagaagtttcaatgatgagcgtgaccaaacattgaatcagttgctgacggaaatggatggatttgagtcagactcgaaggtgattgttgttgcagcaacaaacaggcctgaagtattggattctgccctttgtaggccaggccgcttctcaagaaaaatagttgtaggagaaccagatgaagaaggaaggagaaagatattggctgtacatttgagaggagttcctttggaggaagacactaatcttatttgcgatctcattgcttcactgacaccaggttttgtaggtgctgatcttgcaaacatcatcaatgaagctgctttacttgctgctcgtaggggtggtgaaactgtggccagggaagatgtaatggaagcaattgaaagagcaaagtttggaattaatgataagcaacttaggccgagtacaataagcaaggagctcgggaaaatgttcccatggatgccttctctgatgggaaagaacacaagacaagatggagtgcaagggccactaggatatcaaactctgagctga

SEQ ID NO: 76 - protein, 667 aa, Malus x domestica
  > MDP0000171044
MACCFSVVSNTGFLSVPNKLEFNGGKSKSLGRYRGFYCSSFGFRSAGFHKFRNFQHGLFWNNELRPLMNGNNGVWLKGLNNCCDSRHGLCCYNKIEPLTNANSANKQIHFGKKGDTKLRSLRRRFSLRLRPRLRWLAMRVKRVTIQSVLNGVRAFLQKNIRRVTVVSLVSVILGLSYLFLKLTALPSPKMVPYSELVTSLRNESVTKVLLEEGSRRIYYNTNSRIDRDTQLSEGELPSVQSENVADKVTSDDGSRSSQALNTNVLRNLSATRASTPDWQYLTRKVDHDEKFLLSLMREKGITYSSAPQSVLMSMRTTLITIISLWIPLMPLMWLLYRQLSAANSPARKQRPDKQLVGFEDVEGVDAAKLELMECWEGIPWLATSPITHGDEPFRIVLCLQGAINYNKLGAKLPRGVLLVGPPGTGKTLLARAVAGEAGVPFFSVSASEFVEMFVGRGAARIRDLFNMARKHSPSIIFIDELDAVGTKRGRSFNDERDQTLNQLLTEMDGFESDSKVIVVAATNRPEVLDSALCRPGRFSRKIVVGEPDEEGRRKILAVHLRGVPLEEDTNLICDLIASLTPGFVGADLANIINEAALLAARRGGETVAREDVMEAIERAKFGINDKQLRPSTISKELGKMFPWMPSLMGKNTRQDGVQGPLGYQTLS

SEQ ID NO: 77 - DNA, 2193 bp, Malus x domestica
  > MDP0000282409
atgcaacctccttttatctttatcccggcgggtacacctcttacctgcgtccatccgcgcaacgaacatgtttgtaaggttcatctccgtcgtgttgtcgaaaacgctacatttggcttgacacatattgtagccgagtcttggacaagtgtttttgtagagaaaagcatggcttcttgtttttctgttgtttccaatactgggttcttatctattcccaacaaattagaatttcacggcgaaaagtccaagtctttgggaaggtatagaggtttttgccgtagttcatttgcttttcgttccgtgggwtttcgtaaattgcgaaactttcaacatgggttgttctcgaacaatgagcttaggccactgatgaamggaaacagtggagtttggttagagggattgaatagttgttgtaaatctagacatgggttgtgttgttacaataaaatggagccatttacgaatgcaaatagcgcgaataagcagatccatttggggaagaaaggagacactaagttgaggtctttgaggaggagattttctttgagattgcgcccgagyttgcggtggttagctatgagagtgaaaagagttacaattcagtcagtgttgaatggtgttgggactttcttgcgaaagaacatgagaagagtgacactrgtttctttggtttctgtgatattggggctctgctatttgtttctgaagttaaccgctgtgccctctccgaaaatggttccatattcagaattgataacaasccttcaaaatgagtctgttacaaaagttctacttgaagaggggtctcgtcggatatattataacacaaattctcycgttgatgggggtactcaattgtctgagggagaattgccaagcattgaaggtgagaatgtggccgataaagtcacaagtaatgatggttctcgatctagtcaagcactgaataggaatgtttmgagaaaattatcaccgactcaagcttctacgcctgactggcagtattcgaccagaaaagtagatcatgatgagaaatttttacttagtttgatgagagagaaaggaattacatatygctcggctcctcaatcagttttgatgtcaatgaggactactttgataactataatatctctgtggattcctttgatgccartgatgtggcttctttatcgtcaactttctgctgctaatagcccagctagaaagcggcgacctgataagcagttggttggatttgaagatgttgagggagttgatgctgcgaaattggagcttatggagatagttttatgcttgcaaggagctattaactacaacaaactaggagcaaagttacccagaggtgtattgctggttggtcctccaggaacagggaaaacattacttgcccgtgcagtggctggagaagcaggtgtaccatttttcagtgtttctgccagtgaatttgtggagatgtttgttggaagaggagcagctcgcattagagaccttttcaatatggcaaggaaacattcaccwtcaattgtattcattgatgagcttgatgcagttggaacgaagcgtggcagaagtttraatgatgagcgtgaccaaacattgaatcagttgctgacggaaatggatggatttgagtcagactcaaagmtgattgttgttgcwgcaacaaacaggcccgaagtattggattctgccctttgtaggccaggccgcttctcaagaaaaatagttgtaggagaaccagatgaagaaggaaggagaaagatattggctgtacatttgagaggagttcctttggaggaagacactaatcttatttgcgatctcattgcttcactgacaccaggttttgtaggtgctgatcttgcaaacatcgtcaatgaagctgctttacttgctgctcgtaggggtatacagtacatccaaatmatrttttggttttctgaaattgtacacygatctcaacttcctattaatktatggtctgttgcaggtggtgaaactgtggccagagaagatgtaatggaagcaattgaaagygcaaaatttggaattaatgataagcaacttaggccgagtacaataagcaaggagcttgggaaaatgttcccgtggatgccttctctgatgggaaagaacacaaggcaagacggagtgcaagggcctctaggatatcaaactctgagctga

SEQ ID NO: 78 - protein, 730 aa, Malus x domestica
  VARIANT 125,255,280,321,360,387,551,644,654,661: > Xaa is any amino acid; MDP0000282409
MQPPFIFIPAGTPLTCVHPRNEHVCKVHLRRVVENATFGLTHIVAESWTSVFVEKSMASCFSVVSNTGFLSIPNKLEFHGEKSKSLGRYRGFCRSSFAFRSVGFRKLRNFQHGLFSNNELRPLMXGNSGVWLEGLNSCCKSRHGLCCYNKMEPFTNANSANKQIHLGKKGDTKLRSLRRRFSLRLRPRLRWLAMRVKRVTIQSVLNGVGTFLRKNMRRVTLVSLVSVILGLCYLFLKLTAVPSPKMVPYSELITXLQNESVTKVLLEEGSRRIYYNTNSXVDGGTQLSEGELPSIEGENVADKVTSNDGSRSSQALNRNVXRKLSPTQASTPDWQYSTRKVDHDEKFLLSLMREKGITYXSAPQSVLMSMRTTLITIISLWIPLMPXMWLLYRQLSAANSPARKRRPDKQLVGFEDVEGVDAAKLELMEIVLCLQGAINYNKLGAKLPRGVLLVGPPGTGKTLLARAVAGEAGVPFFSVSASEFVEMFVGRGAARIRDLFNMARKHSPSIVFIDELDAVGTKRGRSFNDERDQTLNQLLTEMDGFESDSKXIVVAATNRPEVLDSALCRPGRFSRKIVVGEPDEEGRRKILAVHLRGVPLEEDTNLICDLIASLTPGFVGADLANIVNEAALLAARRGIQYIQXIFWFSEIVHXSQLPINXWSVAGGETVAREDVMEAIERAKFGINDKQLRPSTISKELGKMFPWMPSLMGKNTRQDGVQGPLGYQTLS

SEQ ID NO: 79 - DNA, 1926 bp, Manihot esculenta
  > Manes.02G063800
atggctactttttctgttgtttgtaataatgggttattgattagtaaagaaaatttagttcatagtggaaaggccaagcctttgaggagatacacaagtgtttgttataattcagtgggagcttacacttttagcaattcccaaaattttatgctttctaggaataagtttaggtcgcttcatcatggaaatagtgaagttcctttattggggttttgtgtttcttgcaagtctcaacgtgggttatcttgtcataacaatactgggccattgataatgaggggagacagaggaaatgaacgaagacatttgggaaaaagagttactactggtttgagaaagcgattttcattgagattgcggccaagattgcggctattgatgataagattgaaaagagtttcagtgaggtcaatgttaaatgattttggaatgttcttgagaaagaatataaagagactgatgctttattcttcaatttctcttgcactggggatatgctacttgttcttgagattaactacattgccatctccaaagattgttccatattcggatttaataacaagccttcaaaatgattccgtttcaaaagttttactcgaagaggggtctcgtcggatttattacaacacaaaatctcagggcattgaaaattttaaagatgcagaagaaagatctccagggatagatgtttcaaatgagaatgcagctggtccagctgcaacaggaagtattgcgagtactgctggccacaaatcaaatatggatatattgaagaagttttcagggtctcgagcttctactcccgagtggaagttttccactaggaaaatagaccatgatgagaaatttcttcttagtttgatgagagaaaagggaaccgtatatagctcagcccctcaatcagcattgatgtcaatgaggaatacattgataaccataatatctctctggatccctttaactcctttgatgtggcttctgtatcgtcaactttctgctgctaatagtcctgcaagaaagcggagacctaataatctgatggttagttttgatgatgtggagggcgttgatgctgctaaggtggagcttatggagatagtttcatgcctgcaaggagctacaaactaccaaaaactgggagcaaagttacctagaggcgtgttgctggttggtcccccaggaacaggaaaaacattgctggcccgtgcggttgctggagaagcaggagtaccatttttcagtgtttctgccagtgaatttgtggagttgtttgttggaagaggagcagctcgcattagagatctctttagtgcagcaaggaaatgtgctccatcaattatatttattgatgaacttgatgcagttggggggaaacgtggtagaagtttcaatgatgagcgtgatcaaacactgaatcagttgctgactgaaatggatggatttgagtcagacatgaaagtgattgttgtggcagcaactaacaggccagaagcattggatccagccctttgtaggcctggtcgattctcaagaaaggtacttgtgggagaacctgatgaagaaggaaggaaaaagatattggctgttcatttgagaggagttcccgtagaagaggacacagatatcatttgtgatctagttgcttctttgacagcaggttttgtaggagctgatctggcaaacattgtcaatgaagctgctttacttgctgctcggagaggtggtgagactgtaacaagggaagatataatggaagctcttgaaagagctaagtttgggatcaatgataggcatctgacgcccaatgcaataagcaaggagctggggaaactctttccatggataccctctatactgggaaggaatgacacaggacaagatggtttgcaggggcctatgggttaccaaactctgagctga

SEQ ID NO: 80 - protein, 641 aa, Manihot esculenta
  > Manes.02G063800
MATFSVVCNNGLLISKENLVHSGKAKPLRRYTSVCYNSVGAYTFSNSQNFMLSRNKFRSLHHGNSEVPLLGFCVSCKSQRGLSCHNNTGPLIMRGDRGNERRHLGKRVTTGLRKRFSLRLRPRLRLLMIRLKRVSVRSMLNDFGMFLRKNIKRLMLYSSISLALGICYLFLRLTTLPSPKIVPYSDLITSLQNDSVSKVLLEEGSRRIYYNTKSQGIENFKDAEERSPGIDVSNENAAGPAATGSIASTAGHKSNMDILKKFSGSRASTPEWKFSTRKIDHDEKFLLSLMREKGTVYSSAPQSALMSMRNTLITIISLWIPLTPLMWLLYRQLSAANSPARKRRPNNLMVSFDDVEGVDAAKVELMEIVSCLQGATNYQKLGAKLPRGVLLVGPPGTGKTLLARAVAGEAGVPFFSVSASEFVELFVGRGAARIRDLFSAARKCAPSIIFIDELDAVGGKRGRSFNDERDQTLNQLLTEMDGFESDMKVIVVAATNRPEALDPALCRPGRFSRKVLVGEPDEEGRKKILAVHLRGVPVEEDTDIICDLVASLTAGFVGADLANIVNEAALLAARRGGETVTREDIMEALERAKFGINDRHLTPNAISKELGKLFPWIPSILGRNDTGQDGLQGPMGYQTLS

SEQ ID NO: 81 - DNA, 1809 bp, Mimulus guttatus
  > Migut.H00557
atggcgtcttttcctatttcttggagtgatgggctcttgattgcacaaaaaagattgaatctttctgtcggaaaatcccaaacactgtccgagaatcgatttacctttctttctagtccttctttactgagttattcctcttataaaccccacagtaggctgttgtgtagcaacaagattaagctagtggtcaatgataagataattgtcgacactcagttggtggttaaagggggaaataataataataataataataatagtagtagtggtggtggtaatagtaatgaattaaggaggaaattttcgctgagattgcgccctaggttacgattattatcgaggagattgaaaaggggttcgatcaagtcggtgctcgatgggttcgcaacttttttgcggaagaatataaaaaaagtgacgttttccacttcggtctcggttgtgttgggcctttgcttcttgtttctcaaagtaaccgcaatgcctactccgaaatcggtgccgtattctgatttgataagcagccttcaaagtggaggtgtggctaaggttctgttcgaagagggcactcgtcggatatactacaatacgaatttatggagcgagaaagattccgagaatgcggtagattctgggaataatgtagatggaaaagatacgtcggttaagaaaaaaagtcgaaattcgccacccgcttgggaatttttgacgagaaagattgaccatgacgagagttaccttctgagtcttatgagggagaggggaacttcgtacggctcctcgcctcagtcggctttgatgtcgatgaggagcatgttgatcaccgtgttgactttatggattcctttgactccgctgatgtggattctttaccggcaactttctgccgccaatagtccggccaagaagaggcggcccgggaacttattggtgtcttttgaagatgttgagggtgtcgattctgccaaagtggagcttatggagatagtatgttgtctccaaggagctattaactacagcaagctaggtgccaaattaccacgaggggtactgctcgtgggccccccaggtacaggaaagacgttactagcacgagctgtcgcaggagaagccggggtgccctttttctcggtttccgctagtgaatttgtcgagttatttgtcggcagaggggcggcccgcataagggatcttttcagcgtcgcaagaaagagtgcgccttcgattattttcatcgacgaactcgacgctgtcggtggtaaacgtggtagaagtttcaatgacgaaagagaccaaacattgaaccagttgctgacggaaatggacggatttgagtcggatataaacgtagttgttatagctgcaacgaatagaccagaggctttggacccggctctgtgtaggcctggtcgattctcgagaaaggtgtatgtaggagaaccagacgaagaaggaaggaaaaagattttagccatacatttaaaaggggttcctctcgaagaagatgtaaccctaatttgcaatttggtttcgtctgttactcaaggctttgtcggcgccgatcttgcaaacattgttaacgaatctgcccttcttgctgctcgcagaggggctgaaaaagtatcgagggaagatataatggaagctatagaaagagcgaaattcggaataaacgagaggcaaacgagcgctactacaataagcaaggaactggttaagttgtttccttggatgccttctcttacgaatagaaacaattcgagaaaagatccacaacctccactcggctatcaaactttaagctaa

SEQ ID NO: 82 - protein, 602 aa, Mimulus guttatus
  > Migut.H00557
MASFPISWSDGLLIAQKRLNLSVGKSQTLSENRFTFLSSPSLLSYSSYKPHSRLLCSNKIKLVVNDKIIVDTQLVVKGGNNNNNNNNSSSGGGNSNELRRKFSLRLRPRLRLLSRRLKRGSIKSVLDGFATFLRKNIKKVTFSTSVSVVLGLCFLFLKVTAMPTPKSVPYSDLISSLQSGGVAKVLFEEGTRRIYYNTNLWSEKDSENAVDSGNNVDGKDTSVKKKSRNSPPAWEFLTRKIDHDESYLLSLMRERGTSYGSSPQSALMSMRSMLITVLTLWIPLTPLMWILYRQLSAANSPAKKRRPGNLLVSFEDVEGVDSAKVELMEIVCCLQGAINYSKLGAKLPRGVLLVGPPGTGKTLLARAVAGEAGVPFFSVSASEFVELFVGRGAARIRDLFSVARKSAPSIIFIDELDAVGGKRGRSFNDERDQTLNQLLTEMDGFESDINVVVIAATNRPEALDPALCRPGRFSRKVYVGEPDEEGRKKILAIHLKGVPLEEDVTLICNLVSSVTQGFVGADLANIVNESALLAARRGAEKVSREDIMEAIERAKFGINERQTSATTISKELVKLFPWMPSLTNRNNSRKDPQPPLGYQTLS

SEQ ID NO: 83 - DNA, 1809 bp, Mimulus guttatus; identical to SEQ ID NO: 81
  > Migut.H00557

SEQ ID NO: 84 - protein, 602 aa, Mimulus guttatus; identical to SEQ ID NO: 82
  > Migut.H00557

SEQ ID NO: 85 - DNA, 1830 bp, Medicago truncatula
  > Medtr6g016600
atggcttgtttttctttaccttgttgtactgggtcatttttgacaccaaataggttaaaaattaagtttaatcaatgctataattttgaaaatgggtttttatggaataagaaatttgagtattatggtagaaaaagtagaaatttttcattgaggtttcaacaaaatagtgttggtaatgaaattgagccacttgttagtagtagaagtagggatgataggaagagtgattatggtaaaggggggcgtggtaacaggttaaagaagagattttcgttgagattgcggccaaggttacggttattggttatgaggatgaaaagggcttctgttgaatctgttttcaatgaggttggagtgtttgttaggaagaatattaggacggtggcgtttgctacttcgttttcgattgtgtttacactttgtttcttgtttttgaaatttacttcacttccgcctgtgaaaatggttccgtattcggacttgattgcgagtcttcgaaatggaaatgtggagaaagttttagttgaagaagggtctcggcgtatatattacaatacggtttctgatgaagaagaatcacaacaagtagtagatgtttcgatggataaggatatagacgaggttgtgagtgagaatgctgtaaaatctggtggaattcctgtgttgaataaattgaagaaattctctaagagacgagcttcagttcctgagtggcagtattccacgagaaaaatagatcatgacgaaaaattccttcttagtttgatgagggaaaaggaggttacatttagctcggctcctcaatctgtgttagcagcgatgaggggtactttgataactgtgatcacactgtggatacctttgattccgttgatgtgggttctatatcgtcaactttctgccgctaatagtccagcgaaaaagcggaaacctaagagtcagacagttggatttgaagatgttcaaggtgttgattctgcaaaagtagaactcatggagatagtttcatgtctgcaaggagatataaattaccaaaagctaggggcaaagctacccagaggagtgttgctagtcggtcctccaggaactggcaagacattacttgcacgtgcagtagcgggggaagcaggtgtaccctttttcacagtatctgctagtgagtttgtagaaatgtttgttggaagaggtgcagctagaatcagagacctgttcagtagagcaaggaaatttgcaccatcaatcatattcattgatgaacttgatgcggttggtggcaagcgtggaagaggcttcaatgaggaacgggaccaaacgctaaatcagttgctgacagaaatggatggatttgaatcagaaatcagagtggttgtcattgcagcaactaaccgaccagaagccttggatccagccctgtgtcgtcccggtcgtttctcgaggaaagtatttgtaggggaacctgatgaagaaggaaggagaaaaatattagctgtgcatttgagaggagttcctttggaggaggatgctaacatcatatgtcatttaattgccacagtcactgatggtctcgtaggtgctgatcttgcaaacatcgtcaatgaatccgctttgctcgctgctcgaagaggtagtgaaactgtggcaagggaagatataatcgaagctattgaaagagcaaaatttggaatcaatgataagcaactaaggtctagcaaaataagcaaggagctaggtaagctgttcccttggatgccatcattgatgggaagaagtgataggaaacaagatgacatacaaggaccattaggttatcaatcattgaattcatga

SEQ ID NO: 86 - protein, 609 aa, Medicago truncatula
  > Medtr6g016600
MACFSLPCCTGSFLTPNRLKIKFNQCYNFENGFLWNKKFEYYGRKSRNFSLRFQQNSVGNEIEPLVSSRSRDDRKSDYGKGGRGNRLKKRFSLRLRPRLRLLVMRMKRASVESVFNEVGVFVRKNIRTVAFATSFSIVFTLCFLFLKFTSLPPVKMVPYSDLIASLRNGNVEKVLVEEGSRRIYYNTVSDEEESQQVVDVSMDKDIDEVVSENAVKSGGIPVLNKLKKFSKRRASVPEWQYSTRKIDHDEKFLLSLMREKEVTFSSAPQSVLAAMRGTLITVITLWIPLIPLMWVLYRQLSAANSPAKKRKPKSQTVGFEDVQGVDSAKVELMEIVSCLQGDINYQKLGAKLPRGVLLVGPPGTGKTLLARAVAGEAGVPFFTVSASEFVEMFVGRGAARIRDLFSRARKFAPSIIFIDELDAVGGKRGRGFNEERDQTLNQLLTEMDGFESEIRVVVIAATNRPEALDPALCRPGRFSRKVFVGEPDEEGRRKILAVHLRGVPLEEDANIICHLIATVTDGLVGADLANIVNESALLAARRGSETVAREDIIEAIERAKFGINDKQLRSSKISKELGKLFPWMPSLMGRSDRKQDDIQGPLGYQSLNS

SEQ ID NO: 87 - DNA, 1734 bp, Oryza sativa
  > LOC_Os04g39190
atggctgccgtggtggcggcggcggcggcttgcttgtcccccgtgtgcgctgccgctgcctccgtcccgcgcgctcgggtttgcttcgtgagcccccccggttcttggagctgcctcgccgcgagcaatggccgcgggttgcttcgcggtgggaatgggatgcggttgcggtggagggcaccggtgcgcgccaaggtggacgaggacaaggaggccgggctggggttccgcgagccggaacggaggaggatgcggctgcggctccggccgcggctgcgcctgctgtggtggcggctccggcggctgtcgccgcgggacctccccggggacgccgcggccgcgctgcggcgcgccgctcgacgcgtgccgcccgccgcggccgcgcccatcgtcctcgcggtcctcctcctcgccgcccgcctcgcgctgcccaagaacgcggccaaggaggtggcctactcggacctcctggctggcctccgcgcgggcgccgtcaccgccgtcgcgttcgaggaggactcccgccgcatctacttccgcagggccgcggacgacgggggcggcagcgacgacgccggcgcgggcgcgggcgaggcccgcagaggcgccgcggcggcggcgaggtggccgtgctacgcgaggagggtgccgcacgacgaggggttcttgctggggctgatgcgggacggcggggtggactaccggtcggcgccgcggccggcggggaggctgctggtggacatgctgagcacgctgctcacgctgtgggtgtcgctgctgccgatgatgtggttcatacagcggcagatgtccgccggtggcggcgcagagaagcggcgccggccgaggaagcagcgagtggggttcgacgatgtccagggcgtcgatgaggccaaggaggaactcgtcgaggtagtgagttgcttgcatggttcactgaactacaagaagctcggagctaaattacccaggggtgttctgcttgttgggcctccaggaactgggaaaactcttctggcaagggcagttgcaggagaggctggaattccctttttctctgtttctgctagtgaatttgttgaagttttcgttggaagaggagcagcccgtgtaagggatttattcaaggaggccaaagaagctgccccatctataattttcattgatgaacttgatgctgtgggtggaagtcgaggtagaagttttaatgatgagagggaccaaactttaaaccagctgcttactgagatggatggttttgactcggacatgaaagttattgtcatggctgctactaataggcctaaagcactggaccctgctctgtgtcgccctggtcggttctcaagaaaagttttggttggtgtgcctgatttggaggggcgaaggaatattttggctgttcatttaagggacgttcctttggaagaggatcctgaaataatttgtgatctggttgctagtttaacaccaggattggttggtgcggatctagcaaacattgtgaatgaggctgcgttactagctgctcgaaggggtggcaatactgtggctcgggaggatattatggatgcaattgagagggagaaatatggggtcaatggtagacaagagaatgctgattctgaaaggcaaggcctcactaaattgtttccatggttacctaaacctggaaatagaccaacaaacccagatgatattggaggagttatgggatatcatacattaagctaa

SEQ ID NO: 88 - protein, 577 aa, Oryza sativa
  > LOC_Os04g39190
MAAVVAAAAACLSPVCAAAASVPRARVCFVSPPGSWSCLAASNGRGLLRGGNGMRLRWRAPVRAKVDEDKEAGLGFREPERRRMRLRLRPRLRLLWWRLRRLSPRDLPGDAAAALRRAARRVPPAAAAPIVLAVLLLAARLALPKNAAKEVAYSDLLAGLRAGAVTAVAFEEDSRRIYFRRAADDGGGSDDAGAGAGEARRGAAAAARWPCYARRVPHDEGFLLGLMRDGGVDYRSAPRPAGRLLVDMLSTLLTLWVSLLPMMWFIQRQMSAGGGAEKRRRPRKQRVGFDDVQGVDEAKEELVEVVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDPALCRPGRFSRKVLVGVPDLEGRRNILAVHLRDVPLEEDPEIICDLVASLTPGLVGADLANIVNEAALLAARRGGNTVAREDIMDAIEREKYGVNGRQENADSERQGLTKLFPWLPKPGNRPTNPDDIGGVMGYHTLS

SEQ ID NO: 89 - DNA, 1752 bp, Oropetium thomaeum
  > Oropetium_20150105_22725
atggcgatggccgccgtagcttctctctactaccctttatccgccacttctctcccgcgggcccagccctccccctcctgcttcacgcgcgccacttcctggaaccgctctgccgcgacatcgagcacgagcagtggtctttgctgcggctgcggggtcccttgcggcttgggttacgcgaggctgcggtcgcggtggaggaagccggttcgcgctaaaatcgacgagttggataaggacagggacgccggtctcgagttccagcccccggaccgcaggaagctgcgcctacgcctgcggccgcgcctgcgcctgctatggtggcggctccggcggctgtcgctgcgggacctcgcgtcagacgccggggccgcgctgcgccgcgccgtccgccgcgtgcctctagcggcagcggcaccggtcctcctggccgttctcctcgccgccgcgcggctcgcgctcccgaagaacacggcccgggaggtggcctactccgatctcgtggcggggcttcgcgagggcaccgtatcagctgccgcgttcgaggaggactcgcgccgcatctacttcagcaagaaggccgtggacgacgacgggtccgacgcgagcgattctggggaaagcgcaggcgaggcgacgaaatggccgtactacgcgcggagggtgcagcacgacgaagggttcctgttggggcttatgcgggagggtggggtggactaccggtcatcgccacggccggcaggtaggttgctggtggacatgctgagcacgctgctcacgctctgggtctcgctgctgcccatgatgtggttcatacaaaggcaaatgtcggctgggggcggctcagagaagcggcgcaagacaaggaagcagagagtgttctttgatgatgttcagggcgtcgatgaagtgaaggaagagcttgtcgagattgtgagctgcttgcatggttcactaaactacaagaagcttggagcaaaattgccgaggggtgttctgcttgttggccctccaggaactgggaagactctgctggcaagggcagttgcaggagaggctggaattccatttttctctgtttcagccagtgagtttgttgaagtttttgttggaagaggtgcagccagggtcagggatttgttcaaggaggccaaagaagctgctccatcaatcatttttattgatgaacttgatgctgtgggtggcagccgaggtagaagttttaatgatgagagagatcaaactctaaaccagctgctcaccgaaatggatggatttgactcagacatgaaagttatcgtcatggctgctactaacagaccaaaagcattggatgctgctctttgtcgacctggccgcttctccagaaaggttgttgttggtgtgcctgatttggagggccgcaaaaagattttggctgttcatctaagggagattcctttagaagaggaccctgaaataattagcgatctgattgctaatgtaaccccaggattggttggtgcagatctagcaaatattgttaacgagtctgccctactagctgcccgaagaggtgcaaaaagtgtagctcgtgaggatattatggatgcaattgagagggagaaatatggagtcaatggtagacaacaaactgatgattctgaaaggcaaggcctcaccaaattatttccatggttacctaaacctggaaataaacaatcaagccctgatgattttggaagattgatgggttatcatacattaagctaa

SEQ ID NO: 90 - protein, 583 aa, Oropetium thomaeum
  > Oropetium_20150105_22725
MAMAAVASLYYPLSATSLPRAQPSPSCFTRATSWNRSAATSSTSSGLCCGCGVPCGLGYARLRSRWRKPVRAKIDELDKDRDAGLEFQPPDRRKLRLRLRPRLRLLWWRLRRLSLRDLASDAGAALRRAVRRVPLAAAAPVLLAVLLAAARLALPKNTAREVAYSDLVAGLREGTVSAAAFEEDSRRIYFSKKAVDDDGSDASDSGESAGEATKWPYYARRVQHDEGFLLGLMREGGVDYRSSPRPAGRLLVDMLSTLLTLWVSLLPMMWFIQRQMSAGGGSEKRRKTRKQRVFFDDVQGVDEVKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGVPDLEGRKKILAVHLREIPLEEDPEIISDLIANVTPGLVGADLANIVNESALLAARRGAKSVAREDIMDAIEREKYGVNGRQQTDDSERQGLTKLFPWLPKPGNKQSSPDDFGRLMGYHTLS

SEQ ID NO: 91 - DNA, 1671 bp, Oropetium thomaeum
  > Oropetium_20150105_26905
atggcgatggccgccgtagcttctctctactaccctttatccgccacttctctcccgcgggcccagccctcccctcctgcttcacgcgcgccacttcctggaaccgctctgccgcgacatcgagcacgagcagtggtctttgctgcggctgcggggtcccttgcggcttgggttacgcgaggctgcggtcgcggtggaggaagccggttcgcgctaaaatcgacgagttggataaggacagggacgccggtctcgagttccagcccccggaccgcaggaagctgcgcctacgcctgcggcccgcgcctgcgcctgctatggtggcggctccggcggctgtcgctgcgggacctcgcgtcagacgccggggccgcgctgcgccgcgccgtccgccgcgtgcctctagcggcagcggcaccggtcctcctggccgttctcctcgccgccggcggctcgcgctcccgaagaacacggcccgggaggtggcctactccgatctcgtggcggggcttcgcgagggcaaccgtatcagctgccgcgttcgaggaggactcgcgccgcatctacttcagcaagaaggccgtggacgacgacgggtccgacgcgagcgattctggggaaagcgcaggcgaggcgacgaaatggccgtactacgcgcggagggtgcagcacgacgaagggttcctgttggggcttatgcgggagggtgggtggactaccggtcatcgccacggccggcaggcaaatgtcggctggggcggctcagagaagcggcgcaagacaaggaagcagagagtgttctttgatgatgttcagggcgtcgatgaagtgaaggaagagcttgtcgagattgtgagctgcttgcatggttcactaaactacaagaagcttggagcaaaattgccgaggggtgttctgcttgttggccctccaggaactgggaagactctgctggcaagggcagttgcaggagaggctggaattccatttttctctgtttcagccagtgagtttgttgaagtttttgttggaagaggtgcagccagggtcagggatttgttcaaggaggccaaagaagctgctccatcaatcatttttattgatgaacttgatgctgtgggtggcagccgaggtagaagttttaatgatgagagagatcaaactctaaaccagctgctcaccgaaatggatggatttgactcagacatgaaagttatcgtcatggctgctactaacagaccaaaagcattggatgctgctctttgtcgacctggccgcttctccagaaaggttgttgttggtgtgcctgatttggagggccgcaaaaagattttggctgttcatctaagggagattcctttagaagaggaccctgaaataattagcgatctgattgctaatgtaaccccaggattggttggtgcagatctagcaaatattgttaacgagtctgccctactagctgcccgaagaggtgcaaaaagtgtagctcgtgaggatattatggatgcaattgagagggagaaatatggagtcaatggtagacaacaaactgatgattctgaaaggcaaggcctcaccaaattatttccatggttacctaaacctggaaataaacaatcaagccctgatgattttggaagattgatgggttatcatacattaagctaa

SEQ ID NO: 92 - protein, 556 aa, Oropetium thomaeum
  > Oropetium_20150105_26905
MAMAAVASLYYPLSATSLPRAQPSPPASRAPLPGTALPRHRARAVVFAAAAGSLAAWVTRGCGRGGGSRFALKSTSWIRTGTPVSSSSPRTAGSCAYACGPRLRLLWWRLRRLSLRDLASDAGAALRRAVRRVPLAAAAPVLLAVLLAAGGSRSRRTRPGRWPTPISWRGFARATVSAAAFEEDSRRIYFSKKAVDDDGSDASDSGESAGEATKWPYYARRVQHDEGFLLGLMREGGWTTGHRHGRQANVGWGGSEKRRKTRKQRVFFDDVQGVDEVKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGVPDLEGRKKILAVHLREIPLEEDPEIISDLIANVTPGLVGADLANIVNESALLAARRGAKSVAREDIMDAIEREKYGVNGRQQTDDSERQGLTKLFPWLPKPGNKQSSPDDFGRLMGYHTLS

SEQ ID NO: 93 - DNA, 1734 bp, Panicum hallii
  > Pahal.G00536
atggcctctctccgccccatatccgccgcgccctccttcccgcgcgctcgcgccgcctgcttcaggagccccgcctcctggagcggcctcgccgcggccgcgaggggcaatgcctatggctgcggctgcacggttccacccgccgcggccgggggtcacgcgaggctgcggacgcggtggaggagccccgtgcgcgccaaggtcgacgaggcggacaaggacgcgggcgcggggctggggctccgcacgcccggccgcaggaggaggccgcggctgcgcctgcggccgcggctgcgccttatgctgtggcggctccggaggctgtcgccgcgggacctcgcggccgcgctgcggcgcgccgtccgccgcgtgcctcccgcggccgcggcgccggtcctcctggcgttcctcctgttcgccgcgcggctcgcgctaccgaagaacgtggcaaaggaggtggcctactcggacctcgtcgcggggctccgcgagggcgccgtagccgccgtcgcgttcgaggaggactcgcgccgcatctacttcagcaagaaggccggggacgccagcggcagcgacgaagcgagcgagcccgagactgatagcgcagccgcggcggcgcccaagtggccgtactacgcgcggagggtgccgcacgacgaggggttcctgctcgggcttatgcgggagggcggggtggactaccggtcggcgccgcggcctgcggggaggctgctggtggacatgctgagcacgctgctcacactctgggtctcgctgctgccgatgatgtggttcatacagcggcagatgtccggtgggggcagtgctgacaagcgacgcaagccgaggaagcagagggtggggtttgacgatgttcagggcgtggatgaagcgaaggaggagcttgtggagatagtgagctgtttgcatggttcattgaactacaagaagcttggagcaaaactgcccaggggtgttctgcttgttggccccccaggaactgggaagactctgctagcaagggcagttgcaggagaggctggaattccatttttctctgtttctgctagtgagtttgttgaagttttcgttggaagaggtgctgctcgggtcagggatttgttcaaggaagccaaagaagctgctccgtcaatcatttttattgatgaacttgatgccgtgggtggaagtagaggtagaagttttaatgatgagagggaccaaactctaaaccagctgcttactgaaatggatggttttgactcggacatgaaagtaattgtcatggcagctactaacaggccaaaagcactggatgctgctctttgtcggcctggccgcttctccagaaaggttgttgttggtgtgcctgatgtggagggccgcaaaaagatcttggctgttcatctaagggatattcctttggaagagaatcctgaaataattagtgatctggttgctaatctaaccccaggactggttggcgcggatctagcaaacattgttaatgaggctgctctactagctgcacgaagaggtgggaatactgtgtcccgggaggatattatggatgcaatcgagagggagaaatacggggtcaatggtagacaagaaatcgatggttctgaaaggcaaggtctcaccaaattatttccatggctacctaagcccggaaataaaccatcaacccctgacgatttccgaggactgatgggttatcagacattaagctaa

SEQ ID NO: 94 - protein, 577 aa, Panicum hallii
  > Pahal.G00536
MASLRPISAAPSFPRARAACFRSPASWSGLAAAARGNAYGCGCTVPPAAAGGHARLRTRWRSPVRAKVDEADKDAGAGLGLRTPGRRRRPRLRLRPRLRLMLWRLRRLSPRDLAAALRRAVRRVPPAAAAPVLLAFLLFAARLALPKNVAKEVAYSDLVAGLREGAVAAVAFEEDSRRIYFSKKAGDASGSDEASEPETDSAAAAAPKWPYYARRVPHDEGFLLGLMREGGVDYRSAPRPAGRLLVDMLSTLLTLWVSLLPMMWFIQRQMSGGGSADKRRKPRKQRVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGVPDVEGRKKILAVHLRDIPLEENPEIISDLVANLTPGLVGADLANIVNEAALLAARRGGNTVSREDIMDAIEREKYGVNGRQEIDGSERQGLTKLFPWLPKPGNKPSTPDDFRGLMGYQTLS

SEQ ID NO: 95 - DNA, 1734 bp, Panicum hallii; identical to SEQ ID NO: 93
  > Pahal.G00536

SEQ ID NO: 96 - protein, 577 aa, Panicum hallii; identical to SEQ ID NO: 94
  > Pahal.G00536

SEQ ID NO: 97 - DNA, 1734 bp, Panicum hallii; identical to SEQ ID NO: 93
  > Pahal.G00536

SEQ ID NO: 98 - protein, 577 aa, Panicum hallii; identical to SEQ ID NO: 94
  > Pahal.G00536

SEQ ID NO: 99 - DNA, 1734 bp, Panicum hallii; identical to SEQ ID NO: 93
  > Pahal.G00536

SEQ ID NO: 100 - protein, 577 aa, Panicum hallii; identical to SEQ ID NO: 94
  > Pahal.G00536

SEQ ID NO: 101 - DNA, 1941 bp, Prunus persica
  > Prupe.3G243600
atggcttgtttttctgttgtttgcaatactgggttcttatctattccaaacaaattggaagttcatggccgaaagtccaagtctttggggagatatagaggtttttgttgtcgttcatttgcttttcgttcgttgggatatcacaaattttgcaactttcaacatgggttgctctggaacaatgagcttaggccactgagtaatggaaaaagtggagttttcttaaagggattcaataatcgttataaatctaaacaggagttgtgttgttacaacaaaattgagccactgacaaatgcaaacagtgcaaataagcagatgcatttgggaaaaaaaggaaacaccaagttaaggtctttgaggaagagattttctttaagattacgcccaaggttgcggttattaactataagattgaaaacagttacgattcgatcagtgttaaatggtattgggaccttcttgagaaagaatataagaagagtgactcttttttcttcaatttctgcaacattggggctgtgctatctgtttttgaagttaacagcagtgccttctccaaaaatggttccatattcagaattgataacaagccttcgaaatgattctgttacgaaagttctacttgaagagggatctcgtcggatatattataacacaaactgtcgcattgctggggatactcatttgtttgatgaagaattgacaaacgtacaaggagagaatatggccgataaggtcacaagtgatgatggttctcgatctgatcaagcattgaatacgaacgtattgaaaaaattatcagtcactcaagcttcaacccctgactggcagtattccactagaaaaatagatcatgatgagaaatttttacttagtttgatgagagagaaaggaattacctatagctcggctcctcaatcagtgttgatgtcaatgaggactactttgataactataatatctctgtggattcctttgctgccattgatgtggcttctttatcgtcaacttactgctggtaatagcccagccaaaaagcggagacctgataatcagtcagttggatttgatgatgttgagggagttgattctgccaaattggagctcatggagatagttttatgcttgcaaggagctattaactataacaaactgggagcaaagttacctagaggtgtattgctggtgggtcctccaggaacagggaaaacattactcgcccgtgcagtggctggagaagcaggtgtaccatttttcactgtttctgccagtgaatttgtggagatgtttgttggaagaggggcagctcgcattagagacctttttaatatggcaaggaaatattcaccatcgatcatattcattgatgagcttgacgcggttggaacaaagcgtggcagaagtttcaatgatgagcgtgaccaaactctgaatcagttgctgacagaaatggatggatttgagtcagactcgaaggtgattgttgttgctgcaacaaacaggcctgaagtattggattctgccctttgtaggccaggccgcttttcaaggaaaatagttgtaggagaaccagatgaagaaggacggagaaagattttggctgtacatttgagaggaattcctttggaggaagacagtaatcttatttgcagcctcattgcttctctgacaccaggttttgtgggtgctgatcttgcaaacattgtcaatgaagctgctttacttgctgctcgtagaggtggtgaaactgtggccagagaagatgtcatggaagcaattgaaagagcaaagtttggaataaatgataagcaacttagacctagtactattagcaaggagcttgggaaaatgttcccatggatgccttctctaatgggaaagaataacgcaagacaagatggattgccagggccactaggatatcaagctctgagttga

SEQ ID NO: 102 - protein, 646 aa, Prunus persica
  > Prupe.3G243600
MACFSVVCNTGFLSIPNKLEVHGRKSKSLGRYRGFCCRSFAFRSLGYHKFCNFQHGLLWNNELRPLSNGKSGVFLKGFNNRYKSKQELCCYNKIEPLTNANSANKQMHLGKKGNTKLRSLRKRFSLRLRPRLRLLTIRLKTVTIRSVLNGIGTFLRKNIRRVTLFSSISATLGLCYLFLKLTAVPSPKMVPYSELITSLRNDSVTKVLLEEGSRRIYYNTNCRIAGDTHLFDEELTNVQGENMADKVTSDDGSRSDQALNTNVLKKLSVTQASTPDWQYSTRKIDHDEKFLLSLMREKGITYSSAPQSVLMSMRTTLITIISLWIPLLPLMWLLYRQLTAGNSPAKKRRPDNQSVGFDDVEGVDSAKLELMEIVLCLQGAINYNKLGAKLPRGVLLVGPPGTGKTLLARAVAGEAGVPFFTVSASEFVEMFVGRGAARIRDLFNMARKYSPSIIFIDELDAVGTKRGRSFNDERDQTLNQLLTEMDGFESDSKVIVVAATNRPEVLDSALCRPGRFSRKIVVGEPDEEGRRKILAVHLRGIPLEEDSNLICSLIASLTPGFVGADLANIVNEAALLAARRGGETVAREDVMEAIERAKFGINDKQLRPSTISKELGKMFPWMPSLMGKNNARQDGLPGPLGYQALS

SEQ ID NO: 103 - DNA, 1941 bp, Prunus persica; identical to SEQ ID NO: 101
  > Prupe.3G243600

SEQ ID NO: 104 - protein, 646 aa, Prunus persica; identical to SEQ ID NO: 102
  > Prupe.3G243600

SEQ ID NO: 105 - DNA, 1449 bp, Prunus persica
  > Prupe.3G243600
atggcttgtttttctgttgtttgcaatactgggttcttatctattccaaacaaattggaagttcatggccgaaagtccaagtctttggggagatatagaggtttttgttgtcgttcatttgcttttcgttcgttgggatatcacaaattttgcaactttcaacatgggttgctctggaacaatgagcttaggccactgagtaatggaaaaagtggagttttcttaaagggattcaataatcgttataaatctaaacaggagttgtgttgttacaacaaaattgagccactgacaaatgcaaacagtgcaaataagcagatgcatttgggaaaaaaaggaaacaccaagttaaggtctttgaggaagagattttctttaagattacgcccaaggttgcggttattaactataagattgaaaacagttacgattcgatcagtgttaaatggtattgggaccttcttgagaaagaatataagaagagtgactcttttttcttcaatttctgcaacattggggctgtgctatctgtttttgaagttaacagcagtgccttctccaaaaatggttccatattcagaattgataacaagccttcgaaatgattctgttacgaaagttctacttgaagagggatctcgtcggatatattataacacaaactgtcgcattgctggggatactcatttgtttgatgaagaattgacaaacgtacaaggagagaatatggccgataaggtcacaagtgatgatggttctcgatctgatcaagcattgaatacgaacgtattgaaaaaattatcagtcactcaagcttcaacccctgactggcagtattccactagaaaaatagatcatgatgagaaatttttacttagtttgatgagagagaaaggaattacctatagctcggctcctcaatcagtgttgatgtcaatgaggactactttgataactataatatctctgtggattcctttgctgccattgatgtggcttctttatcgtcaacttactgctggtaatagcccagccaaaaagcggagacctgataatcagtcagttggatttgatgatgttgagggagttgattctgccaaattggagctcatggagatagttttatgcttgcaaggagctattaactataacaaactgggagcaaagttacctagaggtgtattgctggtgggtcctccaggaacagggaaaacattactcgcccgtgcagtggctggagaagcaggtgtaccatttttcactgtttctgccagtgaatttgtggagatgtttgttggaagaggggcagctcgcattagagacctttttaatatggcaaggaaatattcaccatcgatcatattcattgatgagcttgacgcggttggaacaaagcgtggcagaagtttcaatgatgagcgtgaccaaactctgaatcaggtaatctga

SEQ ID NO: 106 - protein, 482 aa, Prunus persica
  > Prupe.3G243600
MACFSVVCNTGFLSIPNKLEVHGRKSKSLGRYRGFCCRSFAFRSLGYHKFCNFQHGLLWNNELRPLSNGKSGVFLKGFNNRYKSKQELCCYNKIEPLTNANSANKQMHLGKKGNTKLRSLRKRFSLRLRPRLRLLTIRLKTVTIRSVLNGIGTFLRKNIRRVTLFSSISATLGLCYLFLKLTAVPSPKMVPYSELITSLRNDSVTKVLLEEGSRRIYYNTNCRIAGDTHLFDEELTNVQGENMADKVTSDDGSRSDQALNTNVLKKLSVTQASTPDWQYSTRKIDHDEKFLLSLMREKGITYSSAPQSVLMSMRTTLITIISLWIPLLPLMWLLYRQLTAGNSPAKKRRPDNQSVGFDDVEGVDSAKLELMEIVLCLQGAINYNKLGAKLPRGVLLVGPPGTGKTLLARAVAGEAGVPFFTVSASEFVEMFVGRGAARIRDLFNMARKYSPSIIFIDELDAVGTKRGRSFNDERDQTLNQVI

SEQ ID NO: 107 - DNA, 1935 bp, Populus trichocarpa
  > Potri.004G106500
atggcttctttttctgctgtttgcaataacgggttcttgaattttaaactgaactctggagtcaataatggaaatactaaatctttgaagagaaattcaactatttgttacagtccagtgttgatcccttctttgggactttgcagtgctggcaaactccaacatttgttgctttggaggagcaagtttaggtcatcattttatgatggaaaaaccggggttcatttatcggggttttgtggtggttgtaagtctcaaaatgggatgtcttgtaagagctcatttaggccattgagcagtgaaaaaagtagagatgaagagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataagaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatctcgtcgtatatactataacacagactctgtgggtactgaaaactcagaagacaaatcttcagtattaaatcttccaaatgagaatgcagctgaaacagttgcaatagaacgtgttgtgagcaaaactggtctggcatcaagggtagatgtatttaagaaattttcaaggccacgagcttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcacgaaagaggcgatcaaataatcaggaagtcacttttgatgatgtggagggtgttgatgttgctaagatagaactcatggagatagttttgtgtctgcaaggagcaatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcagcaagaaaatcttctccgtcaattatattcatcgatgaacttgatgcagttgggggaaaacgtggtagaagtttcaatgacgaacgagatcaaacactaaaccagttgctgactgaaatggatggttttgagtcagaaatgaaagtggtggttattgcagcaactaataggccggaagctttggatccagccctttgccggcctggtcgcttttcaagaaaggtagttgttggagaaccagatgaagaaggaaggagaaagatcttggctgtacatttgagaggggttcctatagatgaagatgcagatcttatctgtaatctagttgcttcattgacaccagggtttgtaggtgctgatcttgcaaacatcattaatgaagctgctttacttgctgctcgaagaggtggagacattgtgacaagggaagatgtaatggaagcaattgaaagagcaaagtttggaatcggggacaggcaactaaggcccagtacaataagcaaagagctgggcaaattgttcccatggattccttcgttgatgggaacgattgacacaagacaggatggtttgcaaggctctttaggttaccagaccctgagctga

SEQ ID NO: 108 - protein, 644 aa, Populus trichocarpa
  > Potri.004G106500
MASFSAVCNNGFLNFKLNSGVNNGNTKSLKRNSTICYSPVLIPSLGLCSAGKLQHLLLWRSKFRSSFYDGKTGVHLSGFCGGCKSQNGMSCKSSFRPLSSEKSRDEESHLRKRGNGNLRKRFSLRLKTRLRLLKIRLKGVSIRSMLNDFGMFLRRNIRRMTLYTSISVALGMCYLFLRLTALPSPKIVPYSELIASLQNGYVTNVLFEEGSRRIYYNTDSVGTENSEDKSSVLNLPNENAAETVAIERVVSKTGLASRVDVFKKFSRPRASTPEWQFSTRKVDRDEKFLLTLMRAKGTAYSSAPQSILMSIRSLLITIISLWIPLTPMMWLLYRQLSAANSPARKRRSNNQEVTFDDVEGVDVAKIELMEIVLCLQGAMNYQKLGAKLPRGVLLVGPPGTGKTLLARAVAGEAGVPFFSVSASEFVELFVGRGAARIRDLFNAARKSSPSIIFIDELDAVGGKRGRSFNDERDQTLNQLLTEMDGFESEMKVVVIAATNRPEALDPALCRPGRFSRKVVVGEPDEEGRRKILAVHLRGVPIDEDADLICNLVASLTPGFVGADLANIINEAALLAARRGGDIVTREDVMEAIERAKFGIGDRQLRPSTISKELGKLFPWIPSLMGTIDTRQDGLQGSLGYQTLS

SEQ ID NO: 109 - DNA, 1671 bp, Populus trichocarpa
  > Potri.004G106500
atgtcttgtaagagctcatttaggccattgagcagtgaaaaaagtagagatgaagagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataagaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatctcgtcgtatatactataacacagactctgtgggtactgaaaactcagaagacaaatcttcagtattaaatcttccaaatgagaatgcagctgaaacagttgcaatagaacgtgttgtgagcaaaactggtctggcatcaagggtagatgtatttaagaaattttcaaggccacgagcttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcacgaaagaggcgatcaaataatcaggaagtcacttttgatgatgtggagggtgttgatgttgctaagatagaactcatggagatagttttgtgtctgcaaggagcaatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcagcaagaaaatcttctccgtcaattatattcatcgatgaacttgatgcagttgggggaaaacgtggtagaagtttcaatgacgaacgagatcaaacactaaaccagttgctgactgaaatggatggttttgagtcagaaatgaaagtggtggttattgcagcaactaataggccggaagctttggatccagccctttgccggcctggtcgcttttcaagaaaggtagttgttggagaaccagatgaagaaggaaggagaaagatcttggctgtacatttgagaggggttcctatagatgaagatgcagatcttatctgtaatctagttgcttcattgacaccagggtttgtaggtgctgatcttgcaaacatcattaatgaagctgctttacttgctgctcgaagaggtggagacattgtgacaagggaagatgtaatggaagcaattgaaagagcaaagtttggaatcggggacaggcaactaaggcccagtacaataagcaaagagctgggcaaattgttcccatggattccttcgttgatgggaacgattgacacaagacaggatggtttgcaaggctctttaggttaccagaccctgagctga

SEQ ID NO: 110 - protein, 556 aa, Populus trichocarpa
  > Potri.004G106500
MSCKSSFRPLSSEKSRDEESHLRKRGNGNLRKRFSLRLKTRLRLLKIRLKGVSIRSMLNDFGMFLRRNIRRMTLYTSISVALGMCYLFLRLTALPSPKIVPYSELIASLQNGYVTNVLFEEGSRRIYYNTDSVGTENSEDKSSVLNLPNENAAETVAIERVVSKTGLASRVDVFKKFSRPRASTPEWQFSTRKVDRDEKFLLTLMRAKGTAYSSAPQSILMSIRSLLITIISLWIPLTPMMWLLYRQLSAANSPARKRRSNNQEVTFDDVEGVDVAKIELMEIVLCLQGAMNYQKLGAKLPRGVLLVGPPGTGKTLLARAVAGEAGVPFFSVSASEFVELFVGRGAARIRDLFNAARKSSPSIIFIDELDAVGGKRGRSFNDERDQTLNQLLTEMDGFESEMKVVVIAATNRPEALDPALCRPGRFSRKVVVGEPDEEGRRKILAVHLRGVPIDEDADLICNLVASLTPGFVGADLANIINEAALLAARRGGDIVTREDVMEAIERAKFGIGDRQLRPSTISKELGKLFPWIPSLMGTIDTRQDGLQGSLGYQTLS

SEQ ID NO: 111 - DNA, 1935 bp, Populus trichocarpa; identical to SEQ ID NO: 107
  > Potri.004G106500

SEQ ID NO: 112 - protein, 644 aa, Populus trichocarpa; identical to SEQ ID NO: 108
  > Potri.004G106500

SEQ ID NO: 113 - DNA, 1935 bp, Populus trichocarpa; identical to SEQ ID NO: 107
  > Potri.004G106500

SEQ ID NO: 114 - protein, 644 aa, Populus trichocarpa; identical to SEQ ID NO: 108
  > Potri.004G106500

SEQ ID NO: 115 - DNA, 1443 bp, Populus trichocarpa
  > Potri.004G106500
atggcttctttttctgctgtttgcaataacgggttcttgaattttaaactgaactctggagtcaataatggaaatactaaatctttgaagagaaattcaactatttgttacagtccagtgttgatcccttctttgggactttgcagtgctggcaaactccaacatttgttgctttggaggagcaagtttaggtcatcattttatgatggaaaaaccggggttcatttatcggggttttgtggtggttgtaagtctcaaaatgggatgtcttgtaagagctcatttaggccattgagcagtgaaaaaagtagagatgaagagagccatttgaggaaaagaggaaatggtaatttgaggaagaggttttcacttagattgaaaacaagactgcggctattgaagataagattgaagggagtttcgattaggtcgatgttgaatgattttggaatgttcttgagaaggaatataagaagaatgacactttacacttcaatttctgttgcattgggaatgtgctacttgtttttgagattaactgcgttgccatctccaaagattgttccttattcagaattgatagccagccttcaaaatggctatgttacaaacgtcttatttgaagagggatctcgtcgtatatactataacacagactctgtgggtactgaaaactcagaagacaaatcttcagtattaaatcttccaaatgagaatgcagctgaaacagttgcaatagaacgtgttgtgagcaaaactggtctggcatcaagggtagatgtatttaagaaattttcaaggccacgagcttccaccccagagtggcaattttctacaaggaaagtagatcgtgacgagaaatttcttcttactttgatgagagcaaagggaacagcatacagctcagcccctcaatcaattctgatgtcaataaggagtttgcttataactataatatctttgtggattcccttgactcctatgatgtggctcctctatcgccaactttctgctgctaatagcccagcacgaaagaggcgatcaaataatcaggaagtcacttttgatgatgtggagggtgttgatgttgctaagatagaactcatggagatagttttgtgtctgcaaggagcaatgaattatcaaaaactgggagcaaagttacctagaggtgtattgctggttggtcccccaggaacagggaagacattactggcacgtgcagttgctggagaagcaggagtaccatttttcagtgtctctgccagtgaatttgtggagttgtttgttggtagaggagcagctcggattagagatctctttaatgcagcaagaaaatcttctccgtcaattatattcatcgatgaacttgatgcagttgggggaaaacgtggtagaagtttcaatgacgaacgagatcaaacactaaaccaggtaacctaa

SEQ ID NO: 116 - protein, 480 aa, Populus trichocarpa
  > Potri.004G106500
MASFSAVCNNGFLNFKLNSGVNNGNTKSLKRNSTICYSPVLIPSLGLCSAGKLQHLLLWRSKFRSSFYDGKTGVHLSGFCGGCKSQNGMSCKSSFRPLSSEKSRDEESHLRKRGNGNLRKRFSLRLKTRLRLLKIRLKGVSIRSMLNDFGMFLRRNIRRMTLYTSISVALGMCYLFLRLTALPSPKIVPYSELIASLQNGYVTNVLFEEGSRRIYYNTDSVGTENSEDKSSVLNLPNENAAETVAIERVVSKTGLASRVDVFKKFSRPRASTPEWQFSTRKVDRDEKFLLTLMRAKGTAYSSAPQSILMSIRSLLITIISLWIPLTPMMWLLYRQLSAANSPARKRRSNNQEVTFDDVEGVDVAKIELMEIVLCLQGAMNYQKLGAKLPRGVLLVGPPGTGKTLLARAVAGEAGVPFFSVSASEFVELFVGRGAARIRDLFNAARKSSPSIIFIDELDAVGGKRGRSFNDERDQTLNQVT

SEQ ID NO: 117 - DNA, 993 bp, Panicum virgatum
  > Pavir.Ga01423
atgctgagcacgctgctcacgctctgggtctcgctgctgccgatgatgtggttaatacagcggcagttgtccggtgggggcagtgctgacaagcgacgcaagccgaggaagcagagggtggggtttgatgatgttcagggcgtggatgaagccaaggaggagcttgtggagatagtgagctgtttggatggttcattgaactacaagaagcttggagcaaaactgcccaggggtgttctgcttgttggccccccaggaactgggaagactctgctagcaagggcagttgcaggagaggctggaattccatttttctctgtttctgctagtgagtttgttgaagttttcgttggaagaggtgctgctcgggtcagggatttgttcaaggaggccaaagaggctgctccgtcaatcatttttattgatgaacttgatgccgtgggtggaagtagaggtaaaagttttaatgatgagagggaccaaactctaaaccagctgcttactgaaatggatggttttgactcggacatgaaagtaattgtcatggctgctactaacaggccaaaagcactggatgctgctctctgtcggcctggccgcttctccagaaaggttgttgttggtgtgcctgatgtggagggccgcaaaaagatcttggctgttcatctaagagatattcctttggaagaggatcccgaaataattagtgatctggttgctaatctaaccccaggactggttggtgcggatctagcaaacattgttaatgaggctgctctactagctgcacgaagaggtggaaatactgtgtcccgggaggatattatggatgcaatcgagagggagaaatatggggtcaatggtagacaagaaaccgatggttctgaaaggcaaggcttcaccaaattgtttccgtggctacctaagcctggaaataaaccgtcaagccctgatgatctccgaggactgatgggttatcagacattaagctaa

SEQ ID NO: 118 - protein, 330 aa, Panicum virgatum
  > Pavir.Ga01423
MLSTLLTLWVSLLPMMWLIQRQLSGGGSADKRRKPRKQRVGFDDVQGVDEAKEELVEIVSCLDGSLNYKKLGAKLPRGVLLVGPPGTGKTLLARAVAGEAGIPFFSVSASEFVEVFVGRGAARVRDLFKEAKEAAPSIIFIDELDAVGGSRGKSFNDERDQTLNQLLTEMDGFDSDMKVIVMAATNRPKALDAALCRPGRFSRKVVVGVPDVEGRKKILAVHLRDIPLEEDPEIISDLVANLTPGLVGADLANIVNEAALLAARRGGNTVSREDIMDAIEREKYGVNGRQETDGSERQGFTKLFPWLPKPGNKPSSPDDLRGLMGYQTLS

SEQ ID NO: 119 - DNA, 1929 bp, Phaseolus vulgaris
  > Phvul.004G047500
atggcttgtttttctttaccttgtaatactgggtcatttgttgttgttaccccttccatggcccaaagaacatattttggggtgtgtggaggtttgggcactaggtcatttgtttttacttctctagggtttaaccaatgctacaagtttccacatgggtttgtttgggacaagaaggtagggtattgtggtggtagaagtagagttccctcattgagggttccttattgttgcaagaccccgcttggtgtttcaagtaacaacaaaattgagccatttgcgagtagaagcaagggtgagaggaagactcactatggtaagggtgaaggtaataggttgaagaagaggttttcattgagattgcggccaaggttacggttattggctatgagaatgaaaagggcttccattaagtccattttgaatgagttggaggtacttattcgtaagaacattagagcagtggcgttttctgcctcagtttctgttgtgttcagcctttgtttcatgtttctaaaactgacagcactcccccctccgaaatctgttccatattctgacttgattaccagccttcagaatggatatgttgaaaaagttttagttgaagaggggtcccggcgtatatattacaacatgaagtcccaaattgttgaaaatgatcatgtttctggtgaagaatctcaagttgtagatgtttccattgatgcggatgttgataagatggggagtgagagtgcttctagggctggccaaacaccagtggtgaatgtactaaagaaattctctaagacccgagcttcaactcctgaatggcagtattccacaagaaaaatagatcatgatggaaaattccttgttagtttgatgagagaaaatggggttacatatagctctgcccctcaatctgcgttgatgtcaatgaggagtactttgatcactgtgataacactgtggatacctttaattccattgatgtggattctatatcgtcaactttctactgctaatagtcctgcaaggaaacagaggcctaatagtcagacagttggatttgatgatgtggaaggtgttgattctgcaaaagtagagctcatggagatagtttcatgtcttcaaggggatataaattaccgcaagctaggggcaaagctacctagaggcgtgctgctggtgggtcctcctggaactgggaagacactacttgcacgggcagtggcaggggaagcaggtgtaccctttttcactgtatctgcaagtgagtttgtggagttgtttgttggaagaggggcggctagaatcagagaccttttcaactcggcaagaaaatttgcaccatcaatcatattcattgatgaacttgatgcagtaggaggcaagcgtggaagaagtttcaacgacgagcgtgaccagacattaaaccagttgctgacagaaatggatggatttgaatcagagatgagagtggtagtcattgcagcaactaaccggccagaagccttggatccggctctatgtcgccctggtcgtttctcaagaaaagtatatgtaggcgaacctgatgaagaaggaaggagaaaaatattggctgtgcatctgagaggagttcctttggaggaggacactaacatcatttgccatttaattgcttctcttactactggttttgtaggtgctgatttggcaaatattgtcaatgaatccgctttgcttgctgctcgtagaggtagtgaaactgtggcaagggaagatataatggaagcaatagaaagagcaaaatttggaatcaatgataaacaattgaggtctagtaaactaagcaaggagcttaccaagctattcccatggatgccatcattgatgggaaaaaatgaaaggagacaggatgaccagcaaggaccattaggttatcaatcactgagttcatga

SEQ ID NO: 120 - protein, 642 aa, Phaseolus vulgaris
  > Phvul.004G047500
MACFSLPCNTGSFVVVTPSMAQRTYFGVCGGLGTRSFVFTSLGFNQCYKFPHGFVWDKKVGYCGGRSRVPSLRVPYCCKTPLGVSSNNKIEPFASRSKGERKTHYGKGEGNRLKKRFSLRLRPRLRLLAMRMKRASIKSILNELEVLIRKNIRAVAFSASVSVVFSLCFMFLKLTALPPPKSVPYSDLITSLQNGYVEKVLVEEGSRRIYYNMKSQIVENDHVSGEESQVVDVSIDADVDKMGSESASRAGQTPVVNVLKKFSKTRASTPEWQYSTRKIDHDGKFLVSLMRENGVTYSSAPQSALMSMRSTLITVITLWIPLIPLMWILYRQLSTANSPARKQRPNSQTVGFDDVEGVDSAKVELMEIVSCLQGDINYRKLGAKLPRGVLLVGPPGTGKTLLARAVAGEAGVPFFTVSASEFVELFVGRGAARIRDLFNSARKFAPSIIFIDELDAVGGKRGRSFNDERDQTLNQLLTEMDGFESEMRVVVIAATNRPEALDPALCRPGRFSRKVYVGEPDEEGRRKILAVHLRGVPLEEDTNIICHLIASLTTGFVGADLANIVNESALLAARRGSETVAREDIMEAIERAKFGINDKQLRSSKLSKELTKLFPWMPSLMGKNERRQDDQQGPLGYQSLSS

SEQ ID NO: 121 - DNA, 1911 bp, Ricinus communis
  > 29673.t000006
atggctactttttctgttgtttgtaataatgggttattgactaataaacagaatttagaactttatgatggaaagttcaagtctttgaggagatacacaactgtttgttataatggctcggtttatagcaattcccgctatttaatgctttctagggatgggtttaggttactttataatggaaaaagtgaagttcctttgttgggtttttgtgtctgttgcaagtctcaacatggattgttttgtcacagtaaaagaattgggccattgatgaatggagatagaggaaatgaggaaacccatttgagaaaaatagttaataatggtgtgaagaagagattattttcattgaggttaagaccaagattgcgactactcactagaagattgaagagagtttcactgaggtcaatgttaaatgattttggaatgttcttgaaaaagaatataagaagattaacactttatgcttcaattactgttgcacttggtatgtgctacttgtttttgaggttaactgcagttccttctccaaagatcgttccatattcagaattgatatcaagccttcaaagtggttctgttacaaaagttttacttgaagaaggatctcgtcgcatatattacaacatcaaatctcaaggaattgaaaatactgaaaattcagaagagataaatgtttcaaatgaaaatgaagctcatgtagttgcaagaggaggtattgtgagtaccagtggtagagcatcaaaattagatttgttgaagaaattttcagatactcgagcttctactcctgagtggcagtattctacgaggaaaatagaccatgatgagaaatttcttcttagtgtaatgagagaaaagggaacagtgtatggctcagcccctcaatcagttctgatgtctatgaggagcgtgttgataacaataatatctctgtggattcctttaacccctttgatgtggcttttgtaccgtcaactttctgctgccaacagccctgcgaagaagcctcgatttaatagtcgaatggtcaattttgatgatgtggagggtgttgatgctgctaagatagagcttatggagatagtttcatgcatgcaaggagctataaactatcaaaaactaggagcaaagatacctagaggtgtgttacttgtcggtcctccaggaacaggtaaaacattgctggcccgtgcagttgctggagaagcaggagtgccatttttcagtgtttctgccagtgaatttgtggagctgtttgttggaagaggagcagcacgcattagagatctctttaaagtggcaaggaaaagtgctccatcaatcattttcattgatgaactcgatgcagttggaggaaaacgtggtagaagtttcaatgatgagcgtgaccaaacactaaaccagttgttgacagaaatggacggatttgagtcggacatgaaagtggttgttattgcggcaaccaacaggccagaagcattggatcaagccctttgcaggcctggtcgcttttcaagaaaggtacttgtaggagaaccagatgaagaaggaagggaaaagatcctcagtgtacatttgagaggcgttcctctagaagaagacacagatcttatctgtaatctggttgcttctctgacaccaggttttgttggagctgatcttgcaaacattgtcaatgaagctgcattacttgctgctcggagaggtggtgagactgtgacaagggaagatataatggaagcgattgagagagcaaagtttgggatcaatgataggcagctggggcctactgcaataagcaaggagcttgggaaacttttcccatggataccatctttaatgagaaggaataacacaggacaagatggtttgcaagggcctttaggttaccagactctgagctga

SEQ ID NO: 122 - protein, 636 aa, Ricinus communis
  > 29673.t000006
MATFSVVCNNGLLTNKQNLELYDGKFKSLRRYTTVCYNGSVYSNSRYLMLSRDGFRLLYNGKSEVPLLGFCVCCKSQHGLFCHSKRIGPLMNGDRGNEETHLRKIVNNGVKKRLFSLRLRPRLRLLTRRLKRVSLRSMLNDFGMFLKKNIRRLTLYASITVALGMCYLFLRLTAVPSPKIVPYSELISSLQSGSVTKVLLEEGSRRIYYNIKSQGIENTENSEEINVSNENEAHVVARGGIVSTSGRASKLDLLKKFSDTRASTPEWQYSTRKIDHDEKFLLSVMREKGTVYGSAPQSVLMSMRSVLITIISLWIPLTPLMWLLYRQLSAANSPAKKPRFNSRMVNFDDVEGVDAAKIELMEIVSCMQGAINYQKLGAKIPRGVLLVGPPGTGKTLLARAVAGEAGVPFFSVSASEFVELFVGRGAARIRDLFKVARKSAPSIIFIDELDAVGGKRGRSFNDERDQTLNQLLTEMDGFESDMKVVVIAATNRPEALDQALCRPGRFSRKVLVGEPDEEGREKILSVHLRGVPLEEDTDLICNLVASLTPGFVGADLANIVNEAALLAARRGGETVTREDIMEAIERAKFGINDRQLGPTAISKELGKLFPWIPSLMRRNNTGQDGLQGPLGYQTLS

SEQ ID NO: 123 - DNA, 1740 bp, Sorghum bicolor
  > Sobic.006G111600
atgcttgccatggcttcgctctgccccttgtccgccgcgcccactgtcccgcgcgctcgccgtgccgcaagcttcaggagccccgctccctggaccggcctcgccgcgaccgcgagcaatgccagtccctgcggctgtgggcttctgcccgctggaggctacgcgaggctgcggtcgcgtttgaggagccccgtgcgcgccaagatcgacgaggccggcaaggacacgggcgccggtctgggtttccccccgccccgccgccggaagctgcgcctgcgcctgcggccgcggctgcgcctcctgtggtggcggctccggcggctgtcgccgcgggagctcgcggccgacgccggggcagcgctgcggcgcgccgtccgacgcgtgccccccgcggccgccgcgccggtcgtcctggcgctgctcctcgccgtcgcgcggctcgcgctgcccaagaacgtggcccgggaggtggcctactcggacctcgtcgcggggctccgcgagggcgccgtggccgccgtcgcgttcgaggaggactcgcgccgtatctacttcagcaagaaagatggggacgacggcggctccgacacgagcgaggccggtgagagcgctgctgtggcggcgccgaagtggccgtactacgcgcggagggtgtcgcacgatgagggcttcctgctcgggctaatgcgggagggcggggtggactaccgatcggcgccgcggcctgctgggaggctgctcgtcgacatgctgagcacgctgctcacgctctgggtctcgctggtgcccatgatgtggttcatacagcggcagatgtctggtggtggcagtgctgacaagcgacgcaaaccgaggaagcagatggtgggctttgacgatgttcagggtgtagatgaagcgaaggaggagcttgttgagatagtgagctgtctgcatggttcactaaactacaagaagcttggagcaaaattgcccaggggtgttctgcttgttggccctcctggaactgggaagactctgttagcaagggcagttgcaggagaggctggaattccatttttctctgtttctgctagtgagtttgttgaagttttcgttggacgaggtgcagcccgagtcagggatttgttcaaggaggccaaagaagctgccccgtcaatcatttttattgatgaacttgatgctgtgggtggaagtcgaggtagaagttttaatgatgagagggaccaaactctaaaccagctgcttactgaaatggatggttttgactcggacatgaaagttattgtcatggctgctactaacaggccaaaagcactggatgctgctctttgtcgacctggccgcttctccagaaaggttgttgttggtaggcctgatgtggagggccgtaaaaagattctggctgttcatctaagggatattcctttggaagagaatcccgaaataattagtgatctggttgctagagtaactccaggattggtcggtgcagatctagcaaacattgttaatgaggctgctctattagctgcacgaagaggtcggaatactgtgtcccgggaggatattatggatgcaattgagagggagaaatacggggtcaacgggagacaagaaactgatgattctgaaaggcaaggcctcaccaagttatttccatggttacctaaatctggaaataaaccatcaagccctgatgatttccgaggactgatgggttatcatacattaagctaa

SEQ ID NO: 124 - protein, 579 aa, Sorghum bicolor
  > Sobic.006G111600
MLAMASLCPLSAAPTVPRARRAASFRSPAPWTGLAATASNASPCGCGLLPAGGYARLRSRLRSPVRAKIDEAGKDTGAGLGFPPPRRRKLRLRLRPRLRLLWWRLRRLSPRELAADAGAALRRAVRRVPPAAAAPVVLALLLAVARLALPKNVAREVAYSDLVAGLREGAVAAVAFEEDSRRIYFSKKDGDDGGSDTSEAGESAAVAAPKWPYYARRVSHDEGFLLGLMREGGVDYRSAPRPAGRLLVDMLSTLLTLWVSLVPMMWFIQRQMSGGGSADKRRKPRKQMVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGRPDVEGRKKILAVHLRDIPLEENPEIISDLVARVTPGLVGADLANIVNEAALLAARRGRNTVSREDIMDAIEREKYGVNGRQETDDSERQGLTKLFPWLPKSGNKPSSPDDFRGLMGYHTLS

SEQ ID NO: 125 - DNA, 1755 bp, Setaria italica
  > Seita.7G129200
atgccatccatggcttccctctgccccttatccgccgcgccctccttcccgcgcgctcacgccgcccgcttcaggagccccgcttcctgcagcggccacgccgcgaccgcaaggagcaatgcctatgtctgcggctgcggagctccacccgccgcggccaagggttgcgcggggctgcggtcgcggtggaggaaccccgtgcgcgccaaggtcgacgaggcggacaaggacgcgggcgccgggctggggctccccacgcccggccgcaggaggcggcgcctgcgcctgcggccgcggctgcgcctcctgtggtggcggcttcggcggctgtcgccgcgggacctcgcggccgacgccggtgccgcgctgcggcgcgccgtccgccgcgtgccccccgcggctgcggcgccggtcgtcctcgcgctcctcctcttcgccgcgcggctcgcgctgcccaagaacgtcgccaaggaggtggcctactcggacctcgtcgcggggctccgcgagggcgccgtggccgccgccgcgttcgaggaggactcgcgccgcatatacttcagcaagagggccgaggacggcgacgacggcgacgaagcgagcgaggccggtgagaccgagagcgccccggaggcggcgccgaagtggccgtactacgcgcggagggtgccgcacgacgaggggttcctgctcgggcttatgcgggagggcggggtggactaccggtcggcgccgcggcctgcggggagactggtggtggacatgctgagcacgctgctcacgctctgggtctcgctgctgccgatgatgtggttcctacagcggcagctgtctggtgggggcagtgctgacaagcgacgcaagccgaggaagcagagggtggggtttgacgacgttcagggtgtagatgaagcgaaggaggagcttgttgagatagtaagctgtttgcatggttcactgaactacaagaagcttggagcaaaattgcccaggggtgtcctgcttgttggccctccaggaactggaaagactctgctagcaagggctgttgcaggagaggctggaattccatttttctctgtttctgctagtgagtttgttgaagtttttgttggaagaggtgctgcccgggtcagggatttgttcaaggaagccaaagaagctgctccatcaatcatttttattgatgaacttgatgccgtgggtggaagtagaggtagaagttttaacgatgagagggaccaaactctaaatcagctgcttactgaaatggatggttttgactcggacatgaaagttattgtcatggctgctactaacaggccaaaagcactggatgctgctctttgtcggcctggtcgcttctccagaaaggttgttgttggtgtgcctgatgtggagggccgcaaaaagattttggctgttcatctaagggacattcctttggaagaggatcctgaaataatttgtgatctggttgctaatttaaccccaggactggttggagcggatctagcaaacattgttaatgaggctgctctcctagctgcacgaagaggtggtaatactgtttcccgggaggatattatggatgcaatcgagagggagaaatatggggtcaacggcagacaagaaaccaatgactctgaaaggcaaggcctcaccaaattatttccatggctacctaagcctggaaataaaccatcaagccctgatgatttccgaggactgatgggttatcagacattaagctaa

SEQ ID NO: 126 - protein, 584 aa, Setaria italica
  > Seita.7G129200
MPSMASLCPLSAAPSFPRAHAARFRSPASCSGHAATARSNAYVCGCGAPPAAAKGCAGLRSRWRNPVRAKVDEADKDAGAGLGLPTPGRRRRRLRLRPRLRLLWWRLRRLSPRDLAADAGAALRRAVRRVPPAAAAPVVLALLLFAARLALPKNVAKEVAYSDLVAGLREGAVAAAAFEEDSRRIYFSKRAEDGDDGDEASEAGETESAPEAAPKWPYYARRVPHDEGFLLGLMREGGVDYRSAPRPAGRLVVDMLSTLLTLWVSLLPMMWFLQRQLSGGGSADKRRKPRKQRVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGVPDVEGRKKILAVHLRDIPLEEDPEIICDLVANLTPGLVGADLANIVNEAALLAARRGGNTVSREDIMDAIEREKYGVNGRQETNDSERQGLTKLFPWLPKPGNKPSSPDDFRGLMGYQTLS

SEQ ID NO: 127 - DNA, 1395 bp, Setaria italica
  > Seita.7G129200
atgccatccatggcttccctctgccccttatccgccgcgccctccttcccgcgcgctcacgccgcccgcttcaggagccccgcttcctgcagcggccacgccgcgaccgcaaggagcaatgcctatgtctgcggctgcggagctccacccgccgcggccaagggttgcgcggggctgcggtcgcggtggaggaaccccgtgcgcgccaaggtcgacgaggcggacaaggacgcgggcgccgggctggggctccccacgcccggccgcaggaggcggcgcctgcgcctgcggccgcggctgcgcctcctgtggtggcggcttcggcggctgtcgccgcgggacctcgcggccgacgccggtgccgcgctgcggcgcgccgtccgccgcgtgccccccgcggctgcggcgccggtcgtcctcgcgctcctcctcttcgccgcgcggctcgcgctgcccaagaacgtcgccaaggaggtggcctactcggacctcgtcgcggggctccgcgagggcgccgtggccgccgccgcgttcgaggaggactcgcgccgcatatacttcagcaagagggccgaggacggcgacgacggcgacgaagcgagcgaggccggtgagaccgagagcgccccggaggcggcgccgaagtggccgtactacgcgcggagggtgccgcacgacgaggggttcctgctcgggcttatgcgggagggcggggtggactaccggtcggcgccgcggcctgcggggagactggtggtggacatgctgagcacgctgctcacgctctgggtctcgctgctgccgatgatgtggttcctacagcggcagctgtctggtgggggcagtgctgacaagcgacgcaagccgaggaagcagagggtggggtttgacgacgttcagggtgtagatgaagcgaaggaggagcttgttgagatagtaagctgtttgcatggttcactgaactacaagaagcttggagcaaaattgcccaggggtgtcctgcttgttggccctccaggaactggaaagactctgctagcaagggctgttgcaggagaggctggaattccatttttctctgtttctgctagtgagtttgttgaagtttttgttggaagaggtgctgcccgggtcagggatttgttcaaggaagccaaagaagctgctccatcaatcatttttattgatgaacttgatgccgtgggtggaagtagaggtagaagttttaacgatgagagggaccaaactctaaatcagctgcttactgaaatggatggttttgactcggacatgaaagttattgtcatggctgctactaacaggccaaaagcactggatgctgctctttgtcggcctggtcgcttctccagaaaggactggttggagcggatctag

SEQ ID NO: 128 - protein, 464 aa, Setaria italica
  > Seita.7G129200
MPSMASLCPLSAAPSFPRAHAARFRSPASCSGHAATARSNAYVCGCGAPPAAAKGCAGLRSRWRNPVRAKVDEADKDAGAGLGLPTPGRRRRRLRLRPRLRLLWWRLRRLSPRDLAADAGAALRRAVRRVPPAAAAPVVLALLLFAARLALPKNVAKEVAYSDLVAGLREGAVAAAAFEEDSRRIYFSKRAEDGDDGDEASEAGETESAPEAAPKWPYYARRVPHDEGFLLGLMREGGVDYRSAPRPAGRLVVDMLSTLLTLWVSLLPMMWFLQRQLSGGGSADKRRKPRKQRVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKDWLERI

SEQ ID NO: 129 - DNA, 1755 bp, Setaria italica; identical to SEQ ID NO: 125
  > Seita.7G129200

SEQ ID NO: 130 - protein, 584 aa, Setaria italica; identical to SEQ ID NO: 126
  > Seita.7G129200

SEQ ID NO: 131 - DNA, 1395 bp, Setaria italica; identical to SEQ ID NO: 127
  > Seita.7G129200

SEQ ID NO: 132 - protein, 464 aa, Setaria italica; identical to SEQ ID NO: 128
  > Seita.7G129200

SEQ ID NO: 133 - DNA, 1971 bp, Solanum lycopersicum
  > Solyc03g046340.2
atggcatctttacctcttgtttcaaatgacagattattagttactcacaagaaatggaaaccccatactggaaattctgaatcattaagaagtttcaagaatcaatattgctcattacccagttcttgttttacctcaagattttgtatatctcagtctaggtacaagaaatggatgcttcattttgggaattctgacccttttaggagattaaagaatcaaacttgctcattatccaattcttgttttacctcaagttctgttcctttattgggattgaattataggttttgtaaatctcaaagtagattactgcattgtagtactggggttaggtcaatggtaaatgagaagggagatatagatacccatttgaacaaaacaggaagtaataatattcgtgggaagttttcgttgagattgagaccaaggataagattactgtcaagaaggttgaaaagggtatctgttatatgtatgttaaatgattttgggaagtttctgaggaagaactcaagaagagtggcactgtcaacttcaatttctgtgatattgggcttgtgttatttgtttctaagattgacagcaactcctcctccaaaggttgttccatattcagacttgattaccagccttcagggaggatctgtgtcaaaggttcaatttgaggaaggcacacgccgtatttactacaatacgaacttgtggagtcttaaaaatgctcagacaggggaagataattccttagttcccgatgaaagcacgaccatcactgaggaaagcaaagatatagatagcaacaaaggtggcaagaatgtcttcagtaaaatatcaaaagcccaaggttctacccccgtatggcagtttagtacaaggaagatcgatcatgatgaaggatatcttcttagtctaatgagggaaaagggaacagcgtatggctcagcgccacaatcagcacttatgtcaattaggagcttattaattactatgttatccttatggattcctttgactcctataatgtggcttctctatcgtcaactttctgctgccaatagccctgcaagaaaaagaaagccaagtaaccaggtggttggctttaatgatgtggagggtgtggatgctgcaaaagttgagcttatggagatagtgttgtgcctgcgaggagctataaacttcagtaagctaggggcaaagttaccaagaggcgtactgcttgtaggtccaccagggacaggaaaaaccttactagctcgtgctgtggcaggagaagctggagtacccttcttttctgtttctgctagtgaatttgttgaaatgtttgttggaagaggagctgctcgcattagagaccttttcagtgtagcaagaaagaatgctccttcaatcattttcattgatgaactagatgctgtcggaggaaagcgtggcaggagtttcaatgatgagagagaccaaaccttgaatcagttgctcacagagatggatggctttgaatcggacttgaatatcattgtagttgctgctactaatagaccagaagctctagatccagcactatgtcggcctggacgtttctccaggaaaatcttagtgggagaacctgatgaagatggaaggagaaagatcctggctgtacacttgagagaagttccccttgaggaggacctggagcttgtatgtaacctggttgcatctcttacccagggccttgtaggtgctgaccttgccaacattgtcaatgaagctgctttgcttgctgctcggagaggggctgattgtgtgtcaagggaagatataatggaagccatagaaagagcaaaatttgggattaatgataagcagtacacccaaagtgcaataggtaaggaactggagaaactgttcccttgggttccttctttcattaggaagaacagtacaagaagtgatgcatttcaaggacctctggggtatcaagctctcagttga

SEQ ID NO: 134 - protein, 656 aa, Solanum lycopersicum
  > Solyc03g046340.2
MASLPLVSNDRLLVTHKKWKPHTGNSESLRSFKNQYCSLPSSCFTSRFCISQSRYKKWMLHFGNSDPFRRLKNQTCSLSNSCFTSSSVPLLGLNYRFCKSQSRLLHCSTGVRSMVNEKGDIDTHLNKTGSNNIRGKFSLRLRPRIRLLSRRLKRVSVICMLNDFGKFLRKNSRRVALSTSISVILGLCYLFLRLTATPPPKVVPYSDLITSLQGGSVSKVQFEEGTRRIYYNTNLWSLKNAQTGEDNSLVPDESTTITEESKDIDSNKGGKNVFSKISKAQGSTPVWQFSTRKIDHDEGYLLSLMREKGTAYGSAPQSALMSIRSLLITMLSLWIPLTPIMWLLYRQLSAANSPARKRKPSNQVVGFNDVEGVDAAKVELMEIVLCLRGAINFSKLGAKLPRGVLLVGPPGTGKTLLARAVAGEAGVPFFSVSASEFVEMFVGRGAARIRDLFSVARKNAPSIIFIDELDAVGGKRGRSFNDERDQTLNQLLTEMDGFESDLNIIVVAATNRPEALDPALCRPGRFSRKILVGEPDEDGRRKILAVHLREVPLEEDLELVCNLVASLTQGLVGADLANIVNEAALLAARRGADCVSREDIMEAIERAKFGINDKQYTQSAIGKELEKLFPWVPSFIRKNSTRSDAFQGPLGYQALS

SEQ ID NO: 135 - DNA, 1131 bp, Spirodela polyrhiza
  > Spipo5G0023300
atggcggctggggaggaaagaagtagttctctcccgcagtggcagttctccacgaggaagatcgaccatgatgagaacttcctccttactttaatgcgagagaaaggggcggtgtacagctcagctcctcagtcggctcttatggcaatgaggggccttgtgataaccatcttgtccctgtggattcctttgacacctttgatgtggctgctttatcgccaaatctctgcggccaacagccctgctaagaagcgaaggcctactacccagttggtcgactttgatgacgtagagggtgttgatgctgccaaggcagagcttatggagatagtttcatgcttacgaggatccatcaactacaataagctgggagcaaagctgcccagaggcgttctcctgtcgggccccccagggacaggcaaaacgcttctggcacgtgctgttgcaggggaagctggagttccattcttctcggtctctgcaagtgagtttgtagaactgttcgttggaagaggggcagctcgcattcgagacctgttcaaggtggccaggaagtattcaccatcgataatcttcattgatgagcttgatgctgtcggaggaaggcgtgggaggagtttcaatgatgagagggaccagactctgaatcagttattgacggaaatggatggatttgagtccgacatgaaggttattgtcattggtgcaacaaataggcctgatgcacttgatccggccctctgtcggcctggccgcttctcccgaaaggtggtggtgggagagccagattttgatgggaggaggaagatcttgggagtccatctgaggggggtcccgttggaggaggacccacagcttgtgtgcaatcttattgcttctctcactccagggctagtcggagctgaccttgccaacatcgtcaatgaggccgccctgcttactgccagaagaggtggtgaggtggtgactcgagatgatatcatggcggccatagagagagctaaatttggcatcaatgaacggcgattgactcctgaaaccatcggcaaggggcttgagaagctttttccatggataccatctctggtaaagaaggagaacgagtttctgggataccaaacgttaagctga

SEQ ID NO: 136 - protein, 376 aa, Spirodela polyrhiza
  > Spipo5G0023300
MAAGEERSSSLPQWQFSTRKIDHDENFLLTLMREKGAVYSSAPQSALMAMRGLVITILSLWIPLTPLMWLLYRQISAANSPAKKRRPTTQLVDFDDVEGVDAAKAELMEIVSCLRGSINYNKLGAKLPRGVLLSGPPGTGKTLLARAVAGEAGVPFFSVSASEFVELFVGRGAARIRDLFKVARKYSPSIIFIDELDAVGGRRGRSFNDERDQTLNQLLTEMDGFESDMKVIVIGATNRPDALDPALCRPGRFSRKVVVGEPDFDGRRKILGVHLRGVPLEEDPQLVCNLIASLTPGLVGADLANIVNEAALLTARRGGEVVTRDDIMAAIERAKFGINERRLTPETIGKGLEKLFPWIPSLVKKENEFLGYQTLS

SEQ ID NO: 137 - DNA, 1944 bp, Salix purpurea
  > SapurV1A.0472s0120
atggcttctttttcttctgtttgcaataatgggttcttgaatttcaaactgaactctagagtcaatgatggaaataccaaatctttgaagagatattcaactatttgttacagtccagttttgatcccttctttgggactttgcagtgctagcaaactccaacatttgttgctttggaggagcaacgtcaggtcatcattttatgaaggaataaacggggttaatttattggggttttgtggtggttgtaaatctcaaaatgggttgtcttgtaagagttcaattaggcaattgaggagtggaaacggtggagatgaagagagccagttgaggaaaagaggaaatggtaattttaggaagaggttttcattcagattgaaaacaagactgcggctattgaagataagattaaagggagtttcaattaggtccatgttgaatgattttggaatgttcttgagaaggaatataaaaagaatgacactatacacttcaatttctgttgcattgggaatgtgctacttgtttttgagattaactgcattgccatctccaaggattgttccatattcagaattgatagccagccttcaaaatggctatgttacaaaagtcttacttgaagagggatcccgtcgtatatactataacacagactctctgggtactgaaaatactgaaaactcagaagacaaatcttcagtagtagattttccaaatgagaatgcagctgaaacagttgcaatagaaggtgttgtgagcaaaacgggtctggcatcaaggttaaatgtatttaagaaattttcaaggccacgagcttccatcccagagtggcaattttctacaaggaaagtagatcgtgacgagaaatttcttcttactttgatgagagcaaagggaacagcatacagctctgcccctcaatcaattctgatgtcaataaggagtttgcttataactattatatctttgtggattcccttgactcctatgatgtggcttctttatcgccaactttctgctgctaatagcccagcaagaaagaggcgatcaaataatcaggcagtcaattttgatgatgtggagggtgttgatattgctaagatagaactcatggagatagttttgtgtctgcaaggagctatgaattatcaaaaactaggagcaaagttacctagaggtgtattgctggttggtcccccaggaacagggaagacattacttgcacgtgcagttgctggagaagcaggagtaccatttttcagtgtatctgccagtgaatttgtggaaatgtttgttggtagaggagcagctcggattagagatctctttaatgcggcaagaaaatctgctccgtcgattatattcatcgatgaacttgatgcagtagggggaaaacgtggtagaagtttcaatgatgaacgggatcaaacactaaaccagttgctgactgaaatggatggttttgagtcagacatgaaagttgtggttattgcagcaactaataggccagaagcattggatccagccctttgccgtcctggtcgcttttcaagaaaggtagttgttggagaaccagatgaagaaggaaggagaaagatcttggctgtacatttgagaggggttcccctagacgaagattcagatctcatctgtaatcttgttgcttcgctgacaccaggctttgtaggtgcagatcttgcaaacatcattaatgaagctgctttacttgctgctcgaagaggtggagacgttgtgacaagggaagatgtaatggaagcgattgaaagagcaaagtttggaatcagggacaggcaactaaggcccagtacaataagcaaagagctgggcaaattattcccttggattccatcgttgacgggaaggattgacacaagacaggatgatttgcaaggccctttaggttaccagactctgagctga

SEQ ID NO: 138 - protein, 647 aa, Salix purpurea
  > SapurV1A.0472s0120
MASFSSVCNNGFLNFKLNSRVNDGNTKSLKRYSTICYSPVLIPSLGLCSASKLQHLLLWRSNVRSSFYEGINGVNLLGFCGGCKSQNGLSCKSSIRQLRSGNGGDEESQLRKRGNGNFRKRFSFRLKTRLRLLKIRLKGVSIRSMLNDFGMFLRRNIKRMTLYTSISVALGMCYLFLRLTALPSPRIVPYSELIASLQNGYVTKVLLEEGSRRIYYNTDSLGTENTENSEDKSSVVDFPNENAAETVAIEGVVSKTGLASRLNVFKKFSRPRASIPEWQFSTRKVDRDEKFLLTLMRAKGTAYSSAPQSILMSIRSLLITIISLWIPLTPMMWLLYRQLSAANSPARKRRSNNQAVNFDDVEGVDIAKIELMEIVLCLQGAMNYQKLGAKLPRGVLLVGPPGTGKTLLARAVAGEAGVPFFSVSASEFVEMFVGRGAARIRDLFNAARKSAPSIIFIDELDAVGGKRGRSFNDERDQTLNQLLTEMDGFESDMKVVVIAATNRPEALDPALCRPGRFSRKVVVGEPDEEGRRKILAVHLRGVPLDEDSDLICNLVASLTPGFVGADLANIINEAALLAARRGGDVVTREDVMEAIERAKFGIRDRQLRPSTISKELGKLFPWIPSLTGRIDTRQDDLQGPLGYQTLS

SEQ ID NO: 139 - DNA, 1944 bp, Salix purpurea
  > SapurV1A.1792s0020
atggcttctttttcttctgtttgcaataatgggctcttgaatttcaaactgaactctagagtcaatgatggaaataccaaatctttgaagagatattcaactatttgttacagtccagttttgatcccttctttgggactttgcagtgctagcaaactccaacatttgttgctttggaggagcaacgtcaggtcatcattttatgacggaataaacggggtttatttattggggttttgtggtggttgtaaatctcaaaatgggttgtcttgtaagagttcaattaggcaattgaggagtggaaacggtggagatgaagagagccagttgaggaaaagaggaaatggtaattttaggaagaggttttcattcagattgaaaacaagactgcggctattgaagataagattaaagggagtttcaattaggtccatgttgaatgattttggaatgttcttgagaaggaatataaaaagaatgacactatacacttcaatttctgttgcattgggaatgtgctacttgtttttgagattaactgcattgccatctccaaggattgttccatattcagaattgatagccagccttcaaaatggctatgttacaaaagtcttacttgaagagggatcccgtcgtatatactataacacagactctctgggtactgaaaatactgaaaactcagaagacaaatcttcagtagtagattttccaaatgagaatgcagctgaaacagttgcaattgaaggtgttgtgagcaaaacgggtctggcatcaaggttaaatgtatttaagaaattttcaaggccacgagcttccatcccagagtggcaattttctacaaggaaagtagatcgtgacgagaaatttcttcttactttgatgagagcaaagggaacagcatacagctctgcccctcaatcaattctgatgtcaataaggagtttgcttataactattatatctttgtggattcccttgactcctatgatgtggcttctttatcgccaactttctgctgctaatagcccagcaagaaagaggcgatcaaataatcaggcagtcaattttgatgatgtggagggtgttgatattgctaagatagaactcatggagatagttttgtgtctgcaaggagctatgaattatcaaaaactaggagcaaagttacctagaggtgtattgctggttggtcccccaggaacagggaagacattacttgcacgtgcagttgctggagaagcaggagtaccatttttcagtgtatctgccagtgaatttgtggaaatgtttgttggtagaggagcagctcggattagagatctctttaatgcggcaagaaaatctgctccgtcgattatattcatcgatgaacttgatgcagtagggggaaaacgtggtagaagtttcaatgatgaacgggatcaaacactaaaccagttgctgactgaaatggatggttttgagtcagacatgaaagttgtggttattgcagcaactaataggccagaagcattggatccagccctttgccgtcctggtcgcttttcaagaaaggtagttgttggagaaccagatgaagaaggaaggagaaagatcctggctgtgcatttgagaggggttcccttagacgaagattcagatctcatctgtaatcttgttgcttcgctgacaccaggctttgtaggtgctgatcttgcaaacatcattaatgaagctgctttacttgctgctcgaagaggtggagacgttgtgacaagggaagatgtaatggaagcgattgaaagagcaaagtttggaatcagggacaggcaactaaggcccagtacaataagcaaagagctgggcaaattattcccttggattccatcgttgacgggaaggattgacacaagacaggatgatttgcaaggccctttaggttaccagactctgagctga

SEQ ID NO: 140 - protein, 647 aa, Salix purpurea
  > SapurV1A.1792s0020
MASFSSVCNNGLLNFKLNSRVNDGNTKSLKRYSTICYSPVLIPSLGLCSASKLQHLLLWRSNVRSSFYDGINGVYLLGFCGGCKSQNGLSCKSSIRQLRSGNGGDEESQLRKRGNGNFRKRFSFRLKTRLRLLKIRLKGVSIRSMLNDFGMFLRRNIKRMTLYTSISVALGMCYLFLRLTALPSPRIVPYSELIASLQNGYVTKVLLEEGSRRIYYNTDSLGTENTENSEDKSSVVDFPNENAAETVAIEGVVSKTGLASRLNVFKKFSRPRASIPEWQFSTRKVDRDEKFLLTLMRAKGTAYSSAPQSILMSIRSLLITIISLWIPLTPMMWLLYRQLSAANSPARKRRSNNQAVNFDDVEGVDIAKIELMEIVLCLQGAMNYQKLGAKLPRGVLLVGPPGTGKTLLARAVAGEAGVPFFSVSASEFVEMFVGRGAARIRDLFNAARKSAPSIIFIDELDAVGGKRGRSFNDERDQTLNQLLTEMDGFESDMKVVVIAATNRPEALDPALCRPGRFSRKVVVGEPDEEGRRKILAVHLRGVPLDEDSDLICNLVASLTPGFVGADLANIINEAALLAARRGGDVVTREDVMEAIERAKFGIRDRQLRPSTISKELGKLFPWIPSLTGRIDTRQDDLQGPLGYQTLS

SEQ ID NO: 141 - DNA, 1755 bp, Setaria viridis
  > Sevir.7G137600
atgccatccatggcttccctctgccccttatccgccgcgccctccttcccgcgcgctcacgccgcccgcttcaggagccccgcttcctgcagcggccacgccgcgaccgcaaggagcaatgcctatgtctgcggctgcggagctccacccgccgcggccaagggttgcgcggggctgcggtcgcggtggaggaaccccgtgcgcgccaaggtcgacgaggcggacaaggacgcgggcgccgggctggggctccccacgcccggccgcaggaggcggcgcctgcgcctgcggccgcggctgcgcctcctgtggtggcggcttcggcggctgtcgccgcgggacctcgcggccgacgccggtgccgcgctgcggcgcgccgtccgccgcgtgccccccgcggctgcggcgccggtcgtcctcgcgctcctcctcttcgccgcgcggctcgcgctgcccaagaacgtcgccaaggaggtggcctactcggacctcgtcgcggggctccgcgagggcgccgtggccgccgccgcgttcgaggaggactcgcgccgcatctacttcagcaagagggccgaggacggcgacgacggcgacgaagcgagcgaggccggtgagaccgagagcgccccggaggcgccgccgaagtggccgtactacgcgcggagggtgccgcacgacgaggggttcctgctcgggcttatgcgggagggcggggtggactaccggtcggcgccgcggcctgcggggaggctggtggtggacatgctgagcacgctgctcacgctctgggtctcgctgctgccgatgatgtggttcctacagcggcagctgtctggtgggggcagtgctgacaagcgacgcaagccgaggaagcagagggtggggtttgacgacgttcagggtgtagatgaagcgaaggaggagcttgttgagatagtaagctgtttgcatggttcactgaactacaagaagcttggagcaaaattgcccaggggtgtcctgcttgttggccctccaggaactggaaagactctgctagcaagggctgttgcaggagaggctggaattccatttttctctgtttctgctagtgagtttgttgaagtttttgttggaagaggtgctgcccgggtcagggatttgttcaaggaagccaaagaagctgctccatcaatcatttttattgatgaacttgatgccgtgggtggaagtagaggtagaagttttaatgatgagagggaccaaactctaaatcagctgcttactgaaatggatggttttgactcggacatgaaagttattgtcatggctgctactaacaggccaaaagcactggatgctgctctttgtcggcctggtcgcttctccagaaaggttgttgttggtgtgcctgatgtggagggccgcaaaaagattttggctgttcatctaagggacattcctttggaagaggatcctgaaataatttgtgatctggttgctaatttaaccccaggactggttggagcggatctagcaaacattgttaatgaggctgctctcctagctgcacgaagaggtggtaatactgtttcccgggaggatattatggatgcaatcgagagggagaaatatggggtcaacggcagacaagaaaccaatgactctgaaaggcaaggcctcaccaaattatttccatggctacctaagcctggaaataaaccatcaagccctgatgatttccgaggactgatgggttatcagacattaagctaa

SEQ ID NO: 142 - protein, 584 aa, Setaria viridis
  > Sevir.7G137600
MPSMASLCPLSAAPSFPRAHAARFRSPASCSGHAATARSNAYVCGCGAPPAAAKGCAGLRSRWRNPVRAKVDEADKDAGAGLGLPTPGRRRRRLRLRPRLRLLWWRLRRLSPRDLAADAGAALRRAVRRVPPAAAAPVVLALLLFAARLALPKNVAKEVAYSDLVAGLREGAVAAAAFEEDSRRIYFSKRAEDGDDGDEASEAGETESAPEAPPKWPYYARRVPHDEGFLLGLMREGGVDYRSAPRPAGRLVVDMLSTLLTLWVSLLPMMWFLQRQLSGGGSADKRRKPRKQRVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGVPDVEGRKKILAVHLRDIPLEEDPEIICDLVANLTPGLVGADLANIVNEAALLAARRGGNTVSREDIMDAIEREKYGVNGRQETNDSERQGLTKLFPWLPKPGNKPSSPDDFRGLMGYQTLS

SEQ ID NO: 143 - DNA, 1395 bp, Setaria viridis
  > Sevir.7G137600
atgccatccatggcttccctctgccccttatccgccgcgccctccttcccgcgcgctcacgccgcccgcttcaggagccccgcttcctgcagcggccacgccgcgaccgcaaggagcaatgcctatgtctgcggctgcggagctccacccgccgcggccaagggttgcgcggggctgcggtcgcggtggaggaaccccgtgcgcgccaaggtcgacgaggcggacaaggacgcgggcgccgggctggggctccccacgcccggccgcaggaggcggcgcctgcgcctgcggccgcggctgcgcctcctgtggtggcggcttcggcggctgtcgccgcgggacctcgcggccgacgccggtgccgcgctgcggcgcgccgtccgccgcgtgccccccgcggctgcggcgccggtcgtcctcgcgctcctcctcttcgccgcgcggctcgcgctgcccaagaacgtcgccaaggaggtggcctactcggacctcgtcgcggggctccgcgagggcgccgtggccgccgccgcgttcgaggaggactcgcgccgcatctacttcagcaagagggccgaggacggcgacgacggcgacgaagcgagcgaggccggtgagaccgagagcgccccggaggcgccgccgaagtggccgtactacgcgcggagggtgccgcacgacgaggggttcctgctcgggcttatgcgggagggcggggtggactaccggtcggcgccgcggcctgcggggaggctggtggtggacatgctgagcacgctgctcacgctctgggtctcgctgctgccgatgatgtggttcctacagcggcagctgtctggtgggggcagtgctgacaagcgacgcaagccgaggaagcagagggtggggtttgacgacgttcagggtgtagatgaagcgaaggaggagcttgttgagatagtaagctgtttgcatggttcactgaactacaagaagcttggagcaaaattgcccaggggtgtcctgcttgttggccctccaggaactggaaagactctgctagcaagggctgttgcaggagaggctggaattccatttttctctgtttctgctagtgagtttgttgaagtttttgttggaagaggtgctgcccgggtcagggatttgttcaaggaagccaaagaagctgctccatcaatcatttttattgatgaacttgatgccgtgggtggaagtagaggtagaagttttaatgatgagagggaccaaactctaaatcagctgcttactgaaatggatggttttgactcggacatgaaagttattgtcatggctgctactaacaggccaaaagcactggatgctgctctttgtcggcctggtcgcttctccagaaaggactggttggagcggatctag

SEQ ID NO: 144 - protein, 464 aa, Setaria viridis
  > Sevir.7G137600
MPSMASLCPLSAAPSFPRAHAARFRSPASCSGHAATARSNAYVCGCGAPPAAAKGCAGLRSRWRNPVRAKVDEADKDAGAGLGLPTPGRRRRRLRLRPRLRLLWWRLRRLSPRDLAADAGAALRRAVRRVPPAAAAPVVLALLLFAARLALPKNVAKEVAYSDLVAGLREGAVAAAAFEEDSRRIYFSKRAEDGDDGDEASEAGETESAPEAPPKWPYYARRVPHDEGFLLGLMREGGVDYRSAPRPAGRLVVDMLSTLLTLWVSLLPMMWFLQRQLSGGGSADKRRKPRKQRVGFDDVQGVDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKDWLERI

SEQ ID NO: 145 - DNA, 1920 bp, Theobroma cacao
  > Thecc1EG019629
atggcttgtttcacagttgcttgcaataatggattttttgttagtaaagaaaactcggtaattgcaagtggaaaaaccaagtcttttaggagatacaatagcttttctagtagttcattttgctttcattcacttagaatatataattataaccatagcaaaacccagaatttgtttttttctggtgataataggtttaggttggttagcaatggacgaagaaatgaggttcctttattggggtttcaagtttgttgcaaggcacataataggttattgatgaggggaaataatggagataggaaaaccctattagggaaaagagaaagtagtaatgtgaggaaaagattttcattaagattgcgtcccaggttgcggttattaacaataagaatgaagggagtttcggttaggtcaacgttaaatgatatcggaatgtttctacgaaagaatataagaagagttactctctgcagtacaatatctctagcattggcgatgtgctatttgttcttgaaattaactgcattgccatctcctaagattgttccttactcggagttgataacaagcctccagaatagttctgtgacaaaggttttgctagaagagggttctcgtcgtatatatttcaatatggattctaaaagtgctgaagatactcaaaattcagaagaagaatctttggcagtgaatgaatcaattgaaaatgtaactgatatggctgcacaagatgatggtgtggaaggtaggcgattgcaaaaacaaggtctatttaagaaagtttcaagacctcaatcttctacttctgagtggcagtatttgacaagaaaaattgatcatgatgaaaaatttcttcttagtttgatgagagaaaagggaacaacttatagctcagctcctcagtcagttctaatgtcgatgaggagtactttaataacaatactatccttgtgggttcctttaacacctttgatgtggcttctgtatcgtcaactttctgctgctaatagccctgcaagaaagcggcgtccaaataatcagtttattgggtttgatgatgtggagggagttgatactgctaaggcggagcttatggagatagtttcatgccttcagggatctataaattaccaaaaactgggagcaaaattgcctagaggtgtactgctagtgggtccacctggaacagggaagacattactagctcgtgcagtagctggagaggcgggagttccttttttcagcgtatcagctagtgaatttgttgaattgtttgttggaagaggagcagctcgaattagagaccttttcaatgtggcaaggaaatctgcaccgtcgattatatttattgatgaacttgatgcagttggagggaagcgtggtagaagtttcaatgatgaacgagaccaaacattgaaccagttgctgacagaaatggatggatttgagtcagacatgaaagtggttgttattggggcaactaataggccagaagcattggatccagccctctgtcgccctggtcgcttctcgagaaaagtgttggtgggggaaccagatgaggaaggaaggagaaagattttggctgtgcacttgagaggagttcctctagaagaagacaaacaacttattgccgacttagttgcttccttgaccccaggttttgtaggtgctgatcttgcaaacattgtcaatgaagctgctttacttgctgctcgtagaggtgctgaaactttgacaagggaagatataatggaagctgttgagagagctaaatttgggattaatggtaggcagcctagtacaataggcaaagagctagggaaacttttcctatggatgccctccttaatgggaaggagtgatacaagacaggatggtttgcaagggcccttgggctatcaaactctaagctga

SEQ ID NO: 146 - protein, 639 aa, Theobroma cacao
  > Thecc1EG019629
MACFTVACNNGFFVSKENSVIASGKTKSFRRYNSFSSSSFCFHSLRIYNYNHSKTQNLFFSGDNRFRLVSNGRRNEVPLLGFQVCCKAHNRLLMRGNNGDRKTLLGKRESSNVRKRFSLRLRPRLRLLTIRMKGVSVRSTLNDIGMFLRKNIRRVTLCSTISLALAMCYLFLKLTALPSPKIVPYSELITSLQNSSVTKVLLEEGSRRIYFNMDSKSAEDTQNSEEESLAVNESIENVTDMAAQDDGVEGRRLQKQGLFKKVSRPQSSTSEWQYLTRKIDHDEKFLLSLMREKGTTYSSAPQSVLMSMRSTLITILSLWVPLTPLMWLLYRQLSAANSPARKRRPNNQFIGFDDVEGVDTAKAELMEIVSCLQGSINYQKLGAKLPRGVLLVGPPGTGKTLLARAVAGEAGVPFFSVSASEFVELFVGRGAARIRDLFNVARKSAPSIIFIDELDAVGGKRGRSFNDERDQTLNQLLTEMDGFESDMKVVVIGATNRPEALDPALCRPGRFSRKVLVGEPDEEGRRKILAVHLRGVPLEEDKQLIADLVASLTPGFVGADLANIVNEAALLAARRGAETLTREDIMEAVERAKFGINGRQPSTIGKELGKLFLWMPSLMGRSDTRQDGLQGPLGYQTLS

SEQ ID NO: 147 - DNA, 1794 bp, Theobroma cacao
  > Thecc1EG019629
atggaaatagccctctgtccattccattcctcagtttcatccccatgttggcaagctcttaaatccaggtttaggttggttagcaatggacgaagaaatgaggttcctttattggggtttcaagtttgttgcaaggcacataataggttattgatgaggggaaataatggagataggaaaaccctattagggaaaagagaaagtagtaatgtgaggaaaagattttcattaagattgcgtcccaggttgcggttattaacaataagaatgaagggagtttcggttaggtcaacgttaaatgatatcggaatgtttctacgaaagaatataagaagagttactctctgcagtacaatatctctagcattggcgatgtgctatttgttcttgaaattaactgcattgccatctcctaagattgttccttactcggagttgataacaagcctccagaatagttctgtgacaaaggttttgctagaagagggttctcgtcgtatatatttcaatatggattctaaaagtgctgaagatactcaaaattcagaagaagaatctttggcagtgaatgaatcaattgaaaatgtaactgatatggctgcacaagatgatggtgtggaaggtaggcgattgcaaaaacaaggtctatttaagaaagtttcaagacctcaatcttctacttctgagtggcagtatttgacaagaaaaattgatcatgatgaaaaatttcttcttagtttgatgagagaaaagggaacaacttatagctcagctcctcagtcagttctaatgtcgatgaggagtactttaataacaatactatccttgtgggttcctttaacacctttgatgtggcttctgtatcgtcaactttctgctgctaatagccctgcaagaaagcggcgtccaaataatcagtttattgggtttgatgatgtggagggagttgatactgctaaggcggagcttatggagatagtttcatgccttcagggatctataaattaccaaaaactgggagcaaaattgcctagaggtgtactgctagtgggtccacctggaacagggaagacattactagctcgtgcagtagctggagaggcgggagttccttttttcagcgtatcagctagtgaatttgttgaattgtttgttggaagaggagcagctcgaattagagaccttttcaatgtggcaaggaaatctgcaccgtcgattatatttattgatgaacttgatgcagttggagggaagcgtggtagaagtttcaatgatgaacgagaccaaacattgaaccagttgctgacagaaatggatggatttgagtcagacatgaaagtggttgttattggggcaactaataggccagaagcattggatccagccctctgtcgccctggtcgcttctcgagaaaagtgttggtgggggaaccagatgaggaaggaaggagaaagattttggctgtgcacttgagaggagttcctctagaagaagacaaacaacttattgccgacttagttgcttccttgaccccaggttttgtaggtgctgatcttgcaaacattgtcaatgaagctgctttacttgctgctcgtagaggtgctgaaactttgacaagggaagatataatggaagctgttgagagagctaaatttgggattaatggtaggcagcctagtacaataggcaaagagctagggaaacttttcctatggatgccctccttaatgggaaggagtgatacaagacaggatggtttgcaagggcccttgggctatcaaactctaagctga

SEQ ID NO: 148 - protein, 597 aa, Theobroma cacao
  > Thecc1EG019629
MEIALCPFHSSVSSPCWQALKSRFRLVSNGRRNEVPLLGFQVCCKAHNRLLMRGNNGDRKTLLGKRESSNVRKRFSLRLRPRLRLLTIRMKGVSVRSTLNDIGMFLRKNIRRVTLCSTISLALAMCYLFLKLTALPSPKIVPYSELITSLQNSSVTKVLLEEGSRRIYFNMDSKSAEDTQNSEEESLAVNESIENVTDMAAQDDGVEGRRLQKQGLFKKVSRPQSSTSEWQYLTRKIDHDEKFLLSLMREKGTTYSSAPQSVLMSMRSTLITILSLWVPLTPLMWLLYRQLSAANSPARKRRPNNQFIGFDDVEGVDTAKAELMEIVSCLQGSINYQKLGAKLPRGVLLVGPPGTGKTLLARAVAGEAGVPFFSVSASEFVELFVGRGAARIRDLFNVARKSAPSIIFIDELDAVGGKRGRSFNDERDQTLNQLLTEMDGFESDMKVVVIGATNRPEALDPALCRPGRFSRKVLVGEPDEEGRRKILAVHLRGVPLEEDKQLIADLVASLTPGFVGADLANIVNEAALLAARRGAETLTREDIMEAVERAKFGINGRQPSTIGKELGKLFLWMPSLMGRSDTRQDGLQGPLGYQTLS

SEQ ID NO: 149 - DNA, 1434 bp, Theobroma cacao
  > Thecc1EG019629
atggcttgtttcacagttgcttgcaataatggattttttgttagtaaagaaaactcggtaattgcaagtggaaaaaccaagtcttttaggagatacaatagcttttctagtagttcattttgctttcattcacttagaatatataattataaccatagcaaaacccagaatttgtttttttctggtgataataggtttaggttggttagcaatggacgaagaaatgaggttcctttattggggtttcaagtttgttgcaaggcacataataggttattgatgaggggaaataatggagataggaaaaccctattagggaaaagagaaagtagtaatgtgaggaaaagattttcattaagattgcgtcccaggttgcggttattaacaataagaatgaagggagtttcggttaggtcaacgttaaatgatatcggaatgtttctacgaaagaatataagaagagttactctctgcagtacaatatctctagcattggcgatgtgctatttgttcttgaaattaactgcattgccatctcctaagattgttccttactcggagttgataacaagcctccagaatagttctgtgacaaaggttttgctagaagagggttctcgtcgtatatatttcaatatggattctaaaagtgctgaagatactcaaaattcagaagaagaatctttggcagtgaatgaatcaattgaaaatgtaactgatatggctgcacaagatgatggtgtggaaggtaggcgattgcaaaaacaaggtctatttaagaaagtttcaagacctcaatcttctacttctgagtggcagtatttgacaagaaaaattgatcatgatgaaaaatttcttcttagtttgatgagagaaaagggaacaacttatagctcagctcctcagtcagttctaatgtcgatgaggagtactttaataacaatactatccttgtgggttcctttaacacctttgatgtggcttctgtatcgtcaactttctgctgctaatagccctgcaagaaagcggcgtccaaataatcagtttattgggtttgatgatgtggagggagttgatactgctaaggcggagcttatggagatagtttcatgccttcagggatctataaattaccaaaaactgggagcaaaattgcctagaggtgtactgctagtgggtccacctggaacagggaagacattactagctcgtgcagtagctggagaggcgggagttccttttttcagcgtatcagctagtgaatttgttgaattgtttgttggaagaggagcagctcgaattagagaccttttcaatgtggcaaggaaatctgcaccgtcgattatatttattgatgaacttgatgcagttggagggaagcgtggtagaagtttcaatgatgaacgagaccaaacattgaaccaggcactttaa

SEQ ID NO: 150 - protein, 477 aa, Theobroma cacao
  > Thecc1EG019629
MACFTVACNNGFFVSKENSVIASGKTKSFRRYNSFSSSSFCFHSLRIYNYNHSKTQNLFFSGDNRFRLVSNGRRNEVPLLGFQVCCKAHNRLLMRGNNGDRKTLLGKRESSNVRKRFSLRLRPRLRLLTIRMKGVSVRSTLNDIGMFLRKNIRRVTLCSTISLALAMCYLFLKLTALPSPKIVPYSELITSLQNSSVTKVLLEEGSRRIYFNMDSKSAEDTQNSEEESLAVNESIENVTDMAAQDDGVEGRRLQKQGLFKKVSRPQSSTSEWQYLTRKIDHDEKFLLSLMREKGTTYSSAPQSVLMSMRSTLITILSLWVPLTPLMWLLYRQLSAANSPARKRRPNNQFIGFDDVEGVDTAKAELMEIVSCLQGSINYQKLGAKLPRGVLLVGPPGTGKTLLARAVAGEAGVPFFSVSASEFVELFVGRGAARIRDLFNVARKSAPSIIFIDELDAVGGKRGRSFNDERDQTLNQAL

SEQ ID NO: 151 - DNA, 1599 bp, Trifolium pratense
  modified_base 103-144, 1074-1092: > n is a, c, g or t; Tp57577_TGAC_v2_gene27154
atgtatgtagcatcatcagtagataacagaacaaccaatagaaacctttgtgttcatcttcttcctccaacacaagattccacttcttcaattttaccggttnnnnnnnnnnnnnnnnnnnnnnnnnnnnnnnnnnnnnnnnnngcttctgttgaatctgttttgaatgaggtgggaatgtttgttaggaagaatattaggacggtggctttttctacctcgttttcgattgtgttcactctttgtttcatgtttttgaaatttacttcacttccccctgcaaaaattgttccgtattcggacttgattgcaaatattcaagatgggttggttgcgaaagttttagttgaagaggggtctcggcgtatatattacaatacgaaggacaaggttgttgaagatgataaggtttctggtgaagaatcgcaacaagtagtagatgtttcaatggataaggatataagtgtggtggggagtgaggttgcttcaagatcgggccaaattcctgtgttgaataaattgaagaaactttctaagaaacgagcattgattcctgaatggcagtattccacgagaaagatagatcatgacgaaaaattcctttatagtttgatgagagaaaagggggttacgtttagctccgcacctcaatctgcgttaatgtcgatgaggagcattttgataactgtgatcacactgtggatacctttgattccgttgatgtggcttctatatcgtcaactttctgcagctaatagtccagcaaagaagcggaaaccgaatagtgaaacagttggatttgaagatgtccaaggtgttgattctgcaaaagtagaactcatggagatagtttcttgtctgcaaggggatataaattaccaaaaggttggtgcaaagttacctagaggagtgttgctagtcggtcctccaggaactggaaagacattacttgcccgtgcagtagcaggggaagcaggcgtgccctttttcacagtatctgccagcgagtttgtagaaatgtttgtcggaagaggggctgctagaatcagagacctgtttagtacagcaaggaannnnnnnnnnnnnnnnnnncaattgctgacagaaatggatggatttgaatcagagatgagagtggttgttattgcagcaactaatcgaccagaagccttggatgaagccctatgtcgtccgggtcgtttctcaaggaaagtatttgtaggggaacctgatgaagaaggaaggagaaagatattagctgtccatctaaaaggagttcctttagaggaagatgttaacatcatatgtcagttaattgctactcttaccgatggtcttgtaggtgctgatttggcaaacattgtcaatgaatccgcattgcttgccgctcgtagaggtagtgaaactgtggcaagggaagatataatggaagctatcgaaagagcgaagtttggaatcaatgataagcgactaaggtctagtaaaataagcaaagaactcggtaagctattcccttggatgccatcattgatgggaagaagtgacaagagactagatgatatacaaggaccactaggttatcagtcattgagtccatga

SEQ ID NO: 152 - protein, 532 aa, Trifolium pratense
  VARIANT 35-48, 358-364: > Xaa is any amino acid; Tp57577_TGAC_v2_gene27154
MYVASSVDNRTTNRNLCVHLLPPTQDSTSSILPVXXXXXXXXXXXXXXASVESVLNEVGMFVRKNIRTVAFSTSFSIVFTLCFMFLKFTSLPPAKIVPYSDLIANIQDGLVAKVLVEEGSRRIYYNTKDKVVEDDKVSGEESQQVVDVSMDKDISVVGSEVASRSGQIPVLNKLKKLSKKRALIPEWQYSTRKIDHDEKFLYSLMREKGVTFSSAPQSALMSMRSILITVITLWIPLIPLMWLLYRQLSAANSPAKKRKPNSETVGFEDVQGVDSAKVELMEIVSCLQGDINYQKVGAKLPRGVLLVGPPGTGKTLLARAVAGEAGVPFFTVSASEFVEMFVGRGAARIRDLFSTARXXXXXXXQLLTEMDGFESEMRVVVIAATNRPEALDEALCRPGRFSRKVFVGEPDEEGRRKILAVHLKGVPLEEDVNIICQLIATLTDGLVGADLANIVNESALLAARRGSETVAREDIMEAIERAKFGINDKRLRSSKISKELGKLFPWMPSLMGRSDKRLDDIQGPLGYQSLSP

SEQ ID NO: 153 - DNA, 1416 bp, Vitis vinifera
  > GSVIVG01036484001
atggtaaatgaatttggagcattcttgcgtaagcatctaaaaagagtgactcttacaactgcaatttctgttgcactggggctgttctatttgttccttaaactgacaacattgccatctccgaagattgttccatattcggatttagtaacaagcctccagagtggggtagtgacgaatgttctatttgaggagggatctcgtcgcatatactataacatggaccctcaacgtctaaaaaatactcaaacatttgaagaaatagtgcctgtggatgttccaaatgggaatttggatgatggatggcagtactccacaagaaagatagaccatgatgagaactttctgcttagtttgatgagagaaaagggaactgcatacagctcggcaccccaatcggtgttaatgtcaatgaggagtattttgattactattttatctttgtggattcctttaactcctctgatgtggcttctttaccgacaactttctgctgctaatagtcctgcaaaaaaacggcgacctagtagtcagattgtcagttttgatgatgttgagggtgtagacgctgctaaggtggagcttatggagatagtttcatgcctgcaaggagccagtgattacaacaaactaggagcaaagttacctagaggtgtgctgcttgtgggtcctccaggaacagggaagacattactagctcgtgcagtggctggagaagcaggggtaccgtttttcagtgtttctgccagtgaatttgtggaattgtttgttggaagaggggcagctcgtgttagagacctttttaatgtggcaaggaaatgtgcaccatcaatcatattcattgatgaacttgatgctgtaggaggaaagcgtggaagaagcttcaatgatgaacgtgaccaaacactaaaccagttactgactgaaatggatggatttgagtcggacatgaaggtaattgttattgcagcaactaaccggccagaagctttagatgcagctctctgtcggcctggtcgcttctcaaggaaagtacttgttggggaaccagatgaagaagggagaaggaagatcttggctatacatttgagagaagttcctctggaagaagacacacgtcttatctgcaacctagttgcttctctcacccaaggttttgtaggtgctgaccttgcaaacattgtcaatgaagctgctttacttgctggtcgcagaggtggtgaatccgtgacaagggaagatataatggaagctatagaaagagcaaggtttgggatcaacgataagcagtcaaatcctagtacaataagcagggagctcaggaaactgttcccatggatgccatctctaatgggtagtcaggactcaagacagtatgccctgcaagggcccctaggctatcaaactctgagctga

SEQ ID NO: 154 - protein, 471 aa, Vitis vinifera
  > GSVIVG01036484001
MVNEFGAFLRKHLKRVTLTTAISVALGLFYLFLKLTTLPSPKIVPYSDLVTSLQSGVVTNVLFEEGSRRIYYNMDPQRLKNTQTFEEIVPVDVPNGNLDDGWQYSTRKIDHDENFLLSLMREKGTAYSSAPQSVLMSMRSILITILSLWIPLTPLMWLLYRQLSAANSPAKKRRPSSQIVSFDDVEGVDAAKVELMEIVSCLQGASDYNKLGAKLPRGVLLVGPPGTGKTLLARAVAGEAGVPFFSVSASEFVELFVGRGAARVRDLFNVARKCAPSIIFIDELDAVGGKRGRSFNDERDQTLNQLLTEMDGFESDMKVIVIAATNRPEALDAALCRPGRFSRKVLVGEPDEEGRRKILAIHLREVPLEEDTRLICNLVASLTQGFVGADLANIVNEAALLAGRRGGESVTREDIMEAIERARFGINDKQSNPSTISRELRKLFPWMPSLMGSQDSRQYALQGPLGYQTLS

SEQ ID NO: 155 - DNA, 1791 bp, Zostera marina
  > Zosma54g01120
atgacatctttcttttacgttttcccaagttacagtccctttactagtccagatccttctagatttaatcggtcattggttttgtataacgggggcagaatcacagtgccaagtagaaaattgggtgttttttgttttttgggaaatgggtatgtttcaaggtgctcgagagaatgcctctgtagcccccgatgtcagaatcggtgtaggaggaggattgtttctaggattaatagtggtggtgataataggattgattccacagaagatgagagaaccaatggaagaaagaaatttaacttgatggatttctttaaaaacatcgaatcgaatttgagaaagaatcctaaaaaggttatgatttcggccatgatttctgtctctcttgctctttgtatcttgttgctgaagcttacatcaatgcctactcccaagcttgtgtgctattcggattttgtaacaaagcttcaaaatggaggcatttctgaagttttgtttgaagaaggtacaccgcggatattatacaaaaccgtcgcctatgagaaatctgaagagccttctgaggcagtagtagtagtagaaccagaagcaactaaaaagggaagaacaccattcaaattgactatgactgcaattataactgctgcttccaaagcaagaactaagaagaagaataagagtatacctgagtggagtttcaccactaggaagatcaaacataacgaaaacttccttttcgatctgatgagggaaaagggcaccaaatacggttctgctccacaatcacccatagctttactaagaaatgctctgcttgcattgctgacattgtggattccattagctcctatattgtggttgcttcatcgtcaactttcatcatcgaacactgctgctaaaaaaaagcgtcctaaaaatcagcttgttggtttcaatgatgttgaaggtgttgatgctgcaaaagaagagcttttggagattgtgtcatgcttacaaggatccgtcaatttcagcaagttgggagcaagattacccaaaggagtgcttctcgtcggtccacctggaacaggcaaaacattacttgcccgtgctgttgccggagaagcagacgtcccattctttccggtctctgcaagtgagtttgtagagctattcgttggacggggagcatcaaggatcagagacttgtttaaggtagcaagaaagaattcaccctctatagtattcattgatgagcttgacgccgttggtgggaaacgaggtagaagctttaacgatgagcgagatcaaactttaaaccagttacttactgaaatggatggatttgaatccgacacaaatgtgattgtgatagcagcaacaaacagaccagaggcattggatccagctctctgtcgtcccgggagattttctaggagagtctttgtggaagaacctgatttagatggcaggaaaagaatcttaggtgttcatataaaaggtgttccactggaagaagatgctgaaattatttgcagccttatagccagtctcacacctggttttgtcggagccgacttggctaacataattaatgaagctgcattgcttgctgccagaagaggtggtgaggtcgtggttcgcgaagatataatgttggcgattgaaagagccaaatttggaattaaagaaacacgattaacccccgaaacaataggtaaaggtttaggtaaattgtttccacaaatgcctgctttgttcaaaagagatggcagaagtgatgaatacaatggataccaaacattaagttag

SEQ ID NO: 156 - protein, 596 aa, Zostera marina
  > Zosma54g01120
MTSFFYVFPSYSPFTSPDPSRFNRSLVLYNGGRITVPSRKLGVFCFLGNGYVSRCSRECLCSPRCQNRCRRRIVSRINSGGDNRIDSTEDERTNGRKKFNLMDFFKNIESNLRKNPKKVMISAMISVSLALCILLLKLTSMPTPKLVCYSDFVTKLQNGGISEVLFEEGTPRILYKTVAYEKSEEPSEAVVVVEPEATKKGRTPFKLTMTAIITAASKARTKKKNKSIPEWSFTTRKIKHNENFLFDLMREKGTKYGSAPQSPIALLRNALLALLTLWIPLAPILWLLHRQLSSSNTAAKKKRPKNQLVGFNDVEGVDAAKEELLEIVSCLQGSVNFSKLGARLPKGVLLVGPPGTGKTLLARAVAGEADVPFFPVSASEFVELFVGRGASRIRDLFKVARKNSPSIVFIDELDAVGGKRGRSFNDERDQTLNQLLTEMDGFESDTNVIVIAATNRPEALDPALCRPGRFSRRVFVEEPDLDGRKRILGVHIKGVPLEEDAEIICSLIASLTPGFVGADLANIINEAALLAARRGGEVVVREDIMLAIERAKFGIKETRLTPETIGKGLGKLFPQMPALFKRDGRSDEYNGYQTLS

SEQ ID NO: 157 - DNA, 1734 bp, Zea mays
  > GRMZM2G177070
atggcttcgctctgccccttatcagccgcgcccgctttcccgcgcgctgctcgacctcgtgccgcaagcttcaggggacccgcttcctggagcggcctcgcggcgaccgcgagcaatgcctgtgtctgtagctgtggacttctgcccactggaggctacgcgaggctgcggtcgcgactaaggagtcccgtgcgcgccaagatcgacgaggcggacaagggcacgggcgccggtctgggcttcccctcgccacgccgtaggaagctacgcttgcgcctgcgaccacggctgcgcctcttgtggtggcggctccggcggctgtcgccgcgggagctcatgaccgacgccggggccgcgctgcggcgcgccgttcgccgcgtgcctcccgcggccgcggcgccggtcgtcctggcgcttctcctcgccgtggcgcggctcgcgctgcccaagaacgtggcccgggaggtggcctactcggacctcgtcgcggggctccgcgagggcgccgtggccgccgtcgctttcgaggaggactcgcgccgtatctacttcagcaagaacgctggggatgatgttggctccgacacgaacgagaccggtgagaacgctgcggcgccggcgccgaagtggccgtactacgcgcggagggtgccgcacgatgagggcttcttgctcgggctaatgcgggatggccgggtggactaccgttcgtcgccgcggcctgctgggaggctgctcgtggacatgctgagcacgctgctcacgctttgggtctcgctggtgcccgtgatgtggttcatacagcgtcagatgtctggtgcgggcggtgctgacaagcgacgcaaaccgaggaagcagagggtgggctttgatgatgttcagggcatagatgaagccaaggaggagcttgttgagatagtgagctgtctgcatggttcactgaattataagaagcttggagcaaaattgcccaggggtgttctgcttgttggccctcctggaactgggaagactctgctagcaagggcagttgcaggagaggctggaattccgtttttctctgtttctgctagtgaatttgttgaagtttttgttggacgaggtgcagctcgggtaagggatttgttcaaggaggccaaagaagctgccccgtcaatcatttttattgatgaacttgatgccgtgggtggaagtcgaggtagaagttttaatgatgagagggaccaaactctaaaccagctgcttaccgaaatggatggttttgactcggacatgaaagttattgtaatggctgctactaacagaccaaaagcactggatgctgctctttgtcgacctggccgcttctccagaaaggttgttgttggtatgcctgatgtggagggccgcaaaaagattctggatgttcatctaaggaatattcctttggaagaggatcacggaataatcagtgatctggttgctagagtaaccccagggctggtcggcgcagatctagcaaacattgttaatgagtctgctctactagctgcacgaagaggtcggaatactgtgtctcgggaggatattatggatgcaatcgagagggagaaatacggggtcactgggaggcaagaagctgattctgaaaggcaaggcctcaccaagttatttccatggctatctaaacctggaaataaaccatcaagccctgatgatttccgaggattgatgggttatcacacattaagctaa

SEQ ID NO: 158 - protein, 577 aa, Zea mays
  > GRMZM2G177070
MASLCPLSAAPAFPRAARPRAASFRGPASWSGLAATASNACVCSCGLLPTGGYARLRSRLRSPVRAKIDEADKGTGAGLGFPSPRRRKLRLRLRPRLRLLWWRLRRLSPRELMTDAGAALRRAVRRVPPAAAAPVVLALLLAVARLALPKNVAREVAYSDLVAGLREGAVAAVAFEEDSRRIYFSKNAGDDVGSDTNETGENAAAPAPKWPYYARRVPHDEGFLLGLMRDGRVDYRSSPRPAGRLLVDMLSTLLTLWVSLVPVMWFIQRQMSGAGGADKRRKPRKQRVGFDDVQGIDEAKEELVEIVSCLHGSLNYKKLGAKLPRGVLLVGPPGTGKTLLARAVAGEAGIPFFSVSASEFVEVFVGRGAARVRDLFKEAKEAAPSIIFIDELDAVGGSRGRSFNDERDQTLNQLLTEMDGFDSDMKVIVMAATNRPKALDAALCRPGRFSRKVVVGMPDVEGRKKILDVHLRNIPLEEDHGIISDLVARVTPGLVGADLANIVNESALLAARRGRNTVSREDIMDAIEREKYGVTGRQEADSERQGLTKLFPWLSKPGNKPSSPDDFRGLMGYHTLS

SEQ ID NO: 159 - DNA, 22 bp, Artificial Sequence
  >AtFtsHi3 promoter_Topo_F
caccactaacctgaagagactc

SEQ ID NO: 160 - DNA, 20 bp, Artificial Sequence
  >qi-3-RT-PCR FP
ccagacgttaaaccagttgc

SEQ ID NO: 161 - DNA, 19 bp, Artificial Sequence
  >qi-3-RT-PCR RP
cttctggtcggttagttgc

SEQ ID NO: 162 - DNA, 20 bp, Artificial Sequence
  >ACT2-RT-PCR FP
cttgcaccaagcagcatgaa

SEQ ID NO: 163 - DNA, 24 bp, Artificial Sequence
  >ACT2 -RT-PCR FP
ccgatccagacactgtacttcctt

SEQ ID NO: 164 - DNA, 15 bp, Artificial Sequence
  >UBQ5-RT-PCR FP
acgcttcatctcgtc

SEQ ID NO: 165 - DNA, 15 bp, Artificial Sequence
  >UBQ5-RT-PCR FP
ccacaggttgcgtta

SEQ ID NO: 166 - DNA, 19 bp, Artificial Sequence
  >Tubulin-RT-PCR FP
ggtatccaacccgatggca

SEQ ID NO: 167 - DNA, 23 bp, Artificial Sequence
  >Tubulin-RT-PCR FP
tgagcttgtctcgctaaagaatg

SEQ ID NO: 168 - DNA, 21 bp, Artificial Sequence
  >DI21-RT-PCR FP
tcccttactcaatcctgctgc

SEQ ID NO: 169 - DNA, 21 bp, Artificial Sequence
  >DI21-RT-PCR RP
actctccggtgccgttaaatc

SEQ ID NO: 170 - DNA, 21 bp, Artificial Sequence
  >DREB1A-RT-PCR FP
gcgctaaggacatccaaaagg

SEQ ID NO: 171 - DNA, 22 bp, Artificial Sequence
  >DREB1A-RT-PCR RP
gtaaatagcctccaccaacgtc

SEQ ID NO: 172 - DNA, 21 bp, Artificial Sequence
  >NECD3-RT-PCR FP
ttcatctgcgcttcacactcc

SEQ ID NO: 173 - DNA, 21 bp, Artificial Sequence
  >NECD3-RT-PCR RP
gccgctctctggaacaaattc

SEQ ID NO: 174 - DNA, 21 bp, Artificial Sequence
  >COR47-RT-PCR FP
gttggttgtaacggagcatcc

SEQ ID NO: 175 - DNA, 22 bp, Artificial Sequence
  >COR47-RT-PCR RP
ccaaaatccccttcttctcctc

SEQ ID NO: 176 - DNA, 20 bp, Artificial Sequence
  >P5CS RT-PCR FP
agcagcctgtaatgcgatgg

SEQ ID NO: 177 - DNA, 20 bp, Artificial Sequence
  >P5CS RT-PCR RP
aagtgacgcctttggtttgc

SEQ ID NO: 178 - DNA, 19 bp, Artificial Sequence
  >RD22 RT-PCR FP
agggctgtttccactgagg

SEQ ID NO: 179 - DNA, 23 bp, Artificial Sequence
  >RD22 RT-PCR RP
caccacagatttatcgtcagaca

SEQ ID NO: 180 - DNA, 23 bp, Artificial Sequence
  >RD29A RT-PCR FP
gttactgatcccaccaaagaaga

SEQ ID NO: 181 - DNA, 22 bp, Artificial Sequence
  >RD29A RT-PCR RP
ggagactcatcagtcacttcca

SEQ ID NO: 182 - DNA, 17 bp, Artificial Sequence
  >DREB2A RT-PCR FP
gcagtttatgatcagag

SEQ ID NO: 183 - DNA, 20 bp, Artificial Sequence
  >DREB2A RT-PCR FP
aacttcttctacggtctcgt

SEQ ID NO: 184 - DNA, 20 bp, Artificial Sequence
  >RD29B RT-PCR FP
ggagtgaaggagacgcaaca

SEQ ID NO: 185 - DNA, 20 bp, Artificial Sequence
  >RD29B RT-PCR RP
ccacctcctttgtagccgtt

SEQ ID NO: 186 - DNA, 23 bp, Artificial Sequence
  >ftshi3 Reverse for HA-line
gctgagagtttgataacctaacg

SEQ ID NO: 187 - DNA, 22 bp, Artificial Sequence
  >AtFtsHi3Topo_F
caccatggctactttcaatgtt

(19) United States
(12) Patent Application Publication
(10) Pub. No.: US 2025/0340897 A1
(43) Pub. Date: Nov. 6, 2025

(54) GENETICALLY MODIFIED PLANTS WITH IMPROVED YIELD AND DROUGHT TOLERANCE AND METHOD FOR OBTAINING SUCH PLANTS

(71) Applicant: SweTree Technologies AB, Umeå (SE)

(72) Inventors: Christiane Funk, Umeå (SE); Laxmi Mishra, Umeå (SE)

(21) Appl. No.: 18/561,113

(22) PCT Filed: May 20, 2022
(86) PCT No.: PCT/SE2022/050497

§ 371 Date: Nov. 15, 2023

(30) Foreign Application Priority Data
May 21, 2021 (SE) 2150653-0

(51) Int. Cl. C12N 15/82; C12N 9/50

(52) U.S. Cl. CPC C12N 15/8273; C12N 9/63; C12Y 304/24

(57) ABSTRACT

The present invention relates to a method for obtaining a genetically modified plant having increased yield and improved drought tolerance, as compared to a wild type control plant of the same species, comprising: Modifying the genomic DNA in at least one cell of said plant species to increase expression of a FTSHi3 gene thereby obtaining a genetically modified cell; generating a plant from the genetically modified cell to obtain a genetically modified plant; growing said genetically modified plant under conditions which permit development of a plant; and selecting a genetically modified plant having improved drought tolerance. The invention also relates to plants obtainable by such methods, and genetically modified plants exhibiting at least 50% increased expression of a FTSHi3 gene as compared to the wild-type control plant of the same species.

### INCORPORATION BY REFERENCE OF THE SEQUENCE LISTING

This application includes a sequence listing submitted electronically as text file P148281XX00 ST90 2024 Mar. 8 corrected sequence listing.txt; Size: 598,016 bytes; Created: Mar. 8, 2024 and is incorporated herein by reference.

### FIELD OF THE INVENTION

The present invention relates to the field of plant biotechnology and to a method of improving yield and drought tolerance in plants through overexpression of a FtsHi3 pseudo-protease gene, and use of it as a regulator of drought tolerance in plants and to the resulting plants having drought tolerance or improved drought tolerance.

### BACKGROUND

Drought is an environmental stress that can negatively impact plant productivity and crop yields. A plant under stress, such as drought, respond via a series of physiological, cellular, and molecular processes culminating in stress tolerance. These responses include stomatal closure, repression of cell growth and photosynthesis, and activation of respiration.

Several classes of proteases and protease activities are involved in the acclimation of plants to drought reviewed by Vaseva et al., 2011 and by Fanourakis et al., 2020. For example, the senescence-associated subtilisin protease, SASP, is a key regulator in abscisic acid, ABA signalling and drought tolerance.

Filamentation-temperature-sensitive protein H, FtsH, proteases are a family of membrane-bound enzymes present in eubacteria, animals, and plants. FtsH proteases play an essential role in the degradation of both soluble and membrane proteins. All FtsH proteases contain a putative AAA ATPase domain, an ATPase AAA core and an M41 peptidase domain. In addition to active FtsH proteases, pseudo-proteases, termed FtsHi (i for inactive) have been detected in the genomes of plants.

Besides the 12 genes encoding proteolytically active members of the FtsH family in the genome of Arabidopsis, there are five genes coding for members that are assumed to be proteolytically inactive due to mutations in the protease domain; these are termed FtsHi, “i” for inactive. Despite their lack of proteolytic activity, these FtsHi members seem to be important for chloroplast- and plant-development as four out of five homozygous knockout-mutants of FtsHis are embryo-lethal, i.e. a plant without any of these four genes will not grow.

The nature of drought-resistance and tolerance in plants is still not fully understood. There is a need for drought-resistant plants wherein the drought tolerance is increased without side effects such as decreased growth and other drawbacks.

### SUMMARY OF THE INVENTION

The main aspects of the invention are set out in the appended independent claims.

Presently preferred embodiments of the inventions are set out in the appended dependent claims.

### BRIEF DESCRIPTION OF THE DRAWINGS

FIG. 1: A: Rosette diameter measurements were performed from week 2-6 on soil-grown Arabidopsis thaliana plants (WT, FtsHi3 overexpressors “OE” pAtFtsHi3::AtFtsHi3:HA lines 1 and 2, and ftshi3-1 knock-down “kd”) in a growth chamber under SD at 22° C. Asterisks indicate a significant difference (P<0.05, Student's t-test, n=8);

B: Graphical representation of AtFTSHi3 transcript abundance in WT, OE1 and OE2 (FtsHi3 overexpressors “OE” pAtFtsHi3::AtFtsHi3:HA lines 1 and 2), and ftshi3-1 knock-down “kd”. The data are normalized to the expression of housekeeping genes coding for At-UBQ and At-actin. An asterisk indicates significant differences between WT, and OE1 or OE2 seedlings (P<0.05, Student's t-test, n=3); error bars represent the SE

FIG. 2: The water loss rate of 30-day-old WT, OE1 and OE2 lines (FtsHi3 overexpressors “OE” pAtFtsHi3::AtFtsHi3:HA lines 1 and 2). The plants were grown on soil, then their rosettes were detached, and the water loss rate was calculated based on differences to fresh weight. Values are means±SE. Asterisks indicate a significant difference compared to WT (P<0.05, Student's t-test, (n=10)).

FIG. 3: Quantification of ABA (ng/gm) in leaves of watered and drought-stressed WT, OE1 and OE2 (FtsHi3 overexpressors “OE” pAtFtsHi3::AtFtsHi3:HA lines 1 and 2) plants. Values are means±SE. Asterisks indicate the significant difference of ABA (ng/gm) content in watered and drought conditions (P<0.05, Student's t-test, n=4).

FIG. 4: A: Relative expression of ABA-responsive genes in WT, OE1 and OE2 lines (FtsHi3 overexpressors “OE” pAtFtsHi3::AtFtsHi3:HA lines 1 and 2) in watered conditions.

B: Relative expression of ABA-responsive genes in WT, OE1 and OE2 lines (FtsHi3 overexpressors “OE” pAtFtsHi3::AtFtsHi3:HA lines 1 and 2) in drought conditions.

Data are normalized to the expression of actin. Asterisks indicate significant differences compared to WT control (Student's t-test, P<0.05, n=3). Error bars represent the SE.

FIG. 5: Percent Identity Matrix for orthologous FTSHi3 genes, showing sequence identities relative to AtFTSHi3.

FIG. 6: Percent Identity Matrix for orthologous FTSHi3 genes, showing sequence identities relative to PtFTSHi3 (Potra001056g09045).

FIG. 7: Images (top) and quantification (bottom) of the root length and root branching of Arabidopsis thaliana wild-type (WT), FtsHi3 overexpressors (“OE” pAtFtsHi3::AtFtsHi3:HA lines 1 and 2), and ftshi3-1 knock-down (kd) seedlings grown for 8 days on MS medium agar plates supplemented with 1% sucrose, under LD at 22° C. Asterisks indicate a significant difference compared with the WT control (P<0.05, Student's t-test)

FIG. 8: A: Rosette diameter measurements were performed from week 2-6 on soil-grown Arabidopsis thaliana wild-type (WT), 35S::AtFtsHi3::GFP(ftshi3-1) comp lines 1-3, and ftshi3-1 knock-down (kd) plants in a growth chamber under SD at 22° C. Asterisks indicate a significant difference compared with the WT control (P<0.05, Student's t-test, n=8).

B: A: Rosette diameter measurements were performed from week 2-6 on soil-grown Arabidopsis thaliana wild-type (WT), 35S::AtFtsHi3::GFP(WT) lines 3-6, and ftshi3-1 knock-down (kd) plants in a growth chamber under SD at 22° C. Asterisks indicate a significant difference compared with the WT control (P<0.05, Student's t-test, n=8).

FIG. 9: A: Relative AtFTSHi3 transcript abundance in 35S::AtFtsHi3::GFP(WT) lines compared to WT levels, in 10-day-old seedlings. The AtFTSHi3 transcript abundance is normalized to the transcript abundance of housekeeping genes At-UBQ and At-actin. Asterisks indicate significant differences compared to WT control (P<0.05, student's t-test, n=3); error bars represent the SE.

B: Relative AtFTSHi3 transcript abundance in 35S::AtFtsHi3::GFP(ftshi3-1) comp lines compared to WT levels, in 10-day-old seedlings. The AtFTSHi3 transcript abundance is normalized to the transcript abundance of housekeeping genes At-UBQ and At-actin. Asterisks indicate significant differences compared to WT control (P<0.05, student's t-test, n=3); error bars represent the SE.

FIG. 10: A: Top-view photographs of WT and pAtFtsHi3::AtFtsHi3:HA overexpressors (OE) 1 and 2 plants exposed to drought stress for 20 days and re-watered for 14 days.

B: Top-view photographs of WT, ftshi3-1 knock-down (kd), and pAtFtsHi3::AtFtsHi3:HA and 35S::AtFtsHi3::GFP(ftshi3-1) comp lines exposed to drought stress for 20 days, and then re-watered for 14 days.

FIG. 11: A: Measurements of the stomatal density of WT, and pAtFtsHi3::AtFtsHi3:HA overexpressors (OE) 1 and 2 plants. All data values are means±SE of five 5 biological replicates, and thirty 30 stomata per plant were measured. Asterisks indicate significant differences (One-way Anova, Fisher LSD posthoc test; P<0.05).

B: Measurements of the stomatal dimensions length and width of WT, and pAtFtsHi3::AtFtsHi3:HA overexpressors (OE) 1 and 2 plants. All data values are means±SE of five 5 biological replicates, and thirty 30 stomata per plant were measured. Asterisks indicate significant differences (One-way Anova, LSD posthoc test; P<0.05).

C: A, Fresh weight measurements of seedlings of WT, and pAtFtsHi3::AtFtsHi3:HA overexpressors (OE) 1 and 2 grown in the absence of exogenous ABA and mannitol or exposed to 1 μmol or 5 umol ABA, or 200 mM or 500 mM mannitol. Asterisks indicate significant differences compared to WT control (P<0.05, Student's t-test, 3 sets containing fifteen biological replicates per treatment per genotype). Error bars represent the SE.

FIG. 12: A: Relative AtFTSHi3 transcript abundance in seedlings of WT, and pAtFtsHi3::AtFtsHi3:HA (previously identified lines thereafter “pFtsHi3-OE1 and 2”, as well as newly generated lines “pFtsHi3-OE3-11”) lines. The AtFtsHi3 transcript levels are normalized to the transcript abundance of housekeeping genes encoding At-UBQ and At-actin. Asterisks indicate significant differences compared to WT control (P<0.05, Student's t-test, n=3); error bars represent the SE.

B: Relative AtFTSHi3 transcript abundance in seedlings of WT, and 35S::AtFtsHi3::GFP(WT) (newly generated lines “p35S-OE1-7”) lines. The AtFtsHi3 transcript levels are normalized to the transcript abundance of housekeeping genes encoding At-UBQ and At-actin. Asterisks indicate significant differences compared to WT control (P<0.05, Student's t-test, n=3); error bars represent the SE.

FIG. 13: A: Rosette photographs of pFtsHi3-OE, p35S-OE lines and WT at the age of 23 days, grown in SD conditions.

B: Rosette photographs of pFtsHi3-OE, p35S-OE lines and WT at the age of 49 days, grown in SD conditions, including 15 days of drought stress (from day 34 to day 49).

FIG. 14: A: Rosette diameter measurements were performed from week 2-6 on soil-grown WT, and pFtsHi3-OE plants under SD and 22° C. An asterisk indicates significant difference compared with the WT control (P<0.05, Student's t-test, n=15 for pFtsHi3-OE lines 3-11, n=35 for WT, and pFtsHi3-OE lines 1 and 2). Error bars 10 represent the SEM B: Rosette diameter measurements were performed from week 2-6 on soil-grown WT, and p35S-OE plants under SD and 22° C. An asterisk indicates significant difference compared with the WT control (P<0.05, Student's t-test, n=15 for p35S-OE lines 1-7, n=35 for WT). Error bars represent the SE. 15 FIG. 15: A: Rosette fresh weight measurements from 6-week-old soil-grown WT, and pFtsHi3-OE plants under SD and 22° C. An asterisk indicates significant difference compared with the WT control (P<0.05, Student's t-test, n=15 for pFtsHi3-OE lines 3-11, n=35 for WT, and pFtsHi3-OE lines 1 and 2). Error bars represent the SE.

B: Rosette fresh weight measurements from 6-week-old soil-grown WT, and p35S-OE plants under SD and 22° C. An asterisk indicates significant difference compared with the WT control (P<0.05, Student's t-test, n=15 for p35S-OE lines, n=35 for WT). Error bars represent the SE.

C: Rosette dry weight measurements from 6-week-old soil-grown WT, and pFtsHi3-OE plants under SD and 22° C. An asterisk indicates significant difference compared with the WT control (P<0.05, Student's t-test, n=15 for pFtsHi3-OE lines 3-11, n=35 for WT, and pFtsHi3-OE lines 1 and 2). Error bars represent the SE.

D: Rosette dry weight measurements from 6-week-old soil-grown WT, and p35S-OE plants under SD and 22° C. An asterisk indicates significant difference compared with the WT control (P<0.05, Student's t-test, n=15 for p35S-OE lines, n=35 for WT). Error bars represent the SE.

FIG. 16: A: Leaf water content from 6-week-old soil-grown WT, and pFtsHi3-OE plants after drought treatment followed by 14 days of rewatering. An asterisk indicates significant difference compared with the WT control (P<0.05, Student's t-test, n=15 for pFtsHi3-OE lines 3-11, n=35 for WT, and pFtsHi3-OE lines 1 and 2). Error bars represent the SE.

B: Soil moisture content of pots in which a WT, or a pFtsHi3-OE plant had been grown during 6 weeks under SD and 22° C., including drought treatment followed by 14 days of rewatering. An asterisk indicates significant difference compared with the WT control (P<0.05, Student's t-test, n=15 for pFtsHi3-OE lines 3-11, n=35 for WT, and pFtsHi3-OE lines 1 and 2). Error bars represent the SE.

C: Leaf water content from 6-week-old soil-grown WT, and p35S-OE plants after drought treatment followed by 14 days of rewatering. An asterisk indicates significant difference compared with the WT control (P<0.05, Student's t-test, n=15 for p35S-OE lines, n=35 for WT). Error bars represent the SE.

D: Soil moisture content of pots in which a WT, or a p35S-OE plant had been grown during 6 weeks under SD and 22° C., including drought treatment followed by 14 days of rewatering. An asterisk indicates significant difference compared with the WT control (P<0.05, Student's t-test, n=15 for p35S-OE lines, n=35 for WT). Error bars represent the SE.

### DEFINITIONS

All terms and words used in the present specification are intended to have the meaning generally given to them by the person skilled in the art of plant biotechnology. However, a few terms are explained in more detail below in order to avoid ambiguities.

The terms “drought resistance” and “drought tolerance” are used interchangeably herein and are intended to mean the increased ability of a plant, as compared to a control wild-type plant of the same species, to survive an extended period of drought which with a high likelihood kills or severely damages the control wild-type plant, and be able to continue or resume growth on watering after such extended period of drought or the increased ability of a plant, compared to a wild-type plant, to continue growing under conditions when water availability is reduced without having a negative impact on survival.
ABA: Abscisic acid
SD conditions: Short Day conditions (8h light/16h dark)
LD conditions: Long Day conditions (16 h light/8 h dark)
WT: wild type
OE: overexpressor

### Ftshi3-1(Kd): Knock-Down Mutant of FTSHi3

The naming of genes presented in this disclosure originate from the inventors or others' work. In brief, the first two to five letters denotes the plant name in Latin directly followed by the gene name, exemplified by the gene FTSHi3, from Arabidopsis thaliana it is denoted, AtFTSHi3. The same gene from Eucalyptus grandis is denoted EgFTSHi3, and the same gene from aspen (Populus tremula) is denoted PotraFtsHi3 or Potra001056g09045. FTSHi3 is used interchangeably with FtsHi3. When an orthologue gene is known it will follow the name presented at the Phytozome Comparative Plant Genomics Portal (phytozome.jgi.doe.gov) using the latest version of Phytozome. At present the version 12.1 is used. Orthologous gene names in the present disclosure are found in Phytozome.

A “p” in front of a gene denotes that this is the promoter of said gene, for example pRBCS is the promoter of the gene encoding ribulose-1,5-bisphosphate carboxylase small subunit (RBCS).

By “orthologue” or “orthologous polypeptide” is meant a polypeptide expressed by evolutionarily related genes that have a similar nucleic acid sequence, where the polypeptide has similar functional properties. Orthologous genes are structurally related genes, from different species, derived by a speciation event from an ancestral gene. Related to orthologs are paralogs. Paralogous genes are structurally related genes within a single plant species most probably derived by a duplication of a gene. Several different methods are known by those of skill in the art for identifying and defining these functionally homologous sequences.

Orthologous genes from different organisms have highly conserved functions and can be used for identification of genes that could perform the invention in the same way as the genes presented here. Paralogous genes, which have diverged through gene duplication, may encode protein retaining similar functions. Orthologous genes are the product of speciation, the production of new species from a parental species, giving rise to two or more genes with common ancestry and with similar sequence and similar function. These genes, termed orthologous genes, often have an identical function within their host plants and are often interchangeable between species without losing function. Identification of an “orthologue” gene may be done by identifying polypeptides in public databases using the software tool BLAST with one of the polypeptides encoded by a gene. Subsequently additional software programs are used to align and analyze ancestry. The sequence identity between two orthologous genes may be low.

A promoter is said to be an “orthologous promoter” to a promoter in a different species when the respective promoters initiate transcription of orthologous genes in wild type plants of the respective species.

The term “plant” including “crop plants” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

A “woody plant” is a plant that produces wood as a structural tissue.

The terms “substantially identical” or “sequence identity” may indicate a quantitative measure of the degree of identity between two amino acid sequences or two nucleic acids (DNA or RNA) of equal length. When the two sequences to be compared are not of equal length, they are aligned to give the best possible fit, by allowing the insertion of gaps or, alternatively, truncation at the ends of the polypeptide sequences or nucleotide sequences. The “sequence identity” may be presented as percent number, such as at least 40, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% amino acid sequence identity of the entire length, when compared and aligned for maximum correspondence, as measured using a sequence comparison algorithm or by visual inspection.

The sequence identity of the polypeptides of the invention can be calculated as (Nref-Ndif) 100/Nref, wherein Naif is the total number of non-identical residues in the two sequences when aligned and wherein Nref is the number of residues in one of the sequences. The sequence identity between one or more sequence may also be based on alignments using the Clustal W or Clustal X software. In one embodiment of the invention, alignment is performed with the sequence alignment method Clustal X version 2 with default parameters. The parameter set preferably used are for pairwise alignment: Gap open penalty: 10; Gap Extension Penalty: 0.1, for multiple alignment, Gap open penalty is 10 and Gap Extension Penalty is 0.2. Protein Weight matrix is set on Identity. Both Residue-specific and Hydrophobic Penalties are “ON”, Gap separation distance is 4 and End Gap separation is “OFF”, No Use negative matrix and finally the Delay Divergent Cut-off is set to 30%. The Version 2 of Clustal W and Clustal X is described in: Larkin et al. 2007, Clustal W and Clustal X version 2.0. Bioinformatics, 23:2947-2948. The identity between two sequence (protein or nucleic acids) can practically be determined by using different BLAST tools at NCBI (https://www.ncbi.nlm.nih.gov/).

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group Glycine, Alanine, Valine, Leucine, Isoleucine; group Serine, Cysteine, Selenocysteine, Threonine, Methionine; group Proline; group Phenylalanine, Tyrosine, Tryptophan; Group Aspartate, Glutamate, Asparagine, and Glutamine.

In some aspects, the amino acid substantial identity exists over a polypeptide sequences length of 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700 amino acids in the polypeptide with a “sequence identity” as defined above.

In certain aspects, substantial identity exists over a region of nucleic acid sequences of at least about 50 nucleic acid residues, such as at least about 100, 150, 200, 250, 300, 330, 360, 375, 400, 425, 450, 460, 480, 500, 600, 700, 800 such as at least about 900 nucleotides or such as at least about 1 kb, 2 kb, or such as at least about 3 kb.

A gene (nucleic acid molecule comprising a coding sequence) is “operably linked” to a promoter when its transcription is under the control of the promoter and where transcription results in a transcript whose subsequent translation yields the product encoded by the gene.

The term “increasing expression” is intended to encompass well known methods to increase the expression by regulatory sequences, such as promoters, or proteins, such as transcription factors. The terms “increasing expression”, “enhanced expression” and “over-expression” can be used interchangeably in this text. Increased expression may lead to an increased amount of the over-expressed protein/enzyme, which may lead to an increased activity of the protein of interest that contributes to its high efficiency.

The term “yield” as used herein generally refers to a measurable product from a plant, particularly a crop. Yield and yield increase (in comparison to a non-modified starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable. As used herein, the term “improved yield” or the term “increased yield” means any improvement in the yield of any measured plant product, such as grain, fruit, leaf, root, cob or fibre. In accordance with the invention, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, and/or higher dry matter production. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the chimeric genes of the invention, as compared with the bu/acre yield from untransformed soybeans or corn cultivated under the same conditions, is an improved yield in accordance with the invention. The increased or improved yield can be achieved in the absence or presence of stress conditions. For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both. “Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Abiotic stress resulting from drought is of particular relevance to the present invention. The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), harvestable parts weight per area (acre, square meter, or the like); and the like. The harvest index is the ratio of yield biomass to the total cumulative biomass at harvest. Harvest index is relatively stable under many environmental conditions, and so a robust correlation between plant size and grain yield is possible. Measurements of plant size in early development, under standardized conditions in a growth chamber or greenhouse, are standard practices to measure potential yield advantages conferred by the presence of a transgene. Accordingly, the yield of a plant can be increased by improving one or more of the yield-related phenotypes or traits. Such yield-related phenotypes or traits of a plant the improvement of which results in increased yield comprise, without limitation, the increase of the intrinsic yield capacity of a plant, improved nutrient use efficiency, and/or increased stress tolerance. For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like). “Yield” can also refer to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. percent moisture. For example, the term “increased yield” means that a plant, exhibits an increased growth rate, e.g. in the absence or presence of abiotic environmental stress, such as drought, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or 3 seeds. A prolonged growth comprises survival and/or continued growth of the plant, at the moment when the non-transformed wild type organism shows visual symptoms of deficiency and/or death. When the plant of the invention is a corn plant, increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved. When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant. When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant. When the plant of the invention is a cotton plant, increased yield for cotton plants means increased lint yield. Increased lint yield of cotton refers in one embodiment to an increased length of lint. When the plant is a plant belonging to grasses an increased leaf can mean an increased leaf biomass. Said increased yield can typically be achieved by enhancing or improving, one or more yield related traits of the plant. Such yield-related traits of a plant comprise, without limitation, the increase of the intrinsic yield capacity of a plant, improved nutrient use efficiency, and/or increased stress tolerance, in particular increased abiotic stress tolerance, in particular increased drought tolerance. Intrinsic yield capacity of a plant can be, for example, manifested by improving the specific (intrinsic) seed yield (e.g. in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signalling pathway modifications, modification of transcriptional regulation, modification of translational regulation, modification of enzyme activities, and the like); and/or the like.

The term “water use efficiency” (WUE) has been defined in various ways in the literature, but is commonly known as a simple measure for the water productivity of a plant. An increase in water use efficiency is commonly cited as a response mechanism of plants to moderate to severe soil water deficits, and has been the focus of many programs that seek to increase crop tolerance of drought. Different plant species have different inherent water use efficiency.

Water use efficiency is preferably measured by the carbon isotope discrimination analysis for improved drought tolerance. It is known that carbon isotope discrimination is highly correlated with water use efficiency in C3 plants. The isotopic ratio of ¹³C to ¹²C (δ¹³C) in plant tissue is less than the isotopic ratio of ¹³C to ¹²C in the atmosphere, indicating that plants discriminate against ¹³C during photosynthesis. The isotopic ratio δ¹³C varies mainly due to discrimination during diffusion of CO₂ across the stomatal pore, where diffusion of ¹³CO₂ is lower than that of ¹²CO₂, and an additional effect caused by the preference of ribulose bisphosphate carboxylase for ¹²CO₂ over ¹³CO₂. Both processes discriminate against the heavier isotope, 13C, Farquhar, G. D., J. R. Ehleringer, and K. T. Hubick. 1989. Carbon isotope discrimination and photosynthesis. Ann. Rev. Plant Physiol. 40:503-537.

### DETAILED DESCRIPTION OF THE INVENTION

The present inventors have surprisingly found that overexpression of the FTSHi3-gene confers significantly improved drought tolerance to plants in the model species Arabidopsis thaliana. Alternatively, or in addition, overexpression of the FtsHi3 gene confers increased yield.

Consequently, the present invention generally relates to methods comprising modification of the genomic DNA in a plant to overexpress an FTSHi3 gene, for obtaining genetically modified plants having improved drought tolerance, as compared to a wild type (WT) control plant of the same species, as well as to genetically modified plants overexpressing an FTSHi3 gene and having improved drought tolerance.

### Overexpression of FTSHi3 Increases Growth/Yield Compared to WT Plants.

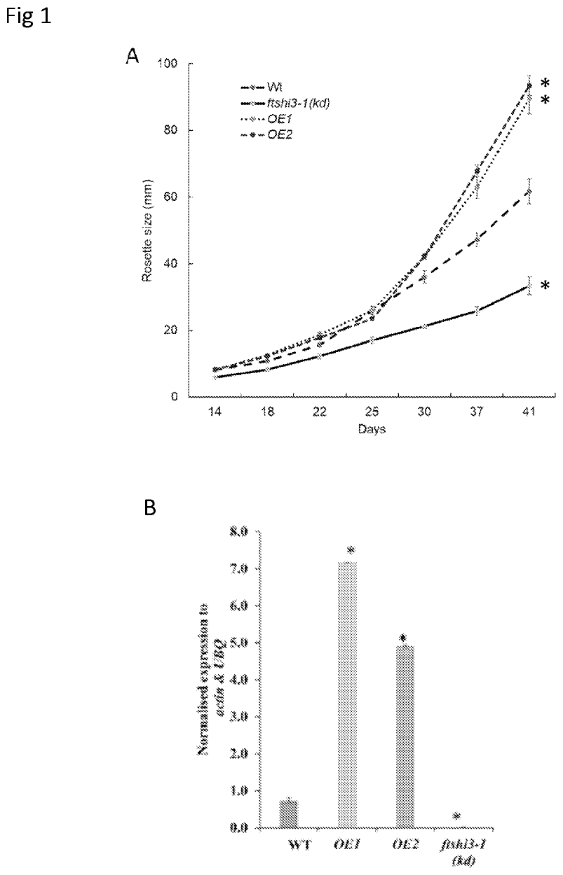

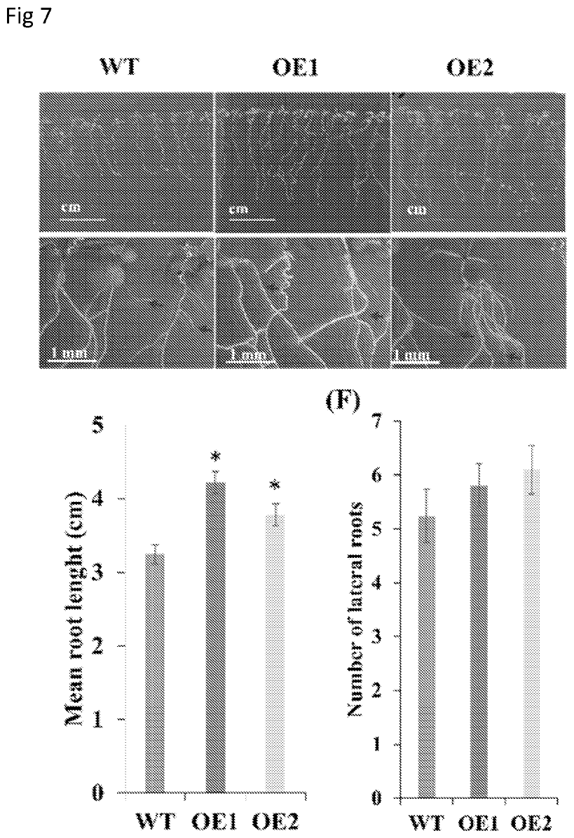

Arabidopsis lines expressing the AtFTSHi3 gene under the control of endogenous AtFTSHi3 promoter were produced as described in the examples. These overexpressing lines (pAtFtsHi3::AtFtsHi3::HA(WT), meaning WT plants transformed with a recombinant DNA construct adding to the genome an extra copy of the AtFTSHi3 gene, with expression under the control of its endogenous promoter) are hereafter designated as OE. Initially, ten independent lines were screened in the T1 generation based on their phenotypes compared to WT and a knock-down mutant (ftshi3-1(kd)) previously described (Mishra et al., 2021) Of the ten independent T1 lines, two representative transgenic lines in T3, OE1 and OE2, were selected for further investigations. Both lines displayed bigger seedlings (FIG. 7) and rosettes (FIG. 1A) compared to WT. The rosette diameters of eight independent rosettes of OE1 and OE2 per line measured from week 2 to 6 grown in SD conditions were significantly bigger than WT (FIG. 1A). qPCR analysis of RNA extracted from 4-week-old plants grown at SD conditions showed higher FTSHi3 transcript levels than WT and ftshi3-1(kd) (FIG. 1B).

Furthermore, significantly larger roots (around 20%) and a slightly increased number of lateral roots were observed in the OE1 and OE2 lines as compared to WT. Chloroplasts in the first true leaves of OE1 and OE2 lines were fully developed and larger than in WT. Increased width and higher length: width ratio as well as more thylakoid membranes in the chloroplasts of OE1 and OE2 lines were also observed. PSII quantum yield (Fv/Fm) performed on these plants grown in SD conditions were similar to WT, while OE1 and OE2 lines displayed slightly higher non-photochemical quenching (NPQ) than WT.

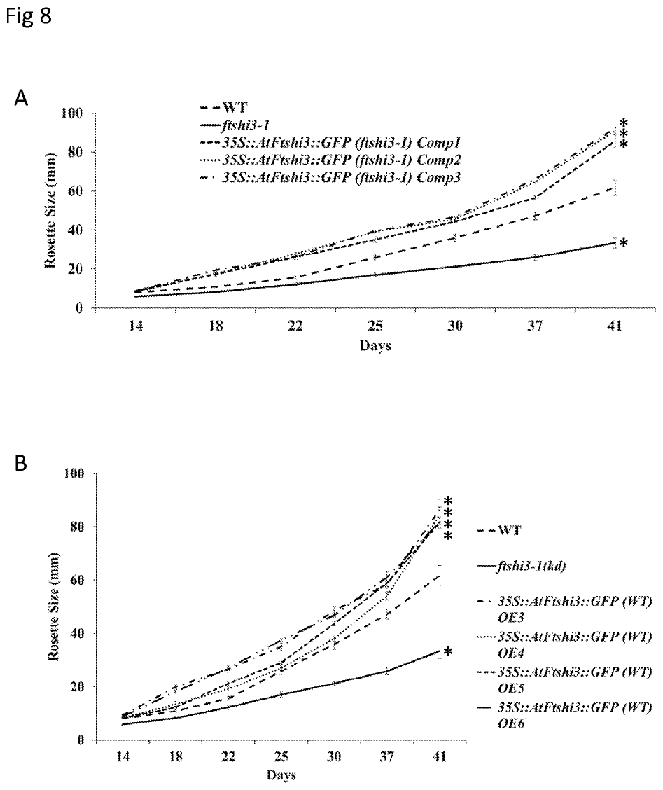

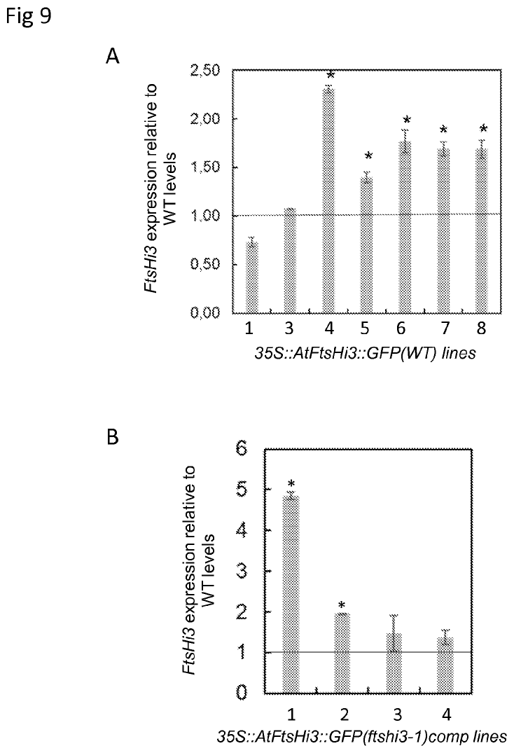

Furthermore WT and ftshi3-1(kd) were transformed by a construct 35S::AtFtsHi3::GFP (construct with SEQ ID NO: 7). The resulting transgenic lines were designated 35S::AtFtsHi3::GFP(WT) OE and 35S::AtFtsHi3::GFP(ftshi3-1) comp lines respectively. Initially, seven to eight independent lines were screened in the T1 generation based on their phenotypes. Of those independent T1 lines, three 35S::AtFtsHi3::GFP(ftshi3-1) comp lines and four 35S::AtFtsHi3::GFP(WT) OE representative transgenic lines in T3 were selected for further investigations. The complementation lines displayed not only WT-like green phenotypes, but were larger than WT (FIG. 8A). Like the OE1 and OE2 lines, the 35S::AtFtsHi3::GFP(WT) OE lines also grew as larger seedlings and developed larger rosettes than WT (FIG. 8B). The rosette diameters of eight independent rosettes per line (OE and comp lines) measured from week 2 to 6 grown in SD conditions were larger than WT. qPCR analysis of RNA extracted from 14-day-seedlings of the 35S::AtFtsHi3::GFP(WT) OE and 35S::AtFtsHi3::GFP(ftshi3-1) comp plants grown at SD conditions showed a varying level of FTSHi3 transcripts compared to WT. 35S::AtFtsHi3::GFP(ftshi3-1) comp1 showed 5× higher level of FTSHi3 transcripts, whereas the 35S::AtFtsHi3::GFP(ftshi3-1) comp lines and 35S::AtFtsHi3::GFP(WT) OE displayed a 2-fold increase (FIG. 9). These experiments collectively demonstrate that the expression of FTSHi3 either by its native promoter or a 35S constitutive promoter is responsible for the plant phenotypes with increased growth and/or improved yield, e.g. increased rosette size and seedling size.

### Improved Plant Drought Tolerance and Reduced Water Loss in Arabidopsis Plants Overexpressing FTSHi3

It was further investigated how plants are affected by overexpression of FTSHi3. Irrigation was stopped for 20 days on four-week-old, soil-grown plants. WT showed an early onset of drought sensitivity compared to ftshi3-1(kd) plants and plants overexpressing FTSHi3 under the control of its endogenous AtFTSHi3 promoter (pAtFtsHi3::AtFtsHi3::HA(WT)) or an 35S promotor (35S::AtFtsHi3::GFP(WT)-OE and 35S::AtFtsHi3::GFP(ftshi3-1) comp in WT and ftshi3-1(kd) background), respectively. After 20 days of drought conditions more than 80% WT plants withered, whereas only 30% of the OE 1&2 (pAtFtsHi3::AtFtsHi3::HA(WT)) plants and 40% of the 35S::AtFtsHi3: GFP(ftshi3-1) comp and 35S::AtFtsHi3: GFP(WT) OE plants showed drought symptoms.

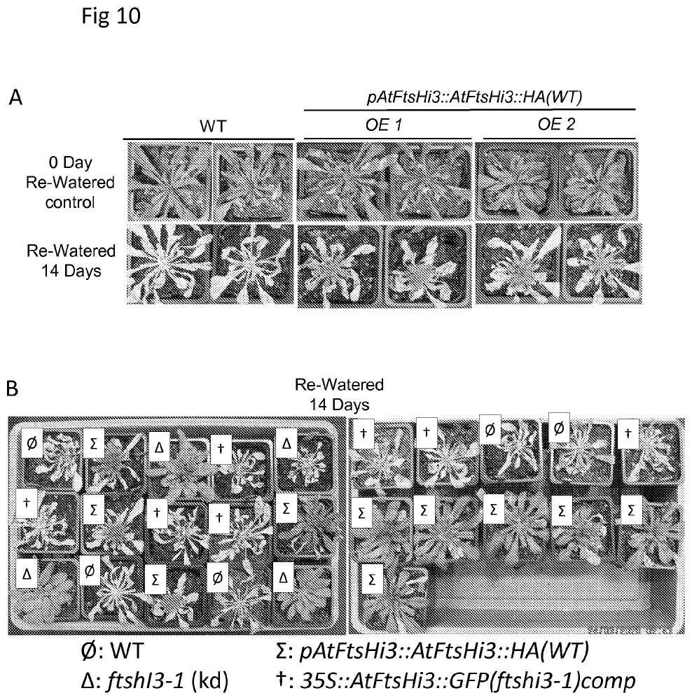

A rewatering experiment was performed to determine the revival frequency on the severely droughted plants across WT, ftshi3-1(kd) and endogenous OE lines (pAtFtsHi3::AtFtsHi3::HA(WT) lines 1 and 2) as well as 35S driven complementation lines (35S::AtFtsHi3::GFP(ftshi3-1) comp lines 1 and 2). The OE lines that showed drought symptoms along with WT, ftshi3-1(kd) and ftshi3-1 (comp-1 &2) (35S::AtFtsHi3::GFP(ftshi3-1) comp lines 1 and 2) plants were re-irrigated with 250 ml of water every day for 14 days. Fifteen pots per tray were randomized and watered every day to test the survival rates of the plants. 90% of WT and ftshi3-1 (comp-1 &2) (35S::AtFtsHi3::GFP(ftshi3-1) comp lines 1 and 2) died during this treatment, whereas 50-75% of the OE (pAtFtsHi3::AtFtsHi3::HA(WT) lines 1 and 2) and ftshi3-1(kd) lines survived (FIG. 10A).

To further compare the drought tolerance between OE lines (pAtFtsHi3::AtFtsHi3::HA(WT) lines 1 and 2) and the WT, ftshi3-1(kd) and (35S::AtFtsHi3::GFP(ftshi3-1) comp) ftshi3-1 (comp-1 &2) the survival rates were evaluated using the ‘same tray’ method where the plants were subjected to watered, half-drought and full drought treatments. The results revealed significant survival of the ftshi3-1(kd) and the OE lines compared to WT and ftshi3-1 (Comp-1 &2) plants in not only the half-drought, but also in the full drought treatments (FIG. 10B).

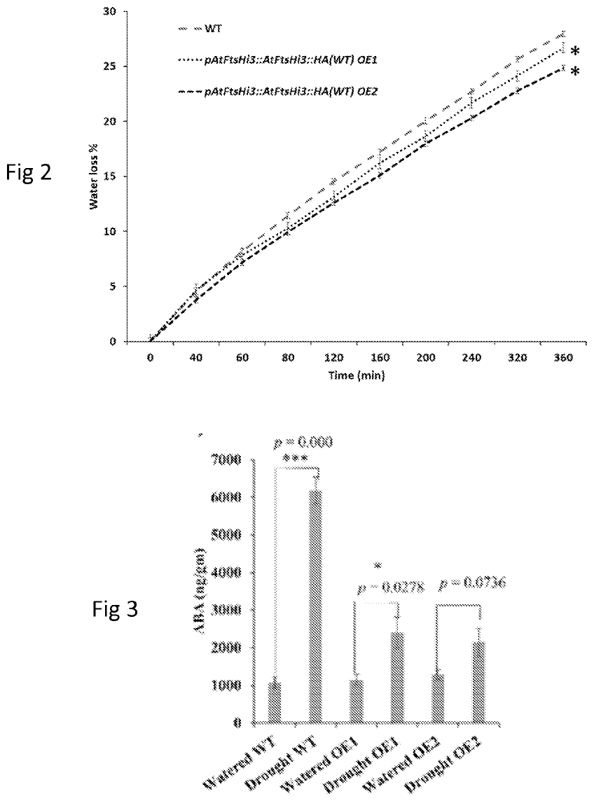

The rate of transpiration was determined by the percentage of water loss in the rosettes separated from the pots. In WT and OE1 and 2 lines, the rate of water loss was similar at the beginning of the experiment, but after about 2 hours, a more substantial water loss was observed in WT than OE plants, which gradually increased with progressing time (FIG. 2).

Arabidopsis Plants Overexpressing FtsHi3 have Lowered Stomatal Density and are Semi-Sensitive to ABA

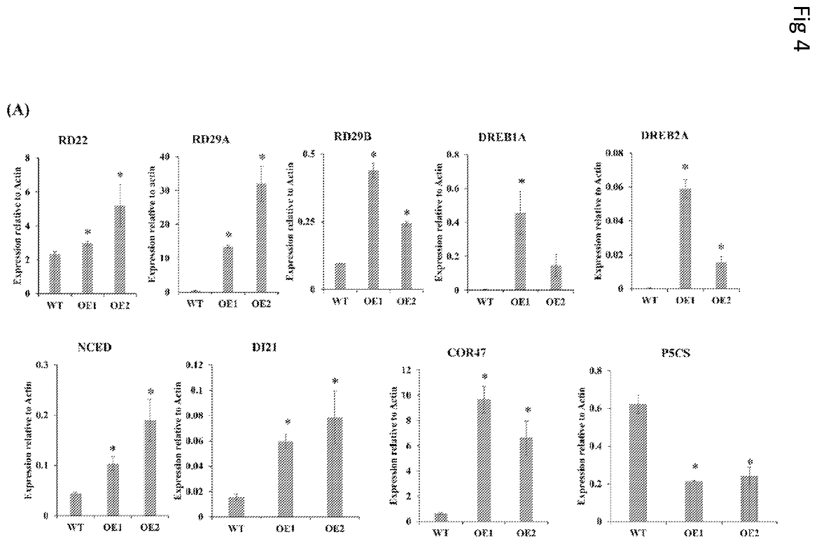

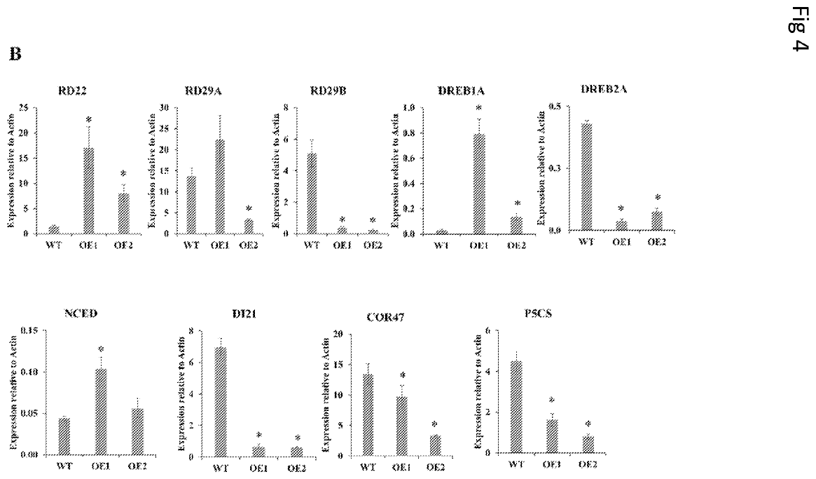

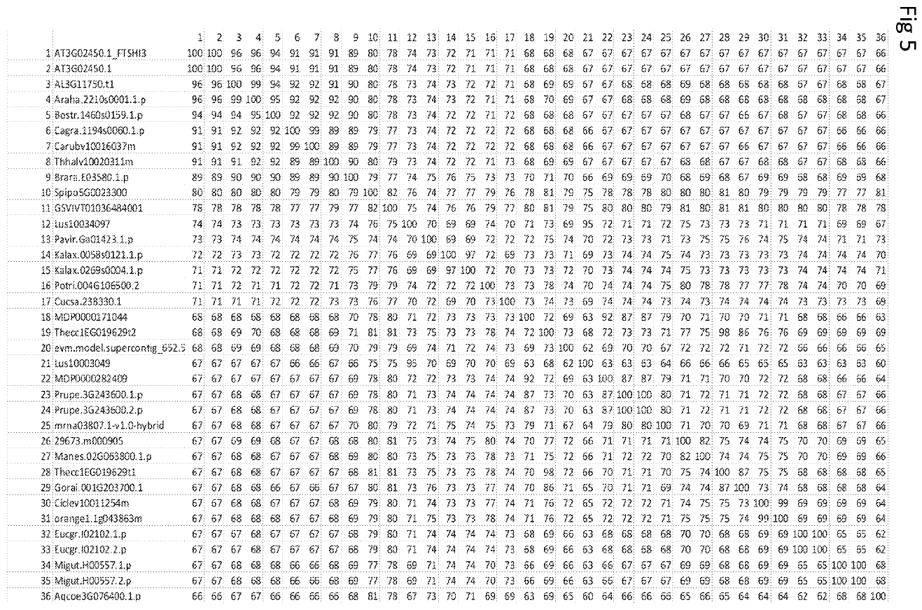

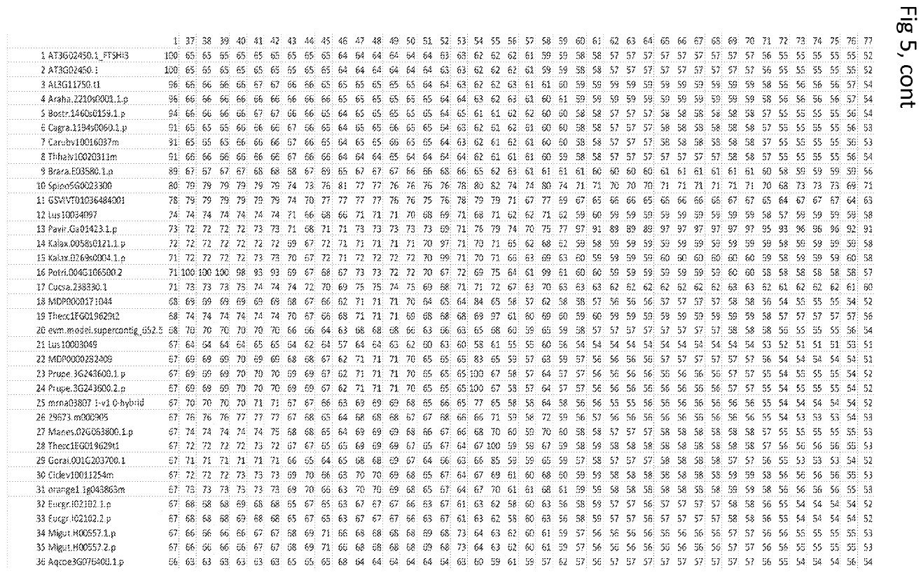

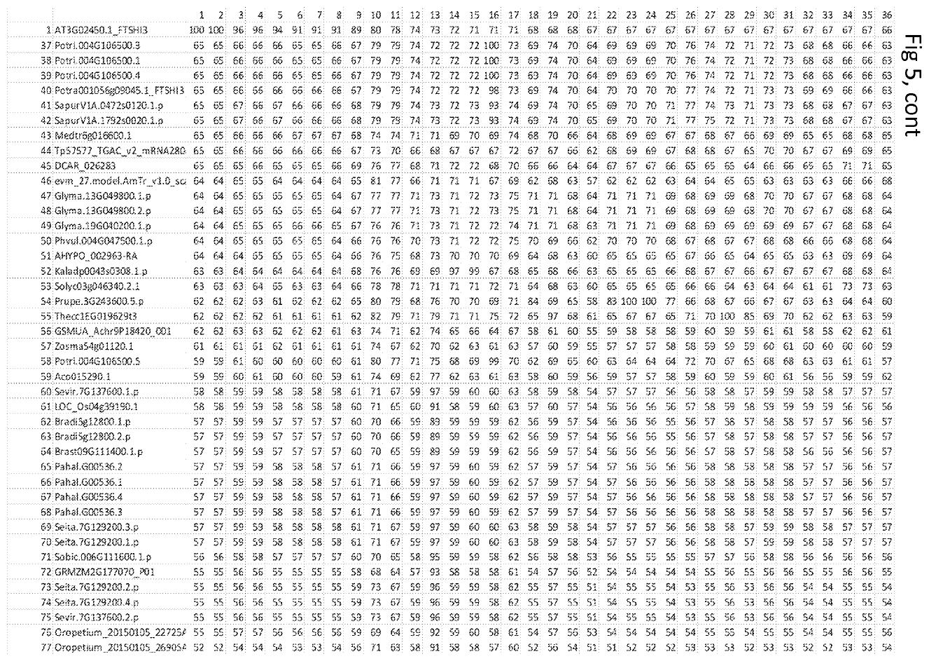

The levels of ABA were further investigated in watered in drought-stressed plants. While the ABA levels (ng/gm) in adult watered plants were similar between all the genotypes (FIG. 3), ABA levels were significantly higher in WT during drought indicating higher drought response. OE1 (pAtFtsHi3::AtFtsHi3::HA(WT) line 1) showed significantly higher ABA levels than OE2 (pAtFtsHi3::AtFtsHi3::HA(WT) line 2) plants in drought, but the ABA levels were elevated even in OE2 plants compared to watered conditions (FIG. 3) indicating a subtle drought response.

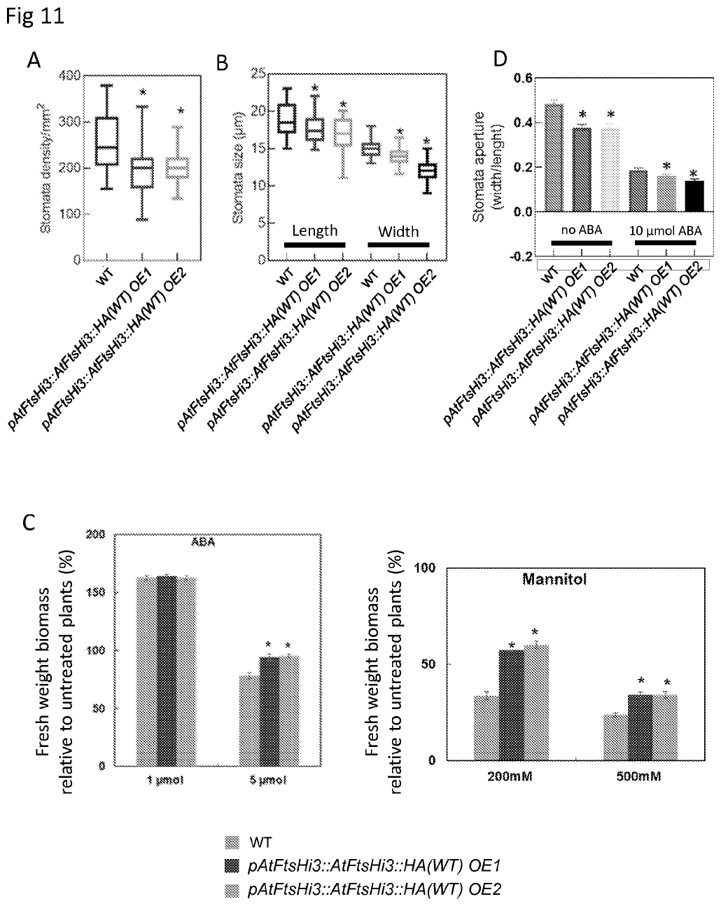

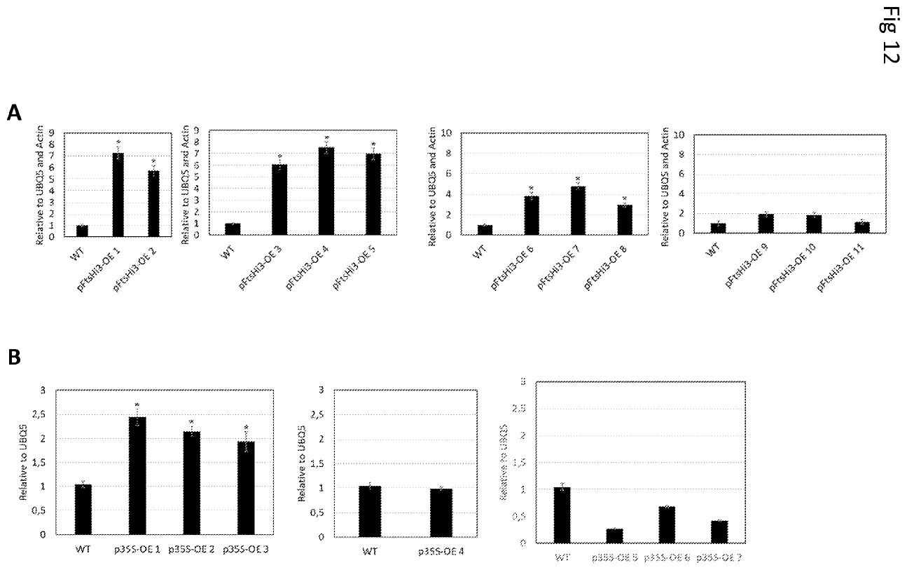

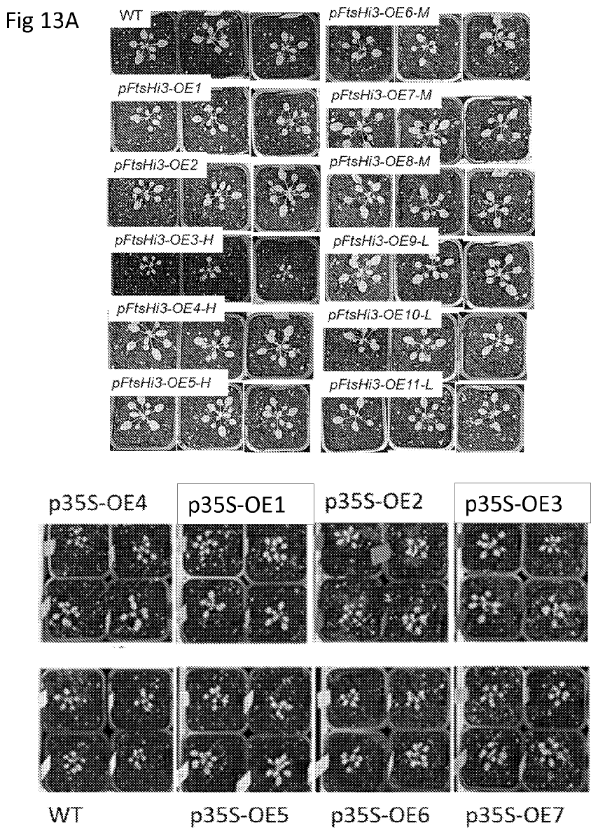

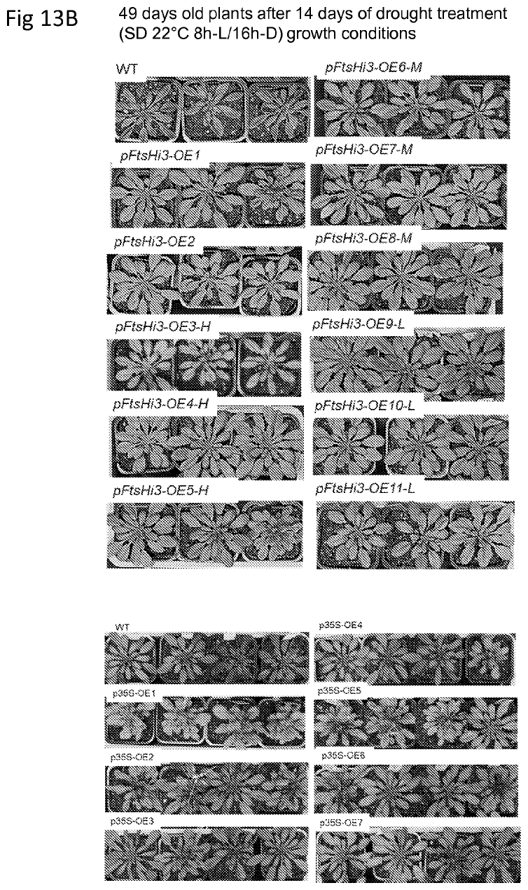

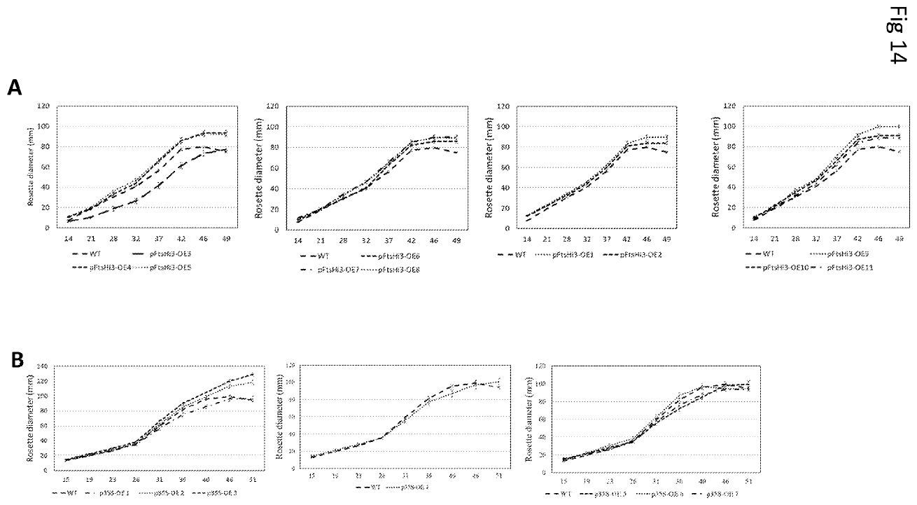

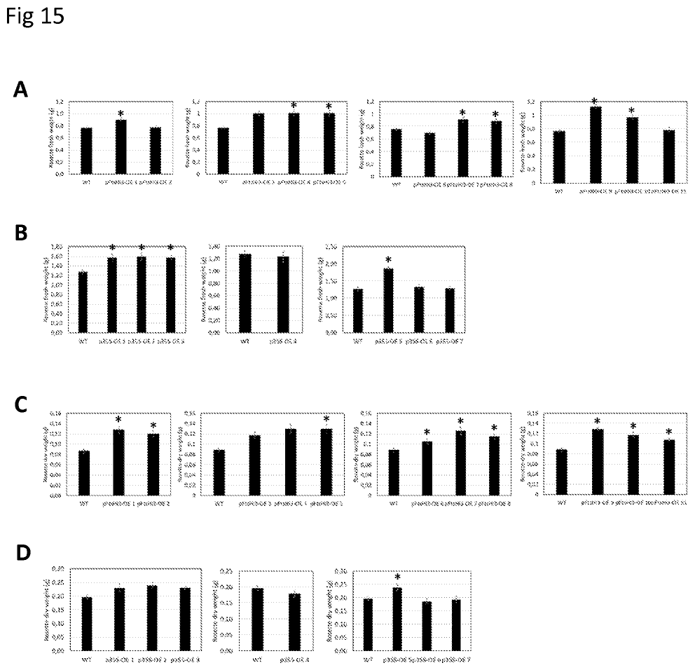

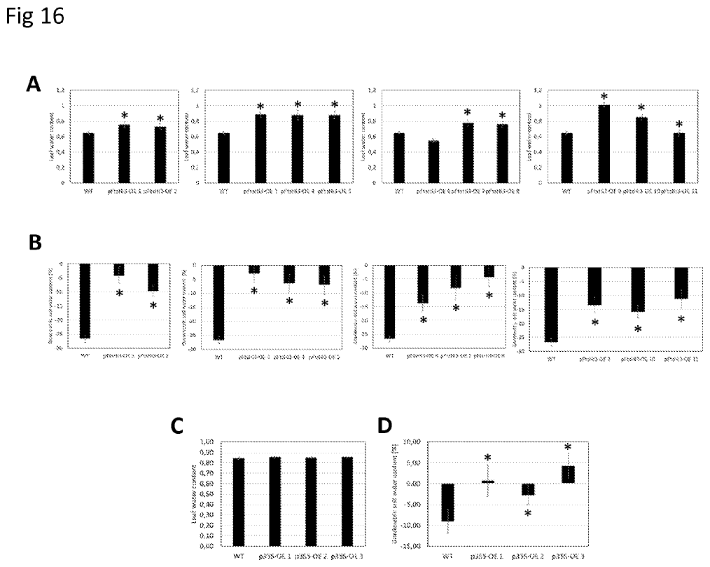

Stomata density and sizes were determined in leaves of WT and the transgenic (pAtFtsHi3::AtFtsHi3::HA(WT)) lines OE1 and OE2. WT plants contained an average of 252 stomata per mm² compared to OE1 and OE2 with 200 and 204 per mm², respectively (FIG. 11A). The average stomatal width to length dimension was 18.9 by 14.9 μm for WT, in contrast to 17.5 by 13.9 μm for OE1 and 16.7 by 12.1 μm for OE2 (FIG. 11B). The differences observed between the length of the stomata and the stomata density per mm² in the OE lines compared to WT were significant.

Dehydration can be age-dependent; therefore, 7-day-old seedlings were grown either in the absence of exogenous ABA and mannitol or exposed to 1 μmol or 5 μmol ABA, or 200 and 500 mM mannitol for seven days. Seedlings of the (pAtFtsHi3::AtFtsHi3::HA(WT)) OE lines showed similar growth to WT seedlings when treated with 1 μmol ABA, whereas in the presence of 5 μmol ABA, 200 mM or 500 mM mannitol the WT seedlings were affected more than OE lines (FIG. 11C). The OE seedlings still showed significant growth compared to the WT.

We further examined the (pAtFtsHi3::AtFtsHi3::HA(WT)) OE line guard cell apertures at the leaves' abaxial side compared to WT in the presence or absence of 10 μmol ABA to understand if the stomatal aperture closure is sensitive to ABA. Response to ABA was determined by calculating the ratio of width/length of the stomata in the presence or absence of exogenous 10 μmol ABA. Stomatal apertures in the absence of ABA treatment were small in OE lines, averaging approximately 0.37 μm, whereas they averaged 0.47 μm in WT (FIG. 11D). When treated with 10 μmol exogenous ABA, stomatal closure was induced, with the stomatal apertures of all genotypes decreasing by over 50% (FIG. 11D).

These results suggest that OE plants' drought tolerance is rather related to their lower stomatal density than ABA sensitivity.

Higher Expression of FTSHi3 Reduces Stomatal Conductance and Increases Water Use Efficiency without Affecting the Photosynthetic Parameters

Gas-exchange parameters during watered and drought conditions were determined under SD conditions (Table 1). We observed stable net photosynthesis (AN, μmol CO₂ m⁻² s⁻¹) and CO₂ concentration inside the leaf (Ci, μmol CO₂ mol⁻¹ air) across the genotypes in the watered condition. The Fv/Fm values of the OE1 and OE2 lines observed above were also similar to the WT plants.

After 14 days of drought treatment WT plants showed drought symptoms, their AN and gs were significantly lower compared to the values found for the (pAtFtsHi3::AtFtsHi3::HA(WT) OE1 and OE2 lines, the 35S::AtFtsHi3::GFP(WT) OE lines and 35S::AtFtsHi3::GFP(ftshi3-1) comp lines (Table 1). The water usage efficiency (WUE intrinsic), i.e. the ratio AN/gs, showed a significant increase in OE1 and OE2 lines, the 35S::AtFtsHi3::GFP(WT) OE lines and 35S::AtFtsHi3::GFP (ftshi3-1) comp lines compared to WT (Table 1) after the exposure to drought treatment.

TABLE 1

| | An | | gs | |
|---|---|---|---|---|
| Genotype | Watered | Drought | Watered | Drought |
| WT | 6.9 ± 0.4 | 0.81 ± 0.2 | 0.093 ± 0.01 | 0.009 ± 0.0 |
| OE1 | 6.49 ± 0.4 | 2.17 ± 0.3 | 0.098 ± 0.01 | 0.053 ± 0.00 |
| (pAtFtsHi3::AtFtsHi3::HA(WT) line 1) | | | | |
| OE2 | 5.09 ± 0.2 | 4.66 ± 0.3 | 0.077 ± 0.01 | 0.057 ± 0.01 |
| (pAtFtsHi3::AtFtsHi3::HA(WT) line 2) | | | | |
| 35S::AtFtshi3::GFP (ftshi3-1) Comp1 | 8.2 ± 0.5 | 3.61 ± 0.2 | 0.106 ± 0.01 | 0.032 ± 0.00 |
| 35S::AtFtshi3::GFP (ftshi3-1) Comp2 | 8.77 ± 0.5 | 4.39 ± 0.3 | 0.112 ± 0.01 | 0.032 ± 0.00 |
| 35S::AtFtshi3::GFP (Wt) OE1 | 8.54 ± 0.2 | 4.42 ± 0.4 | 0.086 ± 0.00 | 0.041 ± 0.00 |
| 35S::AtFtshi3::GFP (Wt) OE2 | 7.09 ± 0.1 | 4.58 ± 0.1 | 0.085 ± 0.00 | 0.047 ± 0.00 |
| 35S::AtFtshi3::GFP (Wt) OE3 | 6.5 ± 0.2 | 4.73 ± 0.3 | 0.075 ± 0.00 | 0.044 ± 0.00 |

| | Ci | | WUE | |
|---|---|---|---|---|
| | Watered | Drought | Watered | Drought |
| WT | 245.36 ± 4.2 | 263.63 ± 13.7 | 86.17 ± 2.8 | 78.98 ± 8.6 |
| OE1 | 264.79 ± 9.3 | 228.7 ± 7.7 | 73.21 ± 6.18 | 124.76 ± 6.04 |
| (pAtFtsHi3::AtFtsHi3::HA(WT) line 1) | | | | |
| OE2 | 265.9 ± 9.5 | 214.8 ± 13.3 | 74.06 ± 6.04 | 130.08 ± 11.75 |
| (pAtFtsHi3::AtFtsHi3::HA(WT) line 2) | | | | |
| 35S::AtFtshi3::GFP (ftshi3-1) Comp1 | 255.69 ± 4.04 | 193.84 ± 24.6 | 78.79 ± 2.75 | 120.74 ± 15.4 |
| 35S::AtFtshi3::GFP (ftshi3-1) Comp2 | 254.86 ± 3.94 | 174.27 ± 8.21 | 79.33 ± 2.58 | 132.72 ± 5.17 |
| 35S::AtFtshi3::GFP (Wt) OE1 | 221.38 ± 3.54 | 209.98 ± 4.36 | 100.08 ± 2.77 | 109.24 ± 3.07 |
| 35S::AtFtshi3::GFP (Wt) OE2 | 245.08 ± 7.38 | 227.86 ± 0.55 | 85.5 ± 4.72 | 97.32 ± 0.33 |
| 35S::AtFtshi3::GFP (Wt) OE3 | | | | |

### Transcript Abundance of ABA Signalling-Related Genes in Arabidopsis Plants Overexpressing FTSHi3

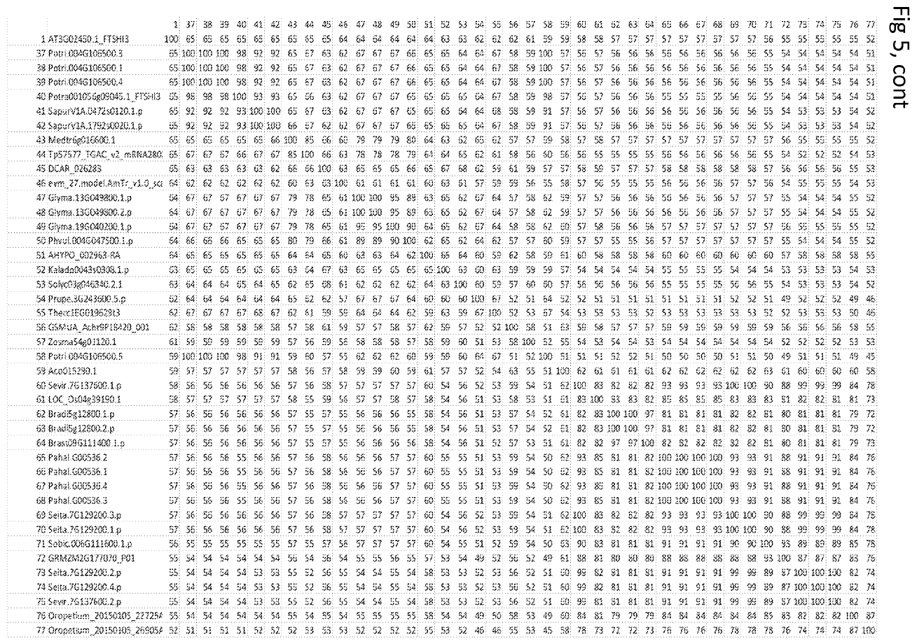

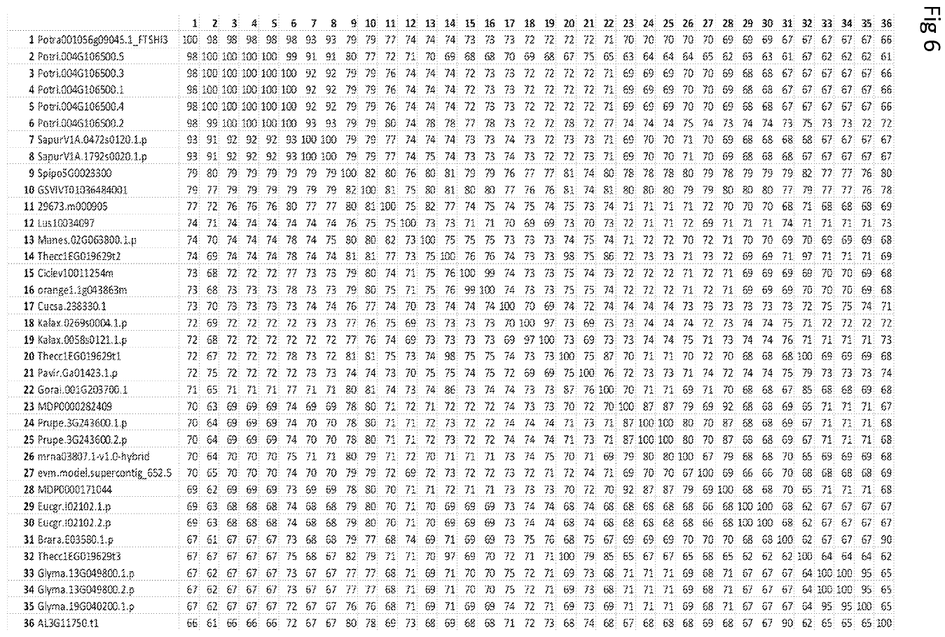

The expression patterns of nine dehydration-induced or ABA-responsive genes-RD22, RD29A, RD29B DREB1A, DREB2A, NCED, DI21, COR47 and P5CS were tested by qPCR in WT and OE plants in watered and drought conditions. The expression of these genes is shown in FIG. 4 relative to the housekeeping gene actin.

A constitutively higher expression of RD22, RD29A, RD29B DREB1A, DREB2A, NCED, DI21, and COR47 genes was observed in (pAtFtsHi3::AtFtsHi3::HA(WT)) OE plants when compared with WT under watered conditions (FIG. 4A). In comparison, only one ABA-dependent gene RD22 as well as the ABA-independent gene DREB1A, but not DREB2A, were significantly up-regulated in OE plants relative to WT under drought conditions (FIG. 4B).

The above mentioned nine dehydration-induced genes are also markers of progressive drought. Therefore, higher WT expression of RD29B DREB2A, DI21, COR47 and P5CS demonstrates the drought-sensitivity of WT.

These data support the observation that higher transcripts of ABA-responsive genes in OE plants in watered condition and elevated endogenous ABA levels in drought conditions could be priming the plants towards better handling the water deficit stress.

### Methods for Obtaining Genetically Modified Plants

A method for obtaining a genetically modified plant having improved yield and/or drought tolerance, as compared to a wild type control plant of the same species, according to the present invention generally comprises the steps:
a. Modifying the genomic DNA in at least one cell of said plant species to increase expression of a FTSHi3 gene thereby obtaining a genetically modified cell;
b. generating a plant from the genetically modified cell to obtain a genetically modified plant; and
c. growing said genetically modified plant under conditions which permit development of a plant; and
d. selecting a genetically modified plant having improved yield and/or drought tolerance.

The modification in step a. may be the introduction of a nucleic acid molecule (e.g. a DNA construct) encoding a FTSHi3 gene product operably linked to a FTSHi3 promoter or a constitutive promoter.

In one embodiment, the sequence including the promoter region and the coding sequence of the FtsHi3 gene from a plant species is incorporated into a nucleic acid molecule that is then introduced into the same plant species.

In a further embodiment the sequence including the promoter region and the coding sequence of the FTSHi3 gene from a first plant species is incorporated into a nucleic acid molecule (e.g. a DNA construct) that is then introduced into a different, second, plant species. In some embodiments, the first and second plant species may be species of the same genus, the same family, or the same order.

In some embodiments, the sequence including the promoter region and the coding sequence of the FTSHi3 gene, respectively, originate from different species and are recombined into a recombinant a nucleic acid molecule encoding a FTSHi3 gene product operably linked to a FTSHi3 promoter (pFTSHi3).

In some embodiments, the promoter is a constitutive promoter as described below. In some embodiments, the promoter is the FTSHi3 promoter from A. thaliana, with a nucleotide sequence according to SEQ ID NO: 5.

In some embodiments, the modification of the genomic DNA in step a. of the method comprises modification of the promoter sequence of the native FTSHi3 gene to increase expression of the FtsHi3 gene product. This generally comprises modification of the nucleotide sequence of the 1*10³-2*10³ nucleotides upstream the native FtsHi3 gene. Such modification may be performed as known in the art, e.g. by use of CRISPR or TALENS.

Genetically modified plants having improved yield and/or drought tolerance, as compared to a control plant of the same species, prepared through technical means as described above, may be further propagated by sexual or asexual propagation or through further technical processes, such as somatic embryogenesis.

Thus, in some aspects, the present invention relates a method for obtaining a genetically modified plant having improved yield and/or drought tolerance, as compared to a control plant of the same species, comprising
Obtaining a first genetically modified plant through the method according to the method according to the invention;
Producing at least one seed, somatic embryo, or vegetatively reproducible material from the first genetically modified plant; and
Obtaining at least one second genetically modified plant from said seed, somatic embryo, or vegetatively reproducible material

### Genetically Modified Plants According to the Invention

In one aspect, the present invention further relates to plants obtainable by the above described methods according to the invention.

In one aspect, the present invention relates to a genetically modified plant exhibiting at least 50% increased expression of a FTSHi3 gene as compared to a wild-type control plant of the same species. In some embodiments, the genetically modified plant exhibits at least 100%, or at least 200%, increased expression of a FTSHi3 gene as compared to a wild-type control plant of the same species.

Such genetically modified plants may be obtained through the methods as described above. The disclosure of the methods above apply mutatis mutandis to the genetically modified plants according to the invention.

In one embodiment, the genetically modified plant comprises a modification of the genomic DNA, as compared to a wild-type control plant of the same species, said modification comprising the presence of a recombinant nucleic acid molecule encoding a FtsHi3 gene product operably linked to a FTSHi3 promoter, or a constitutive promoter.

In one embodiment, the encoded FtsHi3 gene product has the same amino acid sequence as a native FtsHi3 gene product of the same plant species, or the amino acid sequence according to SEQ ID NO: 1.

In one embodiment, the FTSHi3 promoter has a nucleotide sequence corresponding to the nucleotide sequence of the 1-2*103 nucleotides upstream the native FTSHi3 gene in a native genome of a control plant of the same species.

In one embodiment, the FTSHi3 promoter has a nucleotide sequence according to SEQ ID NO: 5.

In one embodiment, the genetically modified plant comprises a modification of the genomic DNA, as compared to a wild-type control plant of the same species, said modification comprising a modification of the nucleotide sequence of the 1*10³-2*10³ nucleotides upstream the native FTSHi3 gene.

In one embodiment, the genetically modified plant has improved drought tolerance, as compared to a wild-type control plant of the same species.

In one embodiment, the genetically modified plant has improved yield, as compared to a wild-type control plant of the same species

In one embodiment, the genetically modified plant comprises has not been obtained exclusively by means of an essentially biological process.

### FTSHi3 Genes

FTSHi3 homologues have been found in a number of eudicots as described in the following table. These orthologous genes can be utilized as FTSHi3 genes in the present invention.

| | | Accession number | SEQ | SEQ |
|---|---|---|---|---|
| | | (Phytozome, | ID NO: | ID NO: |
| Species | Gene name | popgenie.org) | (NA) | (Protein) |
| A. thaliana | AtFtsHi3 | AT3G02450.1_FTSHI3 | 1 | 2 |
| Populus tremula | | Potra001056g09045.1_FTSHI3 | 3 | 4 |
| A. coerulea | Aqcoe3G076400 | Aqcoe3G076400.1.p | 11 | 12 |
| A. comosus | Aco015290 | Aco015290.1 | 13 | 14 |
| A. helleri | Araha.2210s0001 | Araha.2210s0001.1.p | 15 | 16 |
| A. hypochondriacus | AHYPO_002963 | AHYPO_002963-RA | 17 | 18 |
| A. lyrata | AL3G11750 | AL3G11750.t1 | 19 | 20 |
| A. trichopoda | evm_27.TU.AmTr_v1.0_{—} | evm_27.model.AmTr_v1.0_{—} | 21 | 22 |
| | scaffold00006.221 | scaffold00006.221 | | |
| B. distachyon | Bradi5g12800 | Bradi5g12800.1.p | 23 | 24 |
| B. distachyon | Bradi5g12800 | Bradi5g12800.2.p | 25 | 26 |
| B. rapaFPsc | Brara.E03580 | Brara.E03580.1.p | 27 | 28 |
| B. stacei | Brast09G111400 | Brast09G111400.1.p | 29 | 30 |
| B. stricta | Bostr.1460s0159 | Bostr.1460s0159.1.p | 31 | 32 |
| C. clementina | Ciclev10011254m.g | Ciclev10011254m | 33 | 34 |
| C. grandiflora | Cagra.1194s0060 | Cagra.1194s0060.1.p | 35 | 36 |
| C. papaya | evm.TU.supercontig_652.5 | evm.model.supercontig_652.5 | 37 | 38 |
| C. rubella | Carubv10016037m.g | Carubv10016037m | 39 | 40 |
| C. sativus | Cucsa.238330 | Cucsa.238330.1 | 41 | 42 |
| C. sinensis | orange1.1g043863m.g | orange1.1g043863m | 43 | 44 |
| D. carota | DCAR_026283 | DCAR_026283 | 45 | 46 |
| E. grandis | Eucgr.102102 | Eucgr.I02102.1.p | 47 | 48 |
| E. grandis | Eucgr.102102 | Eucgr.I02102.2.p | 49 | 50 |
| E. salsugineum | Thhalv10020311m.g | Thhalv10020311m | 51 | 52 |
| F. vesca | gene03807-v1.0-hybrid | mrna03807.1-v1.0-hybrid | 53 | 54 |
| G. max | Glyma.13G049800 | Glyma.13G049800.1.p | 55 | 56 |
| G. max | Glyma.13G049800 | Glyma.13G049800.2.p | 57 | 58 |
| G. max | Glyma.19G040200 | Glyma.19G040200.1.p | 59 | 60 |
| G. raimondii | Gorai.001G203700 | Gorai.001G203700.1 | 61 | 62 |
| K. fedtschenkoi | Kaladp0043s0308 | Kaladp0043s0308.1.p | 63 | 64 |
| K. laxiflora | Kalax.0058s0121 | Kalax.0058s0121.1.p | 65 | 66 |
| K. laxiflora | Kalax.0269s0004 | Kalax.0269s0004.1.p | 67 | 68 |
| L. usitatissimum | Lus10003049.g | Lus10003049 | 69 | 70 |
| L. usitatissimum | Lus10034097 | Lus10034097 | 71 | 72 |
| M. acuminata | GSMUA_Achr9G18420_001 | GSMUA_Achr9P18420_001 | 73 | 74 |
| M. domestica | MDP0000171044 | MDP0000171044 | 75 | 76 |
| M. domestica | MDP0000282409 | MDP0000282409 | 77 | 78 |
| M. esculenta | Manes.02G063800 | Manes.02G063800.1.p | 79 | 80 |
| M. guttatus | Migut.H00557 | Migut.H00557.1.p | 81 | 82 |
| M. guttatus | Migut.H00557 | Migut.H00557.2.p | 83 | 84 |
| M. truncatula | Medtr6g016600 | Medtr6g016600.1 | 85 | 86 |
| O. sativa | LOC_Os04g39190 | LOC_Os04g39190.1 | 87 | 88 |
| O. thomaeum | Oropetium_20150105_22725 | Oropetium_20150105_22725A | 89 | 90 |
| O. thomaeum | Oropetium_20150105_26905 | Oropetium_20150105_26905A | 91 | 92 |
| P. hallii | Pahal.G00536 | Pahal.G00536.1 | 93 | 94 |
| P. hallii | Pahal.G00536 | Pahal.G00536.2 | 95 | 96 |
| P. hallii | Pahal.G00536 | Pahal.G00536.3 | 97 | 98 |
| P. hallii | Pahal.G00536 | Pahal.G00536.4 | 99 | 100 |
| P. persica | Prupe.3G243600 | Prupe.3G243600.1.p | 101 | 102 |
| P. persica | Prupe.3G243600 | Prupe.3G243600.2.p | 103 | 104 |
| P. persica | Prupe.3G243600 | Prupe.3G243600.5.p | 105 | 106 |
| P. trichocarpa | Potri.004G106500 | Potri.004G106500.1 | 107 | 108 |
| P. trichocarpa | Potri.004G106500 | Potri.004G106500.2 | 109 | 110 |
| P. trichocarpa | Potri.004G106500 | Potri.004G106500.3 | 111 | 112 |
| P. trichocarpa | Potri.004G106500 | Potri.004G106500.4 | 113 | 114 |
| P. trichocarpa | Potri.004G106500 | Potri.004G106500.5 | 115 | 116 |
| P. virgatum | Pavir.Ga01423 | Pavir.Ga01423.1.p | 117 | 118 |
| P. vulgaris | Phvul.004G047500 | Phvul.004G047500.1.p | 119 | 120 |
| R. communis | 29673.t000006 | 29673.m000905 | 121 | 122 |
| S. bicolor | Sobic.006G111600 | Sobic.006G111600.1.p | 123 | 124 |
| S. italica | Seita.7G129200 | Seita.7G129200.1.p | 125 | 126 |
| S. italica | Seita.7G129200 | Seita.7G129200.2.p | 127 | 128 |
| S. italica | Seita.7G129200 | Seita.7G129200.3.p | 129 | 130 |
| S. italica | Seita.7G129200 | Seita.7G129200.4.p | 131 | 132 |
| S. lycopersicum | Solyc03g046340.2 | Solyc03g046340.2.1 | 133 | 134 |
| S. polyrhiza | Spipo5G0023300 | Spipo5G0023300 | 135 | 136 |
| S. purpurea | SapurV1A.0472s0120 | SapurV1A.0472s0120.1.p | 137 | 138 |
| S. purpurea | SapurV1A.1792s0020 | SapurV1A.1792s0020.1.p | 139 | 140 |
| S. viridis | Sevir.7G137600 | Sevir.7G137600.1.p | 141 | 142 |
| S. viridis | Sevir.7G137600 | Sevir.7G137600.2.p | 143 | 144 |
| T. cacao | Thecc1EG019629 | Thecc1EG019629t1 | 145 | 146 |
| T. cacao | Thecc1EG019629 | Thecc1EG019629t2 | 147 | 148 |
| T. cacao | Thecc1EG019629 | Thecc1EG019629t3 | 149 | 150 |
| T. pratense | Tp57577_TGAC_v2_gene27154 | Tp57577_TGAC_v2_mRNA28089 | 151 | 152 |
| V. vinifera | GSVIVG01036484001 | GSVIVT01036484001 | 153 | 154 |
| Z. marina | Zosma54g01120 | Zosma54g01120.1 | 155 | 156 |
| Z. mays | GRMZM2G177070 | GRMZM2G177070_P01 | 157 | 158 |

In some embodiments, the encoded FtsHi3 gene product has the same amino acid sequence as a native FtsHi3 gene product of the same plant species.

In some embodiments, the encoded FtsHi3 gene product has an amino acid sequence that has at least 60% identity to the amino acid sequence according to SEQ ID NO: 2. This includes AL3G11750.t1, Araha.2210s0001.1.p, Bostr.1460s0159.1.p, Cagra.1194s0060.1.p, Carubv10016037m, Thhalv10020311m, Brara.E03580.1.p, Spipo5G0023300, GSVIVT01036484001, Lus10034097, Pavir.Ga01423.1.p, Kalax.0058s0121.1.p, Kalax.0269s0004.1.p, Potri.004G106500.2, Cucsa.238330.1, MDP0000171044, Thecc1EG019629t2, evm.model.supercontig_652.5, Lus10003049, MDP0000282409, Prupe.3G243600.1.p, Prupe.3G243600.2.p, mrna03807.1-v1.0-hybrid, 29673.m000905, Manes.02G063800.1.p, Thecc1EG019629t1, Gorai.001G203700.1, Ciclev10011254m, orange1.1g043863m, Eucgr.102102.1.p, Eucgr.102102.2.p, Migut.H00557.1.p, Migut.H00557.2.p, Aqcoe3G076400.1.p, Potri.004G106500.3, Potri.004G106500.1, Potri.004G106500.4, Potra001056g09045.1_FTSHI3, SapurV1A.0472s0120.1.p, SapurV1A.1792s0020.1.p, Medtr6g016600.1, Tp57577_TGAC_v2_mRNA28089, DCAR_026283, evm_27.model.AmTr_v1.0_scaffold00006.221, Glyma.13G049800.1.p, Glyma.13G049800.2.p, Glyma. 19G040200.1.p, Phvul.004G047500.1.p, AHYPO_002963-RA, Kaladp0043s0308.1.p, Solyc03g046340.2.1, Prupe.3G243600.5.p, Thecc1EG019629t3, GSMUA_Achr9P18420_001, and Zosma54g01120.1. In some embodiments where the encoded FtsHi3 gene product has an amino acid sequence that has at least 60% identity to the amino acid sequence according to SEQ ID NO: 2, the genetically modified plant having improved drought tolerance is a crop plant species. In some embodiments, the encoded FtsHi3 gene product has an amino acid sequence that has at least 60%, such as at least 65%, 70%, 75%, 80%, 90%, or 95% identity to the amino acid sequence according to SEQ ID NO: 2.

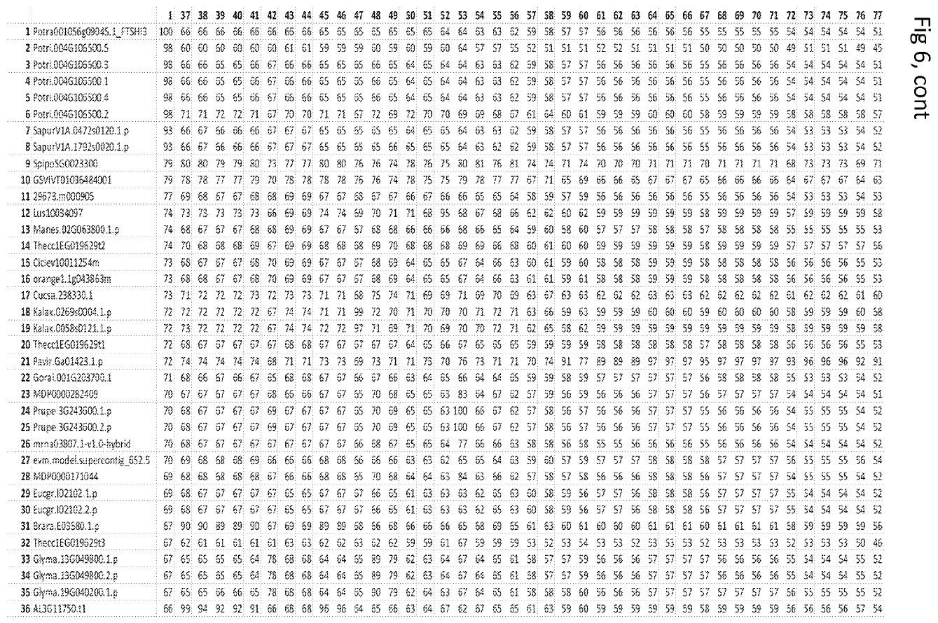

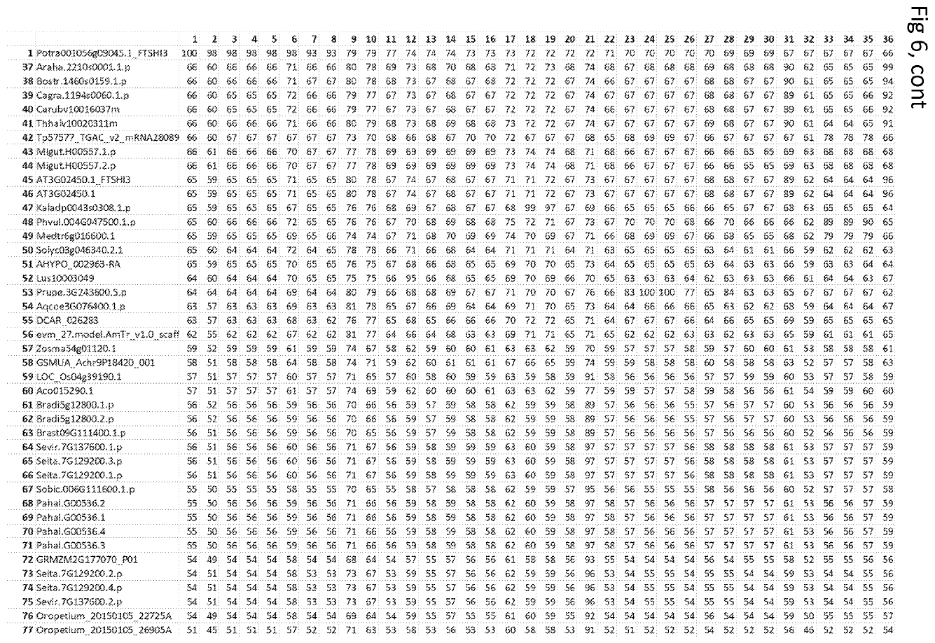

The pairwise sequence identities can be obtained through generation of a Percent Identity Matrix using publicly available software, such as Clustal2.1. Such a matrix is provided in FIG. 5.

In some embodiments, the encoded FtsHi3 gene product has an amino acid sequence that has at least 60% identity to the amino acid sequence according to SEQ ID NO: 4. This includes Potri.004G106500.5, Potri.004G106500.3, Potri.004G106500.1, Potri.004G106500.4, Potri.004G106500.2, SapurV1A.0472s0120.1.p, SapurV1A.1792s0020.1.p, Spipo5G0023300, GSVIVT01036484001, 29673.m000905, Lus10034097, Manes.02G063800.1.p, Thecc1EG019629t2, Ciclev10011254m, orange1.1g043863m, Cucsa.238330.1, Kalax.0269s0004.1.p, Kalax.0058s0121.1.p, Thecc1EG019629t1, Pavir.Ga01423.1.p, Gorai.001G203700.1, MDP0000282409, Prupe.3G243600.1.p, Prupe.3G243600.2.p, mrna03807.1-v1.0-hybrid, evm.model.supercontig_652.5, MDP0000171044, Eucgr.102102.1.p, Eucgr.102102.2.p, Brara.E03580.1.p, Thecc1EG019629t3, Glyma. 13G049800.1.p, Glyma.13G049800.2.p, Glyma.19G040200.1.p, AL3G11750.t1, Araha.2210s0001.1.p, Bostr.1460s0159.1.p, Cagra.1194s0060.1.p, Carubv10016037m, Thhalv10020311m, Tp57577_TGAC_v2_mRNA28089, Migut.H00557.1.p, Migut.H00557.2.p, AT3G02450.1_FTSHI3, AT3G02450.1, Kaladp0043s0308.1.p, Phvul.004G047500.1.p, Medtr6g016600.1, Solyc03g046340.2.1, AHYPO_002963-RA, Lus10003049, Prupe.3G243600.5.p, Aqcoe3G076400.1.p, DCAR_026283, and evm_27.model.AmTr_v1.0_scaffold00006.221. In some embodiments where the encoded FtsHi3 gene product has an amino acid sequence that has at least 60% identity to the amino acid sequence according to SEQ ID NO: 4, the genetically modified plant having improved drought tolerance is a woody plant species, such as a hardwood plant species or a gymnosperm species. In some embodiments, the encoded FtsHi3 gene product has an amino acid sequence that has at least 60%, such as at least 65%, 70%, 75%, 80%, 90%, or 95% identity to the amino acid sequence according to SEQ ID NO: 4.

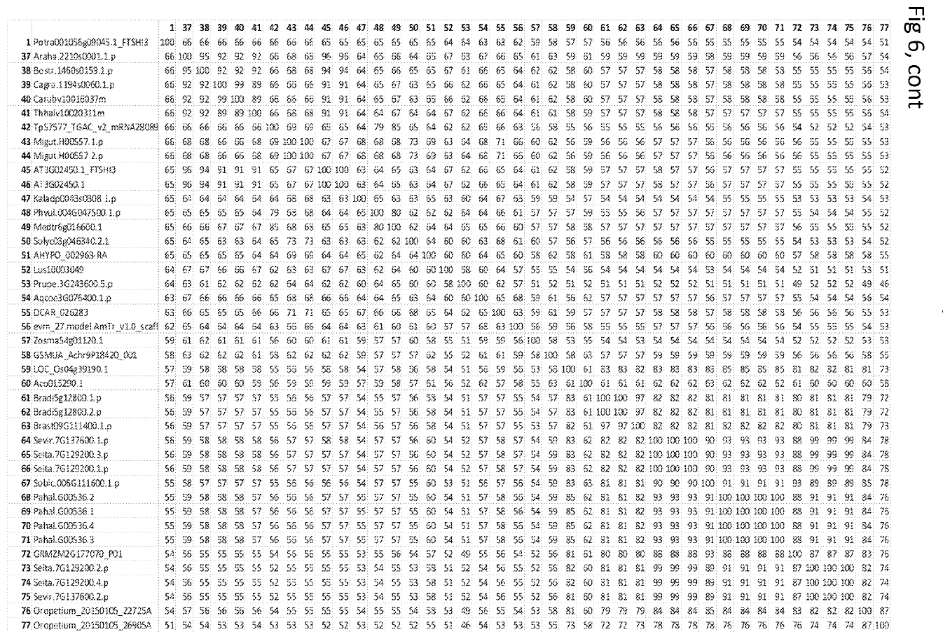

The pairwise sequence identities can be obtained through generation of a Percent Identity Matrix using publicly available software, such as Clustal2.1. Such a matrix is provided in FIG. 6.

### Promoters Useful in the Present Invention

In some embodiments, the present invention makes use of DNA constructs comprising an FtsHi3 gene operably linked to an FtsHi3 promoter or a constitutive promoter.

An FtsHi3 promoter is a promoter that functions as a promoter of an FtsHi3 gene in a wild type plant. Such promoters are found upstream the coding sequence of the gene and generally comprise a sequence of 1-2*10³ nucleotides.

Constitutive promoters useful in promoting expression of genes in plants are well-known in the art and include but is not limited to, the 35S promoter from Cauliflower Mosaic Virus (CaMV), plant ubiquitin, actin and Ribulose-1,5-bisphosphate carboxylase-oxygenase, (RuBisCo).

### Plant Species of Interest

The present invention has been confirmed in the model species A. thaliana and may be implemented in other plant species as well, such as crop plants and woody plants.

The presence of FtsHi3 was investigated across all the families. Thus, in some embodiments, the genetically modified plants of the present invention are eudicots.

### Crop Plants.

Crop plants that can be the subject of the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca 30 arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Woody Plants

The present invention also relates to genetically modified woody plants, such as genetically modified angiosperms, dicotyledonous woody plants, preferably trees.

The invention further relates to genetically modified woody plants from gymnosperms, such as conifer trees.

The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants, such as eucalyptus and plants from the Salicaceae family, such as willow, poplar and aspen including variants thereof, are of particular interest, as these groups include fast-growing species of tree or woody shrub, which are grown specifically to provide timber for building material, raw material for pulping, bio-fuels and/or bio chemicals.

In further embodiments, the genetically modified tree is a conifer tree, such as a member of the order Pinales, with members of the family Cupressaceae, such as Cupressus spp., Juniperus spp., Sequoia spp., Sequoiadendron spp.; with members of the family Taxaceae (Taxus spp.) and with members of the family Pinaceae, such as the genera Abies spp., Cedrus spp., Larix spp., Picea spp., Pinus spp., Pseudotsuga spp., Tsuga spp.

Alternatively, the woody plants which may be selected from the group consisting of cotton, bamboo and rubber plants.

In another embodiment, the genetically modified tree is a deciduous tree including hybrids, and cultivars such as acacia (Acacia spp.), alder (Alnus spp.), birch (Betula spp.), hornbeam (Carpinus spp.), hickory (Carya spp.), chestnut (Castanea spp.), beech (Fagus spp.), walnut (Juglans spp.), oak (Quercus spp.), ash (Fraxinus spp.), poplar (Populus spp.), aspen (Populus spp.), willow (Salix spp.), eucalyptus (Eucalyptus spp.), sycamore (Platanus spp.), maple (Acer spp.), mahogany (Swietenia spp.), sweet gum (Liquidambar spp.). Genetically modified trees of the families Salicaceae and Myrtaceae are preferred, most preferred are genetically modified tree from the genus Eucalyptus and Populus.

In yet another embodiment, the genetically modified tree is a fruit bearing plant, including hybrids, and cultivars such as, apple (Malus spp.), plum (Prunus spp.), pear (Pyrus spp.), orange (Citrus spp.), lemon (Citrus spp.), kiwi fruit (Actinidia spp.), cherry (Prunus spp.), grapevine (Vitis spp.), and fig (Ficus spp.).

In a specific embodiment, the genetically modified tree is a woody plant whose leaves can be eaten as leaf vegetables include Adansonia, Aralia, Moringa, Morus, and Toona species.

### Methods

The skilled person may rely on standard methods and technologies in molecular biotechnology and plant biology in working the present invention. Such methods are described in further detail i.a. in Methods in Enzymology (Elsevier Inc., ISSN: 0076-6879), Molecular Cloning: A Laboratory Manual (4^{th} ed.) (Cold Spring Harbor Laboratory Press, 2012, ISBN 978-1-936113-42-2), Genetic Modification of Plants (eds. Kempken and Jung, 2010, ISBN: 978-3-642-02390-3). Some methods are further generally described below.

### Analysis of Expression Levels

Real-time RT-PCR (qPCR) may be used to compare construct gene expression levels of the modified plant group with corresponding wild type plant group. The expression level of i.a. 26S proteasome regulatory subunit S2, ubiquitin, or actin may be used as a reference to which construct gene expression is normalized. The comparative CT method may be used for calculation of relative construct gene expression level, where the ratio between construction and reference gene expression level is described by (1+E_{target})−CT_{target}/(1+E_{reference})−CT_{reference}, where E_{target} and E_{reference} are the efficiencies of construct and reference gene PCR amplification respectively and CT target and CT reference are the threshold cycles as calculated for construct and reference gene amplification respectively.

### Obtaining Plants

The present invention extends to any plant cell of the above genetically modified, or transgenic plants obtained by the methods described herein, and to all plant parts, including harvestable parts of a plant, seeds, somatic embryos and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. It should be noted that embodiments and features described in the context of one of the aspects of the present invention also apply to the other aspects of the invention. Thus, definitions of one embodiment regard mutatis mutandis to all other embodiments comprising or relating to the one embodiment. When for example definitions are made regarding DNA constructs or sequences, such definitions also apply with respect to methods for producing a plant, vectors, plant cells, plants comprising the DNA construct and vice versa. A DNA construct described in relation to a plant also regards all other embodiments. Details about obtaining maize, soy and Arabidopsis can be found in WO2014195287, hereby included by reference.

### Methods for Enhancing the Productivity of a Plant by Genetic Modification

One or more of the constructs according to the invention may be introduced into a plant cell by transformation.

-Transformation of plant cells

In accordance with some embodiments of the present invention, the method comprises transforming regenerable cells of a plant with a nucleic acid construct or recombinant DNA construct and regenerating a transgenic plant from said transformed cell. Production of stable, fertile transgenic plants is now a routine method.

Various methods are known for transporting the construct into a cell to be transformed. Agrobacterium-mediated transformation is widely used by those skilled in the art to transform tree species, in particular hardwood species such as poplar and Eucalyptus. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

A person of skill in the art will realize that a wide variety of host cells may be employed as recipients for the DNA constructs and vectors according to the invention. Non-limiting examples of host cells include cells in embryonic tissue, callus tissue type I, II, and III, hypocotyls, meristem, root tissue, tissues for expression in phloem, leaf discs, petioles and stem internodes. Once the DNA construct or vector is within the cell, integration into the endogenous genome can occur.

### Selection of Transformed Plant Cells and Regeneration of Plants or Woody Plants

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A selection marker using the D-form of amino acids and based on the fact that plants can only tolerate the L-form offers a fast, efficient and environmentally friendly selection system. Other methods, including screening based on expression level of the construct gene, or by phenotypic comparison with a WT of the same species, can also be used to select transformed plants according to the present invention.

Subsequently, a plant may be regenerated, e.g. from single cells, callus tissue or leaf discs, as is standard in the art. Almost any plant can be entirely regenerated from cells, tissues and organs of the plant. After transformed plants are selected and they are grown to maturity and those plants showing altered growth properties phenotype are identified.

### CRISPR and TALENs

The promotors of the FtsHi3 gene might be mutated using the methods for site-directed mutagenesis such as TALENs or CRISPR/Cas9 to modify the expression of FtsHi3 genes. Such methods are known in the art, see e.g. Chen et al., Journal of Genetics and Genomics, Volume 40, Issue 6, 20 Jun. 2013, Pages 271-279, Belhaj et al., Plant Methods, 2013 Oct. 11; 9(1):39 and Song et al., The Crop Journal, Volume 4, Issue 2, April 2016, Pages 75-82, all included herein by reference.

### Somatic Embryogenesis

Somatic embryogenesis is a vegetative propagation technology, which makes it possible to mass-produce genetically identical individuals through an asexual reproduction of a source explant. This propagation technology is generally a multi-step process.

The somatic embryogenesis technology allows for fast and cost-efficient deployment of plants from the breeding front. The somatic embryogenesis technology also allows for clonal or varietal mixture plantation programs. The primary advantages of clonal or varietal mixture forestry and agriculture are the ability to use the best full sibling sib families and additionally the best individuals within such full-sib families for deployment. The somatic embryogenesis technology allows also for predictable production of planting material disregarding bad weather and pest attacks giving variation in seed production and need for pesticide use, as well as reducing land use for planting material production.

Firstly, high quality mature somatic embryo is placed in a container with controlled air humidity for an optional desiccation step. When the selection of high-quality somatic embryos is done manually it needs a well-trained person. The selection can also be done automatically, which can be done by several different quality criteria, as presented in U.S. Pat. Nos. 7,881,502 and 9,053,353.

Secondly, the high quality mature somatic embryo is placed on germination medium containing an energy source such as sucrose. The germination medium is mostly a sterilised semi-solid media, gel, gelrite and similar. The placement can be done manually or using different robot settings. The mature somatic embryo is then incubated under sterile conditions, in vitro, during a few weeks until root and cotyledons are more developed, optionally the first needles/leaves have developed.

Thirdly, the germinant is transferred to a pot with a growth substrate, in most cases peat is used. The germinant is left to grow for a few weeks to develop into an established plant that can be further transplanted.

Finally, depending on size of the plantlet, it might be transplanted to a larger pot before it can be placed in an indoor or outdoor growth location, or stored in a cold room for in-wintering.

While the above steps are described for conifer species, the steps are essentially the same also for other species. Hence, in one embodiment, somatic embryogenesis can be chosen as the method for the regeneration or the multiplication of plants that have been modified according to the present invention.

### Methods for Detecting Modified Expression of a Gene Encoding a Polypeptide in a Plant or Woody Plant of the Invention

Real-time RT-PCR can be used to compare gene expression, i.e. the mRNA expression levels, in a genetically modified (GM) plant or woody plant with the corresponding non-GM plant or woody plant. The amount of the polynucleotides disclosed herein can be determined using Northern blots, sequencing, RT-PCR or microarrays.

Western blots with immune detection or gel shift assays can be used to measure the expression levels or amounts of a polypeptide expressed in a GM plant or woody plant of the invention. Antibodies raised to the respective polypeptide may be used for specific immune-detection of the expressed polypeptide in tissue derived from a woody plant.

Eucalyptus plants are generated in a similar way, through transformation, regeneration and growth analysis.

The invention is further illustrated below by way of examples. The examples are not intended to restrict the scope of the invention, which is that of the appended claims.

### Methods for Analysis of Drought Phenotype

This method is disclosed for A. thaliana and may be modified for use with other plant species. Water-deficit stress is applied on plants grown on the soil at a relative humidity of 50% and 150 μmol photons m² s⁻¹ for approximately four weeks before treatment. Plants are exposed to short-day conditions (8 h/16 h photoperiod, 22° C./18° C.) or long-day conditions (16 h/8 h photoperiod, 22° C./18° C.).

Severe drought treatment is induced by stopping the irrigation when plants are four weeks old until the genotypes showed drought effects compared to the well-watered controls. These controls are watered daily with 550 ml water per tray (15 pots/tray). The pot weight of samples and controls is determined by weighing plants and soil. After 20 days of severe drought, all genotypes are re-watered daily with 350 ml water for 14 days, and the number of plants resuming their growth is counted. These experiments are performed three times with ten biological replicates each.

### Examples

### Material and Methods

### Plant Material and Growth Conditions

Arabidopsis thaliana ecotype Columbia-0 (wild type, Wt) and ftshi3-1 (GabiKat-555D09-021662) (Kleinboelting et al., 2012) T-DNA insertion was confirmed by polymerase chain reaction (PCR) and sequencing-based methods (Mishra et al., 2021). Primers used for genotyping and sequencing are listed in Table 2.

Arabidopsis wild type (Wt) and mutant seeds were sterilized with 10% NaOCl, washed 4× with sterile water and then stratified for two days at 4° C. The seeds were selected on full strength MS (Murashige & Skoog) agar (Murashige & Skoog, 1962), supplemented with 1% sucrose and 75 ug/l sulfadiazine (for the transgenic seeds specifically). Stress treatments with mannitol or ABA were performed on agar plates seven days post-germination. For drought stress experiment, the seedlings were germinated for 12 days on plates and then were transferred to soil. Seedlings were moved with sterile forceps and allowed to grow in the presence of 200 mM mannitol, 1 or 5 μmol ABA for seven days and collected for dry weight. Other stress experiments were performed as described by (Mishra et al., 2019). Water deficit stress was applied on plants grown on soil in a growth chamber under short-day conditions (8 h/16 h photoperiod, 22° C./18° C.), the relative humidity of 50% and 150 μmol photons m² s⁻¹ for approximately four weeks before treatment. Water stress was imposed by simply withdrawing irrigation until drought effects were observed in the wild type the respective genotypes. The experiments were performed three times.

### Water Stress Analysis

Wild-type and transgenic plants were grown on soil in the growth chamber under short-day conditions (8 h/16 h photoperiod, 22° C./18° C., relative humidity 50% and 120 μmol photons m² s⁻¹) with regular watering for 3-4 weeks before treatment. Long-term water stress analysis was performed by withdrawing watering until the drought effects were observed in the respective genotypes. The experiment was repeated three times to verify the results (Chen et al., 2013).

### Measurements of Dehydration

The percentage of dehydration was determined by cutting and weighing well-watered plants as described by (Seo et al., 2000; Koiwai et al., 2004). Ten WT control plants, the (pAtFtsHi3::AtFtsHi3::HA(WT)) OE 1&2 lines at the age of four weeks were detached, and their fresh weight (FW) was measured. Left on the bench at RT, their weight was re-recorded after designated time intervals. The formula calculated the leaves' relative water content ((FW-Weight at any time point)/FW)*100.

### Phenotypic Characterization

Seedlings of wild type and mutants were investigated using a Leica MZ9.5 Stereomicroscope at day ten days or scanned by Epson Perfection 3200 PHOTOscanner (Japan). Plants grown on soil or exposed to stress condition were photographed at six weeks using a Canon 650D camera.

### Generation of Transgenic Arabidopsis Seedlings

For overexpression studies, genomic DNA fragments were amplified from A. thaliana by Phusion® (Thermo Scientific USA) proofreading polymerase. For native promoter over-expression, a construct containing the amplified FTSHI3 promoter sequence (Predicted on (Knudsen, 1999)) was generated using the primers ‘ftshi3 Promoter Forward’ and ‘ftshi3 Reverse for HA-line’ (Table 2). The pAtftshi3::ftshi3 genomic DNA was cloned into a pENTR/D-TOPO vector and transferred into the destination vector pGWB513 resulting in a 3×HA tagged gene product. The sequence of AtFtsHi3 gene from the genomic DNA fragments was amplified and cloned into pGWB5 vector under the control of CaMV35S promoter. Primers used are listed in (Table 2). The binary plasmids (pAtFtsHi3::AtFtsHi3::HA (construct with SEQ ID NO: 7) and 35S::AtFtsHi3::GFP (construct with SEQ ID NO: 8)) were transformed into electro-competent Agrobacterium tumefaciens (GV3101::pMP90 (pTiC58DT-DNA); Hellens et al., 2000). Wt (Col-0) plants were transformed with these constructs by the floral dip method described by Clough and Bent (1998). The resulting transgenic lines were designated pAtFtsHi3::AtFtsHi3::HA(WT) OE and 35S::AtFtsHi3::GFP(WT) OE, respectively. To investigated complementation, 35S::AtFtsHi3::GFP was transformed in the non-segregating T2 homozygous ftshi3-1 T-DNA line of the GABI-KAT collection, resulting in the transgenic lines that were designated 35S::AtFtsHi3::GFP(ftshi3-1) comp. Presence of the construct in the T1 and T2 generation was confirmed by germinating transgenic seeds on 35 mg/ml hygromycin-B selecting MS agar plates. The experiments were performed on T2 generation seeds.

### Extraction and Quantification of ABA

Sample preparation and extraction for solid Phase Extraction (SPE) and UHPLC-MS/MS were performed as described (Haas et al., 2021)

RNA Extraction, cDNA Synthesis and Quantitative PCR (qPCR)

RNA extraction and cDNA synthesis were performed using Invitrogen™ RNAqueous Total RNA Isolation Kit. Isolated RNA was reverse transcribed into Complementary DNA (cDNA) using Thermo Scientific RevertAid First Strand cDNA Synthesis Kit. Quantitative RT-PCR was performed using a Bio-Rad CFX96 thermocycler. The housing keeping genes (ubiquitin, tubulin and actin (Czechowski et al., 2005)) and gene-specific qPCR primers are listed in Table 2. The data were analysed using the Bio-Rad CFX Manager 3.1 software. RNA was isolated from different tissues from 4 weeks old plants like young flowers, leaves, buds, siliques, and stems and from roots of 2 weeks old plants germinated on MS agar plates.

TABLE 2

| | | | SEQ |
|---|---|---|---|
| | | | ID |
| Primer name | Primer sequence | Purpose | NO: |
| AtFtsHi3 | 5′-CACCACTAACCTGAAGAGACTC- | Cloning | 159 |
| promoter_Topo_F | 3′ | | |
| qi-3-RT-PCR FP | 5′-CCAGACGTTAAACCAGTTGC-3′ | qPCR | 160 |
| qi-3-RT-PCR RP | 5′-CTTCTGGTCGGTTAGTTGC-3′ | qPCR | 161 |
| ACT2-RT-PCR FP | 5′-CTTGCACCAAGCAGCATGAA-3′ | qPCR | 162 |
| ACT2 -RT-PCR FP | 5′ | qPCR | 163 |
| | CCGATCCAGACACTGTACTTCCTT- | | |
| | 3′ | | |
| UBQ5-RT-PCR FP | 5′-ACGCTTCATCTCGTC-3′ | qPCR | 164 |
| UBQ5-RT-PCR FP | 5′-CCACAGGTTGCGTTA-3′ | qPCR | 165 |
| Tubulin-RT-PCR FP | 5′-GGTATCCAACCCGATGGCA-3′ | qPCR | 166 |
| Tubulin-RT-PCR FP | 5′- | qPCR | 167 |
| | TGAGCTTGTCTCGCTAAAGAATG-3′ | | |
| DI21-RT-PCR FP | 5′- | qPCR | 168 |
| | TCC CTT ACT CAA TCC TGC TGC- | | |
| | 3′ | | |
| DI21-RT-PCR RP | 5′-ACT CTC CGG TGC CGT TAA | qPCR | 169 |
| | ATC-3′ | | |
| DREB1A-RT-PCR FP | 5′- | qPCR | 170 |
| | GCG CTA AGG ACA TCC AAA AGG- | | |
| | 3 | | |
| DREB1A-RT-PCR RP | 5′- | qPCR | 171 |
| | GTA AAT AGC CTC CAC CAA CGT | | |
| | C-3′ | | |
| NECD3-RT-PCR FP | 5′- | qPCR | 172 |
| | TTC ATC TGC GCT TCA CAC TCC- | | |
| | 3′ | | |
| NECD3-RT-PCR RP | 5′- | qPCR | 173 |
| | GCC GCT CTC TGG AAC AAA TTC- | | |
| | 3′ | | |
| COR47-RT-PCR FP | 5′- | qPCR | 174 |
| | GTT GGT TGT AAC GGA GCA TCC- | | |
| | 3′ | | |
| COR47-RT-PCR RP | 5′-CCAAAATCCCCTTCTTCTCCT C | qPCR | 175 |
| P5CS RT-PCR FP | 5′-AGCAGCCTGTAATGCGATGG-3′ | qPCR | 176 |
| P5CS RT-PCR RP | 5′-AAGTGACGCCTTTGGTTTGC-3′ | qPCR | 177 |
| RD22 RT-PCR FP | 5′-AGGGCTGTTTCCACTGAGG-3′ | qPCR | 178 |
| RD22 RT-PCR RP | 5′- | qPCR | 179 |
| | CACCACAGATTTATCGTCAGACA-3′ | | |
| RD29A RT-PCR FP | 5′- | qPCR | 180 |
| | GTTACTGATCCCACCAAAGAAGA-3′ | | |
| RD29A RT-PCR RP | 5′-GGAGACTCATCAGTCACTTCCA- | qPCR | 181 |
| | 3′ | | |
| DREB2A RT-PCR FP | 5′-GCAGTTTATGATCAGAG-3′ | qPCR | 182 |
| DREB2A RT-PCR FP | 5′-AACTTCTTCTACGGTCTCGT-3′ | qPCR | 183 |
| RD29B RT-PCR FP | 5′-GGAGTGAAGGAGACGCAACA-3′ | qPCR | 184 |
| RD29B RT-PCR RP | 5′-CCACCTCCTTTGTAGCCGTT-3′ | qPCR | 185 |
| ftshi3 Reverse for | 5′- | Cloning | 186 |
| HA-line | GCTGAGAGTTTGATAACCTAAC | | |
| | G-3′ | | |
| AtFtsHi3Topo_F | 5′- | Cloning | 187 |
| | CACCATGGCTACTTTCAATGTT- | | |
| | 3′ | | |

### Measurements of Stomatal Density and Aperture in Response to ABA Treatment

Four-week-old rosette leaves were excised and peeled by the scotch tape method described in (Lawrence II et al., 2018). Microscopic images of stomata were taken on a Leica DMi8 at 40× magnification. For stomata density, five microscope fields per leaf were evaluated with five replicates per genotype. Stomatal aperture was measured using ImageJ software. Significance (p-value<0.05, student's t-test) of the 45 stomatal aperture measurements was calculated using SPSS software.

### Leaf-Level Gas Exchange

A portable photosynthesis system (Li-6400xt, Li-Cor, Lincoln, NE, USA) was used to determine the photosynthesis rate (assimilation AN) and stomatal conductance (gₛ) as described by (Tomeo & Rosenthal, 2018). The photosynthesis rate (assimilation AN) was measured at a CO₂ concentration (Cr) of 400 μmol mol⁻¹ air and photosynthetically active radiation (PAR) of 1000 μmol photons m⁻² s⁻¹ on the 7th or 8th leaf of the rosette across five biological replicates per treatment per genotype. The leaf chamber temperature was standardized to 25° C., and the airflow to 250 μmol s⁻¹. Gas exchange parameters were determined during the daytime between 11 am to 4 μm. The ratio of AN/gₛ calculates the water use efficiency (WUE) intrinsic.

### Chloroplast Size and Ultrastructure

TEM was used to study the chloroplast morphology of the first true leaf of 12-day-old seedlings of Wt. Sample preparation and microscopy were performed at the Ume\dot{a} Centre for Electron Microscopy (UCEM), Ume\dot{a}, Sweden. The open-source image-processing program ImageJ (Java-based image processing program developed at the NIH) was used for measuring chloroplast length and width.

### Phylogenetic Analysis

Arabidopsis FtsH protein sequences were recovered from UNIPROT (www.uniprot.org). They were used to find ortho homologues in Populous. They were investigated by BLASTP (Altschul et al., 1990) in on Phytozome (phytozome.jgi.doe.gov, v12.1.6) using the parameters described in (Mishra et al., 2019). The Arabidopsis and Populous FtsH protein were selected manually from BLASTP. The protein sequences were aligned using Clustal Omega with default settings to calculate sequence identities and phylogenetic trees were constructed using the neighbour-joining (NJ) method using Jalview Version 2 (Waterhouse et al., 2009). For further analysis and to evaluate evolutionary conservation, only the Arabidopsis FtsHi3 protein was blasted against all 64 species from Phytozome genome portal (phytozome.jgi.doe.gov, v12.1.6) and on popgenie genome portal (https://popgenie.org/). Proteins with highest bit scores (identity percentage of approximately between (60%-75%) to AtFtsHi3 were selected manually from BLASTP, at least one species per family was chosen. The phylogenetic tree was constructed using the neighbour-joining (NJ) method using Jalview Version 2 (Waterhouse et al., 2009).

Transformation of Hybrid Aspen with Arabidopsis FtsHi3 Constructs

In one embodiment, the binary plasmids pAtFtsHi3::AtFtsHi3::HA and 35S::AtFtsHi3::GFP, previously used to over-express the Arabidopsis FtsHi3 gene in Arabidopsis plants, can be transformed into Agrobacterium tumefaciens and can subsequently be used for Agrobacterium-mediated transformation of model woody species hybrid aspen (Populus tremula x Populus tremuloides). The pAtFtsHi3::AtFtsHi3::HA and 35S::AtFtsHi3::GFP binary plasmid constructs, can thereby be tested in hybrid aspen clones (e.g. the commonly used lab clone T89), to provide independent over-expression experiments.

In one embodiment, the transformation and regeneration of transgenic hybrid aspen plants is performed as described in the experimental part of WO2016108750 and in Nilsson et al., 1992 (Transgenic Research 1, 209-220). Typically, 8-10 independent transgenic lines are generated for each construct and clone.

In one embodiment, RNA extraction, cDNA synthesis and quantitative PCR (qPCR) are performed to evaluate the FtsHi3 transcriptional expression levels in the transgenic plant lines as compared to wild type plants of the same species. In hybrid aspen, the expression level of, for example, the 26S proteasome regulatory subunit S2 can be used as a reference gene to which gene expression levels are normalized.

### Construction of Populus FtSHi3 Constructs

Identification of FtsHi3 orthologs can be performed by comparing the Arabidopsis FtsHi3 gene to homologous genes from other plant species, identified via BLAST search with the amino acid sequence encoded by the Arabidopsis FtSHi3 gene (SEQ ID NO: 2, accession number AT3G02450.1) in publicly available genome/protein database resources, such as the popgenie.org and Phytozome databases, followed by a phylogenetic analysis of the identified homologous genes. For example, such a phylogenetic analysis identifies one FtsHi3 ortholog in Populus tremula (PotraFtsHi3, SEQ ID NO: 3 and 4, popgenie.org accession number Potra001056g09045.1).

In one embodiment, a DNA fragment corresponding to the native coding sequence of the Populus tremula FtsHi3 gene (SEQ ID NO: 3), is manufactured by DNA synthesis and flanked by Gateway recombination sites for sub-cloning into destination vectors designed for overexpression of the inserted coding sequence in a plant or a plant cell.

In one embodiment, the PotraFtsHi3 CDS is operably linked to a native FtsHi3 promoter sequence from Populus tremula, such as the DNA fragment presented as SEQ ID NO: 6, corresponding to the genomic sequence 2001 base-pairs upstream of the start codon of the Populus tremula FtsHi3 gene. This promoter sequence can, among other ways, be amplified from genomic DNA via PCR, or manufactured by DNA synthesis and flanked by Gateway recombination sites for sub-cloning purposes.

In one embodiment, the DNA fragments of the native Populus tremula FtsHi3 promoter and coding region become operably linked via sub-cloning both fragments by multisite R4-R2 Gateway recombination into the pK7m24GW, 3 destination plant binary vector. The resulting binary plasmid can be subsequently used for plant transformation by Agrobacterium-mediated transformation.

The sequence for an example of a FtsHi3 native promoter construct is provided in SEQ ID NO: 9 (pPotraFtsHi3::PotraFtsHi3, bases 1-2001 promoter, 2002-2022 recombination site and 2023-3966 FtsHi3 CDS).

In one embodiment, the PotraFtsHi3 CDS is operably linked to the constitutive 35S promoter to drive FtsHi3 over-expression. For this purpose, one approach consists in sub-cloning the PotraFtsHi3 CDS in a Gateway Entry vector harboring the Cauliflower Mosaic Virus 35S promoter upstream of Gateway recombination sites.

The sequence for such hypothetical 35S promoter-driven PotraFtsHi3 CDS construct is provided in SEQ ID NO: 10 (35S: PotraFtsHi3, bases 1-1043 promoter, 1044-1064 recombination site and 1065-3008 FtsHi3 CDS). The resulting binary plasmid can be subsequently used for plant transformation by Agrobacterium-mediated transformation.

Transformation of Arabidopsis with Populus FtsHi3 Constructs

In one embodiment, Arabidopsis thaliana (Col-0) wild type plants are transformed with the pPotraFtsHi3::PotraFtsHi3 and 35S: PotraFtsHi3 constructs to overexpress the Populus tremula FtsHi3 gene in Arabidopsis.

In one embodiment, the Arabidopsis plants are transformed by the floral dip method described by Clough and Bent (1998). Presence of the construct in the subsequent generations is confirmed by germinating transgenic seeds on MS-medium agar plates containing the correct antibiotics for selection of transformants (which is determined by which binary destination vector is used for transformation).

In one embodiment, RNA extraction, cDNA synthesis and quantitative PCR (qPCR) are performed to evaluate the FtsHi3 transcriptional expression levels in the transgenic plant lines as compared to wild type plants of the same species.

In one embodiment, drought tolerance experiments are performed on transgenic and wild type plants, following the same method as described in the examples above and below.

Transformation of Hybrid Aspen with Populus FtsHi3 Constructs

In one embodiment, hybrid aspen (Populus tremula x tremuloides) wild type plants (e.g. of clone T89) are transformed with the pPotraFtsHi3::PotraFtsHi3 and 35S::PotraFtsHi3 constructs in order to overexpress the Populus tremula FtsHi3 CDS.

In one embodiment, the transformation and regeneration of transgenic plants are performed as described in the experimental part of WO2016108750 and in Nilsson et al., 1992 (Transgenic Research 1, 209-220). Typically, 8-10 independent transgenic lines are generated for each construct and clone.

In one embodiment, RNA extraction, cDNA synthesis and quantitative PCR (qPCR) are performed as previously described, to evaluate the FtsHi3 transcriptional expression levels in the transgenic plant lines as compared to wild type levels.

### Hybrid Aspen Greenhouse Experiments

In one embodiment, for each promoter and gene/CDS combination where the Arabidopsis or Populus tremula FtsHi3 gene/CDS is over-expressed, under the control of a FtsHi3 native promoter from the same species, or the 35S promoter, 5-10 of the produced transgenic hybrid aspen lines with 3-5 clonal replicates each are grown together with wild type reference trees in the greenhouse for 8-9 weeks, during which time the trees are monitored for growth and other traits.

In one embodiment, the transgenic and wild type hybrid aspens are grown under both normal watered conditions and under water deficit, whereby drought tolerance is measured and evaluated mutatis mutandis in the same way as in the previously described Arabidopsis drought tolerance experiments.

In one embodiment, after growth and drought tolerance experiments are concluded, the plants are harvested, measured and sampled for growth and drought response traits, such as plant height, width, stem volume, wood density, dry weight of stem and bark and drought response hallmarks.

### ¹³C Discrimination and Water Use Efficiency (WUE)

Transgenic hybrid aspen trees are also analysed for 13C discrimination and water use efficiency according to the method presented in Farquhar et al., 1989 (Ann. Rev. Plant Physiol. 40:503-537) which provides further data on drought stress related characteristics.

To further our understanding of how beneficial overexpression of FtsHi3 can be for plant growth and drought tolerance, we generated a new batch of Arabidopsis thaliana plants overexpressing AtFtsHi3. Isolation and characterization of additional Arabidopsis plants with altered AtFtsHi3 expression verify the already observed correlation between AtFtsHi3 overexpression and drought tolerance. The AtFtsHi3 gene expression was measured in Arabidopsis (Columbia-0) WT plants as well as pAtFtsHi3::AtFtsHi3::HA(WT), and 35S::AtFtsHi3::GFP(WT) transgenic lines, for which multiple independent T3 lines were obtained, described in more detail below.

### Naming and Configuration of the Transgenic Lines

The transgenic lines of this invention have been transformed with the DNA constructs specified in Table 3. This table also holds information of the promoter and gene in the DNA construct, as well as the genotype of the plant used for transformation.

TABLE 3
List of new transgenic lines generated for confirmation of previous results

| Transgenic line name | DNA contruct | Promoter | Gene | Genotype |
|---|---|---|---|---|
| pAtFtsHi3::AtFtsHi3::HA(WT)3 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)4 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)5 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)6 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)7 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)8 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)9 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)10 | pAtFtsHi3::AtFtsHi3::HA | pAtFtsHi3 | AtFtsHi3 | Arabidopsis thaliana Col-0 (WT) |
| pAtFtsHi3::AtFtsHi3::HA(WT)11 | pAtFtsHi3::AtFtsHi3::HA | pAtFtsHi3 | AtFtsHi3 | Arabidopsis thaliana Col-0 (WT) |
| 35S::AtFtsHi3::GFP(WT)1 | 35S::AtFtsHi3::GFP | p35S | AtFtsHi3 | Arabidopsis thaliana Col-0 (WT) |
| 35S::AtFtsHi3::GFP(WT)2 | 35S::AtFtsHi3::GFP | p35S | AtFtsHi3 | Arabidopsis thaliana Col-0 (WT) |
| 35S::AtFtsHi3::GFP(WT)3 | 35S::AtFtsHi3::GFP | p35S | AtFtsHi3 | Arabidopsis thaliana Col-0 (WT) |
| 35S::AtFtsHi3::GFP(WT)4 | 35S::AtFtsHi3::GFP | p35S | AtFtsHi3 | Arabidopsis thaliana Col-0 (WT) |
| 35S::AtFtsHi3::GFP(WT)5 | 35S::AtFtsHi3::GFP | p35S | AtFtsHi3 | Arabidopsis thaliana Col-0 (WT) |
| 35S::AtFtsHi3::GFP(WT)6 | 35S::AtFtsHi3::GFP | p35S | AtFtsHi3 | Arabidopsis thaliana Col-0 (WT) |
| 35S::AtFtsHi3::GFP(WT)7 | 35S::AtFtsHi3::GFP | p35S | AtFtsHi3 | Arabidopsis thaliana Col-0 (WT) |

The transgenic lines of this invention have been named or abbreviated slightly different in the different described experiments, but their names can be used interchangeably, as clarified in the name translation table (Table 4).

TABLE 4
Naming of the newly generated transgenic lines

| | Alternative | Alternative |
|---|---|---|
| Transgenic line name | name 1 | name 2 |
| pAtFtsHi3::AtFtsHi3::HA(WT) line 3 | pFtsHi3-OE3 | pFtsHi3-OE3-H |
| pAtFtsHi3::AtFtsHi3::HA(WT)4 | pFtsHi3-OE4 | pFtsHi3-OE4-H |
| pAtFtsHi3::AtFtsHi3::HA(WT)5 | pFtsHi3-OE5 | pFtsHi3-OE5-H |
| pAtFtsHi3::AtFtsHi3::HA(WT)6 | pFtsHi3-OE6 | pFtsHi3-OE6-M |
| pAtFtsHi3::AtFtsHi3::HA(WT)7 | pFtsHi3-OE7 | pFtsHi3-OE7-M |
| pAtFtsHi3::AtFtsHi3::HA(WT)8 | pFtsHi3-OE8 | pFtsHi3-OE8-M |
| pAtFtsHi3::AtFtsHi3::HA(WT)9 | pFtsHi3-OE9 | pFtsHi3-OE9-L |
| pAtFtsHi3::AtFtsHi3::HA(WT)10 | pFtsHi3-OE10 | pFtsHi3-OE10-L |
| pAtFtsHi3::AtFtsHi3::HA(WT)11 | pFtsHi3-OE11 | pFtsHi3-OE11-L |
| 35S::AtFtsHi3::GFP(WT)1 | p35S-OE1 | |
| 35S::AtFtsHi3::GFP(WT)2 | p35S-OE2 | |
| 35S::AtFtsHi3::GFP(WT)3 | p35S-OE3 | |
| 35S::AtFtsHi3::GFP(WT)4 | p35S-OE4 | |
| 35S::AtFtsHi3::GFP(WT)5 | p35S-OE5 | |
| 35S::AtFtsHi3::GFP(WT)6 | p35S-OE6 | |
| 35S::AtFtsHi3::GFP(WT)7 | p35S-OE7 | |

### Methods

### Phenotypic Characterization

WT plants, together with transgenic plants (Tables 3,4), were grown (one rosette per pot) in short day conditions and exposed to drought stress conditions. Photographs of the plants under watered or drought conditions were taken using a Canon 650D camera.

RNA Extraction, cDNA Synthesis and Quantitative PCR (qPCR)

RNA extraction and cDNA synthesis were performed as described by (Mishra et al. 2021). The house-keeping genes used as reference genes (ubiquitin and actin) and gene-specific qPCR primers are listed in Table 2. The data was analyzed using the Bio-Rad CFX Manager 3.1 software. For each line (and WT), RNA was isolated from different tissues (flowers, leaves, buds, siliques, and stems) from plants grown 4 weeks in soil under long day conditions, or (for roots) 2-week-old seedlings grown on MS agar plates.

### Drought Tolerance Assay

The drought tolerance methods ‘one rosette per pot’ and ‘weighing’ were used as described by (Harb and Pereira, 2011; de Ollas et al., 2019). The growing, drying, rewatering, and photographing processes of the ‘one rosette per pot’ system were adapted from (Harb and Pereira, 2011; de Ollas et al., 2019) to our growth chambers and conditions. The two-week-old plate-grown WT and transgenic seedlings were transplanted into 5 cm square pots containing commercial soil (Hasselfors garden special, Sweden; and Vermiculite Sibelco, Europe in a 3:1 soil: vermiculite mixture) in biological replicates (plants) for pAtFtsHi3::AtFtsHi3::HA(WT) overexpressors 1 and 2 as well as WT, and 15 biological replicates (plants) each of the newly isolated pAtFtsHi3::AtFtsHi3::HA(WT), and 35S::AtFtsHi3::GFP(WT) lines. The plants were grown in short-day conditions (8 h photoperiod, 16h dark period, 22° C. and 18° C., respectively) throughout the experiment. The initial plant daily transpiration was calculated after saturating the pots with 500 ml water (15 pots per tray) and weighing up to 5 to 6 days to determine the soil water volumetric content. The rosette diameter of all plants was measured at intervals of 3-4 days throughout the experiment. When the plants were approximately 4-5 weeks old, water irrigation was stopped until distinguishable drought response symptoms were observed. The pot weights were measured and recorded each day following a previous method (Harb and Pereira, 2011). During the drought stress treatment, the pots were weighed between 9.00 and 11.00 a.m. every day, or every other day, to calculate the progressive water deficit during drought. After 15 days of stress, rewatering was performed for three days, and after rewatering for the ‘one rosette per pot’ system, the rosettes were harvested, and biomass (Fresh weight (FW)) was recorded and they were then packed in glassine bags. The bags were oven-dried at 65° C. for 72 h, and the biomass (dry weight (DW)) was recorded, and leaf water content was calculated according to the equation: ((FW−DW)/FW). The pots were also oven-dried at 65° C. for two weeks and weighed to determine the soil water content by calculating the soil weight difference (Fresh soil weight-dried soil weight) or calculating Gravimetric soil water content (%)=([mass of moist soil (g)]−[mass of oven-dried soil (g)])/[mass of oven-dried soil (g)] 100.

### Results

Characterization of Arabidopsis Plants with Altered AtFtsHi3 Expression

Multiple new independent T3 lines for the pAtFtsHi3::AtFtsHi3::HA(WT) and 35S::AtFtsHi3: GFP(WT) constructs were generated, and designated pFtsHi3-OE and p35S-OE, respectively (Table 4).

AtFtsHi3 transcript levels were determined in the pFtsHi3-OE and p35Si3-OE seedlings and compared to those of the WT control plants. The pFtsHi3-OE lines 3, 4 and 5 showed an approximately 7-fold increase in AtFtsHi3 expression, similar to the previously investigated and described pAtFtsHi3::AtFtsHi3::HA(WT) OE1 and OE2 lines (hereafter referred to as pFtsHi3-OE1 and pFtsHi3-OE2, respectively), whereas OE lines 6, 7, and 8 showed an approximately 4-fold increase in AtFtsHi3 expression compared to WT plants. In addition, lines 9 and 10 showed an approximately 2-fold increase and line 11 showed an AtFtsHi3 expression barely higher than in WT plants (FIG. 12A). Therefore, pFtsHi3-OE lines 3, 4 and 5 are considered as high-overexpression (“H”) lines 6, 7, and 8 as medium-overexpression (“M”) and lines 9, and 11 as low-overexpression (“L”). The 35S::AtFtsHi3::GFP(WT) (designated p35S-OE) lines 1, 2, and 3 showed an increase in AtFtsHi3 expression of 2-fold as compared to WT, while the other p35S-OE lines (4-7) displayed expression levels that do not qualify as overexpression (FIG. 12B).

Morphological and physiological traits were determined on plants grown on soil in short day conditions. Photographs were taken at 23 days (FIG. 13A), and later at 49 days after being exposed to 14 days of drought stress treatment (FIG. 13B). The rosette diameter was measured on 15-35 independent biological replicates (plants) of pFtsHi3-OE, p35S-OE and WT plants from weeks 2 to week 7. Transgenic lines displayed a significantly larger rosette diameter, with an increase up to 20-30% compared to WT (FIGS. 14A and 14B), establishing a substantial positive effect of AtFtshi3 overexpression on the rosette diameter, after 14 days of drought treatment.

AtFtsHi3 Overexpression Confers Drought Tolerance Combined with Increased Plant Growth

To further investigate the effect of AtFtsHi3 overexpression on plant growth and drought tolerance, the newly characterized pFtsHi3-OE and p35S-OE lines were exposed to 14 days of drought stress along with pFtsHi3-OE1, pFtsHi3-OE2 (already shown above to be more drought resistant than WT) and WT. Under well-watered control conditions, pFtsHi3-OE lines appeared to more leaves than WT (not quantified but visually obvious, as in FIG. 13B), flowered earlier than WT, and had larger rosettes than. Under drought treatment, after water was withheld for 14 days, 90% of the WT plants showed severe drought symptoms, while the pFtsHi3-OE and p35S-OE transgenic lines clearly exhibited less wilting and necrosis than WT (FIG. 13B). Furthermore, the transgenic plants' leaves appeared more numerous and remained noticeably more vigorous compared to WT (FIG. 13B). In addition, no significant difference in pot weight (g) was determined between the genotypes exposed to the same treatment and the water deficit evolved similarly over time. We noted that the fresh rosette biomass of pFtsHi3-OE (lines 1, 7, 8, 9, and 10) and p35S-OE (lines 1, 5, 6, and 7) was up to 20-40% higher than that of WT plants after being exposed to drought stress for 14 days and rewatering for three days (FIGS. 15A and 15B). The other pFtsHi3-OE and p35S-OE lines, while not growing significantly better than WT plants, grew equally well as WT, all the while not showing as severe drought symptoms, and recovering better than WT plants after rewatering (FIG. 13B).

We also recorded the dry rosette biomass (fresh weight, FIGS. 15A and 15B, and dry weight, FIGS. 15C and 15D) of all the independent pFtsHi3-OE and p35S-OE lines and WT plants and noted that the native promoter-driven pFtsHi3-OE lines had significantly higher dry biomass, up to 20-30%, than the WT plants (FIGS. 15C and 15D). The p35S-OE lines showed tendencies towards an increase in dry biomass but this observation did not reach statistical significance threshold in this experiment (p=0.058) according to our threshold (p=0.05).

Water moves relatively slowly within soil micropores in any direction from a region of high water potential to an area of low water potential. As an example, water uptake by plant roots lowers the nearby soil water potential. If the water potential of the surrounding soil is higher, perhaps deeper in the ground or between plants, water moves toward the roots upwards and sideways. However, eventually, the plants stay wilted because the soil holds onto the water too tightly for plants to take it up. The soil has reached a permanent wilting point corresponding to a certain threshold of the soil water potential (Voroney, 2019).

The soil appears dry at this point, but it still contains water, though plants cannot take it up from the soil. Soil micropores (micro-cavities that are normally filled with water) are compacted due to water loss (transpiration and evaporation), and when rewatered, the water uptake by these soil micropores is significantly reduced compared to the soil pores that are not compressed thanks to the residual moisture.

Following drought treatment and subsequent rewatering, we measured the leaf water content of the plants (FIGS. 16A and 16C) and the relative soil water content as compared to its water content prior to drought (FIGS. 16B and 16D). The pFtsHi3-OE lines had significantly higher leaf water content, up to 20-40% than the WT plants, after being exposed to drought stress for 14 days and rewatering for three days, indicating positive water holding capacity under drought stress (FIG. 16A). On the other hand, the p35S-OE lines showed similar leaf water content to WT plants (FIG. 16C), but showed a higher relative soil water content (FIG. 16D), indicating a more regulated water uptake from the soil by the roots, despite a similar water deficit. The soil water content in the pots of pFtsHi3-OE lines was also higher than the WT (FIG. 16B).

### REFERENCES

Belhaj et al., Plant Methods, 2013 Oct. 11; 9(1):39
Chen et al., Journal of Genetics and Genomics, Volume 40, Issue 6, 20 Jun. 2013,
Pages 271-279
Clough, S. J., and Bent, A. F. (1998). Floral dip: a simplified method for Agrobacterium-mediated transformation of Arabidopsis thaliana. Plant J 16, 735-743.
De Ollas, C., Segarra-Medina, C., González-Guzmán, M., Puertolas, J., and Gómez-Cadenas, A. (2019). A customizable method to characterize Arabidopsis thaliana transpiration under drought conditions. Plant methods 15, 89-89.
Fanourakis, D., Nikoloudakis, N., Pappi, P., Markakis, E., Doupis, G., Charova, S. N., Delis, C., and Tsaniklidis, G. (2020). The Role of Proteases in Determining Stomatal Development and Tuning Pore Aperture: A Review. Plants (Basel) 9, 340.
Farquhar, G. D., J. R. Ehleringer, and K. T. Hubick. 1989. Carbon isotope discrimination and photosynthesis. Ann. Rev. Plant Physiol. 40:503-537
Haas, J. C., Vergara, A., Serrano, A. R., Mishra, S., Hurry, V., and Street, N. R. (2021). Candidate regulators and target genes of drought stress in needles and roots of Norway spruce. Tree Physiology.
Harb, A., and Pereira, A. (2011). “Screening Arabidopsis Genotypes for Drought Stress Resistance,” in Plant Reverse Genetics: Methods and Protocols, ed. A. Pereira. (Totowa, NJ: Humana Press), 191-198.
Hellens, R. P., Mullineaux, P., & Klee, H. (2000). A guide to Agrobacterium binary Ti vectors. Trends in Plant Science, 5(10), pp. 446-451.
Kleinboelting, N., Huep, G., Kloetgen, A., Viehoever, P., and Weisshaar, B. (2012). GABI-Kat SimpleSearch: new features of the Arabidopsis thaliana T-DNA mutant database. Nucleic acids research 40, D1211-D1215.
Knudsen, S. (1999). Promoter2.0: for the recognition of Polll promoter sequences. Bioinformatics, 15(5):356-61
Larkin et al. 2007, Clustal W and Clustal X version 2.0. Bioinformatics, 23:2947-2948.
Lawrence Ii, S., Pang, Q., Kong, W., and Chen, S. (2018). Stomata tape-peel: an improved method for guard cell sample preparation. JoVE (Journal of Visualized Experiments), e57422.
Lim, C. W., Baek, W., Jung, J., Kim, J. H., & Lee, S. C. (2015). Function of ABA in stomatal defense against biotic and drought stresses. International journal of molecular sciences, 16(7), 15251-15270.Mishra, L. S., Kim, S.-Y., Caddell, D. F., Coleman-Derr, D., and Funk, C. (2020). Loss of Arabidopsis matrix metalloproteinase-5 affects root development and root bacterial communities during drought stress. Physiologia Plantarum n/a, 1-14.
Mishra, L. S., Mielke, K., Wagner, R., and Funk, C. (2019). Reduced expression of the proteolytically inactive FtsH members has impacts on the Darwinian fitness of Arabidopsis thaliana. Journal of experimental botany 70, 2173-2184.
Mishra, L. S., Mishra, S., Caddell, D. F., Coleman-Derr, D., and Funk, C. (2021). The Plastid-Localized AtFtsHi3 Pseudo-Protease of Arabidopsis thaliana Has an Impact on Plant Growth and Drought Tolerance. Frontiers in Plant Science 12.
Nilsson et al., 1992 (Transgenic Research 1, 209-220)
Porra, R., Thompson, W., and Kriedemann, P. (1989). Determination of accurate extinction coefficients and simultaneous equations for assaying chlorophylls a and b extracted with four different solvents: verification of the concentration of chlorophyll standards by atomic absorption spectroscopy. Biochimica et Biophysica Acta (BBA)-Bioenergetics 975, 384-394.
Song et al., The Crop Journal, Volume 4, Issue 2, April 2016, Pages 75-82
Tomeo, N. J., and Rosenthal, D. M. (2018). Photorespiration differs among Arabidopsis thaliana ecotypes and is correlated with photosynthesis. Journal of experimental botany 69, 5191-5204.
Vaseva, I., Sabotič, J., Sustar-Vozlic, J., Meglic, V., Kidrič, M., Demirevska, K., and Simova, L. (2011). “The response of plants to drought stress: The role of dehydrins, chaperones, proteases and protease inhibitors in maintaining cellular protein function.”), 1-46.
Voroney, P. (2019) “Soils for Horse Pasture Management”, in “Horse Pasture Management” (Elsevier, Inc., DOI: 10.1016/B978-0-12-812919-7.00004-4)
WO2014195287
WO2016108750
U.S. Pat. No. 7,881,502
U.S. Pat. No. 9,053,353

1. A method for obtaining a genetically modified plant having improved yield and/or drought tolerance, as compared to a wild type control plant of the same species, comprising:
(a) modifying the genomic DNA in at least one cell of said plant species to increase expression of a FTSHi3 gene thereby obtaining a genetically modified cell;
(b) generating a plant from the genetically modified cell to obtain a genetically modified plant;
(c) growing said genetically modified plant under conditions which permit development of a plant; and
(d) selecting a genetically modified plant having improved drought tolerance.

2. The method of claim 1, wherein the modification of the genomic DNA in step a. comprises introducing a nucleic acid molecule encoding a FTSHi3 gene product operably linked to a FTSHi3 promoter or a constitutive promoter.

3. The method of claim 2, wherein
(i) the encoded FTSHi3 gene product has the same amino acid sequence as a native FTSHi3 gene product of the same plant species, at least 60% identity to the amino acid sequence according to SEQ ID NO: 2, or at least 60% identity to the amino acid sequence according to SEQ ID NO: 4;
(ii) the FTSHi3 promoter has a nucleotide sequence corresponding to the nucleotide sequence of the 1-2*10³ nucleotides located upstream, and proximal to the start codon, of the native FTSHi3 gene in a native genome of a control plant of the same species; and/or
(iii) the FTSHi3 promoter has a nucleotide sequence according to SEQ ID NO: 5.

4.-5. (canceled)

6. The method of claim 1, wherein the modification of the genomic DNA in step a. comprises modification of the nucleotide sequence of the 1*10³-2*10³ nucleotides located upstream, and proximal to the start codon, of the native FTSHi3 gene.

7. The method of claim 6, wherein the modification of the nucleotide sequence of the 1-2*10³ nucleotides located immediately upstream the native FTSHi3 gene is performed by means of CRISPR or TALENS.

8. A method for obtaining a genetically modified plant having improved drought tolerance, as compared to a control plant of the same species, comprising
(a) obtaining a first genetically modified plant through the method according to claim 1;
(b) producing at least one seed, somatic embryo, or vegetatively reproducible material from the first genetically modified plant; and
(c) obtaining at least one second genetically modified plant from said seed, somatic embryo, or vegetatively reproducible material.

9. The method of claim 1, wherein plant species is a crop plant species or a woody plant species, such as a hardwood plant species or a gymnosperm species.

10. The method of claim 9, wherein
(i) the crop plant species is selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca 30 arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp.;
(ii) the woody plant species is a hardwood plant species selected from acacia, eucalyptus, hornbeam, beech, mahogany, walnut, oak, ash, willow, hickory, birch, chestnut, poplar, alder, maple, sycamore, ginkgo, a palm tree and sweet gum; and/or
(iii) the woody plant species is a conifer tree.

11-12. (canceled)

13. A genetically modified plant obtainable by the method of claim 1.

14. A genetically modified plant characterized in that it exhibits at least 50% increased expression of a FTSHi3 gene as compared to the wild-type control plant of the same species.

15. The genetically modified plant of claim 14, comprising a modification of the genomic DNA, as compared to a wild-type control plant of the same species, said modification comprising the presence of a recombinant nucleic acid molecule encoding a FTSHi3 gene product operably linked to a FTSHi3 promoter, or a constitutive promoter.

16. The genetically modified plant of claim 14, wherein (i) the encoded FTSHi3 gene product has the same amino acid sequence as a native FTSHi3 gene product of the same plant species, at least 60% identity to the amino acid sequence according to SEQ ID NO: 2, or at least 60% identity to the amino acid sequence according to SEQ ID NO: 4;
(ii) the FTSHi3 promoter has a nucleotide sequence corresponding to the nucleotide sequence of the 1-2*103 nucleotides located upstream, and proximal to the start codon, of the native FTSHi3 gene in a native genome of a control plant of the same species; and/or
(iii) the FTSHi3 promoter has a nucleotide sequence according to SEQ ID NO: 5.

17-18. (canceled)

19. The genetically modified plant of claim 14, comprising a modification of the genomic DNA, as compared to a wild-type control plant of the same species, said modification comprising a modification of the nucleotide sequence of the 1-2*10³ nucleotides located upstream, and proximal to the start codon, of the native FTSHi3 gene.

20. The genetically modified plant according to of claim 14, having improved drought resistance, as compared to a wild-type control plant of the same species.

21. The genetically modified plant of claim 14, that has not been obtained exclusively by means of an essentially biological process.

22. The method of claim 8, wherein plant species is a crop plant species or a woody plant species, such as a hardwood plant species or a gymnosperm species.

23. The method of claim 22, wherein
(i) the crop plant species is selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca 30 arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp.;
(ii) the woody plant species is a hardwood plant species selected from acacia, eucalyptus, hornbeam, beech, mahogany, walnut, oak, ash, willow, hickory, birch, chestnut, poplar, alder, maple, sycamore, ginkgo, a palm tree and sweet gum; and/or
(iii) the woody plant species is a conifer tree.

24. A genetically modified plant obtainable by the method of claim 8.
